SEQUENCE LISTING (50 sequences)

SEQ ID NO: 1 - protein, 5 aa, Artificial Sequence
  Artificially Synthesized Sequence
DYAMH

SEQ ID NO: 2 - protein, 17 aa, Artificial Sequence
  Artificially Synthesized Sequence
LISIYSDHTNYNQKFQG

SEQ ID NO: 3 - protein, 12 aa, Artificial Sequence
  Artificially Synthesized Sequence
NNYGNYGWYFDV

SEQ ID NO: 4 - protein, 10 aa, Artificial Sequence
  Artificially Synthesized Sequence
SASSSVSYMH

SEQ ID NO: 5 - protein, 7 aa, Artificial Sequence
  Artificially Synthesized Sequence
DTSKLAS

SEQ ID NO: 6 - protein, 9 aa, Artificial Sequence
  Artificially Synthesized Sequence
FQGSGYPYT

SEQ ID NO: 7 - protein, 121 aa, Artificial Sequence
  Artificially Synthesized Sequence
QVQLVQSGAEVKKPGSSVKVSCKASGYTFTDYAMHWVRQAPGQGLEWMGLISIYSDHTNYNQKFQGRVTITADKSTSTAYMELSSLRSEDTAVYYCGRNNYGNYGWYFDVWGQGTTVTVSS

SEQ ID NO: 8 - protein, 106 aa, Artificial Sequence
  Artificially Synthesized Sequence
EIVLTQSPATLSLSPGERATLSCSASSSVSYMHWYQQKPGQAPRLLIYDTSKLASGIPARFSGSGSGTDYTLTISSLEPEDFAVYYCFQGSGYPYTFGQGTKLEIK

SEQ ID NO: 9 - protein, 448 aa, Artificial Sequence
  Artificially Synthesized Sequence
QVQLVQSGAEVKKPGSSVKVSCKASGYTFTDYAMHWVRQAPGQGLEWMGLISIYSDHTNYNQKFQGRVTITADKSTSTAYMELSSLRSEDTAVYYCGRNNYGNYGWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 213 aa, Artificial Sequence
  Artificially Synthesized Sequence
EIVLTQSPATLSLSPGERATLSCSASSSVSYMHWYQQKPGQAPRLLIYDTSKLASGIPARFSGSGSGTDYTLTISSLEPEDFAVYYCFQGSGYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 168 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKM

SEQ ID NO: 12 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 13 - protein, 5 aa, Artificial Sequence
  Artificially Synthesized Sequence
SYIMM

SEQ ID NO: 14 - protein, 17 aa, Artificial Sequence
  Artificially Synthesized Sequence
SIYPSGGITFYADTVKG

SEQ ID NO: 15 - protein, 11 aa, Artificial Sequence
  Artificially Synthesized Sequence
IKLGTVTTVDY

SEQ ID NO: 16 - protein, 14 aa, Artificial Sequence
  Artificially Synthesized Sequence
TGTSSDVGGYNYVS

SEQ ID NO: 17 - protein, 7 aa, Artificial Sequence
  Artificially Synthesized Sequence
DVSNRPS

SEQ ID NO: 18 - protein, 10 aa, Artificial Sequence
  Artificially Synthesized Sequence
SSYTSSSTRV

SEQ ID NO: 19 - protein, 120 aa, Artificial Sequence
  Artificially Synthesized Sequence
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 20 - protein, 110 aa, Artificial Sequence
  Artificially Synthesized Sequence
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 21 - protein, 449 aa, Artificial Sequence
  Artificially Synthesized Sequence
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 216 aa, Artificial Sequence
  Artificially Synthesized Sequence
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 23 - protein, 607 aa, Artificial Sequence
  Artificially Synthesized Sequence
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 24 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 25 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 26 - protein, 136 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 27 - protein, 118 aa, Artificial Sequence [sequence omitted]

SEQ ID NO: 28 - protein, 108 aa, Artificial Sequence
  Artificially Synthesized Sequence
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 29 - protein, 118 aa, Artificial Sequence
  Artificially Synthesized Sequence
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 30 - protein, 21 aa, Artificial Sequence
  Artificially Synthesized Sequence
GGGGSGGGGSGGGGSGGGGSG

SEQ ID NO: 31 - protein, 10 aa, Artificial Sequence
  Artificially Synthesized Sequence
GFTFSDYWMD

SEQ ID NO: 32 - protein, 17 aa, Artificial Sequence
  Artificially Synthesized Sequence
NIDEDGSITEYSPFVKG

SEQ ID NO: 33 - protein, 8 aa, Artificial Sequence
  Artificially Synthesized Sequence
WGRFGFDS

SEQ ID NO: 34 - protein, 15 aa, Artificial Sequence
  Artificially Synthesized Sequence
KSSQSLLSGSFNYLT

SEQ ID NO: 35 - protein, 7 aa, Artificial Sequence
  Artificially Synthesized Sequence
YASTRHT

SEQ ID NO: 36 - protein, 9 aa, Artificial Sequence
  Artificially Synthesized Sequence
HHHYNAPPT

SEQ ID NO: 37 - protein, 116 aa, Artificial Sequence
  Artificially Synthesized Sequence
EVQLVESGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSS

SEQ ID NO: 38 - protein, 111 aa, Artificial Sequence
  Artificially Synthesized Sequence
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIK

SEQ ID NO: 39 - protein, 121 aa, Artificial Sequence
  Artificially Synthesized Sequence
EVQLVESGGGLVKPGGSLRLSCAASGFTFSDYFMHWVRQAPGKGLEWVGVIDTKSFNYATYYSDLVKGRFTISRDDSKNTLYLQMNSLKTEDTAVYYCTATIAVPYYFDYWGQGTLVTVSS

SEQ ID NO: 40 - protein, 106 aa, Artificial Sequence
  Artificially Synthesized Sequence
DIQMTQSPSSLSASVGDRVTITCQASQDISNYLSWYQQKPGKAPKLLIYYTNLLAEGVPSRFSGSGSGTDFTFTISSLQPEDIATYYCQQYYNYRTFGPGTKVDIK

SEQ ID NO: 41 - protein, 124 aa, Artificial Sequence
  Artificially Synthesized Sequence
EVQLVESGGGVVRPGGSLRLSCVASGVTFDDYGMSWVRQAPGKGLEWVSGINWNGGDTDYSDSVKGRFTISRDNAKNSLYLQMNSLRAEDTALYYCARDFYGSGSYYHVPFDYWGQGILVTVSS

SEQ ID NO: 42 - protein, 108 aa, Artificial Sequence
  Artificially Synthesized Sequence
EIVLTQSPGTLSLSPGERATLSCRASQSVSRSYLAWYQQKRGQAPRLLIYGASSRATGIPDRFSGDGSGTDFTLSISRLEPEDFAVYYCHQYDMSPFTFGPGTKVDIK

SEQ ID NO: 43 - protein, 125 aa, Artificial Sequence
  Artificially Synthesized Sequence
QVQLVQSGAEVKKPGASVKVSCKASGYTFTGYYMHWVRQAPGQGLEWMGWINPHSGETIYAQKFQGRVTMTRDTSISTAYMELSSLRSEDTAVYYCARTYYYDTSGYYHDAFDVWGQGTMVTVSS

SEQ ID NO: 44 - protein, 5 aa, Artificial Sequence
  Artificially Synthesized Sequence
GYYMH

SEQ ID NO: 45 - protein, 17 aa, Artificial Sequence
  Artificially Synthesized Sequence
WINPHSGETIYAQKFQG

SEQ ID NO: 46 - protein, 16 aa, Artificial Sequence
  Artificially Synthesized Sequence
TYYYDTSGYYHDAFDV

SEQ ID NO: 47 - protein, 107 aa, Artificial Sequence
  Artificially Synthesized Sequence
DIQMTQSPSSVSASVGDRVTITCRASQGISRLLAWYQQKPGKAPKLLIYVASSLQSGVPSRFSGSGSGTDFTLTISSLQPEDFATYYCQQANSFPWTFGQGTKVEIK

SEQ ID NO: 48 - protein, 11 aa, Artificial Sequence
  Artificially Synthesized Sequence
RASQGISRLLA

SEQ ID NO: 49 - protein, 7 aa, Artificial Sequence
  Artificially Synthesized Sequence
VASSLQS

SEQ ID NO: 50 - protein, 9 aa, Artificial Sequence
  Artificially Synthesized Sequence
QQANSFPWT

(19) United States
(12) Patent Application Publication
(10) Pub. No.: US 2023/0149543 A1
(43) Pub. Date: May 18, 2023

(54) COMBINATION TREATMENT FOR CANCER BASED UPON AN ICOS ANTBODY AND A PD-L1 ANTIBODY TGF-BETS-RECEPTOR FUSION PROTEIN

(71) Applicant: GLAXOSMITHKLINE INTELLECTUAL PROPERTY DEVELOPMENT LIMITED, Brentford (GB)

(72) Inventors: Marc S. BALLAS, Collegeville, PA (US); Catherine E. ELLIS, Collegeville, PA (US); Steven HIRSCHFELD, Collegeville, PA (US)

(21) Appl. No.: 17/916,916

(22) PCT Filed: Apr. 12, 2021
(86) PCT No.: PCT/EP2021/059378

§ 371 Date: Oct. 4, 2022

Related U.S. Application Data
(60) Provisional application No. 63/009,580, filed on Apr. 14, 2020.
(60) Provisional application No. 63/037,797, filed on Jun. 11, 2020.

(51) Int. Cl. A61K 39/395; A61K 38/17; A61P 35/00

(52) U.S. Cl. CPC A61K 39/3955; A61K 38/179; A61P 35/00

(57) ABSTRACT

The invention relates to a method of treating cancer, involving the combination of an ICOS binding protein, a PD-1 inhibitor and a TGF-β inhibitor. In particular, the invention relates to an ICOS binding protein (e.g. an anti-ICOS antibody) and a fusion protein targeting human protein Programmed Death Ligand 1 (PD-L1) or Programmed Cell Death Protein 1 (PD-1), and Transforming Growth Factor β (TGF-β) (e.g. an anti-PD-(L)1(IgG):TGFβR fusion protein, comprising, for example, an anti-PD-L1 antibody and a TGFβRII or a fragment capable of binding to TGF-β).

### SEQUENCE LISTING

The instant application contains a Sequence Listing which has been submitted electronically in ASCII format and is hereby incorporated by reference in its entirety. Said ASCII copy, created on Jun. 11, 2020, is named PB66869_WO_Sequence_Listing.txt and is 50.9 kilobytes in size.

### FIELD OF THE INVENTION

The present invention relates to a method of treating cancer in a mammal and to combinations useful in such treatment. In particular, the present invention relates to a combination of an Inducible T-cell COStimulator) (ICOS) binding protein, a Programmed Cell Death Protein 1 (PD-1) inhibitor and a Transforming Growth Factor β (TGF-β) inhibitor for use in the treatment of cancer.

### BACKGROUND TO THE INVENTION

Effective treatment of hyperproliferative disorders, including cancer, is a continuing goal in the oncology field. Generally, cancer results from the deregulation of the normal processes that control cell division, differentiation and apoptotic cell death and is characterized by the proliferation of malignant cells which have the potential for unlimited growth, local expansion and systemic metastasis. Deregulation of normal processes includes abnormalities in signal transduction pathways and response to factors that differ from those found in normal cells.

Immunotherapies are one approach to treat hyperproliferative disorders. A major hurdle that scientists and clinicians have encountered in the development of various types of cancer immunotherapies has been to break tolerance to self-antigen (cancer) in order to mount a robust anti-tumor response leading to tumor regression. Unlike traditional development of small and large molecule agents that target the tumor, cancer immunotherapies may, among other things, target cells of the immune system that have the potential to generate a memory pool of effector cells to induce more durable effects and minimize recurrences.

Though there have been many recent advances in the treatment of cancer, there remains a need for more effective and/or enhanced treatment of an individual suffering the effects of cancer.

The methods herein that relate to combining therapeutic approaches for enhancing anti-tumor immunity address this need.

### SUMMARY OF THE INVENTION

According to a first aspect of the invention, there is provided a combination of an ICOS binding protein, a PD-1 inhibitor and a TGF-β inhibitor for use in the treatment of cancer According to a second aspect of the invention, there is provided a combination comprising:

(i) an ICOS binding protein; and

(ii) a polypeptide comprising a PD-1 inhibitor and a TGFβR,

for use in the treatment of cancer.

According to another aspect of the invention, there is provided a combination comprising:

(i) an ICOS binding protein; and

(ii) an anti-PD-(L)1(IgG):TGFβR fusion protein,

for use in the treatment of cancer. According to a further aspect of the invention, there is provided a combination comprising: an ICOS binding protein comprising a heavy chain amino acid sequence comprising a CDRH1 of SEQ ID NO:1, a CDRH2 of SEQ ID NO:2, and a CDRH3 of SEQ ID NO:3; and a light chain amino acid sequence comprising a CDRL1 of SEQ ID NO:4, a CDRL2 of SEQ ID NO:5, and a CDRL3 of SEQ ID NO:6; and an anti-PD-(L)1(IgG):TGFβR fusion protein comprising: (i) a PD-L1 binding protein comprising a heavy chain amino acid sequence comprising a CDRH1 of SEQ ID NO:13, a CDRH2 of SEQ ID NO:14, and a CDRH3 of SEQ ID NO:15; and a light chain amino acid sequence comprising a CDRL1 of SEQ ID NO:16, a CDRL2 of SEQ ID NO:17, and a CDRL3 of SEQ ID NO:18; and (ii) human TGFβRII, or a fragment thereof capable of binding to TGF-β, for use in the treatment of a cancer.

According to a further aspect of the invention, there is provided a combination comprising: an ICOS binding protein comprising a heavy chain amino acid sequence at least about 90% identical to the amino acid sequence of SEQ ID NO:9 and a light chain amino acid sequence at least about 90% identical to the amino acid sequence of SEQ ID NO:10; and an anti-PD-(L)1(IgG):TGFβR fusion protein comprising a heavy chain amino acid sequence at least about 90% identical to the amino acid sequence of SEQ ID NO:23 and a light chain amino acid sequence at least about 90% identical to the amino acid sequence of SEQ ID NO:22, for use in the treatment of a cancer.

According to a further aspect of the invention, there is provided an ICOS binding protein comprising: a heavy chain amino acid sequence comprising a CDRH1 of SEQ ID NO:1, a CDRH2 of SEQ ID NO:2, and a CDRH3 of SEQ ID NO:3; and a light chain amino acid sequence comprising a CDRL1 of SEQ ID NO:4, a CDRL2 of SEQ ID NO:5, and a CDRL3 of SEQ ID NO:6, for use in treating cancer in a human, wherein the ICOS binding protein is to be administered in combination with an anti-PD-(L)1(IgG):TGFβR fusion protein comprising: (i) a PD-L1 binding protein comprising a CDRH1 of SEQ ID NO:13, a CDRH2 of SEQ ID NO:14, and a CDRH3 of SEQ ID NO:15; and a light chain amino acid sequence comprising a CDRL1 of SEQ ID NO:16, a CDRL2 of SEQ ID NO:17, and a CDRL3 of SEQ ID NO:18; and (ii) human TGFβRII, or a fragment thereof capable of binding to TGF-β.

According to a further aspect of the invention, there is provided an anti-PD-(L)1(IgG):TGFβR fusion protein comprising: (i) a PD-L1 binding protein comprising a CDRH1 of SEQ ID NO:13, a CDRH2 of SEQ ID NO:14, and a CDRH3 of SEQ ID NO:15; and a light chain amino acid sequence comprising a CDRL1 of SEQ ID NO:16, a CDRL2 of SEQ ID NO:17, and a CDRL3 of SEQ ID NO:18; and (ii) human TGFβRII, or a fragment thereof capable of binding to TGF-β, for use in treating cancer, wherein the anti-PD-(L)1(IgG):TGFβR fusion protein is to be administered in combination with an ICOS binding protein comprising: a CDRH1 of SEQ ID NO:1, a CDRH2 of SEQ ID NO:2, and a CDRH3 of SEQ ID NO:3; and a light chain amino acid sequence comprising a CDRL1 of SEQ ID NO:4, a CDRL2 of SEQ ID NO:5, and a CDRL3 of SEQ ID NO:6

According to one aspect of the invention, there is provided a method for the treatment of cancer in a subject in need thereof, comprising administering a therapeutically effective amount of a combination comprising an ICOS binding protein, a PD-1 inhibitor and a TGF-β inhibitor, to the subject

According to another aspect of the invention, there is provided a method for the treatment of cancer in a subject in need thereof comprising administering a therapeutically effective amount of a combination comprising: (i) an ICOS binding protein; and (ii) a polypeptide comprising a PD-1 inhibitor and a TGFβR, to the subject.

According to another aspect of the invention, there is provided a method for the treatment of cancer in a subject in need thereof comprising administering a therapeutically effective amount of a combination comprising: (i) an ICOS binding protein; and (ii) an anti-PD-(L)1(IgG):TGFβR fusion protein, to the subject.

According to a further aspect of the invention, there is provided a method for the treatment of cancer in a subject in need thereof comprising administering a therapeutically effective amount of a combination comprising: an ICOS binding protein comprising a heavy chain amino acid sequence comprising a CDRH1 of SEQ ID NO:1, a CDRH2 of SEQ ID NO:2, and a CDRH3 of SEQ ID NO:3; and a light chain amino acid sequence comprising a CDRL1 of SEQ ID NO:4, a CDRL2 of SEQ ID NO:5, and a CDRL3 of SEQ ID NO:6; and an anti-PD-(L)1(IgG):TGFβR fusion protein comprising: (i) a PD-L1 binding protein comprising a heavy chain amino acid sequence comprising a CDRH1 of SEQ ID NO:13, a CDRH2 of SEQ ID NO:14, and a CDRH3 of SEQ ID NO:15; and a light chain amino acid sequence comprising a CDRL1 of SEQ ID NO:16, a CDRL2 of SEQ ID NO:17, and a CDRL3 of SEQ ID NO:18; and (ii) human TGFβRII, or a fragment thereof capable of binding to TGF-β, to the subject.

According to a further aspect of the invention, there is provided a use of an ICOS binding protein comprising: a heavy chain amino acid sequence comprising a CDRH1 of SEQ ID NO:1, a CDRH2 of SEQ ID NO:2, and a CDRH3 of SEQ ID NO:3; and a light chain amino acid sequence comprising a CDRL1 of SEQ ID NO:4, a CDRL2 of SEQ ID NO:5, and a CDRL3 of SEQ ID NO:6, in the manufacture of a medicament for use in the treatment of a cancer, wherein the medicament is to be administered in combination with an anti-PD-(L)1(IgG):TGFβRfusion protein comprising: (i) a PD-L1 binding protein comprising: a heavy chain amino acid sequence comprising a CDRH1 of SEQ ID NO:13, a CDRH2 of SEQ ID NO:14; and a CDRH3 of SEQ ID NO:15, and a light chain amino acid sequence comprising a CDRL1 of SEQ ID NO:16, a CDRL2 of SEQ ID NO:17, and a CDRL3 of SEQ ID NO:18; and (ii) human TGFβRII, or a fragment thereof capable of binding to TGF-β.

According to a further aspect of the invention, there is provided a use of an anti-PD-(L)1(IgG):TGFβR fusion protein comprising: (i) a PD-L1 binding protein comprising a heavy chain amino acid sequence comprising a CDRH1 of SEQ ID NO:13, a CDRH2 of SEQ ID NO:14, and a CDRH3 of SEQ ID NO:15; and a light chain amino acid sequence comprising a CDRL1 of SEQ ID NO:16, a CDRL2 of SEQ ID NO:17, and a CDRL3 of SEQ ID NO:18; and (ii) human TGFβRII, or a fragment thereof capable of binding to TGF-β, in the manufacture of a medicament for use in the treatment of a cancer, wherein the medicament is to be administered in combination with an ICOS binding protein comprising a heavy chain amino acid sequence comprising a CDRH1 of SEQ ID NO:1, a CDRH2 of SEQ ID NO:2, and a CDRH3 of SEQ ID NO:3; and a light chain amino acid sequence comprising a CDRL1 of SEQ ID NO:4, a CDRL2 of SEQ ID NO:5, and a CDRL3 of SEQ ID NO:6.

According to one aspect of the invention, there is provided a kit comprising:

(i) an ICOS binding protein;

(ii) a PD-1 inhibitor;

(iii) a TGF-β inhibitor; and alternatively comprising

(iv) instructions for using (i), (ii) and (iii) in combination in the treatment of a cancer in a human.

According to another aspect of the invention, there is provided a kit comprising:

(i) an ICOS binding protein;

(ii) a polypeptide comprising a PD-1 inhibitor and a TGFβR; and alternatively comprising

(iii) instructions for using (i) and (ii) in combination in the treatment of a cancer in a human.

According to another aspect of the invention, there is provided a kit comprising:

(i) an ICOS binding protein;

(ii) an anti-PD-(L)1(IgG):TGFβR fusion protein; and alternatively comprising

(iii) instructions for using (i) and (ii) in combination in the treatment of a cancer in a human.

According to a further aspect of the invention, there is provided a kit comprising: (i) an ICOS binding protein comprising a heavy chain amino acid sequence comprising a CDRH1 of SEQ ID NO:1, a CDRH2 of SEQ ID NO:2, and a CDRH3 of SEQ ID NO:3; and a light chain amino acid sequence comprising a CDRL1 of SEQ ID NO:4, a CDRL2 of SEQ ID NO:5, and a CDRL3 of SEQ ID NO:6; (ii) an anti-PD-(L)1(IgG):TGFβR fusion protein comprising: (a) a PD-L1 binding protein comprising a heavy chain amino acid sequence comprising a CDRH1 of SEQ ID NO:13, a CDRH2 of SEQ ID NO:14, and a CDRH3 of SEQ ID NO:15; and a light chain amino acid sequence comprising a CDRL1 of SEQ ID NO:16, a CDRL2 of SEQ ID NO:17, and a CDRL3 of SEQ ID NO:18; and (b) human TGFβRII, or a fragment thereof capable of binding to TGF-β; and (iii) instructions for using (i) and (ii) in combination in the treatment of a cancer in a human.

### DESCRIPTION OF DRAWINGS/FIGURES

FIGS. 1A-1B Results from an in vivo efficacy study in a murine syngeneic tumor model (EMT-6) showing FIG. 1A) tumor volume growth and FIG. 1B) survival curves for anti-ICOS treatment in combination with M7824 at (A) 54.6 μg, (B) 164 μg and (C) 492 μg.

FIG. 2 Summary of study design described in Example 2.

FIG. 3 Modified Toxicity Probability Interval (mTPI) Dose Decision Rules. Columns provide the numbers of subjects treated at a dose level, and rows provide the corresponding numbers of subjects experiencing DLT (dose limiting toxicity). The entries in the table are dose-finding decisions (i.e. E, S, and D) representing escalating the dose, staying at the same dose, and de-escalating the dose, respectively. In addition, decision U indicates that the current dose level is unacceptable because of high toxicity and should be excluded from further investigation in the study.

FIGS. 4A-4B Time and Events table for Safety, Laboratory, Efficacy, Study Treatment Procedures as described in Example 2. The tables of FIG. 4A and FIG. 4B summarise assessment windows and sequencing of assessments and procedures.

FIGS. 5A-5B Time and Events table for Pharmacokinetics, Immunogenicity, Biomarker Assessments as described in Example 2. The tables of FIG. 5A and FIG. 5B summarise assessment windows and sequencing of assessments and procedures.

FIG. 6 Time and Events table for Patient Reported Outcome Assessments as described in Example 2. The table summarises assessment windows and sequencing of assessments and procedures.

### DETAILED DESCRIPTION OF THE INVENTION

### Definitions

“Antigen binding protein” (ABP) means a protein that binds an antigen, including antibodies or engineered molecules that function in similar ways to antibodies. Such alternative antibody formats include triabody, tetrabody, miniantibody, and a minibody. An ABP also includes antigen binding fragments of such antibodies or other molecules. Further, an ABP may comprise the V_{H} regions of the invention formatted into a full length antibody, a (Fab′)₂ fragment, a Fab fragment, a bi-specific or biparatopic molecule or equivalent thereof (such as scFv, bi- tri- or tetra-bodies, TANDABS etc.), when paired with an appropriate light chain. The ABP may comprise an antibody that is an IgG1, IgG2, IgG3, or IgG4; or IgM; IgA, IgE or IgD or a modified variant thereof. The constant domain of the antibody heavy chain may be selected accordingly. The light chain constant domain may be a kappa or lambda constant domain. The ABP may also be a chimeric antibody of the type described in WO86/01533, which comprises an antigen binding region and a non-immunoglobulin region. The terms “ABP”, “antigen binding protein”, “binding protein”, “antigen binding agent” and “binding agent” are used interchangeably herein. For example, disclosed herein are ICOS binding proteins, PD-L1 binding proteins, and PD-1 binding proteins.

“Antigen binding site” refers to a site on an antigen binding protein which is capable of specifically binding to an antigen, this may be a single variable domain, or it may be paired V_{H}/V_{L} domains as can be found on a standard antibody. Single-chain Fv (scFv) domains can also provide antigen-binding sites.

The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanized, multispecific antibodies, including bispecific antibodies, and heteroconjugate antibodies; a single variable domain (e.g. V_{H}, V_{HH}, V_{L}, domain antibody (DAB)), antigen binding antibody fragments, Fab, F(ab′)₂, Fv, disulphide linked Fv, single chain Fv, disulphide-linked scFv, diabodies, TANDABS, etc. and modified versions of any of the foregoing (for a summary of alternative “antibody” formats see, e.g. Holliger and Hudson, Nature Biotechnology, 2005, Vol 23, No. 9, 1126-1136).

A “chimeric antibody” refers to a type of engineered antibody which contains a naturally-occurring variable region (light chain and heavy chains) derived from a donor antibody in association with light and heavy chain constant regions derived from an acceptor antibody.

A “humanized antibody” refers to a type of engineered antibody having its CDRs derived from a non-human donor immunoglobulin, the remaining immunoglobulin-derived parts of the molecule being derived from one or more human immunoglobulin(s). In addition, framework support residues may be altered to preserve binding affinity (see, e.g. Queen et al. Proc. Natl Acad Sci USA, 86:10029-10032 (1989), Hodgson et al. Bio/Technology, 9:421 (1991)). A suitable human acceptor antibody may be one selected from a conventional database, e.g. the KABAT database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies—see, for example, EP-A-0239400 and EP-A-054951.

The term “fully human antibody” includes antibodies having variable and constant regions (if present) derived from human germline immunoglobulin sequences. The human sequence antibodies of the invention may include amino acid residues not encoded by human germline immunoglobulin sequences (e.g. mutations introduced by random or site-specific mutagenesis in vitro or by somatic mutation in vivo). Fully human antibodies comprise amino acid sequences encoded only by polynucleotides that are ultimately of human origin or amino acid sequences that are identical to such sequences. As meant herein, antibodies encoded by human immunoglobulin-encoding DNA inserted into a mouse genome produced in a transgenic mouse are fully human antibodies since they are encoded by DNA that is ultimately of human origin. In this situation, human immunoglobulin-encoding DNA can be rearranged (to encode an antibody) within the mouse, and somatic mutations may also occur. Antibodies encoded by originally human DNA that has undergone such changes in a mouse are fully human antibodies as meant herein. The use of such transgenic mice makes it possible to select fully human antibodies against a human antigen. As is understood in the art, fully human antibodies can be made using phage display technology wherein a human DNA library is inserted in phage for generation of antibodies comprising human germline DNA sequence.

The term, full, whole or intact antibody, used interchangeably herein, refers to a heterotetrameric glycoprotein with an approximate molecular weight of 150,000 daltons. An intact antibody is composed of two identical heavy chains (HCs) and two identical light chains (LCs) linked by covalent disulphide bonds. This H2L2 structure folds to form three functional domains comprising two antigen-binding fragments, known as ‘Fab’ fragments, and a ‘Fc’ crystallisable fragment. The Fab fragment is composed of the variable domain at the amino-terminus, variable heavy (V_{H}) or variable light (V_{L}), and the constant domain at the carboxyl terminus, CH1 (heavy) and CL (light). The Fc fragment is composed of two domains formed by dimerization of paired CH2 and CH3 regions. The Fc may elicit effector functions by binding to receptors on immune cells or by binding C1q, the first component of the classical complement pathway. The five classes of antibodies IgM, IgA, IgG, IgE and IgD are defined by distinct heavy chain amino acid sequences which are called μ, α, γ, ε and δ respectively, each heavy chain can pair with either a K or A light chain. The majority of antibodies in the serum belong to the IgG class, there are four isotypes of human IgG, IgG1, IgG2, IgG3 and IgG4, the sequences of which differ mainly in their hinge region.

Fully human antibodies can be obtained using a variety of methods, for example using yeast-based libraries or transgenic animals (e.g. mice) which are capable of producing repertoires of human antibodies. Yeast presenting human antibodies on their surface which bind to an antigen of interest can be selected using FACS (Fluorescence-Activated Cell Sorting) based methods or by capture on beads using labelled antigens. Transgenic animals that have been modified to express human immunoglobulin genes can be immunised with an antigen of interest and antigen-specific human antibodies isolated using B-cell sorting techniques. Human antibodies produced using these techniques can then be characterised for desired properties such as affinity, developability and selectivity.

Alternative antibody formats include alternative scaffolds in which the one or more CDRs of the antigen binding protein can be arranged onto a suitable non-immunoglobulin protein scaffold or skeleton, such as an affibody, a SpA scaffold, an LDL receptor class A domain, an avimer (see, e.g. U.S. Patent Application Publication Nos. 2005/0053973, 2005/0089932, 2005/0164301) or an EGF domain.

The term “domain” refers to a folded polypeptide structure that retains its tertiary structure independent of the rest of the polypeptide. Generally domains are responsible for discrete functional properties of polypeptides and in many cases may be added, removed or transferred to other polypeptides without loss of function of the remainder of the protein and/or of the domain.

The term “single variable domain” refers to a folded polypeptide domain comprising sequences characteristic of antibody variable domains. It therefore includes complete antibody variable domains, such as V_{H}, V_{HH} and V_{L}, and modified antibody variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain. A single variable domain is capable of binding an antigen or epitope independently of a different variable region or domain. A “domain antibody” or “DAB” may be considered the same as a “single variable domain”. A single variable domain may be a human single variable domain, but also includes single variable domains from other species such as rodent, nurse shark and Camelid V_{HH} DABS. Camelid V_{HH} are immunoglobulin single variable domain polypeptides that are derived from species including camel, llama, alpaca, dromedary, and guanaco, which produce heavy chain antibodies naturally devoid of light chains. Such V_{HH} domains may be humanized according to standard techniques available in the art, and such domains are considered to be “single variable domains”. As used herein V_{H} includes camelid V_{HH} domains.

The terms “V_{H}” and “V_{L}” are used herein to refer to the heavy chain variable region and light chain variable region respectively of an antigen binding protein.

“CDRs” are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and variable domain regions within full length antigen binding sequences, e.g. within an antibody heavy chain sequence or antibody light chain sequence, are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al. Sequences of Proteins of Immunological Interest, 5th Ed., U.S. Department of Health and Human Services, National Institutes of Health (1991).

It will be apparent to those skilled in the art that there are alternative numbering conventions for amino acid residues in variable domain sequences and full length antibody sequences. There are also alternative numbering conventions for CDR sequences, for example those set out in Chothia et al. (1989) Nature 342: 877-883. The structure and protein folding of the antigen binding protein may mean that other residues are considered part of the CDR sequence and would be understood to be so by a skilled person.

Other numbering conventions for CDR sequences available to a skilled person include “AbM” (University of Bath) and “contact” (University College London) methods. The minimum overlapping region using at least two of the Kabat, Chothia, AbM and contact methods can be determined to provide the “minimum binding unit”. The minimum binding unit may be a sub-portion of a CDR.

CDRs or minimum binding units may be modified by at least one amino acid substitution, deletion or addition, wherein the variant antigen binding protein substantially retains the biological characteristics of the unmodified protein, such as an antibody comprising SEQ ID NO:7 and SEQ ID NO:8.

CDRs or minimum binding units may be modified by at least one amino acid substitution, deletion or addition, wherein the variant antigen binding protein substantially retains the biological characteristics of the unmodified protein, such as an antibody comprising SEQ ID NO:7 and SEQ ID NO:8. It will be appreciated that each of CDR H1, H2, H3, L1, L2, L3 may be modified alone or in combination with any other CDR, in any permutation or combination. In one embodiment, a CDR is modified by the substitution, deletion or addition of up to 3 amino acids, for example 1 or 2 amino acids, for example 1 amino acid. Typically, the modification is a substitution, particularly a conservative substitution (referred herein also as a direct equivalent), for example as shown in Table 1 below.

TABLE 1

| Side chain | Members |
|---|---|
| Hydrophobic | Met, Ala, Val, Leu, Ile |
| Neutral hydrophilic | Cys, Ser, Thr |
| Acidic | Asp, Glu |
| Basic | Asn, Gln, His, Lys, Arg |
| Residues that influence chain orientation | Gly, Pro |
| Aromatic | Trp, Tyr, Phe |

“Percent identity” between a query amino acid sequence and a subject amino acid sequence is the “Identities” value, expressed as a percentage, that is calculated using a suitable algorithm or software, such as BLASTP, FASTA, DNASTAR Lasergene, GeneDoc, Bioedit, EMBOSS needle or EMBOSS infoalign, over the entire length of the query sequence after a pair-wise global sequence alignment has been performed using a suitable algorithm/software such as BLASTP, FASTA, ClustalW, MUSCLE, MAFFT, EMBOSS Needle, T-Coffee, and DNASTAR Lasergene. Importantly, a query amino acid sequence may be described by an amino acid sequence identified in one or more claims herein.

The query sequence may be 100% identical to the subject sequence, or it may include up to a certain integer number of amino acid or nucleotide alterations as compared to the subject sequence such that the % identity is less than 100%. For example, the query sequence is at least 50, 60, 70, 75, 80, 85, 90, 95, 96, 97, 98, or 99% identical to the subject sequence. Such alterations include at least one amino acid deletion, substitution (including conservative and non-conservative substitution), or insertion, and wherein said alterations may occur at the amino- or carboxy-terminal positions of the query sequence or anywhere between those terminal positions, interspersed either individually among the amino acids or nucleotides in the query sequence or in one or more contiguous groups within the query sequence.

The % identity may be determined across the entire length of the query sequence, including the CDRs. Alternatively, the % identity may exclude one or more or all of the CDRs, for example all of the CDRs are 100% identical to the subject sequence and the % identity variation is in the remaining portion of the query sequence, e.g. the framework sequence, so that the CDR sequences are fixed and intact.

The variant sequence substantially retains the biological characteristics of the unmodified protein, such as an agonist for ICOS.

An antigen binding fragment may be provided by means of arrangement of one or more CDRs on non-antibody protein scaffolds. “Protein Scaffold” as used herein includes, but is not limited to, an immunoglobulin (Ig) scaffold, for example an IgG scaffold, which may be a four chain or two chain antibody, or which may comprise only the Fc region of an antibody, or which may comprise one or more constant regions from an antibody, which constant regions may be of human or primate origin, or which may be an artificial chimera of human and primate constant regions.

The protein scaffold may be an Ig scaffold, for example an IgG, or IgA scaffold. The IgG scaffold may comprise some or all the domains of an antibody (i.e. CH1, CH2, CH3, V_{H}, V_{L}). The antigen binding protein may comprise an IgG scaffold selected from IgG1, IgG2, IgG3, IgG4 or IgG4PE. For example, the scaffold may be IgG1. The scaffold may consist of, or comprise, the Fc region of an antibody, or is a part thereof.

The subclass of an antibody in part determines secondary effector functions, such as complement activation or Fc receptor (FcR) binding and antibody dependent cell cytotoxicity (ADCC) (Huber et al. Nature 229(5284): 419-20 (1971); Brunhouse et al. Mol Immunol 16(11): 907-17 (1979)). In identifying the optimal type of antibody for a particular application, the effector functions of the antibodies can be taken into account. For example, hIgG1 antibodies have a relatively long half-life, are very effective at fixing complement, and they bind to both FcγRI and FcγRII. In contrast, human IgG4 antibodies have a shorter half-life, do not fix complement and have a lower affinity for the FcRs. Replacement of serine 228 with a proline (S228P) in the Fc region of IgG4 reduces heterogeneity observed with hIgG4 and extends the serum half-life (Kabat et al. “Sequences of proteins of immunological interest” 5.sup.th Edition (1991); Angal et al. Mol Immunol 30(1): 105-8 (1993)). A second mutation that replaces leucine 235 with a glutamic acid (L235E) eliminates the residual FcR binding and complement binding activities (Alegre et al. J Immunol 148(11): 3461-8 (1992)). The numbering of the hIgG4 amino acids was derived from EU numbering reference: Edelman et al. Proc. Natl. Acad. USA, 63, 78-85 (1969). PMID: 5257969.

The term “donor antibody” refers to an antibody that contributes the amino acid sequences of its variable regions, CDRs, or other functional fragments or analogs thereof to a first immunoglobulin partner. The donor, therefore, provides the altered immunoglobulin coding region and resulting expressed altered antibody with the antigenic specificity and neutralising activity characteristic of the donor antibody.

The term “acceptor antibody” refers to an antibody that is heterologous to the donor antibody, which contributes all (or any portion) of the amino acid sequences encoding its heavy and/or light chain framework regions and/or its heavy and/or light chain constant regions to the first immunoglobulin partner. A human antibody may be the acceptor antibody.

Affinity, also referred to as “binding affinity”, is the strength of binding at a single interaction site, i.e. of one molecule, e.g. an antigen binding protein of the invention, to another molecule, e.g. its target antigen, at a single binding site. The binding affinity of an antigen binding protein to its target may be determined by equilibrium methods (e.g. enzyme-linked immunoabsorbent assay (ELISA) or radioimmunoassay (RIA)), or kinetics (e.g. BIACORE analysis).

Avidity, also referred to as functional affinity, is the cumulative strength of binding at multiple interaction sites, e.g. the sum total of the strength of binding of two molecules (or more, e.g. in the case of a bispecific or multispecific molecule) to one another at multiple sites, e.g. taking into account the valency of the interaction.

As used herein an “immuno-modulator” or “immuno-modulatory agent” refers to any substance, including monoclonal antibodies, that affects the immune system. In some embodiments, the immuno-modulator or immuno-modulatory agent upregulates an aspect of the immune system.

Immuno-modulators can be used as anti-neoplastic agents for the treatment of cancer. For example, immuno-modulators include, but are not limited to, anti-PD-1 antibodies (e.g. OPDIVO/nivolumab, KEYTRUDA/pembrolizumab, LIBTAYO/cemiplimab), anti-PD-L1 antibodies (e.g. BAVENCIO/avelumab, IMFINZI/durvalumab, TECENTRIQ/atezolizumab) and anti-ICOS antibodies.

As used herein the term “agonist” refers to an antigen binding protein including, but not limited to, an antibody, that upon contact with a co-signaling receptor causes one or more of the following: (1) stimulates or activates the receptor, (2) enhances, increases or promotes, induces or prolongs an activity, function or presence of the receptor and/or (3) enhances, increases, promotes or induces the expression of the receptor. Agonist activity can be measured in vitro by various assays know in the art such as, but not limited to, measurement of cell signalling, cell proliferation, immune cell activation markers, cytokine production. Agonist activity can also be measured in vivo by various assays that measure surrogate end points such as, but not limited to, the measurement of T cell proliferation or cytokine production. In one embodiment, the ICOS binding protein is an agonist ICOS binding protein.

The term “TGF-β receptor” (TGFβR), as well as “TGF-β receptor I” (abbreviated as TGFβRI or TGFβR1) or “TGF-β receptor II” (abbreviated as TGFβRII or TGFβR2), are well known in the art. For the purposes of this disclosure, reference to such receptor includes the full receptor and fragments that are capable of binding TGF-β. Preferably, it is the extracellular domain of the receptor or a fragment of the extracellular domain that is capable of binding TGF-β.

The term “TGF-β inhibitor” refers to a molecule that inhibits the interaction between TGF-β and the TGF-β receptor and thereby inhibits the activity of TGF-β. Inhibition in this context need not be complete or 100%. Instead, inhibition means reducing, decreasing or abrogating binding between TGF-β and the TGF-β receptor (TGFβR) and/or reducing, decreasing or abrogating signaling though the TGF-β receptor/the activity of TGF-β. The TGF-β inhibitor may bind to TGF-β or the TGF-β receptor. Preferably, the TGF-β inhibitor binds to TGF-β. A TGF-β inhibitor is preferably a polypeptide or protein. Examples of TGF-β inhibitors include anti-PD-L1/TGFβ Traps and anti-PD-1/TGFβ Traps disclosed herein, as well as soluble TGF-β receptors and other TGF-β binding proteins.

The term “PD-1 inhibitor” refers to a molecule that inhibits the interaction between PD-1 and at least one of its ligands, such as PD-L1 or PD-L2, and thereby inhibits the activity of PD-1. Inhibition in this context need not be complete or 100%. Instead, inhibition means reducing, decreasing or abrogating binding between PD-1 and one or more of its ligands and/or reducing, decreasing or abrogating signaling though the PD-1 receptor/the activity of PD-1. In a preferred embodiment, the PD-1 inhibitor inhibits the interaction between PD-1 and PD-L1. The PD-1 inhibitor may bind to PD-1 or one of its ligands. Preferably, the PD-1 inhibitor binds to PD-L1. A PD-1 inhibitor is preferably a polypeptide or protein. Examples of PD-1 inhibitors include PD-L1 binding proteins, PD-1 binding proteins, anti-PD-L1/TGFβ Traps, anti-PD-1/TGFβ Traps, anti-PD-1 antibodies (e.g. OPDIVO/nivolumab, KEYTRUDA/pembrolizumab, LIBTAYO/cemiplimab), and anti-PD-L1 antibodies (e.g. BAVENCIO/avelumab, IMFINZI/durvalumab, TECENTRIQ/atezolizumab).

Examples of ICOS binding proteins include e.g. feladilimab, 37A10S713, vopratelimab/JTX-2011, ICOS.33 IgG1f S267E, STIM003 and XENP23104.

The term “fusion protein” is well understood in the art and it will be appreciated that the term “polypeptide comprising a PD-1 inhibitor and a TGFβR” as recited herein includes an IgG:TGFβR fusion protein, such as an anti-PD-1(IgG):TGFβR fusion protein or an anti-PD-L1(IgG):TGFβR fusion protein. An IgG:TGFβR fusion protein is an IgG antibody (preferably a monoclonal antibody, preferably in homodimeric form) or an antigen-binding fragment thereof fused to a TGF-β receptor. The nomenclature anti-PD-L1(IgG1):TGFβRII fusion protein indicates an anti-PD-L1 IgG1 antibody, or an antigen-binding fragment thereof, fused to a TGF-β receptor II, preferably a fragment of the extracellular domain thereof that is capable of binding TGF-β. The nomenclature anti-PD-1(IgG1):TGFβRII fusion protein indicates an anti-PD-1 IgG1 antibody, or an antigen-binding fragment thereof, fused to a TGF-β receptor II, preferably a fragment of the extracellular domain thereof that is capable of binding TGF-β. The nomenclature anti-PD-(L)1(IgG):TGFβR fusion protein, indicates an anti-PD-1 IgG antibody or an antigen-binding fragment thereof, or an anti-PD-L1 IgG antibody or an antigen-binding fragment thereof, fused to a TGF-β receptor II, preferably a fragment of the extracellular domain thereof that is capable of binding TGF-β.

“Bintrafusp alfa”, also known as M7824, is well understood in the art. Bintrafusp alfa is an anti-PD-L1 (IgG1):TGFβRII fusion protein and described under the CAS Registry Number 1918149-01-5. It is also described in WO 2015/118175 and further elaborated in Lan et al (Lan et al, “Enhanced preclinical antitumor activity of M7824, a bifunctional fusion protein simultaneously targeting PD-L1 and TGF-β”, Sci. Transl. Med. 10, 2018, p.1-15). In particular, bintrafusp alfa is a fully human IgG1 monoclonal antibody against human PD-L1 fused to the extracellular domain of human TGF-β receptor II (TGFβRII). As such, bintrafusp alfa is a bifunctional fusion protein that simultaneously blocks PD-L1 and TGF-β pathways. In particular, WO 2015/118175 describes bintrafusp alfa on page 34 in Example 1 thereof as follows (bintrafusp alfa is referred to in this passage as “anti-PD-L1/TGFβ Trap”): “Anti-PD-L1/TGFβ Trap is an anti-PD-L1 antibody-TGFβ Receptor II fusion protein. The light chain of the molecule is identical to the light chain of the anti-PD-L1 antibody (SEQ ID NO: 1). The heavy chain of the molecule (SEQ ID NO:3) is a fusion protein comprising the heavy chain of the anti-PD-L1 antibody (SEQ ID NO: 2) genetically fused to via a flexible (Gly₄Ser)₄Gly linker (SEQ ID NO:11) to the N-terminus of the soluble TGFβ Receptor II (SEQ ID NO: 10). At the fusion junction, the C-terminal lysine residue of the antibody heavy chain was mutated to alanine to reduce proteolytic cleavage.”

The term “anti-PD-L1/TGFβ Trap” herein refers to a fusion molecule comprising: 1) an antibody or antigen-binding fragment thereof that is capable of binding PD-L1 and antagonizing the interaction between PD-1 and PD-L1 and 2) a TGFβRII or fragment of TGFβRII that is capable of binding TGF-β and antagonizing the interaction between TGF-β and TGFβRII. In a particular embodiment, anti-PD-L1/TGFβ Trap is one of the fusion molecules disclosed in WO 2015/118175 or WO 2018/205985. For instance, anti-PD-L1/TGFβ Trap may comprise the light chains and heavy chains of SEQ ID NO: 1 and SEQ ID NO: 3 of WO 2015/118175, respectively. In an embodiment, the anti-PD-L1/TGFβ Trap is bintrafusp alfa. In another embodiment, anti-PD-L1/TGFβ Trap is one of the constructs listed in Table 2 of WO 2018/205985, such as construct 9 or 15 thereof. In other embodiments, the antibody having the heavy chain sequence of SEQ ID NO: 11 and the light chain sequence of SEQ ID NO: 12 of WO 2018/205985 is fused via a linking sequence (G₄S)ₓG, wherein x is 4-5, to the TGFβRII extracellular domain sequence of SEQ ID NO: 14 or SEQ ID NO: 15 of WO 2018/205985. In another embodiment, the anti-PD-L1/TGFβ Trap is SHR1701. In a further embodiment, the anti-PD-L1/TGFβ Trap is one of the fusion molecules disclosed in WO 2020/006509. In a preferred embodiment, the anti-PD-L1/TGFβ Trap is Bi-PLB-1, Bi-PLB-2 or Bi-PLB-1.2 disclosed in WO 2020/006509. In a preferred embodiment, the anti-PD-L1/TGFβ Trap is Bi-PLB-1.2 disclosed in WO 2020/006509. In a preferred embodiment, the anti-PD-L1/TGFβ Trap comprises SEQ ID NO:128 and SEQ ID NO:95 disclosed in WO 2020/006509.

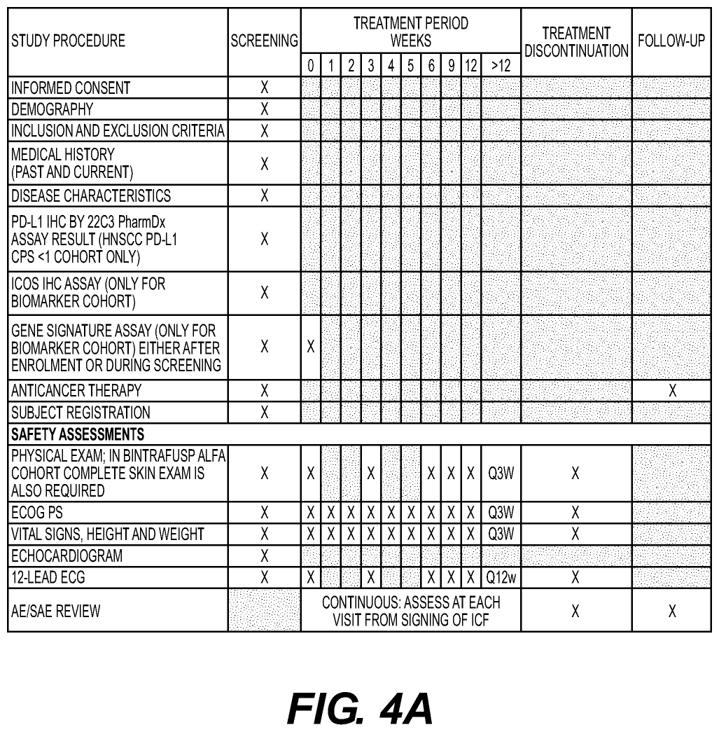

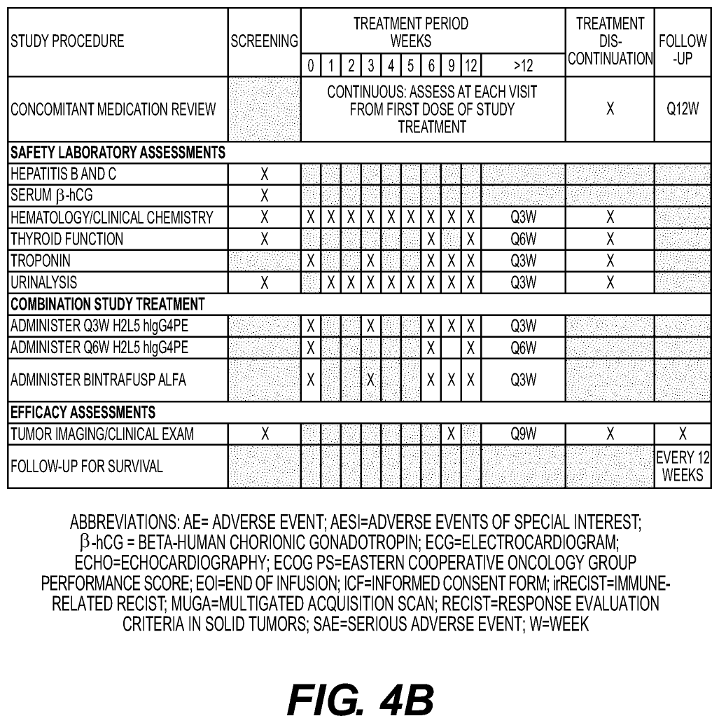

The term “anti-PD-1/TGFβ Trap” refers to a fusion molecule comprising: 1) an antibody or antigen-binding fragment thereof that is capable of binding PD-1 and antagonizing the interaction between PD-1 and PD-L1 and/or PD-1 and PD-L2 and 2) a TGFβRII or fragment of TGFβRII that is capable of binding TGF-β and antagonizing the interaction between TGF-β and TGFβRII. In a particular embodiment, the anti-PD-1/TGFβ Trap is one of the fusion molecules disclosed in WO 2020/014285 that binds both PD-1 and TGF-β, e.g. as depicted in FIG. 4 therein or as described in Example 1, including those identified in Tables 2-9, as specified in table 16, therein, and in particular a fusion protein comprising a sequence that is at least 90% identical to SEQ ID NO:15 or SEQ ID NO:296 and a sequence that is at least 90% identical to SEQ ID NO:16, SEQ ID NO:143, SEQ ID NO:144, SEQ ID NO:145, SEQ ID NO:294 or SEQ ID NO:295 therein. In an embodiment, the anti-PD-1/TGFβ Trap comprises SEQ ID NO:15 and SEQ ID NO:16 of WO 2020/014285. In an embodiment, the anti-PD-1/TGFβ Trap comprises SEQ ID NO:15 and SEQ ID NO:143 of WO 2020/014285. In an embodiment, the anti-PD-1/TGFβ Trap comprises SEQ ID NO:15 and SEQ ID NO:144 of WO 2020/014285. In an embodiment, the anti-PD-1/TGFβ Trap comprises SEQ ID NO:15 and SEQ ID NO:145 of WO 2020/014285. In an embodiment, the anti-PD-1/TGFβ Trap comprises SEQ ID NO:15 and SEQ ID NO:294 of WO 2020/014285. In an embodiment, the anti-PD-1/TGFβ Trap comprises SEQ ID NO:15 and SEQ ID NO:295 of WO 2020/014285. In an embodiment, the anti-PD-1/TGFβ Trap comprises SEQ ID NO:296 and SEQ ID NO:16 of WO 2020/014285. In an embodiment, the anti-PD-1/TGFβ Trap comprises SEQ ID NO:296 and SEQ ID NO:143 of WO 2020/014285. In an embodiment, the anti-PD-1/TGFβ Trap comprises SEQ ID NO:296 and SEQ ID NO:144 of WO 2020/014285. In an embodiment, the anti-PD-1/TGFβ Trap comprises SEQ ID NO:296 and SEQ ID NO:145 of WO 2020/014285. In an embodiment, the anti-PD-1/TGFβ Trap comprises SEQ ID NO:296 and SEQ ID NO:294 of WO 2020/014285. In an embodiment, the anti-PD-1/TGFβ Trap comprises SEQ ID NO:296 and SEQ ID NO:295 of WO 2020/014285. In a further embodiment, the anti-PD-1/TGFβ Trap is one of the fusion molecules disclosed in WO 2020/006509. In a preferred embodiment, the anti-PD-1/TGFβ Trap is Bi-PB-1, Bi-PB-2 or Bi-PB-1.2 disclosed in WO 2020/006509. In a preferred embodiment, the anti-PD-1/TGFβ Trap is Bi-PB-1.2 disclosed in WO 2020/006509. In a preferred embodiment, the anti-PD-1/TGFβ Trap comprises SEQ ID NO:108 and SEQ ID NO:93 disclosed in WO 2020/006509.

By “isolated” it is intended that the molecule, such as an antigen binding protein or nucleic acid, is removed from the environment in which it may be found in nature. For example, the molecule may be purified away from substances with which it would normally exist in nature. For example, the mass of the molecule in a sample may be 95% of the total mass.

The term “expression vector” as used herein means an isolated nucleic acid that can be used to introduce a nucleic acid of interest into a cell, such as a eukaryotic cell or prokaryotic cell, or is a cell free expression system where the nucleic acid sequence of interest is expressed as a peptide chain, such as a protein. Such expression vectors may be, for example, cosmids, plasmids, viral sequences, transposons, and linear nucleic acids comprising a nucleic acid of interest. Once the expression vector is introduced into a cell or cell free expression system (e.g. reticulocyte lysate) the protein encoded by the nucleic acid of interest is produced by the transcription/translation machinery.

Expression vectors within the scope of the disclosure may provide necessary elements for eukaryotic or prokaryotic expression and include viral promoter driven vectors, such as CMV promoter driven vectors, e.g. pcDNA3.1, pCEP4, and their derivatives, Baculovirus expression vectors, Drosophila expression vectors, and expression vectors that are driven by mammalian gene promoters, such as human Ig gene promoters. Other examples include prokaryotic expression vectors, such as T7 promoter driven vectors, e.g. pET41, lactose promoter driven vectors and arabinose gene promoter driven vectors. Those of ordinary skill in the art will recognize many other suitable expression vectors and expression systems.

The term “recombinant host cell” as used herein means a cell that comprises a nucleic acid sequence of interest that was isolated prior to its introduction into the cell. For example, the nucleic acid sequence of interest may be in an expression vector while the cell may be prokaryotic or eukaryotic. Exemplary eukaryotic cells are mammalian cells, such as but not limited to, COS-1, COS-7, HEK293, BHK21, CHO, BSC-1, HepG2, 653, SP2/0, NS0, 293, HeLa, myeloma, lymphoma cells or any derivative thereof. Most preferably, the eukaryotic cell is a HEK293, NS0, SP2/0, or CHO cell. E. coli is an exemplary prokaryotic cell. A recombinant cell according to the disclosure may be generated by transfection, cell fusion, immortalization, or other procedures well known in the art. A nucleic acid sequence of interest, such as an expression vector, transfected into a cell may be extrachromasomal or stably integrated into the chromosome of the cell.

As used herein, the term “effective dose” means that dose of a drug or pharmaceutical agent that will elicit the biological or medical response of a tissue, system, animal or human that is being sought, for instance, by a researcher or clinician. Furthermore, the term “therapeutically effective dose” means any dose that, as compared to a corresponding subject who has not received such dose, results in improved treatment, healing, prevention, or amelioration of a disease, disorder, or side effect, or a decrease in the rate of advancement of a disease or disorder. The term also includes within its scope doses effective to enhance normal physiological function. Therapeutically effective amounts and treatment regimes are generally determined empirically and may be dependent on factors, such as the age, weight, and health status of the patient and disease or disorder to be treated. Such factors are within the purview of the attending physician.

Ranges provided herein, of any type, include all values within a particular range described and values about an endpoint for a particular range.

### Combinations

The present invention relates to a combination comprising an ICOS binding protein, a PD-1 inhibitor and a TGF-β inhibitor. In particular, the invention provides an ICOS binding protein and a polypeptide comprising a PD-1 inhibitor and a TGFβR, such as an IgG:TGFβR fusion protein, for use in the treatment of a cancer, in particular in the treatment of a cancer in a human. In some embodiments, the PD-1 inhibitor is a PD-1 binding protein or a PD-L1 binding protein. Thus, in some embodiments, the IgG:TGFβR fusion protein is an anti-PD-L1(IgG):TGFβRII fusion protein. In an embodiment, the IgG:TGFβR fusion protein comprises SEQ ID NO:22 and SEQ ID NO:23. In an embodiment the IgG:TGFβR fusion protein is bintrafusp alfa. In an embodiment the IgG:TGFβR fusion protein is SHR1701. In an embodiment the IgG:TGFβR fusion protein is preferably one as disclosed in WO 2020/006509. In some embodiments, the IgG:TGFβR fusion protein is an anti-PD-1 (IgG1):TGFβRII fusion protein, preferably one as disclosed in WO 2020/014285 or WO 2020/006509.

Therefore, according to a first aspect of the invention, there is provided a combination comprising an ICOS binding protein, a PD-1 inhibitor and a TGF-β inhibitor for use in the treatment of cancer.

In one embodiment, administration may comprise the ICOS binding protein, followed by the PD-1 inhibitor, followed by the TGF-β inhibitor. In an alternative embodiment, administration may comprise the ICOS binding protein, followed by the TGF-β inhibitor, followed by the PD-1 inhibitor. In an alternative embodiment, administration may comprise the PD-1 inhibitor, followed by the ICOS binding protein, followed by the TGF-β inhibitor. In an alternative embodiment, administration may comprise the PD-1 inhibitor, followed by the TGF-β inhibitor, followed by the ICOS binding protein. In an alternative embodiment, administration may comprise the TGF-β inhibitor, followed by the ICOS binding protein, followed by the PD-1 inhibitor. In an alternative embodiment, administration may comprise the TGF-β inhibitor, followed by the PD-1 inhibitor, followed by the ICOS binding protein.

In a further aspect, there is provided a combination comprising: (i) an ICOS binding protein; and (ii) a polypeptide comprising a PD-1 inhibitor and a TGFβR, for use in the treatment of cancer. In one embodiment, the PD-1 inhibitor is a PD-1 binding protein. In an alternative embodiment, the PD-1 inhibitor is a PD-L1 binding protein.

In one embodiment, administration may comprise the ICOS binding protein, followed by the polypeptide comprising a PD-1 inhibitor and a TGFβR. In an alternative embodiment, administration may comprise the polypeptide comprising a PD-1 inhibitor and a TGFβR, followed by the ICOS binding protein.

In a further aspect, there is provided a combination comprising: (i) an ICOS binding protein; and (ii) an anti-PD-(L)1(IgG):TGFβR fusion protein, for use in the treatment of cancer. Thus, in some embodiments, the polypeptide comprising a PD-1 inhibitor and a TGFβR is an IgG:TGFβR fusion protein. In some embodiments, the polypeptide comprising a PD-1 inhibitor and a TGFβR is an anti-PD-(L)1(IgG):TGFβR fusion protein, such as an anti-PD-L1 (IgG):TGFβR fusion protein or an anti-PD-1(IgG):TGFβR fusion protein. In some embodiments, the IgG:TGFβR fusion protein is an anti-PD-(L)1(IgG):TGFβR fusion protein, such as an anti-PD-L1 (IgG):TGFβR fusion protein or an anti-PD-1(IgG):TGFβR fusion protein. In one embodiment, the IgG:TGFβR fusion protein comprises (a) human TGFβRII, or a fragment thereof capable of binding to TGF-β; and (b) an anti-PD-L1 antibody or an antigen-binding fragment thereof, or an anti-PD-1 antibody or an antigen-binding fragment thereof.

In some embodiments, the anti-PD-(L)1(IgG):TGFβR fusion protein comprises (a) human TGFβRII, or a fragment thereof capable of binding to TGF-β; and (b) an anti-PD-L1 antibody or an antigen-binding fragment thereof, or an anti-PD-1 antibody or an antigen-binding fragment thereof. In one embodiment, the anti-PD-(L)1(IgG):TGFβR fusion protein comprises (a) human TGFβRII, or a fragment thereof capable of binding to TGF-β; and (b) an anti-PD-L1 antibody or an antigen-binding fragment thereof, and is an anti-PD-L1(IgG):TGFβRII fusion protein. In another embodiment, the anti-PD-(L)1(IgG):TGFβR fusion protein comprises (a) human TGFβRII, or a fragment thereof capable of binding to TGF-β; and (b) an anti-PD-1 antibody or an antigen-binding fragment thereof, and is an anti-PD-1(IgG):TGFβRII fusion protein.

In one embodiment, administration may comprise the ICOS binding protein, followed by the anti-PD-(L)1(IgG):TGFβR fusion protein. In an alternative embodiment, administration may comprise the anti-PD-(L)1(IgG):TGFβR fusion protein, followed by the ICOS binding protein.

In one embodiment, administration may comprise the ICOS binding protein, followed by the anti-PD-(L)1(IgG):TGFβR fusion protein. In an alternative embodiment, administration may comprise the anti-PD-(L)1(IgG):TGFβR fusion protein, followed by the ICOS binding protein.

In a further aspect, there is provided a combination comprising: an ICOS binding protein comprising a heavy chain amino acid sequence comprising a CDRH1 of SEQ ID NO:1, a CDRH2 of SEQ ID NO:2, and a CDRH3 of SEQ ID NO:3; and a light chain amino acid sequence comprising a CDRL1 of SEQ ID NO:4, a CDRL2 of SEQ ID NO:5, and a CDRL3 of SEQ ID NO:6; and an anti-PD-(L)1(IgG):TGFβR fusion protein comprising: (i) a PD-L1 binding protein comprising a heavy chain amino acid sequence comprising a CDRH1 of SEQ ID NO:13, a CDRH2 of SEQ ID NO:14, and a CDRH3 of SEQ ID NO:15; and a light chain amino acid sequence comprising a CDRL1 of SEQ ID NO:16, a CDRL2 of SEQ ID NO:17, and a CDRL3 of SEQ ID NO:18; and (ii) human TGFβRII, or a fragment thereof capable of binding TGF-β, for use in the treatment of a cancer.

In another aspect, there is provided a combination comprising: an ICOS binding protein comprising a heavy chain amino acid sequence at least about 90% identical to the amino acid sequence of SEQ ID NO:9 and a light chain amino acid sequence at least about 90% identical to the amino acid sequence of SEQ ID NO:10; and an anti-PD-(L)1(IgG):TGFβR fusion protein comprising: (i) a PD-L1 binding protein comprising a heavy chain amino acid sequence at least about 90% identical to the amino acid sequence of SEQ ID NO:21 and a light chain amino acid sequence at least about 90% identical to the amino acid sequence of SEQ ID NO:22; and (ii) human TGFβRII, or a fragment thereof capable of binding to TGF-β, for use in the treatment of a cancer.

In another aspect, there is provided a combination comprising: an ICOS binding protein comprising a heavy chain amino acid sequence at least about 90% identical to the amino acid sequence of SEQ ID NO:9 and a light chain amino acid sequence at least about 90% identical to the amino acid sequence of SEQ ID NO:10; and an anti-PD-(L)1(IgG):TGFβR fusion protein comprising a heavy chain amino acid sequence at least about 90% identical to the amino acid sequence of SEQ ID NO:23 and a light chain amino acid sequence at least about 90% identical to the amino acid sequence of SEQ ID NO:22, for use in the treatment of a cancer.

In another aspect, there is provided an ICOS binding protein comprising a heavy chain amino acid sequence comprising a CDRH1 of SEQ ID NO:1, a CDRH2 of SEQ ID NO:2, and a CDRH3 of SEQ ID NO:3; and a light chain amino acid sequence comprising a CDRL1 of SEQ ID NO:4, a CDRL2 of SEQ ID NO:5, and a CDRL3 of SEQ ID NO:6, for use in treating cancer in a human, wherein the ICOS binding protein is to be administered in combination with an anti-PD-(L)1(IgG):TGFβR fusion protein comprising: (i) a PD-L1 binding protein comprising a CDRH1 of SEQ ID NO:13, a CDRH2 of SEQ ID NO:14, and a CDRH3 of SEQ ID NO:15; and a light chain amino acid sequence comprising a CDRL1 of SEQ ID NO:16, a CDRL2 of SEQ ID NO:17, and a CDRL3 of SEQ ID NO:18; and (ii) human TGFβRII, or a fragment thereof capable of binding to TGF-β.

In another aspect, there is provided an anti-PD-(L)1(IgG):TGFβR fusion protein comprising: (i) a PD-L1 binding protein comprising a CDRH1 of SEQ ID NO:13, a CDRH2 of SEQ ID NO:14, and a CDRH3 of SEQ ID NO:15; and a light chain amino acid sequence comprising a CDRL1 of SEQ ID NO:16, a CDRL2 of SEQ ID NO:17, and a CDRL3 of SEQ ID NO:18; and (ii) human TGFβRII, or a fragment thereof capable of binding to TGF-β, for use in treating cancer, wherein the anti-PD-(L)1(IgG):TGFβR fusion protein is to be administered in combination with an ICOS binding protein comprising: a heavy chain amino acid sequence comprising a CDRH1 of SEQ ID NO:1, a CDRH2 of SEQ ID NO:2, and a CDRH3 of SEQ ID NO:3; and a light chain amino acid sequence comprising a CDRL1 of SEQ ID NO:4, a CDRL2 of SEQ ID NO:5, and a CDRL3 of SEQ ID NO:6.

The term “combination” of the invention described herein refers to at least two therapeutic agents (i.e. antigen binding proteins or inhibitors). It will be understood that references to a “combination” include embodiments where the two therapeutic agents are administered concurrently (i.e. simultaneously) or sequentially. Therefore, the individual therapeutic agents of the combination of the invention, and pharmaceutical compositions comprising such therapeutic agents may be administered together or separately. When administered separately, this may occur simultaneously or sequentially in any order (by the same or by different routes of administration). In a preferred embodiment, the ICOS binding protein is administered first. Such sequential administration may be close in time or remote in time. The dose of a therapeutic agent of the invention or pharmaceutically acceptable salt thereof and the further therapeutically active agent(s) and the relative timings of administration will be selected in order to achieve the desired combined therapeutic effect.

The administration of the combinations of the invention may be advantageous over the individual therapeutic agents in that the combinations may provide one or more of the following improved properties when compared to the individual administration of a single therapeutic agent alone: i) a greater anticancer effect than the most active single agent, ii) synergistic or highly synergistic anticancer activity, iii) a dosing protocol that provides enhanced anticancer activity with reduced side effect profile, iv) a reduction in the toxic effect profile, v) an increase in the therapeutic window, and/or vi) an increase in the bioavailability of one or both of the therapeutic agents.

In one embodiment, each antigen binding protein in a combination is individually formulated into its own pharmaceutical composition and each of the pharmaceutical compositions are administered to treat cancer. In this embodiment, each of the pharmaceutical compositions may have the same or different carriers, diluents or excipients. For example, in one embodiment, a first pharmaceutical composition contains an ICOS binding protein, a second pharmaceutical composition contains an anti-PD-(L)1(IgG):TGFβR fusion protein, and the first and second pharmaceutical compositions are both administered to treat cancer.

In one embodiment, each binding protein in the combination is formulated together into a single pharmaceutical composition and administered to treat cancer. For example, in one embodiment, a single pharmaceutical composition contains both an ICOS binding protein and an anti-PD-(L)1(IgG):TGFβR fusion protein and is administered as a single pharmaceutical composition to treat cancer.

Antigen Binding Proteins and Antibodies that Bind ICOS Agents directed to ICOS in any of the aspects or embodiments of the present invention include a monoclonal antibody (mAb), or antigen binding fragment thereof, that specifically binds to ICOS. In some embodiments, the mAb to ICOS specifically binds to human ICOS. In one embodiment, the ICOS binding protein is a monoclonal antibody or antigen binding fragment thereof. The mAb may be a human antibody, a humanized antibody or a chimeric antibody, and may include a human constant region. The human constant region is selected from the group consisting of IgG1, IgG2, IgG3 and IgG4 constant regions, and in preferred embodiments, the human constant region is an IgG1 or IgG4 constant region. The antigen binding fragment may be selected from the group consisting of Fab, Fab′-SH, F(ab′)₂, scFv and Fv fragments.

As used herein “ICOS” means any Inducible T-cell costimulator protein. Pseudonyms for ICOS (Inducible T-cell COStimulator) include AILIM; CD278; CVID1, JTT-1 or JTT-2, MGC39850, or 8F4. ICOS is a CD28-superfamily costimulatory molecule that is expressed on activated T cells. The protein encoded by this gene belongs to the CD28 and CTLA-4 cell-surface receptor family. It forms homodimers and plays an important role in cell-cell signaling, immune responses, and regulation of cell proliferation. The amino acid sequence of human ICOS (isoform 2) (Accession No.: UniProtKB—Q9Y6W8-2) is shown below as SEQ ID NO:11.

(SEQ ID NO: 11)
MKSGLWYFFLFCLRIKVLTGEINGSANYEMFIFHNGGVQILCKYPDIVQQ
FKMQLLKGGQILCDLTKTKGSGNTVSIKSLKFCHSQLSNNSVSFFLYNLD
HSHANYYFCNLSIFDPPPFKVTLTGGYLHIYESQLCCQLKFWLPIGCAAF
VVVCILGCILICWLTKKM

The amino acid sequence of human ICOS (isoform 1) (Accession No.: UniProtKB—Q9Y6W8-1) is shown below as SEQ ID NO:12.

(SEQ ID NO: 12)
MKSGLWYFFLFCLRIKVLTGEINGSANYEMFIFHNGGVQILCKYPDIVQQ
FKMQLLKGGQILCDLTKTKGSGNTVSIKSLKFCHSQLSNNSVSFFLYNLD
HSHANYYFCNLSIFDPPPFKVTLTGGYLHIYESQLCCQLKFWLPIGCAAF
VVVCILGCILICWLTKKKYSSSVHDPNGEYMFMRAVNTAKKSRLTDVTL

Activation of ICOS occurs through binding by ICOS-L (B7RP-1/B7-H2). Neither B7-1 nor B7-2 (ligands for CD28 and CTLA4) bind or activate ICOS. However, ICOS-L has been shown to bind weakly to both CD28 and CTLA-4 (Yao et al. “B7-H2 is a costimulatory ligand for CD28 in human”, Immunity, 34(5); 729-40 (2011)). Expression of ICOS appears to be restricted to T cells. ICOS expression levels vary between different T cell subsets and on T cell activation status. ICOS expression has been shown on resting TH17, T follicular helper (TFH) and regulatory T (Treg) cells; however, unlike CD28; it is not highly expressed on naïve TH1 and TH2 effector T cell populations (Paulos et al. “The inducible costimulator (ICOS) is critical for the development of human Th17 cells”, Sci Transl Med, 2(55); 55ra78 (2010)). ICOS expression is highly induced on CD4+ and CD8+ effector T cells following activation through TCR engagement (Wakamatsu et al. “Convergent and divergent effects of costimulatory molecules in conventional and regulatory CD4+ T cells”, Proc Natl Acad Sci USA, 110(3); 1023-8 (2013)). Co-stimulatory signalling through ICOS receptor only occurs in T cells receiving a concurrent TCR activation signal (Sharpe A H and Freeman G J. “The B7-CD28 Superfamily”, Nat. Rev Immunol, 2(2); 116-26 (2002)). In activated antigen specific T cells, ICOS regulates the production of both TH1 and TH2 cytokines including IFN-γ, TNF-α, IL-10, IL-4, IL-13 and others. ICOS also stimulates effector T cell proliferation, albeit to a lesser extent than CD28 (Sharpe A H and Freeman G J. “The B7-CD28 Superfamily”, Nat. Rev Immunol, 2(2); 116-26 (2002)).

By “agent directed to ICOS” is meant any chemical compound or biological molecule capable of binding to ICOS. In some embodiments, the agent directed to ICOS is an ICOS binding protein. In some other embodiments, the agent directed to ICOS is an ICOS agonist. In some embodiments, the ICOS binding protein is an agonist ICOS binding protein.

The term “ICOS binding protein” as used herein refers to a protein that binds to ICOS, including an antibody or an antigen binding fragment thereof, or engineered molecules that function in similar ways to antibodies that are capable of binding to ICOS. In one embodiment, the antibody is a monoclonal antibody. In some instances, the ICOS is human ICOS. The term “ICOS binding protein” can be used interchangeably with “ICOS binding agent”, “ICOS antigen binding protein” or “ICOS antigen binding agent”. Thus, as is understood in the art, anti-ICOS antibodies and/or ICOS antigen binding proteins would be considered ICOS binding proteins. This definition does not include the natural cognate ligand or receptor. References to ICOS binding proteins, in particular anti-ICOS antibodies, includes antigen binding portions or fragments thereof. As used herein “antigen binding portion” of an ICOS binding protein includes any portion of the ICOS binding protein capable of binding to ICOS, including but not limited to, an antigen binding antibody fragment.

In one embodiment, the ICOS binding proteins of the present invention comprise any one or a combination of the following CDRs:

(SEQ ID NO: 1)
CDRH1: DYAMH
(SEQ ID NO: 2)
CDRH2: LISIYSDHTNYNQKFQG
(SEQ ID NO: 3)
CDRH3: NNYGNYGWYFDV
(SEQ ID NO: 4)
CDRL1: SASSSVSYMH
(SEQ ID NO: 5)
CDRL2: DTSKLAS
(SEQ ID NO: 6)
CDRL3: FQGSGYPYT

In one embodiment, the ICOS binding protein comprises a heavy chain variable region CDR1 (“CDRH1”) comprising an amino acid sequence with one or two amino acid variation(s) (“CDR variant”) to the amino acid sequence set forth in SEQ ID NO:1.

In one embodiment, the ICOS binding protein comprises a heavy chain variable region CDR2 (“CDRH2”) comprising an amino acid sequence with five or fewer, such as four or fewer, three or fewer, two or fewer, or one amino acid variation(s) (“CDR variant”) to the amino acid sequence set forth in SEQ ID NO:2. In a further embodiment, the CDRH2 comprises an amino acid sequence with one or two amino acid variation(s) to the amino acid sequence set forth in SEQ ID NO:2.

In one embodiment, the ICOS binding protein comprises a heavy chain variable region CDR3 (“CDRH3”) comprising an amino acid sequence with one or two amino acid variation(s) (“CDR variant”) to the amino acid sequence set forth in SEQ ID NO:3.

In one embodiment, the ICOS binding protein comprises a light chain variable region CDR1 (“CDRL1”) comprising an amino acid sequence with three or fewer, such as one or two amino acid variation(s) (“CDR variant”) to the amino acid sequence set forth in SEQ ID NO:4.

In one embodiment, the ICOS binding protein comprises a light chain variable region CDR2 (“CDRL2”) comprising an amino acid sequence with one or two amino acid variation(s) (“CDR variant”) to the amino acid sequence set forth in SEQ ID NO:5.

In one embodiment, the ICOS binding protein comprises a light chain variable region CDR3 (“CDRL3”) comprising an amino acid sequence with three or fewer, such as one or two amino acid variation(s) (“CDR variant”) to the amino acid sequence set forth in SEQ ID NO:6.

In one embodiment, the ICOS binding protein comprises a CDRH1 comprising an amino acid sequence with up to one amino acid variation to the amino acid sequence set forth in SEQ ID NO:1; a CDRH2 comprising an amino acid sequence with up to five amino acid variations to the amino acid sequence set forth in SEQ ID NO:2; a CDRH3 comprising an amino acid sequence with up to one amino acid variation to the amino acid sequence set forth in SEQ ID NO:3; a CDRL1 comprising an amino acid sequence with up to three amino acid variations to the amino acid sequence set forth in SEQ ID NO:4; a CDRL2 comprising an amino acid sequence with up to one amino acid variation to the amino acid sequence set forth in SEQ ID NO:5; and/or a CDRL3 comprising an amino acid sequence with up to three amino acid variations to the amino acid sequence set forth in SEQ ID NO:6.

In one embodiment of the present invention the ICOS binding protein comprises CDRH1 (SEQ ID NO:1), CDRH2 (SEQ ID NO:2), and CDRH3 (SEQ ID NO:3) in the heavy chain variable region having the amino acid sequence set forth in SEQ ID NO:7. ICOS binding proteins of the present invention comprising the humanized heavy chain variable region set forth in SEQ ID NO:7 are designated as “H2.” In some embodiments, the anti-ICOS antibodies of the present invention comprise a heavy chain variable region having at least 90% sequence identity to SEQ ID NO:7. Suitably, the ICOS binding proteins of the present invention may comprise a heavy chain variable region having about 85%, 86%, 87%, 88%, 89%, 90%, 91%, 92%, 93%, 94%, 95%, 96%, 97%, 98%, 99%, or 100% sequence identity to SEQ ID NO:7.

Humanized heavy chain (V_{H}) variable region (H2):
QVQLVQSGAEVKKPGSSVKVSCKASGYTFTDYAMHWVRQAPGQGLEWMGL
ISIYSDHTNYNQKFQGRVTITADKSTSTAYMELSSLRSEDTAVYYCGRNN
YGNYGWYFDVWGQGTTVTVSS (SEQ ID NO: 7; underlined
amino acid residues correspond to the positions of
CDRs).

In one embodiment, the ICOS binding protein comprises a heavy chain variable region (“V_{H}”) comprising an amino acid sequence with at least about 90%, 91%, 92%, 93%, 94%, 95%, 96%, 97%, 98%, 99% or 100% sequence identity to the amino acid sequence set forth in SEQ ID NO:7. In one embodiment, the V_{H} comprises an amino acid sequence with at least one amino acid variation to the amino acid sequence set forth in SEQ ID NO:7, such as between 1 and 5, such as between 1 and 3, in particular up to 2 amino acid variations to the amino acid sequence set forth in SEQ ID NO:7.

In one embodiment of the present invention the ICOS binding protein comprises CDRL1 (SEQ ID NO:4), CDRL2 (SEQ ID NO:5), and CDRL3 (SEQ ID NO:6) in the light chain variable region having the amino acid sequence set forth in SEQ ID NO:8. ICOS binding proteins of the present invention comprising the humanized light chain variable region set forth in SEQ ID NO:8 are designated as “L5.” Thus, an ICOS binding protein of the present invention comprising the heavy chain variable region of SEQ ID NO:7 and the light chain variable region of SEQ ID NO:8 can be designated as H2L5 herein.

In some embodiments, the ICOS binding proteins of the present invention comprise a light chain variable region having at least 90% sequence identity to the amino acid sequence set forth in SEQ ID NO:8. Suitably, the ICOS binding proteins of the present invention may comprise a light chain variable region having about 85%, 86%, 87%, 88%, 89%, 90%, 91%, 92%, 93%, 94%, 95%, 96%, 97%, 98%, 99%, or 100% sequence identity to SEQ ID NO:8.

Humanized light chain (V_{L}) variable region (L5):
EIVLTQSPATLSLSPGERATLSCSASSSVSYMHWYQQKPGQAPRLLIYDT
SKLASGIPARFSGSGSGTDYTLTISSLEPEDFAVYYCFQGSGYPYTFGQG
TKLEIK (SEQ ID NO: 8; underlined amino acid
residues correspond to the positions of CDRs).

In one embodiment, the ICOS binding protein comprises a light chain variable region (“V_{L}”) comprising an amino acid sequence with at least about 90%, 91%, 92%, 93%, 94%, 95%, 96%, 97%, 98%, 99% or 100% sequence identity to the amino acid sequence set forth in SEQ ID NO:8. In one embodiment, the V_{L} comprises an amino acid sequence with at least one amino acid variation to the amino acid sequence set forth in SEQ ID NO:8, such as between 1 and 5, such as between 1 and 3, in particular up to 2 amino acid variations to the amino acid sequence set forth in SEQ ID NO:8.

In one embodiment, the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the ICOS binding protein comprises a V_{H} with the amino acid sequence set forth in SEQ ID NO:7; and a V_{L} with the amino acid sequence set forth in SEQ ID NO:8.

In one embodiment, the ICOS binding protein comprises a V_{H} comprising an amino acid sequence of SEQ ID NO:7 and a V_{L} comprising an amino acid sequence of SEQ ID NO:8 In one embodiment, the ICOS binding protein comprises a V_{H} comprising an amino acid sequence with at least about 90%, 91%, 92%, 93%, 94%, 95%, 96%, 97%, 98%, 99% or 100% sequence identity to the amino acid sequence set forth in SEQ ID NO:7; and a V_{L} comprising an amino acid sequence with at least about 90%, 91%, 92%, 93%, 94%, 95%, 96%, 97%, 98%, 99% or 100% sequence identity to the amino acid sequence set forth in SEQ ID NO:8.

In one embodiment, the ICOS binding protein is a humanized monoclonal antibody comprising a heavy chain (HC) amino acid sequence having at least 90%, 91%, 92,%, 93%, 94%, 95%, 96%, 97%, 98%, 99% or 100% sequence identity to the amino acid sequence set forth in SEQ ID NO:9.

(SEQ ID NO: 9)
QVQLVQSGAEVKKPGSSVKVSCKASGYTFTDYAMHWVRQAPGQGLEWMGL
ISIYSDHTNYNQKFQGRVTITADKSTSTAYMELSSLRSEDTAVYYCGRNN
YGNYGWYFDVWGQGTTVTVSSASTKGPSVFPLAPCSRSTSESTAALGCLV
KDYFPEPVTVSWNSGALTSGVHTFPAVLQSSGLYSLSSVVTVPSSSLGTK
TYTCNVDHKPSNTKVDKRVESKYGPPCPPCPAPEFEGGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK

In one embodiment, the HC comprises an amino acid sequence with at least one amino acid variation to the amino acid sequence set forth in SEQ ID NO:9, such as between 1 and 10, such as between 1 and 7, in particular up to 6 amino acid variations to the amino acid sequence set forth in SEQ ID NO:9. In a further embodiment, the HC comprises one, two, three, four, five, six or seven amino acid variations to the amino acid sequence set forth in SEQ ID NO:9.

In one embodiment, the ICOS binding protein is a humanized monoclonal antibody comprising a light chain (LC) amino acid sequence having at least 90%, 91%, 92,%, 93%, 94%, 95%, 96%, 97%, 98%, 99% or 100% sequence identity to the amino acid sequence set forth in SEQ ID NO:10.

(SEQ ID NO: 10)
EIVLTQSPATLSLSPGERATLSCSASSSVSYMHWYQQKPGQAPRLLIYDT
SKLASGIPARFSGSGSGTDYTLTISSLEPEDFAVYYCFQGSGYPYTFGQG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

In one embodiment, the LC comprises an amino acid sequence with at least one amino acid variation to the amino acid sequence set forth in SEQ ID NO:10, such as between 1 and 10, such as between 1 and 5, in particular up to 3 amino acid variations to the amino acid sequence set forth in SEQ ID NO:10. In a further embodiment, the LC comprises one, two or three amino acid variations to the amino acid sequence set forth in SEQ ID NO:10.

In one embodiment, the ICOS binding protein comprises a HC comprising an amino acid sequence with at least about 90%, 91%, 92%, 93%, 94%, 95%, 96%, 97%, 98%, 99% or 100% sequence identity to the amino acid sequence set forth in SEQ ID NO:9; and a LC comprising an amino acid sequence with at least about 90%, 91%, 92%, 93%, 94%, 95%, 96%, 97%, 98%, 99% or 100% sequence identity to the amino acid sequence set forth in SEQ ID NO:10. Therefore, the antibody is an antibody with a heavy chain at least about 90% identical to the heavy chain amino acid sequence of SEQ ID NO:9 and/or with a light chain at least about 90% identical to the light chain amino acid sequence of SEQ ID NO:10.

In one embodiment, the ICOS binding protein comprises a heavy chain amino acid sequence at least about 90% identical to the amino acid sequence of SEQ ID NO:9 and/or a light chain amino acid sequence at least about 90% identical to the amino acid sequence of SEQ ID NO:10.

In one embodiment, the ICOS binding protein comprises a heavy chain sequence of SEQ ID NO:9 and a light chain sequence of SEQ ID NO:10.

In one embodiment there is provided an ICOS binding protein comprising a heavy chain constant region such that has reduced ADCC and/or complement activation or effector functionality.

In one such embodiment the heavy chain constant region may comprise a naturally disabled constant region of IgG2 or IgG4 isotype or a mutated IgG1 constant region.

In one embodiment, the ICOS binding protein comprises an IgG4 Fc region comprising the amino acid substitutions S228P and L235E or functional equivalents thereof. In one embodiment, the ICOS binding protein comprises an IgG4 Fc region comprising the replacement S229P and L236E. Such embodiments may have the designation IgG4PE. Thus, an ICOS binding protein having the heavy chain variable region H2 and the light chain variable region L5 and an IgG4PE Fc region will be designated as H2L5 IgG4PE or synonymously as H2L5 hIgG4PE.

In one embodiment, the ICOS binding protein is feladilimab. In one embodiment, the ICOS binding protein is H2L5. In one embodiment, the ICOS binding protein is H2L5 hIgG4PE. H2L5 hIgG4PE comprises CDR sequences as set out in SEQ ID NOS: 1-6, variable heavy chain and variable light chain sequences as set out in SEQ ID NO:7 and SEQ ID NO: 8, respectively, and heavy chain and light chain sequences as set out in SEQ ID NO:9 and SEQ ID NO:10, respectively.

Antibodies to ICOS and methods of using in the treatment of disease are described, for instance, in WO2012/131004, US2011/0243929, and US2016/0215059. US2016/0215059 is incorporated by reference herein. CDRs for murine antibodies to human ICOS having agonist activity are shown in PCT/EP2012/055735 (WO2012/131004). Antibodies to ICOS are also disclosed in WO2008/137915, WO2010/056804, EP1374902, EP1374901, and EP1125585. Agonist antibodies to ICOS or ICOS binding proteins are disclosed in WO2012/13004, WO2014/033327, WO2016/120789, US2016/0215059, and US2016/0304610. Exemplary antibodies in US2016/0304610 include 37A10S713. Sequences of 37A10S713 are reproduced below as SEQ ID NOS:31-38.

37A10S713 V_{H} CDR1: GFTFSDYWMD (SEQ ID NO: 31)
37A10S713 V_{H} CDR2: NIDEDGSITEYSPFVKG (SEQ ID NO:
32)
37A10S713 V_{H} CDR3: WGRFGFDS (SEQ ID NO: 33)
37A10S713 V_{L} CDR1: KSSQSLLSGSFNYLT (SEQ ID NO:
34)
37A10S713 V_{L} CDR2: YASTRHT (SEQ ID NO: 35)
37A10S713 V_{L} CDR3: HHHYNAPPT (SEQ ID NO: 36)
37A10S713 heavy chain variable region:
EVQLVESGGLVQPGGSLRLSCAASGFTFSDYWMDWVRQAPGKGLVWVSNI
DEDGSITEYSPFVKGRFTISRDNAKNTLYLQMNSLRAEDTAVYYCTRWGR
FGFDSWGQGTLVTVSS (SEQ ID NO: 37; underlined amino
acid residues correspond to the positions of CDRs)
37A10S713 light chain variable region:
DIVMTQSPDSLAVSLGERATINCKSSQSLLSGSFNYLTWYQQKPGQPPKL
LIFYASTRHTGVPDRFSGSGSGTDFTLTISSLQAEDVAVYYCHHHYNAPP
TFGPGTKVDIK (SEQ ID NO: 38; underlined amino acid
residues correspond to the positions of CDRs)

In an embodiment, the ICOS binding protein is vopratelimab. In one embodiment, the ICOS binding protein is JTX-2011.

Exemplary antibodies in US2018/0289790 include ICOS.33 IgG1f S267E. Sequences of ICOS.33 IgG1f S267E are reproduced below as SEQ ID NOS:39-40:

ICOS.33 IgG1f S267E heavy chain variable domain:
(SEQ ID NO: 39)
EVQLVESGGGLVKPGGSLRLSCAASGFTFSDYFMHWVRQAPGKGLEWVGV
IDTKSFNYATYYSDLVKGRFTISRDDSKNTLYLQMNSLKTEDTAVYYCTA
TIAVPYYFDYWGQGTLVTVSS
ICOS.33 IgG1f S267E light chain variable domain:
(SEQ ID NO: 40)
DIQMTQSPSSLSASVGDRVTITCQASQDISNYLSWYQQKPGKAPKLLIYY
TNLLAEGVPSRHSGSGSGTDFTFTISSLQPEDIATYYCQQYYNYRTFGPG
TKVDIK

In one embodiment, the ICOS binding protein is BMS-986226.

Exemplary antibodies in WO2018/029474 include STIM003. Sequences of STIM003 are reproduced below as SEQ ID NOS: 41-42.

STIM003 heavy chain variable domain:
(SEQ ID NO: 41)
EVQLVESGGGVVRPGGSLRLSCVASGVTFDDYGMSWVRQAPGKGLEWVSG
INWNGGDTDYSDSVKGRFTISRDNAKNSLYLQMNSLRAEDTALYYCARDF
YGSGSYYHVPFDYWGQGILVTVSS
STIM003 light chain variable domain:
(SEQ ID NO: 42)
EIVLTQSPGTLSLSPGERATLSCRASQSVSRSYLAWYQQKRGQAPRLLIY
GASSRATGIPDRFSGDGSGTDFTLSISRLEPEDFAVYYCHQYDMSPFTFG
PGTKVDIK

In one embodiment, the ICOS binding protein is KY-1044.

Exemplary antibodies in WO2018/045110 include XENP23104. Sequences of the ICOS binding Fab side ([ICOS]_H0.66_L0) of XENP23104 are reproduced below as SEQ ID NOS: 43-50.

XENP23104 [ICOS]_H0.66_L0 heavy chain variable
domain:
QVQLVQSGAEVKKPGASVKVSCKASGYTFTGYYMHWVRQAPGQGLEWMGW
INPHSGETIYAQKFQGRVTMTRDTSISTAYMELSSLRSEDTAVYYCARTY
YYDTSGYYHDAFDVWGQGTMVTVSS (SEQ ID NO: 43; under-
lined amino acid residues correspond to the
positions of CDRs).
XENP23104 [ICOS]_H0.66_L0 V_{H} CDR1: GYYMH (SEQ ID
NO: 44)
XENP23104 [ICOS]_H0.66_L0 V_{H} CDR2:
WINPHSGETIYAQKFQG (SEQ ID NO: 45)
XENP23104 [ICOS]_H0.66_L0 V_{H} CDR3:
TYYYDTSGYYHDAFDV (SEQ ID NO: 46)
XENP23104 [ICOS]_H0.66_L0 light chain variable
domain:
DIQMTQSPSSVSASVGDRVTITCRASQGISRLLAWYQQKPGKAPKLLIYV
ASSLQSGVPSRFSGSGSGTDFTLTISSLQPEDFATYYCQQANSFPWTFGQ
GTKVEIK (SEQ ID NO: 47; underlined amino acid
residues correspond to the positions of CDRs).
XENP23104 [ICOS]_H0.66_L0 V_{L} CDR1: RASQGISRLLA
(SEQ ID NO: 48)
XENP23104 [ICOS]_H0.66_L0 V_{L} CDR2: VASSLQS (SEQ ID
NO: 49)
XENP23104 [ICOS]_H0.66_L0 V_{L} CDR3: QQANSFPWT (SEQ
ID NO: 50)

As used herein “ICOS-L” and “ICOS Ligand” are used interchangeably and refer to the membrane bound natural ligand of human ICOS. ICOS ligand is a protein that in humans is encoded by the ICOSLG gene. ICOSLG has also been designated as CD275 (cluster of differentiation 275). Pseudonyms for ICOS-L include B7RP-1 and B7-H2.

### IgG:TGFβR Fusion Proteins

The present invention is directed to a combination including a polypeptide comprising a PD-1 inhibitor and a TGFβR, such as an anti-PD-(L)1(IgG):TGFβR fusion protein, preferably an anti-PD-L1(IgG1):TGFβRII fusion protein or an anti-PD-1 (IgG1):TGFβRII fusion protein.

The present invention features a combination including a polypeptide which comprises a PD-1 inhibitor (e.g. an antibody, or an antigen-binding fragment thereof, that binds PD-1 or PD-L1) and a TGFβR, or a fragment thereof capable of binding TGF-β (e.g. human TGFβRII or a fragment thereof capable of binding TGF-β, such as a soluble fragment).

Thus, the present invention features a combination including a fusion protein which comprises (a) human TGFβRII, or a fragment thereof capable of binding TGFβ (e.g. a soluble fragment); and (b) an antibody, or an antigen-binding fragment thereof, that binds PD-L1 (e.g. any of the PD-L1 antibodies or antibody fragments described herein).

The polypeptides and fusion proteins in any of the aspects or embodiments of the present invention preferably include a soluble cytokine receptor, TGFβR, tethered to a PD-1 inhibitor (including a PD-1 binding protein). In some embodiments, the TGFβR is TGFβRII (also referred to as TGFβR2).

In some embodiments, the PD-1 inhibitor is a PD-L1 binding protein (including a monoclonal antibody (mAb), or antigen binding fragment thereof, which specifically binds to PD-L1). In other embodiments, the PD-1 inhibitor is a PD-1 binding protein (including a monoclonal antibody (mAb), or antigen binding fragment thereof, which specifically binds to PD-1). In one embodiment, the PD-L1 or PD-1 binding protein is a monoclonal antibody or antigen binding fragment thereof. In some embodiments, the mAb specifically binds to human PD-L1 or PD-1. The mAb may be a human antibody, a humanized antibody or a chimeric antibody, and may include a human constant region. The human constant region is selected from the group consisting of IgG1, IgG2, IgG3 and IgG4 constant regions, and in preferred embodiments, the human constant region is an IgG1 or IgG4 constant region. In a further embodiment, the PD-L1 or PD-1 binding protein is an immunoglobulin G4 (IgG4) monoclonal antibody, in particular an IgG4 humanized monoclonal antibody. The antigen binding fragment may be selected from the group consisting of Fab, Fab′-SH, F(ab′)₂, scFv and Fv fragments.

The programmed death I (PD-1)/PD-L1 axis is an important mechanism for tumor immune evasion Effector T cells chronically sensing antigen take on an exhausted phenotype marked by PD-1 expression, a state under which tumor cells engage by upregulating PD-L1. Additionally, in the tumor microenvironment, myeloid cells, macrophages. parenchymal cells and T cells upregulate PD-1. Blocking the axis restores the effector function in these T cells. The anti-PD-L1/TGFβ trap also binds TGF-β (1, 2 and 3 isoforms), which is an inhibitory cytokine produced in the tumor microenvironment by cells including apoptotic neutrophils, myeloid-derived suppressor cells, T cells and tumor cells. Inhibition of TGF-β by soluble TGFβRII reduced malignant mesothelioma in a manner that was associated with increases in CD8+ T cell anti-tumor effects. The absence of TGF-β1 produced by activated CD4+ T cells and Treg cells has been shown to inhibit tumor growth, and protect mice from spontaneous cancer. Thus, TGF-β appears to be important for tumor immune evasion.

TGF-β has growth inhibitory effects on normal epithelial cells, functioning as a regulator of epithelial cell homeostasis, and it acts as a tumor suppressor during early carcinogenesis. As tumors progress toward malignancy, the growth inhibitory effects of TGF-β on the tumor are lost via mutation in one or more TGF-β pathway signaling components or through oncogenic reprogramming. Upon loss of sensitivity to TGF-β inhibition, the tumor continues to produce high levels of TGF-β, which then serve to promote tumor growth. The TGF-β cytokine is overexpressed in various cancer types with correlation to tumor stage. Many types of cells in the tumor microenvironment produce TGF-β including the tumor cells themselves, immature myeloid cells, regulatory T cells, and stromal fibroblasts; these cells collectively generate a large reservoir of TGF-β in the extracellular matrix. TGF-β signaling contributes to tumor progression by promoting metastasis, stimulating angiogenesis, and suppressing innate and adaptive anti-tumor immunity. As a broadly immunosuppressive factor, TGF-β directly down-regulates the effector function of activated cytotoxic T cells and NK cells and potently induces the differentiation of naïve CD4+ T cells to the immunosuppressive regulatory T cells (Treg) phenotype. In addition, TGF-β polarizes macrophages and neutrophils to a wound-healing phenotype that is associated with production of immunosuppressive cytokines. As a therapeutic strategy, neutralization of TGF-β activity has the potential to control tumor growth by restoring effective anti-tumor immunity, blocking metastasis, and inhibiting angiogenesis.

Combining these pathways, PD-1 or PD-L1, and TGF-β, is attractive as an anti-tumor approach. Concomitant PD-1 and TGF-β blockade can restore pro-inflammatory cytokines. Anti-PD-L1/TGFβ trap includes, for example, an extracellular domain of the human TGF-β receptor TGFβRII covalently joined via a glycine/serine linker to the C terminus of each heavy chain of the fully human IgG1 anti-PD-L1 antibody. Given the emerging picture for PD-1/PD-L1 class, in which responses are apparent, but with room for increase in effect size, it is envisaged that co-targeting a complementary immune modulation step will improve tumor response. A similar TGF-targeting agent, fresolimumab, which is a monoclonal antibody targeting TGF-β-1, 2 and 3, showed initial evidence of tumor response in a Phase I trial in subjects with melanoma.

As used herein, an “agent directed to PD-L1” or “agent directed to PDL1” means any chemical compound or biological molecule capable of binding to PD-L1. In some embodiments, the agent directed to PD-L1 is a PD-L1 binding protein.

The term “PD-L1 binding protein” or “PDL1 binding protein” as used herein refers to antibodies and other protein constructs, such as domains, which are capable of binding to PD-L1. In some instances, the PD-L1 is human PD-L1. The term “PD-L1 binding protein” can be used interchangeably with “PD-L1 binding agent”, “PD-L1 antigen binding protein” or “PD-L1 antigen binding agent”. Thus, as is understood in the art, anti-PD-L1 antibodies and/or PD-L1 antigen binding proteins would be considered PD-L1 binding proteins. This definition does not include the natural cognate ligand. References to PD-L1 binding proteins includes antigen binding portions or fragments thereof. As used herein “antigen binding portion” of a PD-L1 binding protein would include any portion of the PD-L1 binding protein capable of binding to PD-L1, including but not limited to, an antigen binding antibody fragment.

As used herein, an “agent directed to PD-1” or “agent directed to PD1” means any chemical compound or biological molecule capable of binding to PD-1. In some embodiments, the agent directed to PD-1 is a PDL1 binding protein.

The term “PD-1 binding protein” or “PD1 binding protein” as used herein refers to antibodies and other protein constructs, such as domains that are capable of binding to PD-1. In some instances, the PD-1 is human PD-1. The term “PD-1 binding protein” can be used interchangeably with “PD-1 binding agent”, “PD-1 antigen binding protein” or “PD-1 antigen binding agent”. Thus, as is understood in the art, anti-PD-1 antibodies and/or PD-1 antigen binding proteins would be considered PD-1 binding proteins. This definition does not include the natural cognate ligand. References to PD-1 binding proteins includes antigen binding portions or fragments thereof. As used herein “antigen binding portion” of a PD-1 binding protein would include any portion of the PD-1 binding protein capable of binding to PD-1, including but not limited to, an antigen binding antibody fragment.

In one embodiment, the PD-L1 binding proteins of the present invention comprise any one or a combination of the following CDRs:

(SEQ ID NO: 13)
CDRH1: SYIMM
(SEQ ID NO: 14)
CDRH2: SIYPSGGITFYADTVKG
(SEQ ID NO: 15)
CDRH3: IKLGTVTTVDY
(SEQ ID NO: 16)
CDRL1: TGTSSDVGGYNYVS
(SEQ ID NO: 17)
CDRL2: DVSNRPS
(SEQ ID NO: 18)
CDRL3: SSYTSSSTRV

In one embodiment, the PD-L1 binding protein comprises a heavy chain variable region CDR1 (“CDRH1”) comprising an amino acid sequence with one or two amino acid variation(s) (“CDR variant”) to the amino acid sequence set forth in SEQ ID NO:13.

In one embodiment, the PD-L1 binding protein comprises a heavy chain variable region CDR2 (“CDRH2”) comprising an amino acid sequence with five or fewer, such as four or fewer, three or fewer, two or fewer, or one amino acid variation(s) (“CDR variant”) to the amino acid sequence set forth in SEQ ID NO:14. In a further embodiment, the CDRH2 comprises an amino acid sequence with one or two amino acid variation(s) to the amino acid sequence set forth in SEQ ID NO:14.

In one embodiment, the PD-L1 binding protein comprises a heavy chain variable region CDR3 (“CDRH3”) comprising an amino acid sequence with one or two amino acid variation(s) (“CDR variant”) to the amino acid sequence set forth in SEQ ID NO:15.

In one embodiment, the PD-L1 binding protein comprises a light chain variable region CDR1 (“CDRL1”) comprising an amino acid sequence with three or fewer, such as one or two amino acid variation(s) (“CDR variant”) to the amino acid sequence set forth in SEQ ID NO:16.

In one embodiment, the PD-L1 binding protein comprises a light chain variable region CDR2 (“CDRL2”) comprising an amino acid sequence with one or two amino acid variation(s) (“CDR variant”) to the amino acid sequence set forth in SEQ ID NO:17.

In one embodiment, the PD-L1 binding protein comprises a light chain variable region CDR3 (“CDRL3”) comprising an amino acid sequence with three or fewer, such as one or two amino acid variation(s) (“CDR variant”) to the amino acid sequence set forth in SEQ ID NO:18. In a particular embodiment, the CDRL3 comprises an amino acid sequence with one amino acid variation to the amino acid sequence set forth in SEQ ID NO:18.

In one embodiment, the PD-L1 binding protein comprises a CDRH1 comprising an amino acid sequence with up to one amino acid variation to the amino acid sequence set forth in SEQ ID NO:13; a CDRH2 comprising an amino acid sequence with up to five amino acid variations to the amino acid sequence set forth in SEQ ID NO:14; a CDRH3 comprising an amino acid sequence with up to one amino acid variation to the amino acid sequence set forth in SEQ ID NO:15; a CDRL1 comprising an amino acid sequence with up to three amino acid variations to the amino acid sequence set forth in SEQ ID NO:16; a CDRL2 comprising an amino acid sequence with up to one amino acid variation to the amino acid sequence set forth in SEQ ID NO:17; and/or a CDRL3 comprising an amino acid sequence with up to three amino acid variations to the amino acid sequence set forth in SEQ ID NO:18.

In one embodiment of the present invention the PD-L1 binding protein comprises CDRH1 (SEQ ID NO:13), CDRH2 (SEQ ID NO:14), and CDRH3 (SEQ ID NO:15) in the heavy chain variable region having the amino acid sequence set forth in SEQ ID NO:19. In some embodiments, the PD-L1 binding proteins of the present invention comprise a heavy chain variable region having at least 90% sequence identity to SEQ ID NO:19. Suitably, the PD-L1 binding proteins of the present invention may comprise a heavy chain variable region having about 85%, 86%, 87%, 88%, 89%, 90%, 91%, 92%, 93%, 94%, 95%, 96%, 97%, 98%, 99%, or 100% sequence identity to SEQ ID NO:19.

PD-L1 binding protein heavy chain (V_{H}) variable
region:
(SEQ ID NO: 19)
EVQLLESGGGLVQPGGSLRLSCAASGFTFSSYIMMWVRQAPGKGLEWVSS
IYPSGGITFYADTVKGRFTISRDNSKNTLYLQMNSLRAEDTAVYYCARIK
LGTVTTVDYWGQGTLVTVSS

In one embodiment, the PD-L1 binding protein comprises a heavy chain variable region (“V_{H}”) comprising an amino acid sequence with at least about 90%, 91%, 92%, 93%, 94%, 95%, 96%, 97%, 98%, 99% or 100% sequence identity to the amino acid sequence set forth in SEQ ID NO:19 In one embodiment, the V_{H} comprises an amino acid sequence with at least one amino acid variation to the amino acid sequence set forth in SEQ ID NO:19, such as between 1 and 5, such as between 1 and 3, in particular up to 2 amino acid variations to the amino acid sequence set forth in SEQ ID NO:19.

In one embodiment of the present invention the PD-L1 binding protein comprises CDRL1 (SEQ ID NO:16), CDRL2 (SEQ ID NO:17), and CDRL3 (SEQ ID NO:18) in the light chain variable region having the amino acid sequence set forth in SEQ ID NO:20. In one embodiment, a PD-L1 binding protein of the present invention comprises the heavy chain variable region of SEQ ID NO:19 and the light chain variable region of SEQ ID NO:20.

In some embodiments, the PD-L1 binding proteins of the present invention comprise a light chain variable region having at least 90% sequence identity to the amino acid sequence set forth in SEQ ID NO:20. Suitably, the PD-L1 binding proteins of the present invention may comprise a light chain variable region having about 85%, 86%, 87%, 88%, 89%, 90%, 91%, 92%, 93%, 94%, 95%, 96%, 97%, 98%, 99%, or 100% sequence identity to SEQ ID NO:20.

PD-L1 binding protein light chain (V_{L}) variable
region:
(SEQ ID NO: 20)
QSALTQPASVSGSPGQSITISCTGTSSDVGGYNYVSWYQQHPGKAPKLMI
YDVSNRPSGVSNRFSGSKSGNTASLTISGLQAEDEADYYCSSYTSSSTRV
FGTGTKVTVL

In one embodiment, the PD-L1 binding protein comprises a light chain variable region (“V_{L}”) comprising an amino acid sequence with at least about 90%, 91%, 92%, 93%, 94%, 95%, 96%, 97%, 98%, 99% or 100% sequence identity to the amino acid sequence set forth in SEQ ID NO:20. In one embodiment, the V_{L} comprises an amino acid sequence with at least one amino acid variation to the amino acid sequence set forth in SEQ ID NO:20, such as between 1 and 5, such as between 1 and 3, in particular up to 2 amino acid variations to the amino acid sequence set forth in SEQ ID NO:20.

In one embodiment, the PD-L1 binding protein comprises a V_{H} comprising an amino acid sequence with at least about 90%, 91%, 92%, 93%, 94%, 95%, 96%, 97%, 98%, 99% or 100% sequence identity to the amino acid sequence set forth in SEQ ID NO:19; and a V_{L} comprising an amino acid sequence with at least about 90%, 91%, 92%, 93%, 94%, 95%, 96%, 97%, 98%, 99% or 100% sequence identity to the amino acid sequence set forth in SEQ ID NO:20. In one embodiment, the PD-L1 binding protein comprises a V_{H} at least about 90% identical to the amino acid sequence of SEQ ID NO:19 and/or a V_{L} at least about 90% identical to the amino acid sequence of SEQ ID NO:20.

In one embodiment, a PD-L1 binding protein comprises a V_{H} with the amino acid sequence set forth in SEQ ID NO:19, and a V_{L} with the amino acid sequence set forth in SEQ ID NO:20.

In one embodiment, the PD-L1 binding protein is a monoclonal antibody comprising a heavy chain (HC) amino acid sequence having at least 90%, 91%, 92,%, 93%, 94%, 95%, 96%, 97%, 98%, 99% or 100% sequence identity to the amino acid sequence set forth in SEQ ID NO:21.

(SEQ ID NO: 21)
EVQLLESGGGLVQPGGSLRLSCAASGFTFSSYIMMWVRQAPGKGLEWVSS
IYPSGGITFYADTVKGRFTISRDNSKNTLYLQMNSLRAEDTAVYYCARIK
LGTVTTVDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG

In one embodiment, the HC comprises an amino acid sequence with at least one amino acid variation to the amino acid sequence set forth in SEQ ID NO:21, such as between 1 and 10, such as between 1 and 7, in particular up to 6 amino acid variations to the amino acid sequence set forth in SEQ ID NO:21. In a further embodiment, the HC comprises one, two, three, four, five, six or seven amino acid variations to the amino acid sequence set forth in SEQ ID NO:21.

In one embodiment, the PD-L1 binding protein is a monoclonal antibody comprising a light chain (LC) amino acid sequence having at least 90%, 91%, 92,%, 93%, 94%, 95%, 96%, 97%, 98%, 99% or 100% sequence identity to the amino acid sequence set forth in SEQ ID NO:22.

(SEQ ID NO: 22)
QSALTQPASVSGSPGQSITISCTGTSSDVGGYNYVSWYQQHPGKAPKLMI
YDVSNRPSGVSNRFSGSKSGNTASLTISGLQAEDEADYYCSSYTSSSTRV
FGTGTKVTVLGQPKANPTVTLFPPSSEELQANKATLVCLISDFYPGAVTV
AWKADGSPVKAGVETTKPSKQSNNKYAASSYLSLTPEQWKSHRSYSCQVT
HEGSTVEKTVAPTECS

In one embodiment, the LC comprises an amino acid sequence with at least one amino acid variation to the amino acid sequence set forth in SEQ ID NO:22, such as between 1 and 10, such as between 1 and 5, in particular up to 3 amino acid variations to the amino acid sequence set forth in SEQ ID NO:22. In a further embodiment, the LC comprises one, two or three amino acid variations to the amino acid sequence set forth in SEQ ID NO:22.

In one embodiment, the PD-L1 binding protein comprises a HC comprising an amino acid sequence with at least about 90%, 91%, 92%, 93%, 94%, 95%, 96%, 97%, 98%, 99% or 100% sequence identity to the amino acid sequence set forth in SEQ ID NO:21; and a LC comprising an amino acid sequence with at least about 90%, 91%, 92%, 93%, 94%, 95%, 96%, 97%, 98%, 99% or 100% sequence identity to the amino acid sequence set forth in SEQ ID NO:22. Therefore, the antibody is an antibody with a heavy chain at least about 90% identical to the heavy chain amino acid sequence of SEQ ID NO:21 and/or with a light chain at least about 90% identical to the light chain amino acid sequence of SEQ ID NO:22.

In one embodiment, the PD-L1 binding protein comprises a heavy chain amino acid sequence at least about 90% identical to the amino acid sequence of SEQ ID NO:21 and/or a light chain amino acid sequence at least about 90% identical to the amino acid sequence of SEQ ID NO:22.

In one embodiment, the PD-L1 binding protein comprises a heavy chain sequence of SEQ ID NO:21 and a light chain sequence of SEQ ID NO:22.

Thus, in some embodiments, the PD-1 inhibitor is a PD-L1 binding protein, such as an anti-PD-L1 antibody.

PD-L1 is a B7 family member that is expressed on many cell types, including APCs and activated T cells (Yamazaki et al. (2002) J. Immunol. 169:5538). PD-L1 binds to both PD-1 and B7-1. Both binding of T-cell-expressed B7-1 by PD-L1 and binding of T-cell-expressed PD-L1 by B7-1 result in T cell inhibition (Butte et al. (2007) Immunity 27:111). There is also evidence that, like other B7 family members, PD-L1 can also provide costimulatory signals to T cells (Subudhi et al. (2004) J. Clin. Invest. 113:694; Tamura et al. (2001) Blood 97:1809). PD-L1 (human PD-L1 cDNA is composed of the base sequence shown by EMBL/GenBank Acc. No. AF233516 and mouse PD-L1 cDNA is composed of the base sequence shown by NM.sub.-021893) that is a ligand of PD-1 is expressed in so-called antigen-presenting cells (APCs) such as activated monocytes and dendritic cells (Journal of Experimental Medicine (2000), vol. 19, issue 7, p 1027-1034). These cells present interaction molecules that induce a variety of immuno-inductive signals to T lymphocytes, and PD-L1 is one of these molecules that induce the inhibitory signal by PD-1. It has been revealed that PD-L1 ligand stimulation suppressed the activation (cellular proliferation and induction of various cytokine production) of PD-1 expressing T lymphocytes. PD-L1 expression has been confirmed in not only immunocompetent cells but also a certain kind of tumor cell lines (cell lines derived from monocytic leukemia, cell lines derived from mast cells, cell lines derived from hepatic carcinomas, cell lines derived from neuroblasts, and cell lines derived from breast carcinomas) (Nature Immunology (2001), vol. 2, issue 3, p. 261-267).

Anti-PD-L1 antibodies and methods of making the same are known in the art. Such antibodies to PD-L1 may be polyclonal or monoclonal, and/or recombinant, and/or humanized, and/or fully human. PD-L1 antibodies are in development as immuno-modulatory agents for the treatment of cancer.

PD-L1 antibodies are disclosed in U.S. Pat. Nos. 9,212,224; 8,779,108; 8,552,154; 8,383,796; and 8,217,149; US Patent Publication No. 2011/02808707, WO2013/079174 and WO2013/019906. Additional exemplary antibodies to PD-L1 (also referred to as CD274 or B7-H1) and methods for use are disclosed in U.S. Pat. Nos. 8,168,179; 7,943,743; 7,595,048; and WO2014/055897, WO2013/019906 and WO2010/077634. Specific anti-human PD-L1 monoclonal antibodies useful in the treatment method, medicaments and uses of the present invention include MPDL3280A, BMS-936559, MED14736, MSB0010718C.

Atezolizumab is a fully humanized monoclonal anti-PD-L1 antibody commercially available as TECENTRIQ. Atezolizumab is indicated for the treatment of some locally advanced or metastatic urothelial carcinomas. Atezolizumab blocks the interaction of PD-L1 with PD-1 and CD80. Avelumab is an anti-PD-L1 antibody commercially available as BAVENCIO.

Durvalumab (previously known as MEDI4736) is a human monoclonal antibody directed against PD-L1. Durvalumab blocks the interaction of PD-L1 with PD-1 and CD80. Durvalumab is commercially available as IMFINZI.

Antibodies to PD-L1 (also referred to as CD274 or B7-H1) and methods for use are disclosed in U.S. Pat. Nos. 7,943,743; 8,383,796; 8,168,179; and 7,595,048; US2013/0034559 and WO2014/055897. PD-L1 antibodies are in development as immuno-modulatory agents for the treatment of cancer.

Further exemplary anti-PD-L1 antibodies that can be used in fusion proteins are described in US patent application publication US 2010/0203056. In one embodiment, the PD-L1 binding protein is MPDL3280A. Sequences of MPDL3280A are reproduced below as SEQ ID NOS: 27-28.

MPDL3280A heavy chain variable domain:
(SEQ ID NO: 27)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS
MPDL3280A light chain variable domain:
(SEQ ID NO: 28)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR

In one embodiment, the PD-L1 binding protein is YW243.55S70. The sequence of the heavy chain variable domain of YW243.55S70 is reproduced below as SEQ ID NO: 29:

YW243.55S70 heavy chain variable domain:
(SEQ ID NO: 29)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSA

The TGFβR of the IgG:TGFβR fusion protein or the anti-PD-(L)1(IgG):TGFβR fusion protein is preferably TGFβRI or TGFβRII, more preferably it is TGFβRII. In some embodiments, it is an IgG:TGFβRII fusion protein or an anti-PD-(L)1(IgG):TGFβR fusion protein, respectively, wherein the IgG has a pI of 8.5-9.5 whereas the TGFβRII has a pI of 4.6-5.4. In some embodiments, it is an anti-PD-L1(IgG):TGFβRII fusion protein, such as an anti-PD-L1 (IgG1):TGFβRII or an anti-PD-L1(IgG4):TGFβRII. Most preferably, it is an anti-PD-L1(IgG1):TGFβRII.

The TGFβRII may be a soluble extracellular domain of TGFβRII or a fragment thereof that is capable of binding TGF-β. Preferably, the TGFβRII lacks the cytoplasmic domain of TGFβRII. In some embodiments, the TGFβRII corresponds to the wild-type human TGF-β Receptor Type 2 Isoform A sequence (e.g. the amino acid sequence of NCBI Reference Sequence (RefSeq) Accession No. NP_001020018 (SEQ ID NO:24)), or the wild-type human TGF-β Receptor Type 2 Isoform B sequence (e.g., the amino acid sequence of NCBI RefSeq Accession No. NP_003233 (SEQ ID NO:25)).

Preferably, the TGFβRII comprises or consists of a sequence corresponding to SEQ ID NO: 26 or a fragment thereof capable of binding TGF-β. For instance, the TGFβRII may correspond to the full-length sequence of SEQ ID NO: 26:

(SEQ ID NO: 26)
IPPHVQKSVNNDMIVTDNNGAVKFPQLCKFCDVRFSTCDNQKSCMSNCSI
TSICEKPQEVCVAVWRKNDENITLETVCHDPKLPYHDFILEDAASPKCIM
KEKKKPGETFFMCSCSSDECNDNIIFSEEYNTSNPD

Alternatively, it may have an N-terminal deletion. For instance, amino acids 1-26 of the N-terminus of SEQ ID NO:26 may be deleted, such as 14-21 or 14-26 of the most N-terminal amino acids. In some embodiments, the N-terminal 14, 19 or 21 amino acids of SEQ ID NO:26 are deleted.

Preferably, the TGFβRII has at least 80% sequence identity, at least 90% sequence identity, or at least 95% sequence identity to the amino acid sequence of SEQ ID NO:26.

In some embodiments, the TGFβRII has an amino acid sequence that does not differ in more than 25 amino acids from SEQ ID NO:26.

In some embodiments, the TGFβR of the anti-PD-(L)1(IgG):TGFβR fusion protein has greater than or equal to 90% sequence identity, such as greater than or equal to 92% sequence identity, greater than or equal to 95% sequence identity, greater than or equal to 99% sequence identity, or 100% sequence identity with the amino acid sequence of the TGFβR of bintrafusp alfa. Preferably, the TGFβR of the anti-PD-(L)1(IgG):TGFβR fusion protein has an amino acid sequence with not more than 50, not more than 40, or not more than 25 amino acid residues different from the TGFβR of bintrafusp alfa. The TGFβR of the anti-PD-(L)1(IgG):TGFβR fusion protein preferably has between 100-160 amino acid residues, more preferably 110-140 amino acid residues. In some embodiments, the amino acid sequence of the TGFβR is selected from the group consisting of a sequence corresponding to positions 1-136 of the TGFβR of bintrafusp alfa, a sequence corresponding to positions 20-136 of the TGFβR of bintrafusp alfa and a sequence corresponding to positions 22-136 of the TGFβR of bintrafusp alfa.

In some embodiments, the TGFβR of the anti-PD-(L)1(IgG):TGFβR fusion protein has greater than or equal to 98% sequence identity with the amino acid sequence of the TGFβR of bintrafusp alfa, and the CH3 domain of the anti-PD-(L)1(IgG):TGFβR fusion protein has greater than or equal to 92% sequence identity with the amino acid sequence of the CH3 domain of bintrafusp alfa. In some embodiments, the TGFβR of the anti-PD-(L)1(IgG):TGFβR fusion protein has not more than 25 amino acid residues different from the amino acid sequence of the TGFβR of bintrafusp alfa, and the CH3 domain of the anti-PD-(L)1(IgG):TGFβR fusion protein has not more than 4 amino acid residues different from the amino acid sequence of the CH3 domain of bintrafusp alfa.

In some embodiments, the anti-PD-(L)1(IgG):TGFβR fusion protein comprises a linker between the IgG and TGFβR, which linker preferably comprises between 5 and 50 amino acid residues, between 10 and 30 amino acid residues, or between 20 and 27 amino acid residues. Preferably such a linker comprises at most two different types of amino acid residue. In some embodiments, the linker comprises glycine amino acid residues and/or serine amino acid residues. In some embodiments, such a linker is defined by the formula (GlyₓSer)_{y}Gly, where x is an integer between 1 and 6, and y is an integer between 2 and 7. Preferably x is 4. Preferably y is 4 or 5. In some embodiments, the linker is defined by the formula (GlyₓSer)_{y}Gly, wherein x is 4 and y is either 4 or 5. In some embodiments, the linker comprises the amino acid sequence of SEQ ID NO:30.

The anti-PD-(L)1(IgG):TGFβR fusion protein is preferably an anti-PD-(L)1(IgG):TGFβRII fusion protein comprising a TGFβRII fused at the N-terminus thereof to the C-terminus of an IgG antibody, optionally via a linker. Therefore, in one embodiment, the fusion protein comprises a HC amino acid sequence having at least 90%, 91%, 92,%, 93%, 94%, 95%, 96%, 97%, 98%, 99% or 100% sequence identity to the amino acid sequence set forth in SEQ ID NO:23.

(SEQ ID NO: 23)
EVQLLESGGGLVQPGGSLRLSCAASGFTFSSYIMMWVRQAPGKGLEWVSS
IYPSGGITFYADTVKGRFTISRDNSKNTLYLQMNSLRAEDTAVYYCARIK
LGTVTTVDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGA
GGGGSGGGGSGGGGSGGGGSGIPPHVQKSVNNDMIVTDNNGAVKFPQLCK
FCDVRFSTCDNQKSCMSNCSITSICEKPQEVCVAVWRKNDENITLETVCH
DPKLPYHDFILEDAASPKCIMKEKKKPGETFFMCSCSSDECNDNIIFSEE
YNTSNPD

Thus, in one embodiment, the fusion protein may comprise the amino acid sequence of SEQ ID NO:23.

In one embodiment, the HC comprises an amino acid sequence with at least one amino acid variation to the amino acid sequence set forth in SEQ ID NO:23, such as between 1 and 10, such as between 1 and 7, in particular up to 6 amino acid variations to the amino acid sequence set forth in SEQ ID NO:23. In a further embodiment, the HC comprises one, two, three, four, five, six or seven amino acid variations to the amino acid sequence set forth in SEQ ID NO:23.

In one embodiment, the fusion protein comprises a HC comprising an amino acid sequence with at least about 90%, 91%, 92%, 93%, 94%, 95%, 96%, 97%, 98%, 99% or 100% sequence identity to the amino acid sequence set forth in SEQ ID NO:23; and a LC comprising an amino acid sequence with at least about 90%, 91%, 92%, 93%, 94%, 95%, 96%, 97%, 98%, 99% or 100% sequence identity to the amino acid sequence set forth in SEQ ID NO:22.

In one embodiment, the fusion protein comprises a heavy chain amino acid sequence at least about 90% identical to the amino acid sequence of SEQ ID NO:23 and/or a light chain amino acid sequence at least about 90% identical to the amino acid sequence of SEQ ID NO:22.

In one embodiment, the fusion protein comprises a heavy chain sequence of SEQ ID NO:23 and a light chain sequence of SEQ ID NO:22.

The fusion protein may comprise the HC and LC described herein which, when combined with the HC, forms an antigen-binding site that binds PD-L1. Therefore, in one embodiment, the fusion protein may include (a) two polypeptides, each having an amino acid sequence consisting of the amino acid sequence of SEQ ID NO:23 (i.e. two HC), and (b) two additional polypeptides each having an amino acid sequence consisting of the amino acid sequence of SEQ ID NO:22 (i.e. two LC).

In some embodiments, the IgG:TGFβR fusion protein is one of the IgG:TGFβR fusion proteins disclosed in WO 2015/118175 or WO 2018/205985. For instance, the IgG:TGFβR fusion protein may comprise the light chains and heavy chains of SEQ ID NO: 1 and SEQ ID NO: 3 of WO 2015/118175, respectively. In another embodiment, the IgG:TGFβR fusion protein is one of the constructs listed in Table 2 of WO 2018/205985, such as construct 9 or 15 thereof.

In one embodiment, the IgG:TGFβR fusion protein is characterised by:
a TGFβR having greater than or equal to 95% sequence identity with the amino acid sequence of the TGFβR of bintrafusp alfa;
a CH3 domain having greater than or equal to 92% sequence identity with the amino acid sequence of the CH3 domain of bintrafusp alfa;
a CH1 domain having greater than or equal to 90% sequence identity with the amino acid sequence of the CH1 domain of bintrafusp alfa; and
a CH2 domain having greater than or equal to 90% sequence identity with the amino acid sequence of the CH2 domain of bintrafusp alfa.

In one embodiment, the IgG:TGFβR fusion protein is characterised by:
a TGFβR having not more than 25 amino acid residues different from the amino acid sequence of the TGFβR of bintrafusp alfa;
a CH3 domain having not more than 4 amino acid residues different from the amino acid sequence of the CH3 domain of bintrafusp alfa;
a CH1 domain having not more than 7 amino acid residues different from the amino acid sequence of the CH1 domain of bintrafusp alfa; and
a CH2 domain having not more than 8 amino acid residues different from the amino acid sequence of the CH2 domain of bintrafusp alfa.

### Methods of Treatment

The inhibitors and antigen binding proteins described herein may also be used in methods of treatment. It will be appreciated by those skilled in the art that references herein to treatment refer to the treatment of established conditions. However, compositions of the invention may, depending on the condition, also be useful in the prevention of certain diseases. The inhibitors and antigen binding proteins described herein can be used in an effective amount for therapeutic, prophylactic or preventative treatment. A therapeutically effective amount of the inhibitors and antigen binding proteins described herein is an amount effective to ameliorate or reduce one or more symptoms of, or to prevent or cure, the disease.

In one aspect, there is provided a method of treating cancer in a human in need thereof, the method comprising administering to the human an ICOS binding protein. In another aspect, there is provided an ICOS binding protein for use in treating cancer. In a further aspect, there is provided use of an ICOS binding protein in the manufacture of a medicament for treating cancer. There is disclosed a pharmaceutical kit comprising an ICOS binding protein.

In one aspect, there is provided a method of treating cancer in a human in need thereof, the method comprising administering to the human a PD-1 inhibitor. In another aspect, there is provided a PD-1 inhibitor for use in treating cancer. In a further aspect, there is provided use of a PD-1 inhibitor in the manufacture of a medicament for treating cancer. There is disclosed a pharmaceutical kit comprising a PD-1 inhibitor.

In one aspect, there is provided a method of treating cancer in a human in need thereof, the method comprising administering to the human a TGF-β inhibitor. In another aspect, there is provided a TGF-β inhibitor for use in treating cancer. In a further aspect, there is provided use of a TGF-β inhibitor in the manufacture of a medicament for treating cancer. There is disclosed a pharmaceutical kit comprising a TGF-β inhibitor.

In one aspect, there is provided a method of treating cancer in a human in need thereof, the method comprising administering to the human a polypeptide comprising a PD-1 inhibitor and a TGFβR. In another aspect, there is provided a polypeptide comprising a PD-1 inhibitor and a TGFβR for use in treating cancer. In a further aspect, there is provided use of a polypeptide comprising a PD-1 inhibitor and a TGFβR in the manufacture of a medicament for treating cancer. There is disclosed a pharmaceutical kit comprising a polypeptide comprising a PD-1 inhibitor and a TGFβR.

In a further aspect, there is provided a method of treating cancer in a human in need thereof, the method comprising administering to the human an anti-PD-(L)1(IgG):TGFβR fusion protein. In another aspect, there is provided an anti-PD-(L)1(IgG):TGFβR fusion protein for use in treating cancer. In a further aspect, there is provided use of an anti-PD-(L)1(IgG):TGFβR fusion protein in the manufacture of a medicament for treating cancer. There is disclosed a pharmaceutical kit comprising an anti-PD-(L)1(IgG):TGFβR fusion protein.

In one embodiment, the inhibitors/polypeptides/fusion protein/binding proteins are administered simultaneously/concurrently. In an alternative embodiment, the inhibitors/polypeptides/fusion protein/binding proteins are administered sequentially (e.g. a first regimen administered prior to administration of any doses of a second regimen).

In one aspect, there is provided a method of treating cancer in a human in need thereof, the method comprising administering to the human an ICOS binding protein and a polypeptide comprising a PD-1 inhibitor and a TGFβR. In a further aspect, there is provided an ICOS binding protein and a polypeptide comprising a PD-1 inhibitor and a TGFβR for concurrent or sequential use in treating cancer. In another aspect, there is provided an ICOS binding protein for use in treating cancer is provided, wherein the ICOS binding protein is to be administered concurrently or sequentially with a polypeptide comprising a PD-1 inhibitor and a TGFβR. In one aspect, there is provided the use of an ICOS binding protein in the manufacture of a medicament for treating cancer, wherein the ICOS binding protein is to be administered concurrently or sequentially with a polypeptide comprising a PD-1 inhibitor and a TGFβR. In another aspect, there is provided a pharmaceutical kit comprising an ICOS binding protein and a polypeptide comprising a PD-1 inhibitor and a TGFβR.

In one aspect, there is provided a method of treating cancer in a human in need thereof, the method comprising administering to the human an ICOS binding protein and an anti-PD-(L)1(IgG):TGFβR fusion protein. In a further aspect, there is provided an ICOS binding protein and an anti-PD-(L)1(IgG):TGFβR fusion protein for concurrent or sequential use in treating cancer. In another aspect, there is provided an ICOS binding protein for use in treating cancer is provided, wherein the ICOS binding protein is to be administered concurrently or sequentially with an anti-PD-(L)1(IgG):TGFβR fusion protein. In one aspect, there is provided the use of an ICOS binding protein in the manufacture of a medicament for treating cancer, wherein the ICOS binding protein is to be administered concurrently or sequentially with an anti-PD-(L)1(IgG):TGFβR fusion protein. In another aspect, there is provided a pharmaceutical kit comprising an ICOS binding protein and an anti-PD-(L)1(IgG):TGFβR fusion protein.

In one embodiment the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the ICOS binding protein comprises one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:4; CDRL2 as set forth in SEQ ID NO:5 and/or CDRL3 as set forth in SEQ ID NO:6 or a direct equivalent of each CDR wherein a direct equivalent has no more than two amino acid substitutions in said CDR. In one embodiment, the ICOS binding protein comprises a heavy chain variable region comprising one or more of SEQ ID NO:1; SEQ ID NO:2; and SEQ ID NO:3 and wherein said ICOS binding protein comprises a light chain variable region comprising one or more of SEQ ID NO:4; SEQ ID NO:5, and SEQ ID NO:6. In one embodiment, the ICOS binding protein comprises a heavy chain variable region comprising SEQ ID NO:1; SEQ ID NO:2; and SEQ ID NO:3 and wherein said ICOS binding protein comprises a light chain variable region comprising SEQ ID NO:4; SEQ ID NO:5, and SEQ ID NO:6. In one embodiment, the ICOS binding protein comprises a V_{H} domain comprising the amino acid sequence set forth in SEQ ID NO:7 and a V_{L} domain comprising the amino acid sequence as set forth in SEQ ID NO:8. In one embodiment, the ICOS binding protein comprises a heavy chain comprising the amino acid sequence set forth in SEQ ID NO:9 and a light chain comprising the amino acid sequence as set forth in SEQ ID NO:10.

In one embodiment the PD-1 inhibitor is a PD-L1 binding protein. In one embodiment, the PD-L1 binding protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20 wherein said PD-L1 binding protein specifically binds to human PD-L1. In one embodiment, the PD-L1 binding protein comprises one or more of: CDRH1 as set forth in SEQ ID NO:13; CDRH2 as set forth in SEQ ID NO:14; CDRH3 as set forth in SEQ ID NO:15; CDRL1 as set forth in SEQ ID NO:16; CDRL2 as set forth in SEQ ID NO:17 and/or CDRL3 as set forth in SEQ ID NO:18 or a direct equivalent of each CDR wherein a direct equivalent has no more than two amino acid substitutions in said CDR. In one embodiment, the PD-L1 binding protein comprises a heavy chain variable region comprising one or more of SEQ ID NO:13; SEQ ID NO:14; and SEQ ID NO:15 and wherein said PD-L1 binding protein comprises a light chain variable region comprising one or more of SEQ ID NO:16; SEQ ID NO:17, and SEQ ID NO:18. In one embodiment, the PD-L1 binding protein comprises a heavy chain variable region comprising SEQ ID NO:13; SEQ ID NO:14; and SEQ ID NO:15 and wherein said PD-L1 binding protein comprises a light chain variable region comprising SEQ ID NO:16; SEQ ID NO:17, and SEQ ID NO:18.

In one embodiment, the PD-L1 binding protein comprises a V_{H} domain comprising the amino acid sequence set forth in SEQ ID NO:19 and a V_{L} domain comprising the amino acid sequence as set forth in SEQ ID NO:20. In one embodiment, the PD-L1 binding protein comprises a heavy chain comprising the amino acid sequence set forth in SEQ ID NO:21 and a light chain comprising the amino acid sequence as set forth in SEQ ID NO:22.

In one embodiment the anti-PD-L1(IgG):TGFβR fusion protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20 wherein said anti-PD-L1(IgG):TGFβR fusion protein specifically binds to human PD-L1. In one embodiment, the anti-PD-L1(IgG):TGFβR fusion protein comprises one or more of: CDRH1 as set forth in SEQ ID NO:13; CDRH2 as set forth in SEQ ID NO:14; CDRH3 as set forth in SEQ ID NO:15; CDRL1 as set forth in SEQ ID NO:16; CDRL2 as set forth in SEQ ID NO:17 and/or CDRL3 as set forth in SEQ ID NO:18 or a direct equivalent of each CDR wherein a direct equivalent has no more than two amino acid substitutions in said CDR. In one embodiment, the anti-PD-L1(IgG):TGFβR fusion protein comprises a heavy chain variable region comprising one or more of SEQ ID NO:13; SEQ ID NO:14; and SEQ ID NO:15 and wherein said anti-PD-L1(IgG):TGFβR fusion protein comprises a light chain variable region comprising one or more of SEQ ID NO:16; SEQ ID NO:17, and SEQ ID NO:18. In one embodiment, the anti-PD-L1(IgG):TGFβR fusion protein comprises a heavy chain variable region comprising SEQ ID NO:13; SEQ ID NO:14; and SEQ ID NO:15 and wherein said anti-PD-L1(IgG):TGFβR fusion protein comprises a light chain variable region comprising SEQ ID NO:16; SEQ ID NO:17, and SEQ ID NO:18. In one embodiment, the anti-PD-L1(IgG):TGFβR fusion protein comprises a V_{H} domain comprising the amino acid sequence set forth in SEQ ID NO:19 and a V_{L} domain comprising the amino acid sequence as set forth in SEQ ID NO:20.

In one embodiment, the anti-PD-L1(IgG):TGFβR fusion protein comprises a heavy chain comprising the amino acid sequence set forth in SEQ ID NO:21 and a light chain comprising the amino acid sequence as set forth in SEQ ID NO:22. In one embodiment, the anti-PD-L1(IgG):TGFβR fusion protein comprises human TGFβRII, or a fragment thereof capable of binding to TGF-β. In a further embodiment, the anti-PD-L1(IgG):TGFβR fusion protein comprises a heavy chain comprising the amino acid sequence set forth in SEQ ID NO:23 and a light chain comprising the amino acid sequence as set forth in SEQ ID NO:22.

### Dosage

In one aspect, the method comprises administering a therapeutically effective amount of a combination as described herein (e.g. comprising an ICOS binding protein and a polypeptide comprising a PD-1 inhibitor and a TGFβR, or comprising an ICOS binding protein and an anti-PD-(L)1(IgG):TGFβR fusion protein) to a subject in need thereof.

In some embodiments, a therapeutically effective dose of the ICOS binding protein is a dose of about 0.01-1000 mg (e.g. a dose about 0.01 mg; a dose about 0.08 mg; a dose about 0.1 mg; a dose about 0.24 mg; a dose about 0.8 mg; a dose about 1 mg; a dose about 2.4 mg; a dose about 7.2 mg; a dose about 8 mg; a dose about 10 mg; a dose about 20 mg; a dose about 24 mg; a dose about 30 mg; a dose about 40 mg; a dose about 48 mg; a dose about 50 mg; a dose about 60 mg; a dose about 70 mg; a dose about 72 mg; a dose about 80 mg; a dose about 90 mg; a dose about 100 mg; a dose about 160 mg; a dose about 200 mg; a dose about 240 mg; a dose about 300 mg; a dose about 320 mg; a dose about 400 mg; a dose about 480 mg; a dose about 500 mg; a dose about 600 mg; a dose about 700 mg; a dose about 720 mg; a dose about 800 mg; a dose about 900 mg; or a dose about 1000 mg;

In some embodiments, a therapeutically effective dose of the ICOS binding protein is a dose of about 0.001 mg/kg to about 10 mg/kg. In some embodiments, a therapeutically effective dose is about 0.001 mg/kg. In some embodiments, a therapeutically effective dose is about 0.003 mg/kg. In some embodiments, a therapeutically effective dose is about 0.01 mg/kg. In some embodiments, a therapeutically effective dose is about 0.03 mg/kg. In some embodiments, a therapeutically effective dose is about 0.1 mg/kg. In some embodiments, a therapeutically effective dose is about 0.3 mg/kg. In some embodiments, a therapeutically effective dose is about 0.6 mg/kg. In some embodiments, a therapeutically effective dose is about 1 mg/kg. In some embodiments, a therapeutically effective dose is about 2 mg/kg. In some embodiments, a therapeutically effective dose is about 3 mg/kg. In some embodiments, a therapeutically effective dose is about 4 mg/kg; about 5 mg/kg; about 6 mg/kg; about 7 mg/kg; about 8 mg/kg; about 9 mg/kg or 10 mg/kg. In some embodiments, a therapeutically effective dose is a dose about 500 mg. In some embodiments, a therapeutically effective dose is about 800 mg. In some embodiments, a therapeutically effective dose is about 1000 mg.

In some embodiments, a therapeutically effective dose of a polypeptide comprising a PD-1 inhibitor and a TGFβR is a dose of about 0.01-3000 mg (e.g. a dose about 0.01 mg; a dose about 0.08 mg; a dose about 0.1 mg; a dose about 0.24 mg; a dose about 0.8 mg; a dose about 1 mg; a dose about 2.4 mg; a dose about 8 mg; a dose about 10 mg; a dose about 20 mg; a dose about 24 mg; a dose about 30 mg; a dose about 40 mg; a dose about 48 mg; a dose about 50 mg; a dose about 60 mg; a dose about 70 mg; a dose about 80 mg; a dose about 90 mg; a dose about 100 mg; a dose about 160 mg; a dose about 200 mg; a dose about 240 mg; a dose about 300 mg; a dose about 400 mg; a dose about 500 mg; a dose about 600 mg; a dose about 700 mg; a dose about 800 mg; a dose about 900 mg; a dose about 1000 mg; a dose about 1100 mg; a dose about 1200 mg; a dose about 1300 mg; a dose about 1400 mg; a dose about 1500 mg; a dose about 1600 mg; a dose about 1700 mg; a dose about 1800 mg; a dose about 1900 mg; a dose about 2000 mg; a dose about 2100 mg; a dose about 2200 mg; a dose about 2300 mg; a dose about 2400 mg; a dose about 2500 mg; a dose about 2600 mg; a dose about 2700 mg; a dose about 2800 mg; a dose about 2900 mg; or a dose about 3000 mg). In some embodiments, a therapeutically effective dose is about 0.001 mg/kg. In some embodiments, a therapeutically effective does is about 0.003 mg/kg. In some embodiments, a therapeutically effective dose is about 0.01 mg/kg. In some embodiments, a therapeutically effective dose is about 0.03 mg/kg. In some embodiments, a therapeutically effective dose is about 0.1 mg/kg. In some embodiments, a therapeutically effective dose is about 0.3 mg/kg. In some embodiments, a therapeutically effective dose is about 1 mg/kg. In some embodiment, a therapeutically effective dose is about 2 mg/kg. In some embodiments, a therapeutically effective dose is about 3 mg/kg. In some embodiments, a therapeutically effective dose is about 10 mg/kg. In some embodiments, a therapeutically effective dose is about 30 mg/kg. In some embodiments, a therapeutically effective dose is a dose about 500 mg. In some embodiments, a therapeutically effective dose is about 1200 mg. In some embodiments, a therapeutically effective dose is about 2400 mg.

In some embodiments, a therapeutically effective dose of an anti-PD-(L)1(IgG):TGFβR is a dose of about 0.01-3000 mg (e.g. a dose about 0.01 mg; a dose about 0.08 mg; a dose about 0.1 mg; a dose about 0.24 mg; a dose about 0.8 mg; a dose about 1 mg; a dose about 2.4 mg; a dose about 8 mg; a dose about 10 mg; a dose about 20 mg; a dose about 24 mg; a dose about 30 mg; a dose about 40 mg; a dose about 48 mg; a dose about 50 mg; a dose about 60 mg; a dose about 70 mg; a dose about 80 mg; a dose about 90 mg; a dose about 100 mg; a dose about 160 mg; a dose about 200 mg; a dose about 240 mg; a dose about 300 mg; a dose about 400 mg; a dose about 500 mg; a dose about 600 mg; a dose about 700 mg; a dose about 800 mg; a dose about 900 mg; a dose about 1000 mg; a dose about 1100 mg; a dose about 1200 mg; a dose about 1300 mg; a dose about 1400 mg; a dose about 1500 mg; a dose about 1600 mg; a dose about 1700 mg; a dose about 1800 mg; a dose about 1900 mg; a dose about 2000 mg; a dose about 2100 mg; a dose about 2200 mg; a dose about 2300 mg; a dose about 2400 mg; a dose about 2500 mg; a dose about 2600 mg; a dose about 2700 mg; a dose about 2800 mg; a dose about 2900 mg; or a dose about 3000 mg). In some embodiments, a therapeutically effective dose is about 0.001 mg/kg. In some embodiments, a therapeutically effective does is about 0.003 mg/kg. In some embodiments, a therapeutically effective dose is about 0.01 mg/kg. In some embodiments, a therapeutically effective dose is about 0.03 mg/kg. In some embodiments, a therapeutically effective dose is about 0.1 mg/kg. In some embodiments, a therapeutically effective dose is about 0.3 mg/kg. In some embodiments, a therapeutically effective dose is about 1 mg/kg. In some embodiment, a therapeutically effective dose is about 2 mg/kg. In some embodiments, a therapeutically effective dose is about 3 mg/kg. In some embodiments, a therapeutically effective dose is about 10 mg/kg. In some embodiments, a therapeutically effective dose is about 30 mg/kg. In some embodiments, a therapeutically effective dose is a dose about 500 mg. In some embodiments, a therapeutically effective dose is about 1200 mg. In some embodiments, a therapeutically effective dose is about 2400 mg.

In one embodiment, the combination is administered once every 2-6 weeks (e.g. 2, 3 or 4 weeks, in particular 3 weeks). In one embodiment, the combination is administered for once every 2 weeks. In one embodiment, the combination is administered for once every 3 weeks. In one embodiment, the combination is administered for once every 6 weeks. In one embodiment, the combination is administered for once every 3 weeks for 2-6 dosing cycles (e.g. the first 3, 4, or 5 dosing cycles, in particular, the first 4 dosing cycles).

If desired, the effective daily dose of a (therapeutic) combination may be administered as two, three, four, five, six or more doses administered separately at appropriate intervals throughout the day, optionally, in unit dosage forms. Pharmaceutical formulations may be presented in unit dose forms containing a predetermined amount of active ingredient per unit dose. As is known to those skilled in the art, the amount of active ingredient per dose will depend on the condition being treated, the route of administration and the age, weight and condition of the patient. It is especially advantageous to formulate parenteral compositions in dosage unit form for ease of administration and uniformity of dosage. Dosage unit form as used herein refers to physically discrete units suited as unitary dosages for the mammalian subjects, in particular human subjects, to be treated; each unit containing a predetermined quantity of active compound calculated to produce the desired therapeutic effect in association with the required pharmaceutical carrier.

The present disclosure provides methods of treating cancer comprising administering to a patient in need of treatment one or both (or more) of the inhibitors/binding proteins/polypeptides/fusion proteins in the combination at a first dose at a first interval for a first period; and administering to the patient one or both (or more) of the binding proteins in the combination at a second dose at a second interval for a second period. There may be a rest period between the first and second periods in which one or both (or more) of the inhibitors/binding proteins/polypeptides/fusion proteins in the combination is/are not administered to the patient. In some embodiments, there is a rest period between the first period and second period. In some embodiments, the rest period is between 1 day and 30 days. In some embodiments, the rest period is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days. In some embodiments, the rest period is 1 week, 2 weeks, 3 weeks, 4 weeks, 5 weeks, 6 weeks, 7 weeks, 9 weeks, 10 weeks, 11 weeks, 12 weeks, 13 weeks, 14 weeks or 15 weeks.

With respect to the PD-1 inhibitor/TGF-βinhibitor/polypeptide comprising a PD-1 inhibitor and a TGFβR/anti-PD-(L)1(IgG):TGFβR, in some embodiments, the first dose and second dose are the same. In some embodiments, the first dose and the second dose are 1200 mg. In some embodiments, the first dose and the second dose are 2400 mg. In some embodiments, the first dose and second dose are different. In some embodiments, the first dose is about 1200 mg and the second dose is 2400 mg. In some embodiments, the first dose is about 2400 mg and the second dose is 1200 mg.

In some embodiments, the first interval and second interval are the same. In some embodiments, the first interval and the second interval are once every two weeks. In some embodiments, the first interval and the second interval are once every three weeks. In some embodiments, the first interval and the second interval are once every six weeks. In some embodiments, the first interval and the second interval are different. In some embodiments, the first interval is once every two weeks and the second interval is once every three weeks. In some embodiments, the first interval is once every three weeks and the second interval is once every six weeks.

With respect to the ICOS binding protein (e.g. an anti-ICOS antibody, an agonist anti-ICOS antibody, H2L5, H2L5 hIgG4PE, or feladilimab). in some embodiments, the first interval and the second interval are the same. In some embodiments, the first interval is once every three weeks and the second interval is once every three weeks. In some embodiments, the combination is administered at the first dose of 24 mg once every three weeks for the first period of 2-6 dosing cycles (e.g. the first 3, 4, or 5 dosing cycles, in particular, the first 4 dosing cycles), and at the second dose of 80 mg once every three weeks until therapy is discontinued (e.g. due to disease progression, an adverse event, or as determined by a physician). In some embodiments, the combination is administered at the first dose of 24 mg once every three weeks for the first three dosing cycles, and at the second dose of 80 mg once every three weeks or more until therapy is discontinued (e.g. due to disease progression, an adverse event, or as determined by a physician). In some embodiments, the combination is administered at the first dose of 24 mg once every three weeks for the first four dosing cycles, and at the second dose of 80 mg once every three weeks or more until therapy is discontinued (e.g. due to disease progression, an adverse event, or as determined by a physician). In some embodiments, the combination is administered at the first dose of 24 mg once every three weeks for the first five dosing cycles, and at the second dose of 80 mg once every three weeks or more until therapy is discontinued (e.g. due to disease progression, an adverse event, or as determined by a physician).

With respect to the PD-1 inhibitor/TGF-βinhibitor/polypeptide comprising a PD-1 inhibitor and a TGFβR/anti-PD-(L)1(IgG):TGFβR, in some embodiments, the first interval and the second interval are different. In some embodiments, the first interval is once every two weeks and the second interval is once every three weeks. In some embodiments, the combination is administered at the first dose of 1200 mg once every two weeks for the first period of 2-6 dosing cycles (e.g. the first 3, 4, or 5 dosing cycles, in particular, the first 4 dosing cycles), and at the second dose of 2400 mg once every three weeks until therapy is discontinued (e.g. due to disease progression, an adverse event, or as determined by a physician). In some embodiments, the combination is administered at the first dose of 1200 mg once every two weeks for the first three dosing cycles, and at the second dose of 2400 mg once every three weeks or more until therapy is discontinued (e.g. due to disease progression, an adverse event, or as determined by a physician). In some embodiments, the combination is administered at the first dose of 1200 mg once every two weeks for the first four dosing cycles, and at the second dose of 2400 mg once every three weeks or more until therapy is discontinued (e.g. due to disease progression, an adverse event, or as determined by a physician). In some embodiments, the combination is administered at the first dose of 1200 mg once every two weeks for the first five dosing cycles, and at the second dose of 2400 mg once every three weeks or more until therapy is discontinued (e.g. due to disease progression, an adverse event, or as determined by a physician).

In some embodiments, the combination is administered at an administration interval (or treatment cycle) of once a week (Q1W), once every 2 weeks (Q2W), once every 3 weeks (Q3W), once every 4 weeks (Q4W), once every 5 weeks (Q5W), or once every 6 weeks (Q6W). In some embodiments, the combination is administered at an administration interval (or treatment cycle) of once a week (Q1W). In some embodiments, the combination is administered at an administration interval (or treatment cycle) of once every 2 weeks (Q2W). In some embodiments, the combination is administered at an administration interval (or treatment cycle) of once every three weeks (Q3W). In some embodiments, the combination is administered at an administration interval (or treatment cycle) of once every 4 weeks (Q4W). In some embodiments, the combination is administered at an administration interval (or treatment cycle) of once every 5 weeks (Q5W). In some embodiments, the combination is administered at an administration interval (or treatment cycle) of once every 6 weeks (Q6W). In some embodiments, the combination is administered for a period of at least about 2, 3, 4, 5, 6, 7, 8, 9, 10, 11, 12, 13, 14, 15, 16, 17, 18, 19, 20 weeks, or more. In some embodiments, the combination is administered on the first day of a treatment cycle or within 1, 2, or 3 days of the first day of a treatment cycle.

In some embodiments, the combination described herein is administered according to dosing regimens demonstrated to achieve a clinical benefit for the patient. In some embodiments, a clinical benefit is stable disease (“SD”), a partial response (“PR”) and/or a complete response (“CR”). In some embodiments, a clinical benefit is stable disease (“SD”). In some embodiments, a clinical benefit is a partial response (“PR”). In some embodiments, a clinical benefit is a complete response (“CR”). In some embodiments, PR or CR is determined in accordance with Response Evaluation Criteria in Solid Tumors (RECIST). In some embodiments, the combination is administered for a longer period to maintain clinical benefit.

In one aspect there is provided a method of treating cancer in a human, the method comprising administering to the human an ICOS binding protein (or antigen binding portion thereof) at a dose of about 0.08 mg to about 240 mg and administering to the human a polypeptide comprising a PD-1 inhibitor and a TGFβR. In one embodiment, the ICOS binding protein is administered at a dose of 0.08 mg, 0.24 mg, 0.8 mg, 2.4 mg, 8 mg, 24 mg, 48 mg, 80 mg, 160 mg or 240 mg, in particular 24 mg, 48 mg, 80 mg or 160 mg. In one aspect there is provided a method of treating cancer in a human, the method comprising administering to the human a polypeptide comprising a PD-1 inhibitor and a TGFβR at a dose of about 500 mg to about 3000 mg and administering to the human an ICOS binding protein (or antigen binding portion thereof). In one embodiment, the polypeptide comprising a PD-1 inhibitor and a TGFβR is administered at a dose of 2400 mg. In one embodiment, the polypeptide comprising a PD-1 inhibitor and a TGFβR is administered at a dose of 1200 mg. In one embodiment, there is a method of treating cancer in a human, the method comprising administering to the human an ICOS binding protein at a dose of about 0.08 mg to about 240 mg and administering to the human a polypeptide comprising a PD-1 inhibitor and a TGFβR at a dose of about 500 mg to about 3000 mg. In another embodiment, the ICOS binding protein is administered at a dose of 24 mg, 48 mg, 80 mg, 160 mg or 240 mg and the polypeptide comprising a PD-1 inhibitor and a TGFβR is administered at a dose of 1200 mg or 2400 mg. In another embodiment, the ICOS binding protein is administered at a dose of 24 mg, 48 mg, 80 mg, 160 mg or 240 mg and the polypeptide comprising a PD-1 inhibitor and a TGFβR is administered at a dose of 2400 mg. In another embodiment, the ICOS binding protein is administered at a dose of 24 mg, 48 mg, 80 mg, 160 mg or 240 mg and the polypeptide comprising a PD-1 inhibitor and a TGFβR is administered at a dose of 1200 mg.

In one aspect there is provided a method of treating cancer in a human, the method comprising administering to the human an ICOS binding protein (or antigen binding portion thereof) at a dose of about 0.08 mg to about 240 mg and administering to the human an anti-PD-(L)1(IgG):TGFβR fusion protein. In one embodiment, the ICOS binding protein is administered at a dose of 0.08 mg, 0.24 mg, 0.8 mg, 2.4 mg, 8 mg, 24 mg, 48 mg, 80 mg, 160 mg or 240 mg, in particular 24 mg, 48 mg, 80 mg or 160 mg. In one aspect there is provided a method of treating cancer in a human, the method comprising administering to the human an anti-PD-(L)1(IgG):TGFβR fusion protein at a dose of about 500 mg to about 3000 mg and administering to the human an ICOS binding protein (or antigen binding portion thereof). In one embodiment, the anti-PD-(L)1(IgG):TGFβR fusion protein is administered at a dose of 2400 mg. In one embodiment, the anti-PD-(L)1(IgG):TGFβR fusion protein is administered at a dose of 1200 mg. In one embodiment, there is a method of treating cancer in a human, the method comprising administering to the human an ICOS binding protein at a dose of about 0.08 mg to about 240 mg and administering to the human an anti-PD-(L)1(IgG):TGFβR fusion protein at a dose of about 500 mg to about 3000 mg. In another embodiment, the ICOS binding protein is administered at a dose of 24 mg, 48 mg, 80 mg, 160 mg or 240 mg and the anti-PD-(L)1(IgG):TGFβR fusion protein is administered at a dose of 1200 mg or 2400 mg. In another embodiment, the ICOS binding protein is administered at a dose of 24 mg, 48 mg, 80 mg, 160 mg or 240 mg and the anti-PD-(L)1 (IgG):TGFβR fusion protein is administered at a dose of 2400 mg. In another embodiment, the ICOS binding protein is administered at a dose of 24 mg, 48 mg, 80 mg, 160 mg or 240 mg and the anti-PD-(L)1 (IgG):TGFβR fusion protein is administered at a dose of 1200 mg.

In one aspect, there is provided an ICOS binding protein and a polypeptide comprising a PD-1 inhibitor and a TGFβR for concurrent (i.e. simultaneous) or sequential use in treating cancer, wherein the ICOS binding protein is to be administered at a dose of about 0.08 mg to about 240 mg. In one embodiment, the ICOS binding protein is administered at a dose of 8 mg, 24 mg, 48 mg, 80 mg, 160 mg or 240 mg. In one aspect, there is provided an ICOS binding protein and a polypeptide comprising a PD-1 inhibitor and a TGFβR for concurrent (i.e. simultaneous) or sequential use in treating cancer, wherein the polypeptide comprising a PD-1 inhibitor and a TGFβR is to be administered at a dose of about 500 mg to about 3000 mg. In one embodiment, the polypeptide comprising a PD-1 inhibitor and a TGFβR is administered at a dose of 2400 mg. In one embodiment, the polypeptide comprising a PD-1 inhibitor and a TGFβR is administered at a dose of 1200 mg.

In one aspect, there is provided an ICOS binding protein and an anti-PD-(L)1(IgG):TGFβR fusion protein for concurrent (i.e. simultaneous) or sequential use in treating cancer, wherein the ICOS binding protein is to be administered at a dose of about 0.08 mg to about 240 mg. In one embodiment, the ICOS binding protein is administered at a dose of 8 mg, 24 mg, 48 mg, 80 mg, 160 mg or 240 mg. In one aspect, there is provided an ICOS binding protein and an anti-PD-(L)1 (IgG):TGFβR fusion protein for concurrent (i.e. simultaneous) or sequential use in treating cancer, wherein the anti-PD-(L)1(IgG):TGFβR fusion protein is to be administered at a dose of about 500 mg to about 3000 mg. In one embodiment, the anti-PD-(L)1(IgG):TGFβR fusion protein is administered at a dose of 2400 mg. In one embodiment, the anti-PD-(L)1(IgG):TGFβR fusion protein is administered at a dose of 1200 mg.

In one embodiment, there is provided an ICOS binding protein and a polypeptide comprising a PD-1 inhibitor and a TGFβR for concurrent or sequential use in treating cancer, wherein the ICOS binding protein is to be administered at a dose of about 0.08 mg to about 240 mg and the polypeptide comprising a PD-1 inhibitor and a TGFβR is to be administered at a dose of about 500 mg to about 3000 mg. In another embodiment, the ICOS binding protein is administered at a dose of 24 mg, 48 mg, 80 mg or 160 mg, and the polypeptide comprising a PD-1 inhibitor and a TGFβR is administered at a dose of 2400 mg. In another embodiment, the ICOS binding protein is administered at a dose of 24 mg, 48 mg, 80 mg or 160 mg, and the polypeptide comprising a PD-1 inhibitor and a TGFβR is administered at a dose of 1200 mg.

In one embodiment, there is provided an ICOS binding protein and an anti-PD-(L)1 (IgG):TGFβR fusion protein for concurrent or sequential use in treating cancer, wherein the ICOS binding protein is to be administered at a dose of about 0.08 mg to about 240 mg and the anti-PD-(L)1(IgG):TGFβR fusion protein is to be administered at a dose of about 500 mg to about 3000 mg. In another embodiment, the ICOS binding protein is administered at a dose of 24 mg, 48 mg, 80 mg or 160 mg, and the anti-PD-(L)1(IgG):TGFβR fusion protein is administered at a dose of 2400 mg. In another embodiment, the ICOS binding protein is administered at a dose of 24 mg, 48 mg, 80 mg or 160 mg, and the anti-PD-(L)1(IgG):TGFβR fusion protein is administered at a dose of 1200 mg.

In another aspect, an ICOS binding protein for use in treating cancer is provided, wherein the ICOS binding protein is to be administered at a dose of about 0.08 mg to about 240 mg and is to be administered concurrently (i.e. simultaneously) or sequentially with a polypeptide comprising a PD-1 inhibitor and a TGFβR. In one embodiment, the ICOS binding protein is administered at a dose of 8 mg, 24 mg, 48 mg, 80 mg, 160 mg or 240 mg. In another aspect, a polypeptide comprising a PD-1 inhibitor and a TGFβR for use in treating cancer is provided, wherein the polypeptide comprising a PD-1 inhibitor and a TGFβR is to be administered at a dose of about 500 mg to about 3000 mg and is to be administered concurrently (i.e. simultaneously) or sequentially with an ICOS binding protein. In one embodiment, the polypeptide comprising a PD-1 inhibitor and a TGFβR is administered at a dose of 2400 mg. In one embodiment, the polypeptide comprising a PD-1 inhibitor and a TGFβR is administered at a dose of 1200 mg. In one embodiment, the ICOS binding protein is to be administered at a dose of about 0.08 mg to about 240 mg and is to be administered concurrently or sequentially with a polypeptide comprising a PD-1 inhibitor and a TGFβR at a dose of about 500 mg to about 3000 mg. In another embodiment, the ICOS binding protein is administered at a dose of 24 mg, 48 mg, 80 mg or 160 mg, and the polypeptide comprising a PD-1 inhibitor and a TGFβR is administered at a dose of 2400 mg. In another embodiment, the ICOS binding protein is administered at a dose of 24 mg, 48 mg, 80 mg or 160 mg, and the polypeptide comprising a PD-1 inhibitor and a TGFβR is administered at a dose of 1200 mg.

In another aspect, an ICOS binding protein for use in treating cancer is provided, wherein the ICOS binding protein is to be administered at a dose of about 0.08 mg to about 240 mg and is to be administered concurrently (i.e. simultaneously) or sequentially with an anti-PD-(L)1(IgG):TGFβR fusion protein. In one embodiment, the ICOS binding protein is administered at a dose of 8 mg, 24 mg, 48 mg, 80 mg, 160 mg or 240 mg. In another aspect, an anti-PD-(L)1(IgG):TGFβR fusion protein for use in treating cancer is provided, wherein the anti-PD-(L)1(IgG):TGFβR fusion protein is to be administered at a dose of about 500 mg to about 3000 mg and is to be administered concurrently (i.e. simultaneously) or sequentially with an ICOS binding protein. In one embodiment, the anti-PD-(L)1(IgG):TGFβR fusion protein is administered at a dose of 2400 mg. In one embodiment, the anti-PD-(L)1(IgG):TGFβR fusion protein is administered at a dose of 1200 mg. In one embodiment, the ICOS binding protein is to be administered at a dose of about 0.08 mg to about 240 mg and is to be administered concurrently or sequentially with a anti-PD-(L)1(IgG):TGFβR fusion protein at a dose of about 500 mg to about 3000 mg. In another embodiment, the ICOS binding protein is administered at a dose of 24 mg, 48 mg, 80 mg or 160 mg, and the anti-PD-(L)1(IgG):TGFβR fusion protein is administered at a dose of 2400 mg. In another embodiment, the ICOS binding protein is administered at a dose of 24 mg, 48 mg, 80 mg or 160 mg, and the anti-PD-(L)1(IgG):TGFβR fusion protein is administered at a dose of 1200 mg.

In another aspect, there is provided use of an ICOS binding protein in the manufacture of a medicament for treating cancer, wherein the ICOS binding protein is to be administered at a dose of about 0.08 mg to about 240 mg and is to be administered concurrently or sequentially with a polypeptide comprising a PD-1 inhibitor and a TGFβR. In one embodiment, the ICOS binding protein is administered at a dose of 8 mg, 24 mg, 48 mg, 80 mg, 160 mg or 240 mg. In another aspect, there is provided use of a polypeptide comprising a PD-1 inhibitor and a TGFβR in the manufacture of a medicament for treating cancer, wherein the polypeptide comprising a PD-1 inhibitor and a TGFβR is to be administered at a dose of about 500 mg to about 3000 mg and is to be administered concurrently or sequentially with an ICOS binding protein. In one embodiment, the polypeptide comprising a PD-1 inhibitor and a TGFβR is administered at a dose of 2400 mg. In one embodiment, the polypeptide comprising a PD-1 inhibitor and a TGFβR is administered at a dose of 1200 mg. In one embodiment, there is a use of an ICOS binding protein in the manufacture of a medicament for treating cancer, wherein the ICOS binding protein is to be administered at a dose of about 0.08 mg to about 240 mg and is to be administered concurrently or sequentially with a polypeptide comprising a PD-1 inhibitor and a TGFβR at a dose of about 500 mg to about 3000 mg. In another embodiment, the ICOS binding protein is administered at a dose of 24 mg, 48 mg, 80 mg or 160 mg, and the polypeptide comprising a PD-1 inhibitor and a TGFβR is administered at a dose of 2400 mg. In another embodiment, the ICOS binding protein is administered at a dose of 24 mg, 48 mg, 80 mg or 160 mg, and the polypeptide comprising a PD-1 inhibitor and a TGFβR is administered at a dose of 1200 mg.

In another aspect, there is provided use of an ICOS binding protein in the manufacture of a medicament for treating cancer, wherein the ICOS binding protein is to be administered at a dose of about 0.08 mg to about 240 mg and is to be administered concurrently or sequentially with an anti-PD-(L)1(IgG):TGFβR fusion protein. In one embodiment, the ICOS binding protein is administered at a dose of 8 mg, 24 mg, 48 mg, 80 mg, 160 mg or 240 mg. In another aspect, there is provided use of an anti-PD-(L)1(IgG):TGFβR fusion protein in the manufacture of a medicament for treating cancer, wherein the anti-PD-(L)1(IgG):TGFβR fusion protein is to be administered at a dose of about 500 mg to about 3000 mg and is to be administered concurrently or sequentially with an ICOS binding protein. In one embodiment, the anti-PD-(L)1(IgG):TGFβR fusion protein is administered at a dose of 2400 mg. In one embodiment, the anti-PD-(L)1(IgG):TGFβR fusion protein is administered at a dose of 1200 mg. In one embodiment, there is a use of an ICOS binding protein in the manufacture of a medicament for treating cancer, wherein the ICOS binding protein is to be administered at a dose of about 0.08 mg to about 240 mg and is to be administered concurrently or sequentially with an anti-PD-(L)1(IgG):TGFβR fusion protein at a dose of about 500 mg to about 3000 mg. In another embodiment, the ICOS binding protein is administered at a dose of 24 mg, 48 mg, 80 mg or 160 mg, and the anti-PD-(L)1(IgG):TGFβR fusion protein is administered at a dose of 2400 mg. In another embodiment, the ICOS binding protein is administered at a dose of 24 mg, 48 mg, 80 mg or 160 mg, and the anti-PD-(L)1(IgG):TGFβR fusion protein is administered at a dose of 1200 mg.

In one aspect, there is provided a pharmaceutical kit comprising about 0.08 mg to about 240 mg of an ICOS binding protein and a polypeptide comprising a PD-1 inhibitor and a TGFβR. In a further embodiment, the pharmaceutical kit comprises about 24 mg, about 48 mg, about 80 mg or about 160 mg of the ICOS binding protein. In one embodiment, the pharmaceutical kit comprises about 500 mg to about 3000 mg of the polypeptide comprising a PD-1 inhibitor and a TGFβR. In a further embodiment, the pharmaceutical kit comprises about 2400 mg of the polypeptide comprising a PD-1 inhibitor and a TGFβR. In a further embodiment, the pharmaceutical kit comprises about 1200 mg of the polypeptide comprising a PD-1 inhibitor and a TGFβR. In one embodiment, the polypeptide comprising a PD-1 inhibitor and a TGFβR is bintrafusp alfa.

In one aspect, there is provided a pharmaceutical kit comprising about 500 mg to about 3000 mg of a polypeptide comprising a PD-1 inhibitor and a TGFβR and an ICOS binding protein. In one embodiment, the pharmaceutical kit comprises about 0.08 mg to about 240 mg of the ICOS binding protein. In a further embodiment, the pharmaceutical kit comprises about 8 mg, about 24 mg or about 48 mg of the ICOS binding protein. In an further embodiment, the pharmaceutical kit comprises about 80 mg or about 160 mg of the ICOS binding protein.

In one embodiment, the pharmaceutical kit comprises the ICOS binding protein at a concentration of 10 mg/mL. In one embodiment, the pharmaceutical kit comprises the polypeptide comprising a PD-1 inhibitor and a TGFβR at a concentration of about 20 mg/mL to about 125 mg/mL. In a further embodiment, the pharmaceutical kit comprises the polypeptide comprising a PD-1 inhibitor and a TGFβR at a concentration of 20 mg/mL to 50 mg/mL. In one embodiment, the polypeptide comprising a PD-1 inhibitor and a TGFβR is at a concentration of 10 mg/mL. In one embodiment, the polypeptide comprising a PD-1 inhibitor and a TGFβR is at a concentration of 20 mg/mL. In one embodiment, the polypeptide comprising a PD-1 inhibitor and a TGFβR is at a concentration of 30 mg/mL. In one embodiment, the polypeptide comprising a PD-1 inhibitor and a TGFβR is at a concentration of 40 mg/mL. In another embodiment, the polypeptide comprising a PD-1 inhibitor and a TGFβR is at a concentration of 50 mg/mL.

In one aspect, there is provided a pharmaceutical kit comprising about 0.08 mg to about 240 mg of an ICOS binding protein and an anti-PD-(L)1(IgG):TGFβR fusion protein. In a further embodiment, the pharmaceutical kit comprises about 24 mg, about 48 mg, about 80 mg or about 160 mg of the ICOS binding protein. In one embodiment, the pharmaceutical kit comprises about 500 mg to about 3000 mg of the anti-PD-(L)1(IgG):TGFβR fusion protein. In a further embodiment, the pharmaceutical kit comprises about 2400 mg of the anti-PD-(L)1(IgG):TGFβR fusion protein. In a further embodiment, the pharmaceutical kit comprises about 1200 mg of the anti-PD-(L)1(IgG):TGFβR fusion protein. In one embodiment, the anti-PD-(L)1(IgG):TGFβR fusion protein is bintrafusp alfa.

In one aspect, there is provided a pharmaceutical kit comprising about 500 mg to about 3000 mg of an anti-PD-(L)1(IgG):TGFβR fusion protein and an ICOS binding protein. In one embodiment, the pharmaceutical kit comprises about 0.08 mg to about 240 mg of the ICOS binding protein. In a further embodiment, the pharmaceutical kit comprises about 8 mg, about 24 mg or about 48 mg of the ICOS binding protein. In an further embodiment, the pharmaceutical kit comprises about 80 mg or about 160 mg of the ICOS binding protein.

In one embodiment, the pharmaceutical kit comprises the ICOS binding protein at a concentration of 10 mg/mL. In one embodiment, the pharmaceutical kit comprises the anti-PD-(L)1(IgG):TGFβR fusion protein at a concentration of about 20 mg/mL to about 125 mg/mL. In a further embodiment, the pharmaceutical kit comprises the anti-PD-(L)1(IgG):TGFβR fusion protein at a concentration of 20 mg/mL to 50 mg/mL. In one embodiment, the anti-PD-(L)1(IgG):TGFβR fusion protein is at a concentration of 10 mg/mL. In one embodiment, the anti-PD-(L)1(IgG):TGFβR fusion protein is at a concentration of 20 mg/mL. In one embodiment, the anti-PD-(L)1(IgG):TGFβR fusion protein is at a concentration of 30 mg/mL. In one embodiment, the anti-PD-(L)1(IgG):TGFβR fusion protein is at a concentration of 40 mg/mL. In another embodiment, the anti-PD-(L)1(IgG):TGFβR fusion protein is at a concentration of 50 mg/mL.

In another aspect, there is provided a pharmaceutical formulation comprising an ICOS binding protein at a concentration of 10 mg/mL. In another aspect, there is provided a pharmaceutical formulation comprising a polypeptide comprising a PD-1 inhibitor and a TGFβR at a concentration of about 20 mg/mL to about 125 mg/mL. In a further embodiment, the pharmaceutical formulation comprises a polypeptide comprising a PD-1 inhibitor and a TGFβR at a concentration of 20 mg/mL to 50 mg/mL. In one embodiment, the polypeptide comprising a PD-1 inhibitor and a TGFβR is at a concentration of 10 mg/mL In one embodiment, the polypeptide comprising a PD-1 inhibitor and a TGFβR is at a concentration of 20 mg/mL. In one embodiment, the polypeptide comprising a PD-1 inhibitor and a TGFβR is at a concentration of 30 mg/mL. In one embodiment, the polypeptide comprising a PD-1 inhibitor and a TGFβR is at a concentration of 40 mg/mL In another embodiment, the polypeptide comprising a PD-1 inhibitor and a TGFβR is at a concentration of 50 mg/mL. Thus, in one embodiment, the pharmaceutical formulation comprises an ICOS binding protein at a concentration of 10 mg/ml and a polypeptide comprising a PD-1 inhibitor and a TGFβR at a concentration of about 20 mg/mL to about 125 mg/mL. In a further embodiment, the pharmaceutical formulation comprises an ICOS binding protein at a concentration of 10 mg/ml and a polypeptide comprising a PD-1 inhibitor and a TGFβR at a concentration of 20 mg/mL to 50 mg/mL. In one embodiment, the pharmaceutical formulation comprises an ICOS binding protein at a concentration of 10 mg/ml and a polypeptide comprising a PD-1 inhibitor and a TGFβR at a concentration of 10 mg/mL. In one embodiment, the pharmaceutical formulation comprises an ICOS binding protein at a concentration of 10 mg/ml and a polypeptide comprising a PD-1 inhibitor and a TGFβR at a concentration of 20 mg/mL. In one embodiment, the pharmaceutical formulation comprises an ICOS binding protein at a concentration of 10 mg/ml and a polypeptide comprising a PD-1 inhibitor and a TGFβR at a concentration of 30 mg/mL. In one embodiment, the pharmaceutical formulation comprises an ICOS binding protein at a concentration of 10 mg/ml and a polypeptide comprising a PD-1 inhibitor and a TGFβR at a concentration of 40 mg/mL. In another embodiment, the pharmaceutical formulation comprises an ICOS binding protein at a concentration of 10 mg/ml and a polypeptide comprising a PD-1 inhibitor and a TGFβR at a concentration of 50 mg/mL.

In another aspect, there is provided a pharmaceutical formulation comprising an ICOS binding protein at a concentration of 10 mg/mL. In another aspect, there is provided a pharmaceutical formulation comprising an anti-PD-(L)1(IgG):TGFβR fusion protein at a concentration of about 20 mg/mL to about 125 mg/mL. In a further embodiment, the pharmaceutical formulation comprises an anti-PD-(L)1(IgG):TGFβR fusion protein at a concentration of 20 mg/mL to 50 mg/mL. In one embodiment, the anti-PD-(L)1(IgG):TGFβR fusion protein is at a concentration of 10 mg/mL In one embodiment, the anti-PD-(L)1(IgG):TGFβR fusion protein is at a concentration of 20 mg/mL. In one embodiment, the anti-PD-(L)1(IgG):TGFβR fusion protein is at a concentration of 30 mg/mL. In one embodiment, the anti-PD-(L)1(IgG):TGFβR fusion protein is at a concentration of 40 mg/mL In another embodiment, the anti-PD-(L)1(IgG):TGFβR fusion protein is at a concentration of 50 mg/mL.

Thus, in one embodiment, the pharmaceutical formulation comprises an ICOS binding protein at a concentration of 10 mg/ml and an anti-PD-(L)1(IgG):TGFβR fusion protein at a concentration of about 20 mg/mL to about 125 mg/mL. In a further embodiment, the pharmaceutical formulation comprises an ICOS binding protein at a concentration of 10 mg/ml and an anti-PD-(L)1(IgG):TGFβR fusion protein at a concentration of 20 mg/mL to 50 mg/mL. In one embodiment, the pharmaceutical formulation comprises an ICOS binding protein at a concentration of 10 mg/ml and an anti-PD-(L)1(IgG):TGFβR fusion protein at a concentration of 10 mg/mL. In one embodiment, the pharmaceutical formulation comprises an ICOS binding protein at a concentration of 10 mg/ml and an anti-PD-(L)1(IgG):TGFβR fusion protein at a concentration of 20 mg/mL. In one embodiment, the pharmaceutical formulation comprises an ICOS binding protein at a concentration of 10 mg/ml and an anti-PD-(L)1(IgG):TGFβR fusion protein at a concentration of 30 mg/mL. In one embodiment, the pharmaceutical formulation comprises an ICOS binding protein at a concentration of 10 mg/ml and an anti-PD-(L)1(IgG):TGFβR fusion protein at a concentration of 40 mg/mL. In another embodiment, the pharmaceutical formulation comprises an ICOS binding protein at a concentration of 10 mg/ml and an anti-PD-(L)1(IgG):TGFβR fusion protein at a concentration of 50 mg/mL.

In some embodiments, the ICOS binding protein is administered at a dose of about 0.08-800 mg (e.g. a dose about 0.08 mg; a dose about 0.24 mg; a dose about 0.8 mg; a dose about 2.4 mg; a dose about 8 mg; a dose about 16 mg; a dose about 24 mg; a dose about 32 mg; a dose about 40 mg; a dose about 48 mg; a dose about 56 mg; a dose about 64 mg; a dose about 72 mg; a dose about 80 mg; a dose about 88 mg; a dose about 96 mg; a dose about 100 mg; a dose about 160 mg; a dose about 200 mg; a dose about 240 mg; a dose about 300 mg; a dose about 400 mg; a dose about 500 mg; a dose about 600 mg; a dose about 700 mg or a dose about 800 mg). In some embodiments, the ICOS binding protein is administered at a dose of about 0.08-240 mg. In further embodiments, the ICOS binding protein is administered at a dose of about 0.001-10 mg/kg (e.g. a dose about 0.001 mg/kg, a dose about 0.003 mg/kg, a dose about 0.01 mg/kg, a dose about 0.03 mg/kg, a dose about 0.1 mg/kg, a dose about 0.3 mg/kg, a dose about 0.6 mg/kg, a dose about 1.0 mg/kg, a dose about 2.0 mg/kg, a dose about 3.0 mg/kg, a dose about 6 mg/kg or a dose about 10 mg/kg). In some embodiments, the ICOS binding protein is administered at a dose of about 0.001-3 mg/kg. In some embodiments, the ICOS binding protein is administered at a dose of about 0.3 mg/kg. In some embodiments, the ICOS binding protein is administered at a dose of about 1 mg/kg.

In some embodiments, the ICOS binding protein is administered at a dose of about 3 mg/kg. In some embodiments, the ICOS binding protein is administered at a dose of about 24 mg. In some embodiments, the ICOS binding protein is administered at a dose of about 48 mg. In some embodiments, the ICOS binding protein is administered at a dose of about 72 mg. In some embodiments, the ICOS binding protein is administered at a dose of about 80 mg. In some embodiments, the ICOS protein is administered at a dose of about 96 mg. In some embodiments, the ICOS protein is administered at a dose of about 120 mg. In some embodiments, the ICOS protein is administered at a dose of about 148 mg. In some embodiments, the ICOS binding protein is administered at a dose of about 160 mg. In some embodiments, the ICOS binding protein is administered at a dose of about 240 mg. In some embodiments, the ICOS protein is administered at a dose of about 320 mg. In some embodiments, the ICOS protein is administered at a dose of about 480 mg.

In one embodiment, the dose of the ICOS binding protein is in the range of about 0.08 mg to about 800 mg. In another embodiment, the dose of the ICOS binding protein is in the range of about 0.8 mg to about 240 mg.

In another embodiment, the dose of the ICOS binding protein is in the range of about 8 mg to about 80 mg. In another embodiment, the dose of the ICOS binding protein is about 0.08 mg, about 0.24 mg, about 0.48 mg, about 0.8 mg, about 1.6 mg, about 2.4 mg, about 8 mg, about 24 mg, about 48 mg, about 80 mg, about 160 mg or about 240 mg. In one embodiment, the dose of ICOS binding protein is about 24 mg, about 48 mg, about 80 mg or about 160 mg. In one embodiment, the dose of the ICOS binding protein is at least about 24 mg. In one embodiment, the dose of the ICOS binding protein is at least about 48 mg.

In one embodiment, the ICOS binding protein is administered once every 2-6 weeks (e.g. 2, 3 or 4 weeks, in particular 3 weeks). In one embodiment the ICOS binding protein is administered for once every 3 weeks for 2-6 dosing cycles (e.g. the first 3, 4, or 5 dosing cycles, in particular, the first 4 dosing cycles).

In one embodiment, the ICOS binding protein is vopratelimab. In one embodiment, vopratelimab is administered at 0.03 mg/kg, 0.1 mg/kg or 0.3 mg/kg. In one embodiment, vopratelimab is administered every 3 weeks. In another embodiment, the dosing amount and interval between doses of vopratelimab is pulsatile.

In some embodiments, the polypeptide comprising a PD-1 inhibitor and a TGFβR is administered at a dose of about 500-3000 mg (e.g. a dose about 500 mg; a dose about 600 mg; a dose about 700 mg; a dose about 800 mg; a dose about 900 mg; a dose about 1000 mg; a dose about 1100 mg; a dose about 1200 mg; a dose about 1300 mg; a dose about 1400 mg; a dose about 1500 mg; a dose about 1600 mg; a dose about 1700 mg; a dose about 1800 mg; a dose about 1900 mg; a dose about 2000 mg; a dose about 2100 mg; a dose about 2200 mg; a dose about 2300 mg; a dose about 2400 mg; a dose of about 2500 mg; a dose of about 2600 mg; a dose of about 2700 mg; a dose of about 2800 mg; a dose of about 2900 mg; or a dose of about 3000 mg). In some embodiments, the polypeptide comprising a PD-1 inhibitor and a TGFβR is administered at a dose of about 12.5 mg/kg. In some embodiments, the polypeptide comprising a PD-1 inhibitor and a TGFβR is administered at a dose of about 15 mg/kg. In some embodiments, the polypeptide comprising a PD-1 inhibitor and a TGFβR is administered at a dose of about 30 mg/kg. In some embodiments, the polypeptide comprising a PD-1 inhibitor and a TGFβR is administered at a dose of about 1000 mg. In some embodiments, the polypeptide comprising a PD-1 inhibitor and a TGFβR is administered at a dose of about 1200 mg. In some embodiments, the polypeptide comprising a PD-1 inhibitor and a TGFβR is administered at a dose of about 2400 mg.

In one embodiment, the polypeptide comprising a PD-1 inhibitor and a TGFβR is administered once every 2-6 weeks (e.g. 2, 3 or 4 weeks, in particular 2 weeks or 3 weeks). In one embodiment the polypeptide comprising a PD-1 inhibitor and a TGFβR is administered for once every 2 weeks for 2-6 dosing cycles (e.g. the first 3, 4, or 5 dosing cycles, in particular, the first 4 dosing cycles). In one embodiment the polypeptide comprising a PD-1 inhibitor and a TGFβR is administered for once every 3 weeks for 2-6 dosing cycles (e.g. the first 3, 4, or 5 dosing cycles, in particular, the first 4 dosing cycles).

In some embodiments, the anti-PD-(L)1(IgG):TGFβR fusion protein is administered at a dose of about 500-3000 mg (e.g. a dose about 500 mg; a dose about 600 mg; a dose about 700 mg; a dose about 800 mg; a dose about 900 mg; a dose about 1000 mg; a dose about 1100 mg; a dose about 1200 mg; a dose about 1300 mg; a dose about 1400 mg; a dose about 1500 mg; a dose about 1600 mg; a dose about 1700 mg; a dose about 1800 mg; a dose about 1900 mg; a dose about 2000 mg; a dose about 2100 mg; a dose about 2200 mg; a dose about 2300 mg; a dose about 2400 mg; a dose of about 2500 mg; a dose of about 2600 mg; a dose of about 2700 mg; a dose of about 2800 mg; a dose of about 2900 mg; or a dose of about 3000 mg). In some embodiments, the anti-PD-(L)1(IgG):TGFβR fusion protein is administered at a dose of about 12.5 mg/kg. In some embodiments, the anti-PD-(L)1(IgG):TGFβR fusion protein is administered at a dose of about 15 mg/kg. In some embodiments, the anti-PD-(L)1(IgG):TGFβR fusion protein is administered at a dose of about 30 mg/kg. In some embodiments, the anti-PD-(L)1(IgG):TGFβR fusion protein is administered at a dose of about 1000 mg. In some embodiments, the anti-PD-(L)1(IgG):TGFβR fusion protein is administered at a dose of about 1200 mg. In some embodiments, the anti-PD-(L)1(IgG):TGFβR fusion protein is administered at a dose of about 2400 mg.

In one embodiment, the anti-PD-(L)1(IgG):TGFβR fusion protein is administered once every 2-6 weeks (e.g. 2, 3 or 4 weeks, in particular 2 weeks or 3 weeks). In one embodiment the anti-PD-(L)1(IgG):TGFβR fusion protein is administered for once every 2 weeks for 2-6 dosing cycles (e.g. the first 3, 4, or 5 dosing cycles, in particular, the first 4 dosing cycles). In one embodiment the anti-PD-(L)1(IgG):TGFβR fusion protein is administered for once every 3 weeks for 2-6 dosing cycles (e.g. the first 3, 4, or 5 dosing cycles, in particular, the first 4 dosing cycles).

In one embodiment, the polypeptide comprising a PD-1 inhibitor and a TGFβR is administered at a dose of 1200 mg every 2 weeks. In one embodiment, polypeptide comprising a PD-1 inhibitor and a TGFβR is administered at a dose of 15 mg/kg every 2 weeks. In one embodiment, the polypeptide comprising a PD-1 inhibitor and a TGFβR is administered at a dose of 2400 mg every 3 weeks. In one embodiment, polypeptide comprising a PD-1 inhibitor and a TGFβR is administered at a dose of 30 mg/kg every 3 weeks.

In one embodiment, the anti-PD-(L)1(IgG):TGFβR fusion protein is administered at a dose of 1200 mg every 2 weeks. In one embodiment, anti-PD-(L)1(IgG):TGFβR fusion protein is administered at a dose of 15 mg/kg every 2 weeks. In one embodiment, the anti-PD-(L)1(IgG):TGFβR fusion protein is administered at a dose of 2400 mg every 3 weeks. In one embodiment, anti-PD-(L)1(IgG):TGFβR fusion protein is administered at a dose of 30 mg/kg every 3 weeks.

In one embodiment, the polypeptide comprising a PD-1 inhibitor and a TGFβR, or anti-PD-(L)1(IgG):TGFβR fusion protein, is bintrafusp alfa. In one embodiment, bintrafusp alfa is administered at a dose of 1200 mg every 2 weeks. In one embodiment, bintrafusp alfa is administered at a dose of 15 mg/kg every 2 weeks. In one embodiment, bintrafusp alfa is administered at a dose of 2400 mg every 3 weeks. In one embodiment, bintrafusp alfa is administered at a dose of 30 mg/kg every 3 weeks.

Fixed doses may be tested assuming a typical median weight of 80 kg.

Therapeutic monoclonal antibodies are often dosed based on body-size due to the concept that this reduces inter-subject variability in drug exposure. However, body-weight dependency of PK parameters does not always explain the observed variability in the exposure of monoclonal antibodies (Zhao et al. Annals of Oncology. (2017) 28:2002-2008). The advantage of body-weight based versus fixed dosing in the study provide in the Examples was evaluated through population PK modelling and simulation efforts. A preliminary population PK model was developed from monotherapy dose escalation (data up to doses of 1 mg/kg; n=19 subjects).

Simulations were performed by considering body weight distribution in the simulations were based on the observed distribution in the preliminary dataset. At the 5th percentile of body weight (40-47 kg), there was a 70-100% increase in median steady-state AUC(0-); H2L5 IgG4PE exposures higher than these increases have been evaluated in the current Phase 1 study with the 3 mg/kg dose regimen. At the 95th percentile of body weight (107-118 kg), there was a 23-32% decrease in median steady-state AUC (0-) as compared to the median 80 kg exposure providing adequate receptor occupancy (RO) with the minimal lowering of exposure. A similar outcome is expected for steady-state Cmax and trough concentrations between body weight-based and fixed dosing.

Overall, these preliminary population PK simulations indicate that using fixed dosing would result in a similar range of exposures as that of body weight-based dosing. Also, fixed dosing offers the advantage of reduced dosing errors, reduced drug wastage, shorten preparation time, and improve ease of administration. Thus, switching to a fixed dose based on a reference body weight of 80 kg is reasonable and appropriate.

It is to be understood that where mg/kg is used, this is mg/kg of body weight. In one embodiment, the dose of the ICOS binding protein is between about 0.001 mg/kg to about 3.0 mg/kg. In another embodiment, the dose of the ICOS binding protein is about 0.001 mg/kg, about 0.003 mg/kg, about 0.01 mg/kg, about 0.03 mg/kg, about 0.1 mg/kg, about 0.3 mg/kg, about 1.0 mg/kg, about 3.0 mg/kg, or about 10 mg/kg. In one embodiment, the dose of ICOS binding protein is about 0.3 mg/kg. In another embodiment, the dose of the ICOS binding protein is at least 3.0 mg/kg. In one embodiment, the dose of the ICOS binding protein is in the range of about 0.001 mg/kg to about 10 mg/kg. In one embodiment, the dose of the ICOS binding protein is about 0.1 mg/kg to about 1.0 mg/kg. In one embodiment, the dose of the ICOS binding protein is about 0.1 mg/kg. In one embodiment, the dose of the ICOS binding protein is at least 0.1 mg/kg. In another embodiment, the dose of the ICOS binding protein is about 0.3 mg/kg. In another embodiment, the dose of the ICOS binding protein is about 1 mg/kg. In one embodiment, the dose of the ICOS binding protein is about 3 mg/kg. In one embodiment, a fixed dose of ICOS binding protein may be administered, assuming a typical median weight of 80 kg.

In one embodiment, the dose of ICOS binding protein is increased during the treatment regimen. In one embodiment an initial dose of about 0.001 mg/kg, about 0.003 mg/kg, about 0.01 mg/kg, about 0.03 mg/kg, about 0.1 mg/kg, about 0.3 mg/kg, about 1.0 mg/kg is increased to about 0.003 mg/kg, about 0.01 mg/kg, about 0.03 mg/kg, about 0.1 mg/kg, about 0.3 mg/kg, about 1.0 mg/kg, about 3.0 mg/kg or at least 3.0 mg/kg. In one embodiment, an initial dose of 0.1 mg/kg is increased to 1 mg/kg. In one embodiment, an initial dose of 0.3 mg/kg is increased to 1 mg/kg. In one embodiment, the initial dose of 0.6 mg/kg is increased to 2 mg/kg.

In one embodiment, the ICOS binding protein is administered at 0.1 mg/kg×3 doses then 1 mg/kg. In one embodiment, the ICOS binding protein is administered at about 0.001 mg/kg, about 0.003 mg/kg, about 0.01 mg/kg, about 0.03 mg/kg, about 0.1 mg/kg, about 0.3 mg/kg, about 1.0 mg/kg, or about 3.0 mg/kg then increased to about 0.01 mg/kg, about 0.03 mg/kg, about 0.1 mg/kg, about 0.3 mg/kg, about 1.0 mg/kg, about 3.0 mg/kg or about 10 mg/kg.

In one embodiment, the dose of the polypeptide comprising a PD-1 inhibitor and a TGFβR is between about 6.25 mg/kg to about 37.5 mg/kg. In another embodiment, the dose of the polypeptide comprising a PD-1 inhibitor and a TGFβR is about 6.25 mg/kg, about 12.5 mg/kg, about 15 mg/kg, about 18.75 mg/kg, about 25.0 mg/kg, about 30 mg/kg or about 37.5 mg/kg. In another embodiment, the dose of the polypeptide comprising a PD-1 inhibitor and a TGFβR is at least 6.25 mg/kg. In one embodiment, the dose of the polypeptide comprising a PD-1 inhibitor and a TGFβR is in the range of about 15 mg/kg to about 30 mg/kg. In one embodiment, the dose of the polypeptide comprising a PD-1 inhibitor and a TGFβR is about 30 mg/kg. In one embodiment, a fixed dose of polypeptide comprising a PD-1 inhibitor and a TGFβR may be administered, assuming a typical median weight of 80 kg.

In one embodiment, the dose of the polypeptide comprising a PD-1 inhibitor and a TGFβR is increased during the treatment regimen. In one embodiment, the initial dose of about 15 mg/kg is increased to about 30 mg/kg.

In one embodiment, the dose of the anti-PD-(L)1(IgG):TGFβR fusion protein is between about 6.25 mg/kg to about 37.5 mg/kg. In another embodiment, the dose of the anti-PD-(L)1(IgG):TGFβR fusion protein is about 6.25 mg/kg, about 12.5 mg/kg, about 15 mg/kg, about 18.75 mg/kg, about 25.0 mg/kg, about 30 mg/kg or about 37.5 mg/kg. In another embodiment, the dose of the anti-PD-(L)1(IgG):TGFβR fusion protein is at least 6.25 mg/kg. In one embodiment, the dose of the anti-PD-(L)1(IgG):TGFβR fusion protein is in the range of about 15 mg/kg to about 30 mg/kg. In one embodiment, the dose of the anti-PD-(L)1(IgG):TGFβR fusion protein is about 30 mg/kg. In one embodiment, a fixed dose of anti-PD-(L)1(IgG):TGFβR fusion protein may be administered, assuming a typical median weight of 80 kg.

In one embodiment, the dose of the anti-PD-(L)1(IgG):TGFβR fusion protein is increased during the treatment regimen. In one embodiment, the initial dose of about 15 mg/kg is increased to about 30 mg/kg.

In one embodiment, the ICOS binding protein is administered once every 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days. In one embodiment, the polypeptide comprising a PD-1 inhibitor and a TGFβR, or the anti-PD-(L)1(IgG):TGFβR fusion protein, is administered once every 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days.

In one embodiment, the ICOS binding protein is administered once every week, once every two weeks, once every three weeks, once every four weeks, once every five weeks or once every six weeks. In one embodiment, the ICOS binding protein is administered once every three weeks. In one embodiment, the ICOS binding protein is administered once every six weeks. In one embodiment, the ICOS binding protein is administered once every three weeks or once every six weeks until disease progression. In one embodiment, the ICOS binding protein is administered once every three weeks for 35 cycles.

In one embodiment, the polypeptide comprising a PD-1 inhibitor and a TGFβR is administered once every week, once every two weeks, once every three weeks, once every four weeks, once every five weeks or once every six weeks. In one embodiment, the polypeptide comprising a PD-1 inhibitor and a TGFβR is administered once every three weeks. In one embodiment, the polypeptide comprising a PD-1 inhibitor and a TGFβR is administered once every six weeks. In one embodiment, the polypeptide comprising a PD-1 inhibitor and a TGFβR is administered once every three weeks or once every six weeks until disease progression. In one embodiment, the polypeptide comprising a PD-1 inhibitor and a TGFβR is administered once every three weeks for 35 cycles.

In one embodiment, the anti-PD-(L)1(IgG):TGFβR fusion protein is administered once every week, once every two weeks, once every three weeks, once every four weeks, once every five weeks or once every six weeks. In one embodiment, the anti-PD-(L)1(IgG):TGFβR fusion protein is administered once every three weeks. In one embodiment, the anti-PD-(L)1(IgG):TGFβR fusion protein is administered once every six weeks. In one embodiment, the anti-PD-(L)1(IgG):TGFβR fusion protein is administered once every three weeks or once every six weeks until disease progression. In one embodiment, the anti-PD-(L)1(IgG):TGFβR fusion protein is administered once every three weeks for 35 cycles.

In one embodiment, the ICOS binding protein and/or polypeptide comprising a PD-1 inhibitor and a TGFβR, or anti-PD-(L)1(IgG):TGFβR fusion protein, is administered every two weeks up to 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 cycles. In one embodiment, the ICOS binding protein, and/or polypeptide comprising a PD-1 inhibitor and a TGFβR, or anti-PD-(L)1(IgG):TGFβR fusion protein, is administered every two weeks up to 35 cycles. In one embodiment, the ICOS binding protein and/or polypeptide comprising a PD-1 inhibitor and a TGFβR, or anti-PD-(L)1(IgG):TGFβR fusion protein, is administered every three weeks up to 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 cycles. In one embodiment, the ICOS binding protein and/or polypeptide comprising a PD-1 inhibitor and a TGFβR, or anti-PD-(L)1(IgG):TGFβR fusion protein, is administered every three weeks up to 35 cycles. In one embodiment, the ICOS binding protein and/or polypeptide comprising a PD-1 inhibitor and a TGFβR, or anti-PD-(L)1(IgG):TGFβR fusion protein, is administered every six weeks up to 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 cycles. In one embodiment, the ICOS binding protein and/or polypeptide comprising a PD-1 inhibitor and a TGFβR, or anti-PD-(L)1(IgG):TGFβR fusion protein is administered every six weeks up to 35 cycles.

The individual components of the combinations disclosed herein may be administered either in separate or combined form (e.g. as pharmaceutical formulations) by any convenient route.

For some therapeutic agents (i.e. binding proteins), suitable routes include oral, rectal, nasal, topical (including buccal and sublingual), vaginal, and parenteral (including subcutaneous, intramuscular, intravenous, intradermal, intrathecal, and epidural). It will be appreciated that the preferred route may vary with, for example, the condition of the recipient of the combination and the cancer to be treated. It will also be appreciated that each of the agents administered may be administered by the same or different routes and that the therapeutic agents may be formulated together or in separate pharmaceutical compositions.

In one embodiment, one or more binding agents of a combination of the invention are administered intravenously. In a further embodiment, the one or more binding agents of a combination of the invention are administered by intravenous infusion. In another embodiment, one or more therapeutic agents of a combination of the invention are administered intratumorally. In another embodiment, one or more binding agents of a combination of the invention are administered orally. In another embodiment, one or more binding agents of a combination of the invention are administered systemically, e.g. intravenously, and one or more other therapeutic agents of a combination of the invention are administered intratumorally. In another embodiment, all of the therapeutic agents of a combination of the invention are administered systemically, e.g. intravenously. In an alternative embodiment, all of the therapeutic agents of the combination of the invention are administered intratumorally. In any of the embodiments, e.g. in this paragraph, the therapeutic agents of the invention may be administered as one or more pharmaceutical compositions.

In one embodiment, the ICOS binding protein is administered via intravenous (IV) infusion. In one embodiment, the polypeptide comprising a PD-1 inhibitor and a TGFβR is administered via IV infusion. In one embodiment, the anti-PD-(L)1(IgG):TGFβR fusion protein is administered via IV infusion.

In one embodiment, the ICOS binding protein is administered via IV infusion at a dose of about 0.08 mg, about 0.24 mg, about 0.48 mg, about 0.8 mg, about 1.6 mg, about 2.4 mg, about 8 mg, about 24 mg, about 48 mg, about 80 mg, about 160 mg or about 240 mg every three weeks. In one embodiment, the ICOS binding protein is administered at a dose of 24 mg or 80 mg via IV infusion every three weeks. In one embodiment, the ICOS binding protein is administered at a dose of 0.3 mg/kg or 1 mg/kg via IV infusion every three weeks. In one embodiment, the ICOS binding protein is administered via IV infusion at a dose of about 8 mg, about 24 mg, about 48 mg, about 80 mg, about 160 mg or about 240 mg every six weeks. In one embodiment, the ICOS binding protein is administered at a dose of 48 mg or 160 mg via IV infusion every six weeks. In one embodiment, the ICOS binding protein is administered at a dose of 0.6 mg/kg or 2 mg/kg via IV infusion every six weeks.

In one embodiment, the polypeptide comprising a PD-1 inhibitor and a TGFβR is administered via IV infusion at a dose of about 500 mg, about 700 mg, about 1000 mg, about 1200 mg, about 1500 mg, about 1800 mg, about 2000 mg, about 2400 mg, about 2600 mg, about 3000 mg every two weeks. In one embodiment, the polypeptide comprising a PD-1 inhibitor and a TGFβR is administered at a dose of 1200 mg via IV infusion every two weeks. In one embodiment, the polypeptide comprising a PD-1 inhibitor and a TGFβR is administered at a dose of about 15 mg/kg via IV infusion every two weeks. In one embodiment, the polypeptide comprising a PD-1 inhibitor and a TGFβR is administered via IV infusion at a dose of about 500 mg, about 700 mg, about 1000 mg, about 1200 mg, about 1500 mg, about 1800 mg, about 2000 mg, about 2400 mg, about 2600 mg, about 3000 mg every three weeks. In one embodiment, the polypeptide comprising a PD-1 inhibitor and a TGFβR is administered at a dose of 2400 mg via IV infusion every three weeks. In one embodiment, the polypeptide comprising a PD-1 inhibitor and a TGFβR is administered at a dose of about 30 mg/kg via IV infusion every three weeks. In one embodiment, the polypeptide comprising a PD-1 inhibitor and a TGFβR is administered via IV infusion at a dose of about 1000 mg, 1400 mg, 2000 mg, 2400 mg, about 3000 mg, about 3600 mg, about 4000 mg, about 4800 mg, about 5200 mg, about 6000 mg every six weeks. In one embodiment, the polypeptide comprising a PD-1 inhibitor and a TGFβR is administered at a dose of 4800 mg via IV infusion every six weeks. In one embodiment, the polypeptide comprising a PD-1 inhibitor and a TGFβR is administered at a dose of about 60 mg/kg via IV infusion every six weeks.

In one embodiment, the anti-PD-(L)1(IgG):TGFβR fusion protein is administered via IV infusion at a dose of about 500 mg, about 700 mg, about 1000 mg, about 1200 mg, about 1500 mg, about 1800 mg, about 2000 mg, about 2400 mg, about 2600 mg, about 3000 mg every two weeks. In one embodiment, the anti-PD-(L)1(IgG):TGFβR fusion protein is administered at a dose of 1200 mg via IV infusion every two weeks. In one embodiment, the anti-PD-(L)1(IgG):TGFβR fusion protein is administered at a dose of about 15 mg/kg via IV infusion every two weeks. In one embodiment, the anti-PD-(L)1(IgG):TGFβR fusion protein is administered via IV infusion at a dose of about 500 mg, about 700 mg, about 1000 mg, about 1200 mg, about 1500 mg, about 1800 mg, about 2000 mg, about 2400 mg, about 2600 mg, about 3000 mg every three weeks. In one embodiment, the anti-PD-(L)1(IgG):TGFβR fusion protein is administered at a dose of 2400 mg via IV infusion every three weeks. In one embodiment, the anti-PD-(L)1(IgG):TGFβR fusion protein is administered at a dose of about 30 mg/kg via IV infusion every three weeks. In one embodiment, the anti-PD-(L)1(IgG):TGFβR fusion protein is administered via IV infusion at a dose of about 1000 mg, 1400 mg, 2000 mg, 2400 mg, about 3000 mg, about 3600 mg, about 4000 mg, about 4800 mg, about 5200 mg, about 6000 mg every six weeks. In one embodiment, the anti-PD-(L)1(IgG):TGFβR fusion protein is administered at a dose of 4800 mg via IV infusion every six weeks. In one embodiment, the anti-PD-(L)1(IgG):TGFβR fusion protein is administered at a dose of about 60 mg/kg via IV infusion every six weeks.

In one embodiment, the ICOS binding protein is administered at a dose of 0.3 mg/kg via IV infusion every three weeks and the polypeptide comprising a PD-1 inhibitor and a TGFβR, or anti-PD-(L)1(IgG):TGFβR fusion protein, is administered at a dose of 1200 mg via IV infusion every two weeks. In one embodiment, the ICOS binding protein is administered at a dose of 0.3 mg/kg via IV infusion every three weeks and the polypeptide comprising a PD-1 inhibitor and a TGFβR, or anti-PD-(L)1(IgG):TGFβR fusion protein, is administered at a dose of 15 mg/kg via IV infusion every two weeks. In one embodiment, the ICOS binding protein is administered at a dose of 24 mg via IV infusion every three weeks, and the polypeptide comprising a PD-1 inhibitor and a TGFβR, or anti-PD-(L)1(IgG):TGFβR fusion protein, is administered at a dose of 1200 mg via IV infusion every two weeks. In one embodiment, the ICOS binding protein is administered at a dose of 24 mg via IV infusion every three weeks and the polypeptide comprising a PD-1 inhibitor and a TGFβR, or anti-PD-(L)1(IgG):TGFβR fusion protein, is administered at a dose of 15 mg/kg via IV infusion every two weeks. In one embodiment, the ICOS binding protein is administered at a dose of 80 mg via IV infusion every three weeks and the polypeptide comprising a PD-1 inhibitor and a TGFβR, or anti-PD-(L)1(IgG):TGFβR fusion protein, is administered at a dose of 1200 mg via IV infusion every two weeks. In one embodiment, the ICOS binding protein is administered at a dose of 80 mg via IV infusion every three weeks and the polypeptide comprising a PD-1 inhibitor and a TGFβR, or anti-PD-(L)1(IgG):TGFβR fusion protein, is administered at a dose of 15 mg/kg via IV infusion every two weeks.

In one embodiment, the ICOS binding protein is administered at a dose of 0.3 mg/kg via IV infusion every three weeks and the polypeptide comprising a PD-1 inhibitor and a TGFβR, or anti-PD-(L)1(IgG):TGFβR fusion protein, is administered at a dose of 2400 mg via IV infusion every three weeks. In one embodiment, the ICOS binding protein is administered at a dose of 0.3 mg/kg via IV infusion every three weeks and the polypeptide comprising a PD-1 inhibitor and a TGFβR, or anti-PD-(L)1(IgG):TGFβR fusion protein, is administered at a dose of 30 mg/kg via IV infusion every three weeks. In one embodiment, the ICOS binding protein is administered at a dose of 24 mg via IV infusion every three weeks and the polypeptide comprising a PD-1 inhibitor and a TGFβR, or anti-PD-(L)1(IgG):TGFβR fusion protein, is administered at a dose of 2400 mg via IV infusion every three weeks. In one embodiment, the ICOS binding protein is administered at a dose of 24 mg via IV infusion every three weeks and the polypeptide comprising a PD-1 inhibitor and a TGFβR, or anti-PD-(L)1(IgG):TGFβR fusion protein, is administered at a dose of 30 mg/kg via IV infusion every three weeks. In one embodiment, the ICOS binding protein is administered at a dose of 80 mg via IV infusion every three weeks and the polypeptide comprising a PD-1 inhibitor and a TGFβR, or anti-PD-(L)1(IgG):TGFβR fusion protein, is administered at a dose of 2400 mg via IV infusion every three weeks. In one embodiment, the ICOS binding protein is administered at a dose of 80 mg via IV infusion every three weeks and the polypeptide comprising a PD-1 inhibitor and a TGFβR, or anti-PD-(L)1(IgG):TGFβR fusion protein, is administered at a dose of 30 mg/kg via IV infusion every three weeks.

In one embodiment, the polypeptide comprising a PD-1 inhibitor and a TGFβR, or anti-PD-(L)1(IgG):TGFβR fusion protein, is administered once every two weeks. In one embodiment, the polypeptide comprising a PD-1 inhibitor and a TGFβR, or anti-PD-(L)1(IgG):TGFβR fusion protein, is bintrafusp alfa. In one embodiment, 1200 mg of bintrafusp alfa is administered via IV infusion every 2 weeks. In a further embodiment, 15 mg/kg of bintrafusp alfa is administered via IV infusion every 2 weeks. In one embodiment, the polypeptide comprising a PD-1 inhibitor and a TGFβR, or anti-PD-(L)1(IgG):TGFβR fusion protein, is administered once every three weeks. In one embodiment, the polypeptide comprising a PD-1 inhibitor and a TGFβR, or anti-PD-(L)1(IgG):TGFβR fusion protein, is bintrafusp alfa. In one embodiment, 2400 mg of bintrafusp alfa is administered via IV infusion every 3 weeks. In a further embodiment, 30 mg/kg of bintrafusp alfa is administered via IV infusion every 3 weeks.

In some embodiments, the patient is first administered the ICOS binding protein as a monotherapy regimen and then the ICOS binding protein with the polypeptide comprising a PD-1 inhibitor and a TGFβR, or anti-PD-(L)1(IgG):TGFβR fusion protein, as a combination therapy regimen. In some embodiments, the patient is first administered the polypeptide comprising a PD-1 inhibitor and a TGFβR, or anti-PD-(L)1(IgG):TGFβR fusion protein, as a monotherapy regimen and then the ICOS binding protein with the polypeptide comprising a PD-1 inhibitor and a TGFβR, or anti-PD-(L)1(IgG):TGFβR fusion protein, as a combination therapy regimen.

In some embodiments, the patient is first administered the ICOS binding protein at a dose of about 0.08 mg to about 800 mg as a monotherapy regimen and then the ICOS binding protein at a dose of about 0.08 mg to about 800 mg with the polypeptide comprising a PD-1 inhibitor and a TGFβR, or anti-PD-(L)1(IgG):TGFβR fusion protein, at a dose of 500 mg to 3000 mg as a combination therapy regimen. In one embodiment, the patient is first administered the ICOS binding protein at a dose of about 8 mg, about 24 mg, about 48 mg, about 80 mg, about 160 mg or about 240 mg as a monotherapy regimen and then the ICOS binding protein at a dose of about 8 mg, about 24 mg, about 48 mg, about 80 mg, about 160 mg or about 240 mg with the polypeptide comprising a PD-1 inhibitor and a TGFβR, or anti-PD-(L)1(IgG):TGFβR fusion protein, at a dose of 500 mg to 3000 mg as a combination therapy regimen. In one embodiment, the patient is first administered the ICOS binding protein at a dose of 24 mg as a monotherapy regimen and then the ICOS binding protein at a dose of 24 mg with the polypeptide comprising a PD-1 inhibitor and a TGFβR, or anti-PD-(L)1(IgG):TGFβR fusion protein, at a dose of 2400 mg as a combination therapy regimen. In one embodiment, the patient is first administered the ICOS binding protein at a dose of 80 mg as a monotherapy regimen and then the ICOS binding protein at a dose of 80 mg with the polypeptide comprising a PD-1 inhibitor and a TGFβR, or anti-PD-(L)1(IgG):TGFβR fusion protein, at a dose of 2400 mg as a combination therapy regimen.

In a further embodiment, the patient is first administered the ICOS binding protein at a dose of 24 mg as a monotherapy regimen every 3 weeks for up to 1, 2, 3, 4, 5, 6, 7, 8, 9, 10, 11, 12, 13 or 14 cycles and then the ICOS binding protein at a dose of 24 mg with the polypeptide comprising a PD-1 inhibitor and a TGFβR, or anti-PD-(L)1(IgG):TGFβR fusion protein, at a dose of 2400 mg as a combination therapy regimen every 3 weeks for up to 1, 2, 3, 4, 5, 6, 7, 8, 9, 10, 11, 12, 13 or 14 cycles. In a further embodiment, the patient is first administered the ICOS binding protein at a dose of 80 mg as a monotherapy regimen every 3 weeks for up to 1, 2, 3, 4, 5, 6, 7, 8, 9, 10, 11, 12, 13 or 14 cycles and then the ICOS binding protein at a dose of 80 mg with the polypeptide comprising a PD-1 inhibitor and a TGFβR, or anti-PD-(L)1(IgG):TGFβR fusion protein, at a dose of 2400 mg as a combination therapy regimen every 3 weeks for up to 1, 2, 3, 4, 5, 6, 7, 8, 9, 10, 11, 12, 13 or 14 cycles.

In a further embodiment, the patient is first administered the ICOS binding protein at a dose of 24 mg as a monotherapy regimen every 3 weeks for up to 1, 2, 3, 4, 5, 6, 7, 8, 9, 10, 11, 12, 13 or 14 cycles and then the ICOS binding protein at a dose of 24 mg every 3 weeks with the polypeptide comprising a PD-1 inhibitor and a TGFβR, or anti-PD-(L)1(IgG):TGFβR fusion protein, at a dose of 1200 mg as a combination therapy regimen every 2 weeks for up to 1, 2, 3, 4, 5, 6, 7, 8, 9, 10, 11, 12, 13 or 14 cycles. In a further embodiment, the patient is first administered the ICOS binding protein at a dose of 80 mg as a monotherapy regimen every 3 weeks for up to 1, 2, 3, 4, 5, 6, 7, 8, 9, 10, 11, 12, 13 or 14 cycles and then the ICOS binding protein at a dose of 80 mg every 3 weeks with the polypeptide comprising a PD-1 inhibitor and a TGFβR, or anti-PD-(L)1(IgG):TGFβR fusion protein, at a dose of 1200 mg as a combination therapy regimen every 2 weeks for up to 1, 2, 3, 4, 5, 6, 7, 8, 9, 10, 11, 12, 13 or 14 cycles.

In some embodiments, the patient is first administered the ICOS binding protein at a dose of about 0.001 mg/kg to about 10 mg/kg as a monotherapy regimen and then the ICOS binding protein at a dose of about 0.001 mg/kg to about 10 mg/kg with the polypeptide comprising a PD-1 inhibitor and a TGFβR, or anti-PD-(L)1(IgG):TGFβR fusion protein, at a dose of 6.25 mg/kg to 37.5 mg/kg as a combination therapy regimen. In one embodiment, the patient is first administered the ICOS binding protein at a dose of 0.3 mg/kg as a monotherapy regimen and then the ICOS binding protein at a dose of 0.3 mg/kg with the polypeptide comprising a PD-1 inhibitor and a TGFβR, or anti-PD-(L)1(IgG):TGFβR fusion protein, at a dose of 30 mg/kg as a combination therapy regimen. In one embodiment, the patient is first administered the ICOS binding protein at a dose of 1 mg/kg as a monotherapy regimen and then the ICOS binding protein at a dose of 1 mg/kg with the polypeptide comprising a PD-1 inhibitor and a TGFβR, or anti-PD-(L)1(IgG):TGFβR fusion protein, at a dose of 30 mg/kg as a combination therapy regimen. In one embodiment, the patient is first administered the ICOS binding protein at a dose of 0.3 mg/kg as a monotherapy regimen and then the ICOS binding protein at a dose of 0.3 mg/kg with the polypeptide comprising a PD-1 inhibitor and a TGFβR, or anti-PD-(L)1(IgG):TGFβR fusion protein, at a dose of 15 mg/kg as a combination therapy regimen. In one embodiment, the patient is first administered the ICOS binding protein at a dose of 1 mg/kg as a monotherapy regimen and then the ICOS binding protein at a dose of 1 mg/kg with the polypeptide comprising a PD-1 inhibitor and a TGFβR, or anti-PD-(L)1(IgG):TGFβR fusion protein, at a dose of 15 mg/kg as a combination therapy regimen.

In a further embodiment, the patient is first administered the ICOS binding protein at a dose of 0.3 mg/kg as a monotherapy regimen every 3 weeks for up to 1, 2, 3, 4, 5, 6, 7, 8, 9, 10, 11, 12, 13 or 14 cycles and then the ICOS binding protein at a dose of 0.3 mg/kg with the polypeptide comprising a PD-1 inhibitor and a TGFβR, or anti-PD-(L)1(IgG):TGFβR fusion protein, at a dose of 30 mg/kg as a combination therapy regimen every 3 weeks for up to 1, 2, 3, 4, 5, 6, 7, 8, 9, 10, 11, 12, 13 or 14 cycles. In a further embodiment, the patient is first administered the ICOS binding protein at a dose of 1 mg/kg as a monotherapy regimen every 3 weeks for up to 1, 2, 3, 4, 5, 6, 7, 8, 9, 10, 11, 12, 13 or 14 cycles and then the ICOS binding protein at a dose of 1 mg/kg with the polypeptide comprising a PD-1 inhibitor and a TGFβR, or anti-PD-(L)1(IgG):TGFβR fusion protein, at a dose of 30 mg/kg as a combination therapy regimen every 3 weeks for up to 1, 2, 3, 4, 5, 6, 7, 8, 9, 10, 11, 12, 13 or 14 cycles.

In a further embodiment, the patient is first administered the ICOS binding protein at a dose of 0.3 mg/kg as a monotherapy regimen every 3 weeks for up to 1, 2, 3, 4, 5, 6, 7, 8, 9, 10, 11, 12, 13 or 14 cycles and then the ICOS binding protein at a dose of 0.3 mg/kg every 3 weeks with the polypeptide comprising a PD-1 inhibitor and a TGFβR, or anti-PD-(L)1(IgG):TGFβR fusion protein, at a dose of 15 mg/kg, as a combination therapy regimen every 2 weeks for up to 1, 2, 3, 4, 5, 6, 7, 8, 9, 10, 11, 12, 13 or 14 cycles. In a further embodiment, the patient is first administered the ICOS binding protein at a dose of 1 mg/kg as a monotherapy regimen every 3 weeks for up to 1, 2, 3, 4, 5, 6, 7, 8, 9, 10, 11, 12, 13 or 14 cycles and then the ICOS binding protein at a dose of 1 mg/kg every 3 weeks with the polypeptide comprising a PD-1 inhibitor and a TGFβR, or anti-PD-(L)1(IgG):TGFβR fusion protein, at a dose of 15 mg/kg as a combination therapy regimen every 2 weeks for up to 1, 2, 3, 4, 5, 6, 7, 8, 9, 10, 11, 12, 13 or 14 cycles.

In some embodiments, the patient is first administered the polypeptide comprising a PD-1 inhibitor and a TGFβR, or anti-PD-(L)1(IgG):TGFβR fusion protein, at a dose of 500 mg to 3000 mg as a monotherapy regimen and then the polypeptide comprising a PD-1 inhibitor and a TGFβR, or anti-PD-(L)1(IgG):TGFβR fusion protein, at a dose of 500 mg to 3000 mg, with the ICOS binding protein at a dose of about 0.08 mg to about 800 mg, as a combination therapy regimen. In one embodiment, the patient is first administered the polypeptide comprising a PD-1 inhibitor and a TGFβR, or anti-PD-(L)1(IgG):TGFβR fusion protein, at a dose of 500 mg to 3000 mg, and then the polypeptide comprising a PD-1 inhibitor and a TGFβR, or anti-PD-(L)1(IgG):TGFβR fusion protein, at a dose of 500 mg to 3000 mg, with the ICOS binding protein at a dose of about 8 mg, about 24 mg, about 48 mg, about 80 mg, about 160 mg or about 240 mg, as a combination therapy regimen. In one embodiment, the patient is first administered the polypeptide comprising a PD-1 inhibitor and a TGFβR, or anti-PD-(L)1(IgG):TGFβR fusion protein, at a dose of 1200 mg and then the polypeptide comprising a PD-1 inhibitor and a TGFβR, or anti-PD-(L)1(IgG):TGFβR fusion protein, at a dose of 1200 mg, with the ICOS binding protein at a dose of 24 mg, as a combination therapy regimen. In one embodiment, the patient is first administered the polypeptide comprising a PD-1 inhibitor and a TGFβR, or anti-PD-(L)1(IgG):TGFβR fusion protein, at a dose of 1200 mg and then the polypeptide comprising a PD-1 inhibitor and a TGFβR, or anti-PD-(L)1(IgG):TGFβR fusion protein, at a dose of 1200 mg with the ICOS binding protein at a dose of 80 mg, as a combination therapy regimen. In one embodiment, the patient is first administered the polypeptide comprising a PD-1 inhibitor and a TGFβR, or anti-PD-(L)1(IgG):TGFβR fusion protein, at a dose of 1200 mg and then the polypeptide comprising a PD-1 inhibitor and a TGFβR, or anti-PD-(L)1(IgG):TGFβR fusion protein, at a dose of 2400 mg with the ICOS binding protein at a dose of 24 mg, as a combination therapy regimen. In one embodiment, the patient is first administered the polypeptide comprising a PD-1 inhibitor and a TGFβR, or anti-PD-(L)1(IgG):TGFβR fusion protein, at a dose of 1200 mg and then the polypeptide comprising a PD-1 inhibitor and a TGFβR, or anti-PD-(L)1(IgG):TGFβR fusion protein, at a dose of 2400 mg, with the ICOS binding protein at a dose of 80 mg, as a combination therapy regimen. In one embodiment, the patient is first administered the polypeptide comprising a PD-1 inhibitor and a TGFβR, or anti-PD-(L)1(IgG):TGFβR fusion protein, at a dose of 2400 mg and then the polypeptide comprising a PD-1 inhibitor and a TGFβR, or anti-PD-(L)1(IgG):TGFβR fusion protein, at a dose of 2400 mg, with the ICOS binding protein at a dose of 24 mg, as a combination therapy regimen. In one embodiment, the patient is first administered the polypeptide comprising a PD-1 inhibitor and a TGFβR, or anti-PD-(L)1(IgG):TGFβR fusion protein, at a dose of 2400 mg and then the polypeptide comprising a PD-1 inhibitor and a TGFβR, or anti-PD-(L)1(IgG):TGFβR fusion protein, at a dose of 2400 mg, with the ICOS binding protein at a dose of 80 mg, as a combination therapy regimen.

In a further embodiment, the patient is first administered the polypeptide comprising a PD-1 inhibitor and a TGFβR, or anti-PD-(L)1(IgG):TGFβR fusion protein, at a dose of 1200 mg as a monotherapy regimen every 2 weeks for up to 1, 2, 3, 4, 5, 6, 7, 8, 9, 10, 11, 12, 13 or 14 cycles and then the polypeptide comprising a PD-1 inhibitor and a TGFβR, or anti-PD-(L)1(IgG):TGFβR fusion protein, at a dose of 2400 mg, with the ICOS binding protein at a dose of 24 mg, as a combination therapy regimen every 3 weeks for up to 1, 2, 3, 4, 5, 6, 7, 8, 9, 10, 11, 12, 13 or 14 cycles. In a further embodiment, the patient is first administered the polypeptide comprising a PD-1 inhibitor and a TGFβR, or anti-PD-(L)1(IgG):TGFβR fusion protein, at a dose of 1200 mg as a monotherapy regimen every 2 weeks for up to 1, 2, 3, 4, 5, 6, 7, 8, 9, 10, 11, 12, 13 or 14 cycles and then the polypeptide comprising a PD-1 inhibitor and a TGFβR, or anti-PD-(L)1(IgG):TGFβR fusion protein, at a dose of 2400 mg, with the ICOS binding protein at a dose of 80 mg, as a combination therapy regimen every 3 weeks for up to 1, 2, 3, 4, 5, 6, 7, 8, 9, 10, 11, 12, 13 or 14 cycles. In a further embodiment, the patient is first administered the polypeptide comprising a PD-1 inhibitor and a TGFβR or anti-PD-(L)1(IgG):TGFβR fusion protein at a dose of 2400 mg as a monotherapy regimen every 3 weeks for up to 1, 2, 3, 4, 5, 6, 7, 8, 9, 10, 11, 12, 13 or 14 cycles and then the polypeptide comprising a PD-1 inhibitor and a TGFβR or anti-PD-(L)1(IgG):TGFβR fusion protein at a dose of 2400 mg with the ICOS binding protein at a dose of 24 mg as a combination therapy regimen every 3 weeks for up to 1, 2, 3, 4, 5, 6, 7, 8, 9, 10, 11, 12, 13 or 14 cycles. In a further embodiment, the patient is first administered the polypeptide comprising a PD-1 inhibitor and a TGFβR or anti-PD-(L)1(IgG):TGFβR fusion protein at a dose of 2400 mg as a monotherapy regimen every 3 weeks for up to 1, 2, 3, 4, 5, 6, 7, 8, 9, 10, 11, 12, 13 or 14 cycles and then the polypeptide comprising a PD-1 inhibitor and a TGFβR or anti-PD-(L)1(IgG):TGFβR fusion protein at a dose of 2400 mg with the ICOS binding protein at a dose of 80 mg as a combination therapy regimen every 3 weeks for up to 1, 2, 3, 4, 5, 6, 7, 8, 9, 10, 11, 12, 13 or 14 cycles.

In some embodiments, the patient is first administered the polypeptide comprising a PD-1 inhibitor and a TGFβR, or anti-PD-(L)1(IgG):TGFβR fusion protein, at a dose of 6.25 mg/kg to 37.5 mg/kg as a monotherapy regimen and then the polypeptide comprising a PD-1 inhibitor and a TGFβR, or anti-PD-(L)1(IgG):TGFβR fusion protein, at a dose of 6.25 mg/kg to 37.5 mg/kg, with the ICOS binding protein at a dose of about 0.001 mg/kg to about 10 mg/kg, as a combination therapy regimen.

In one embodiment, the patient is first administered the polypeptide comprising a PD-1 inhibitor and a TGFβR, or anti-PD-(L)1(IgG):TGFβR fusion protein, at a dose of 15 mg/kg as a monotherapy regimen and then the polypeptide comprising a PD-1 inhibitor and a TGFβR, or anti-PD-(L)1(IgG):TGFβR fusion protein, at a dose of 30 mg/kg, with the ICOS binding protein at a dose of 0.3 mg/kg as a combination therapy regimen. In one embodiment, the patient is first administered the polypeptide comprising a PD-1 inhibitor and a TGFβR, or anti-PD-(L)1(IgG):TGFβR fusion protein, at a dose of 15 mg/kg as a monotherapy regimen and then the polypeptide comprising a PD-1 inhibitor and a TGFβR, or anti-PD-(L)1(IgG):TGFβR fusion protein, at a dose of 30 mg/kg, with the ICOS binding protein at a dose of 1 mg/kg as a combination therapy regimen. In one embodiment, the patient is first administered the polypeptide comprising a PD-1 inhibitor and a TGFβR, or anti-PD-(L)1(IgG):TGFβR fusion protein, at a dose of 30 mg/kg as a monotherapy regimen and then the polypeptide comprising a PD-1 inhibitor and a TGFβR, or anti-PD-(L)1(IgG):TGFβR fusion protein, at a dose of 30 mg/kg, with the ICOS binding protein at a dose of 0.3 mg/kg as a combination therapy regimen. In one embodiment, the patient is first administered the polypeptide comprising a PD-1 inhibitor and a TGFβR, or anti-PD-(L)1(IgG):TGFβR fusion protein, at a dose of 30 mg/kg as a monotherapy regimen and then the polypeptide comprising a PD-1 inhibitor and a TGFβR, or anti-PD-(L)1(IgG):TGFβR fusion protein, at a dose of 30 mg/kg with the ICOS binding protein at a dose of 1 mg/kg as a combination therapy regimen.

In a further embodiment, the patient is first administered the polypeptide comprising a PD-1 inhibitor and a TGFβR, or anti-PD-(L)1(IgG):TGFβR fusion protein, at a dose of 15 mg/kg as a monotherapy regimen every 2 weeks for up to 1, 2, 3, 4, 5, 6, 7, 8, 9, 10, 11, 12, 13 or 14 cycles and then the polypeptide comprising a PD-1 inhibitor and a TGFβR, or anti-PD-(L)1(IgG):TGFβR fusion protein, at a dose of 30 mg/kg, with the ICOS binding protein at a dose of 0.3 mg/kg as a combination therapy regimen every 3 weeks for up to 1, 2, 3, 4, 5, 6, 7, 8, 9, 10, 11, 12, 13 or 14 cycles. In a further embodiment, the patient is first administered the polypeptide comprising a PD-1 inhibitor and a TGFβR, or anti-PD-(L)1(IgG):TGFβR fusion protein, at a dose of 15 mg/kg as a monotherapy regimen every 2 weeks for up to 1, 2, 3, 4, 5, 6, 7, 8, 9, 10, 11, 12, 13 or 14 cycles and then the polypeptide comprising a PD-1 inhibitor and a TGFβR, or anti-PD-(L)1(IgG):TGFβR fusion protein, at a dose of 30 mg/kg, with the ICOS binding protein at a dose of 1 mg/kg as a combination therapy regimen every 3 weeks for up to 1, 2, 3, 4, 5, 6, 7, 8, 9, 10, 11, 12, 13 or 14 cycles. In a further embodiment, the patient is first administered the polypeptide comprising a PD-1 inhibitor and a TGFβR, or anti-PD-(L)1(IgG):TGFβR fusion protein, at a dose of 30 mg/kg as a monotherapy regimen every 3 weeks for up to 1, 2, 3, 4, 5, 6, 7, 8, 9, 10, 11, 12, 13 or 14 cycles and then the polypeptide comprising a PD-1 inhibitor and a TGFβR, or anti-PD-(L)1(IgG):TGFβR fusion protein, at a dose of 30 mg/kg, with the ICOS binding protein at a dose of 0.3 mg/kg as a combination therapy regimen every 3 weeks for up to 1, 2, 3, 4, 5, 6, 7, 8, 9, 10, 11, 12, 13 or 14 cycles. In a further embodiment, the patient is first administered the polypeptide comprising a PD-1 inhibitor and a TGFβR, or anti-PD-(L)1(IgG):TGFβR fusion protein, at a dose of 30 mg/kg as a monotherapy regimen every 3 weeks for up to 1, 2, 3, 4, 5, 6, 7, 8, 9, 10, 11, 12, 13 or 14 cycles and then the polypeptide comprising a PD-1 inhibitor and a TGFβR, or anti-PD-(L)1(IgG):TGFβR fusion protein, at a dose of 30 mg/kg, with the ICOS binding protein at a dose of 1 mg/kg, as a combination therapy regimen every 3 weeks for up to 1, 2, 3, 4, 5, 6, 7, 8, 9, 10, 11, 12, 13 or 14 cycles.

It will be understood that between first administration to the patient of an ICOS binding protein, and/or a polypeptide comprising a PD-1 inhibitor and a TGFβR, or an anti-PD-(L)1(IgG):TGFβR fusion protein, as a monotherapy and the administration of the ICOS binding protein and/or a polypeptide comprising a PD-1 inhibitor and a TGFβR, or an anti-PD-(L)1(IgG):TGFβR fusion protein, as a combination therapy as described herein, a period of no treatment or no administration may be performed, such as for a defined number of cycles. For example, after first administration with a monotherapy, the patient may be administered no treatment for 1 cycle or 2 cycles of 3 weeks, 6 weeks or 12 weeks before being administered a combination therapy as described herein. Thus, in one embodiment, the patient is first administered an ICOS binding protein as a monotherapy as described herein, then administered no treatment for 1 cycle or 2 cycles of 3 weeks, 6 weeks or 12 weeks, before the patient is administered an ICOS binding protein with a polypeptide comprising a PD-1 inhibitor and a TGFβR, or an anti-PD-(L)1(IgG):TGFβR fusion protein, as a combination therapy as described herein. In one embodiment, the patient is first administered a polypeptide comprising a PD-1 inhibitor and a TGFβR, or an anti-PD-(L)1(IgG):TGFβR fusion protein, as a monotherapy as described herein, then administered no treatment for 1 cycle or 2 cycles of 3 weeks, 6 weeks or 12 weeks, before the patient is administered a polypeptide comprising a PD-1 inhibitor and a TGFβR, or an anti-PD-(L)1(IgG):TGFβR fusion protein, with an ICOS binding protein as a combination therapy as described herein.

In one aspect, there is provided a method of treating cancer in a human in need thereof, the method comprising administering to the human an ICOS binding protein at a dose of about 0.08 mg to about 240 mg and administering to the human a polypeptide comprising a PD-1 inhibitor and a TGFβR, or an anti-PD-(L)1(IgG):TGFβR fusion protein, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the ICOS binding protein is administered at a dose of about 24 mg to about 160 mg, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the ICOS binding protein is administered at a dose of 24 mg, 48 mg, 80 mg or 160 mg. In one embodiment, the ICOS binding protein is administered at a dose of 24 mg, 48 mg, 80 mg or 160 mg; and the polypeptide comprising a PD-1 inhibitor and a TGFβR, or the anti-PD-(L)1(IgG):TGFβR fusion protein, is administered at a dose of 1200 mg. In another embodiment, the ICOS binding protein is administered at a dose of 24 mg, 48 mg, 80 mg or 160 mg, and the polypeptide comprising a PD-1 inhibitor and a TGFβR, or the anti-PD-(L)1(IgG):TGFβR fusion protein, is administered at a dose of 2400 mg. In one embodiment, the polypeptide comprising a PD-1 inhibitor and a TGFβR, or the anti-PD-(L)1(IgG):TGFβR fusion protein, is bintrafusp alfa. In one embodiment, the ICOS binding protein comprises one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:4; CDRL2 as set forth in SEQ ID NO:5 and/or CDRL3 as set forth in SEQ ID NO:6 or a direct equivalent of each CDR wherein a direct equivalent has no more than two amino acid substitutions in said CDR.

In one embodiment, the ICOS binding protein comprises a heavy chain variable region comprising one or more of SEQ ID NO:1; SEQ ID NO:2; and SEQ ID NO:3 and wherein said ICOS binding protein comprises a light chain variable region comprising one or more of SEQ ID NO:4; SEQ ID NO:5, and SEQ ID NO:6. In one embodiment, the ICOS binding protein comprises a heavy chain variable region comprising SEQ ID NO:1; SEQ ID NO:2; and SEQ ID NO:3 and wherein said ICOS binding protein comprises a light chain variable region comprising SEQ ID NO:4; SEQ ID NO:5, and SEQ ID NO:6. In one embodiment, the ICOS binding protein comprises a V_{H} domain comprising the amino acid sequence set forth in SEQ ID NO:7 and a V_{L} domain comprising the amino acid sequence as set forth in SEQ ID NO:8. In one embodiment, the ICOS binding protein comprises a heavy chain comprising the amino acid sequence set forth in SEQ ID NO:9 and a light chain comprising the amino acid sequence as set forth in SEQ ID NO:10.

In one aspect, there is provided a method of treating cancer in a human in need thereof, the method comprising administering to the human a polypeptide comprising a PD-1 inhibitor and a TGFβR, or an anti-PD-(L)1(IgG):TGFβR fusion protein, at a dose of about 500 mg to about 3000 mg and administering to the human an ICOS binding protein, wherein the PD-1 inhibitor or an anti-PD-(L)1(IgG):TGFβR fusion protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20, wherein said PD-1 inhibitor or anti-PD-(L)1(IgG):TGFβR fusion protein specifically binds to human PD-L1. In one embodiment, the polypeptide comprising a PD-1 inhibitor and a TGFβR, or the anti-PD-(L)1(IgG):TGFβR fusion protein, is administered at a dose of about 1200 mg, wherein the PD-1 inhibitor or anti-PD-(L)1(IgG):TGFβR fusion protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20, wherein said PD-1 inhibitor or anti-PD-(L)1(IgG):TGFβR fusion protein specifically binds to human PD-L1. In another embodiment, the polypeptide comprising a PD-1 inhibitor and a TGFβR, or the anti-PD-(L)1(IgG):TGFβR fusion protein, is administered at a dose of about 2400 mg, wherein the PD-1 inhibitor or anti-PD-(L)1(IgG):TGFβR fusion protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20, wherein said PD-1 inhibitor or anti-PD-(L)1(IgG):TGFβR fusion protein specifically binds to human PD-L1. In another embodiment, the ICOS binding protein is administered at a dose of 24 mg, 48 mg, 80 mg or 160 mg; and the polypeptide comprising a PD-1 inhibitor and a TGFβR, or the anti-PD-(L)1(IgG):TGFβR fusion protein, is administered at a dose of 1200 mg. In another embodiment, the ICOS binding protein is administered at a dose of 24 mg, 48 mg, 80 mg or 160 mg; and the polypeptide comprising a PD-1 inhibitor and a TGFβR, or the anti-PD-(L)1(IgG):TGFβR fusion protein, is administered at a dose of 2400 mg. In one embodiment, the PD-1 inhibitor or the anti-PD-(L)1(IgG):TGFβR fusion protein comprises one or more of: CDRH1 as set forth in SEQ ID NO:13; CDRH2 as set forth in SEQ ID NO:14; CDRH3 as set forth in SEQ ID NO:15; CDRL1 as set forth in SEQ ID NO:16; CDRL2 as set forth in SEQ ID NO:17 and/or CDRL3 as set forth in SEQ ID NO:18 or a direct equivalent of each CDR wherein a direct equivalent has no more than two amino acid substitutions in said CDR. In one embodiment, the PD-1 inhibitor or the anti-PD-(L)1(IgG):TGFβR fusion protein comprises a heavy chain variable region comprising one or more of SEQ ID NO:13; SEQ ID NO:14; and SEQ ID NO:15 and wherein said PD-1 inhibitor or the anti-PD-(L)1(IgG):TGFβR fusion protein comprises a light chain variable region comprising one or more of SEQ ID NO:16; SEQ ID NO:17, and SEQ ID NO:18. In one embodiment, the PD-1 inhibitor or the anti-PD-(L)1(IgG):TGFβR fusion protein comprises a heavy chain variable region comprising SEQ ID NO:13; SEQ ID NO:14; and SEQ ID NO:15 and wherein said PD-1 inhibitor or the anti-PD-(L)1(IgG):TGFβR fusion protein comprises a light chain variable region comprising SEQ ID NO:16; SEQ ID NO:17, and SEQ ID NO:18. In one embodiment, the PD-1 inhibitor or the anti-PD-(L)1(IgG):TGFβR fusion protein comprises a V_{H} domain comprising the amino acid sequence set forth in SEQ ID NO:19 and a V_{L} domain comprising the amino acid sequence as set forth in SEQ ID NO:20. In one embodiment, the PD-1 inhibitor or the anti-PD-(L)1(IgG):TGFβR fusion protein comprises a heavy chain comprising the amino acid sequence set forth in SEQ ID NO:21 and a light chain comprising the amino acid sequence as set forth in SEQ ID NO:22. In one embodiment, the polypeptide comprising a PD-1 inhibitor and a TGFβR, or the anti-PD-(L)1(IgG):TGFβR fusion protein, comprises human TGFβRII, or a fragment thereof capable of binding to TGFβ. In one embodiment, the polypeptide comprising a PD-1 inhibitor and a TGFβR, or the anti-PD-(L)1(IgG):TGFβR fusion protein, is bintrafusp alfa.

In one aspect, there is provided an ICOS binding protein and a polypeptide comprising a PD-1 inhibitor and a TGFβR, or the anti-PD-(L)1(IgG):TGFβR fusion protein, for concurrent or sequential use in treating cancer, wherein the ICOS binding protein is to be administered at a dose of about 0.08 mg to about 240 mg; and the polypeptide comprising a PD-1 inhibitor and a TGFβR, or the anti-PD-(L)1(IgG):TGFβR fusion protein, is to be administered at a dose of about 500 mg to about 3000 mg, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the ICOS binding protein is to be administered at a dose of about 24 mg to about 160 mg and the polypeptide comprising a PD-1 inhibitor and a TGFβR, or the anti-PD-(L)1(IgG):TGFβR fusion protein, is to be administered at a dose of about 1200 mg, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8, wherein said ICOS binding protein specifically binds to human ICOS. In another embodiment, the ICOS binding protein is to be administered at a dose of about 24 mg to about 160 mg; and the polypeptide comprising a PD-1 inhibitor and a TGFβR, or the anti-PD-(L)1(IgG):TGFβR fusion protein, is to be administered at a dose of about 2400 mg, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the ICOS binding protein is administered at a dose of 24 mg, 48 mg, 80 mg or 160 mg. In one embodiment, the ICOS binding protein is administered at a dose of 24 mg, 48 mg, 80 mg or 160 mg; and the polypeptide comprising a PD-1 inhibitor and a TGFβR, or the anti-PD-(L)1(IgG):TGFβR fusion protein, is administered at a dose of 1200 mg. In another embodiment, the ICOS binding protein is administered at a dose of 24 mg, 48 mg, 80 mg or 160 mg, and the polypeptide comprising a PD-1 inhibitor and a TGFβR, or the anti-PD-(L)1(IgG):TGFβR fusion protein, is administered at a dose of 2400 mg. In one embodiment, the polypeptide comprising a PD-1 inhibitor and a TGFβR, or the anti-PD-(L)1(IgG):TGFβR fusion protein, is bintrafusp alfa. In one embodiment, the ICOS binding protein comprises one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:4; CDRL2 as set forth in SEQ ID NO:5 and/or CDRL3 as set forth in SEQ ID NO:6 or a direct equivalent of each CDR wherein a direct equivalent has no more than two amino acid substitutions in said CDR. In one embodiment, the ICOS binding protein comprises a heavy chain variable region comprising one or more of SEQ ID NO:1; SEQ ID NO:2; and SEQ ID NO:3 and wherein said ICOS binding protein comprises a light chain variable region comprising one or more of SEQ ID NO:4; SEQ ID NO:5, and SEQ ID NO:6. In one embodiment, the ICOS binding protein comprises a heavy chain variable region comprising SEQ ID NO:1; SEQ ID NO:2; and SEQ ID NO:3 and wherein said ICOS binding protein comprises a light chain variable region comprising SEQ ID NO:4; SEQ ID NO:5, and SEQ ID NO:6. In one embodiment, the ICOS binding protein comprises a V_{H} domain comprising the amino acid sequence set forth in SEQ ID NO:7 and a V_{L} domain comprising the amino acid sequence as set forth in SEQ ID NO:8. In one embodiment, the ICOS binding protein comprises a heavy chain comprising the amino acid sequence set forth in SEQ ID NO:9 and a light chain comprising the amino acid sequence as set forth in SEQ ID NO:10.

In one aspect, there is provided a polypeptide comprising a PD-1 inhibitor and a TGFβR, or a anti-PD-(L)1(IgG):TGFβR fusion protein; and an ICOS binding protein for concurrent or sequential use in treating cancer, wherein the polypeptide comprising a PD-1 inhibitor and a TGFβR, or a anti-PD-(L)1(IgG):TGFβR fusion protein, is to be administered at a dose of about 500 mg to about 3000 mg and the ICOS binding protein is to be administered at a dose of about 0.08 mg to about 240 mg, wherein the PD-1 inhibitor or the anti-PD-(L)1(IgG):TGFβR fusion protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20, wherein said PD-1 inhibitor or anti-PD-(L)1(IgG):TGFβR fusion protein specifically binds to human PD-L1. In one embodiment, the polypeptide comprising a PD-1 inhibitor and a TGFβR, or the anti-PD-(L)1(IgG):TGFβR fusion protein, is to be administered at a dose of about 1200 mg; and the ICOS binding protein is to be administered at a dose of about 8 mg to about 160 mg, wherein the PD-1 inhibitor or the anti-PD-(L)1(IgG):TGFβR fusion protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20, wherein said PD-1 inhibitor or anti-PD-(L)1(IgG):TGFβR fusion protein specifically binds to human PD-L1. In one embodiment, the polypeptide comprising a PD-1 inhibitor and a TGFβR, or the anti-PD-(L)1(IgG):TGFβR fusion protein, is to be administered at a dose of about 2400 mg and the ICOS binding protein is to be administered at a dose of about 8 mg to about 160 mg, wherein the PD-1 inhibitor or the anti-PD-(L)1(IgG):TGFβR fusion protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20, wherein said PD-1 inhibitor or anti-PD-(L)1(IgG):TGFβR fusion protein specifically binds to human PD-L1. In one embodiment, the polypeptide comprising a PD-1 inhibitor and a TGFβR, or the anti-PD-(L)1(IgG):TGFβR fusion protein, is administered at a dose of 1200 mg. In one embodiment, the polypeptide comprising a PD-1 inhibitor and a TGFβR, or the anti-PD-(L)1(IgG):TGFβR fusion protein, is administered at a dose of 2400 mg. In another embodiment, the ICOS binding protein is administered at a dose of 24 mg, 48 mg, 80 mg or 160 mg; and the polypeptide comprising a PD-1 inhibitor and a TGFβR, or the anti-PD-(L)1(IgG):TGFβR fusion protein, is administered at a dose of 1200 mg. In another embodiment, the ICOS binding protein is administered at a dose of 24 mg, 48 mg, 80 mg or 160 mg; and the polypeptide comprising a PD-1 inhibitor and a TGFβR, or the anti-PD-(L)1(IgG):TGFβR fusion protein, is administered at a dose of 2400 mg. In one embodiment, the PD-1 inhibitor or the anti-PD-(L)1(IgG):TGFβR fusion protein comprises one or more of: CDRH1 as set forth in SEQ ID NO:13; CDRH2 as set forth in SEQ ID NO:14; CDRH3 as set forth in SEQ ID NO:15; CDRL1 as set forth in SEQ ID NO:16; CDRL2 as set forth in SEQ ID NO:17 and/or CDRL3 as set forth in SEQ ID NO:18 or a direct equivalent of each CDR wherein a direct equivalent has no more than two amino acid substitutions in said CDR. In one embodiment, the PD-1 inhibitor or the anti-PD-(L)1(IgG):TGFβR fusion protein comprises a heavy chain variable region comprising one or more of SEQ ID NO:13; SEQ ID NO:14; and SEQ ID NO:15 and wherein said PD-1 inhibitor or the anti-PD-(L)1(IgG):TGFβR fusion protein comprises a light chain variable region comprising one or more of SEQ ID NO:16; SEQ ID NO:17, and SEQ ID NO:18. In one embodiment, the PD-1 inhibitor or the anti-PD-(L)1(IgG):TGFβR fusion protein comprises a heavy chain variable region comprising SEQ ID NO:13; SEQ ID NO:14; and SEQ ID NO:15 and wherein said PD-1 inhibitor or the anti-PD-(L)1(IgG):TGFβR fusion protein comprises a light chain variable region comprising SEQ ID NO:16; SEQ ID NO:17, and SEQ ID NO:18. In one embodiment, the PD-1 inhibitor or the anti-PD-(L)1(IgG):TGFβR fusion protein comprises a V_{H} domain comprising the amino acid sequence set forth in SEQ ID NO:19 and a V_{L} domain comprising the amino acid sequence as set forth in SEQ ID NO:20. In one embodiment, the PD-1 inhibitor or the anti-PD-(L)1(IgG):TGFβR fusion protein comprises a heavy chain comprising the amino acid sequence set forth in SEQ ID NO:21 and a light chain comprising the amino acid sequence as set forth in SEQ ID NO:22. In one embodiment, the polypeptide comprising a PD-1 inhibitor and a TGFβR, or the anti-PD-(L)1(IgG):TGFβR fusion protein, comprises human TGFβRII, or a fragment thereof capable of binding to TGFβ. In one embodiment, the polypeptide comprising a PD-1 inhibitor and a TGFβR, or the anti-PD-(L)1(IgG):TGFβR fusion protein, is bintrafusp alfa.

In another aspect, an ICOS binding protein for use in treating cancer is provided, wherein the ICOS binding protein is to be administered at a dose of about 0.08 mg to about 240 mg and is to be administered concurrently or sequentially with a polypeptide comprising a PD-1 inhibitor and a TGFβR, or an anti-PD-(L)1(IgG):TGFβR fusion protein, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8, wherein said ICOS binding protein specifically binds to human ICOS. In one embodiment, the ICOS binding protein is to be administered at a dose of about 24 mg to about 160 mg and is to be administered concurrently or sequentially with a polypeptide comprising a PD-1 inhibitor and a TGFβR, or an anti-PD-(L)1(IgG):TGFβR fusion protein, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the ICOS binding protein is administered at a dose of 24 mg, 48 mg, 80 mg or 160 mg. In another embodiment, the ICOS binding protein is administered at a dose of 24 mg, 48 mg, 80 mg or 160 mg; and the polypeptide comprising a PD-1 inhibitor and a TGFβR, or a anti-PD-(L)1(IgG):TGFβR fusion protein, is administered at a dose of 1200 mg. In one embodiment, the ICOS binding protein is administered at a dose of 24 mg, 48 mg, 80 mg or 160 mg. In another embodiment, the ICOS binding protein is administered at a dose of 24 mg, 48 mg, 80 mg or 160 mg; and the polypeptide comprising a PD-1 inhibitor and a TGFβR, or a anti-PD-(L)1(IgG):TGFβR fusion protein, is administered at a dose of 2400 mg. In one embodiment, the polypeptide comprising a PD-1 inhibitor and a TGFβR, or the anti-PD-(L)1(IgG):TGFβR fusion protein, is bintrafusp alfa. In one embodiment, the ICOS binding protein comprises one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:4; CDRL2 as set forth in SEQ ID NO:5 and/or CDRL3 as set forth in SEQ ID NO:6 or a direct equivalent of each CDR wherein a direct equivalent has no more than two amino acid substitutions in said CDR. In one embodiment, the ICOS binding protein comprises a heavy chain variable region comprising one or more of SEQ ID NO:1; SEQ ID NO:2; and SEQ ID NO:3 and wherein said ICOS binding protein comprises a light chain variable region comprising one or more of SEQ ID NO:4; SEQ ID NO:5, and SEQ ID NO:6. In one embodiment, the ICOS binding protein comprises a heavy chain variable region comprising SEQ ID NO:1; SEQ ID NO:2; and SEQ ID NO:3 and wherein said ICOS binding protein comprises a light chain variable region comprising SEQ ID NO:4; SEQ ID NO:5, and SEQ ID NO:6. In one embodiment, the ICOS binding protein comprises a V_{H} domain comprising the amino acid sequence set forth in SEQ ID NO:7 and a V_{L} domain comprising the amino acid sequence as set forth in SEQ ID NO:8. In one embodiment, the ICOS binding protein comprises a heavy chain comprising the amino acid sequence set forth in SEQ ID NO:9 and a light chain comprising the amino acid sequence as set forth in SEQ ID NO:10.

In another aspect, a polypeptide comprising a PD-1 inhibitor and a TGFβR, or an anti-PD-(L)1(IgG):TGFβR fusion protein, for use in treating cancer is provided, wherein the polypeptide comprising a PD-1 inhibitor and a TGFβR, or the anti-PD-(L)1(IgG):TGFβR fusion protein, is to be administered at a dose of about 500 mg to about 3000 mg and is to be administered concurrently or sequentially with an ICOS binding protein, wherein the PD-1 inhibitor or the anti-PD-(L)1(IgG):TGFβR fusion protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20, wherein said PD-1 inhibitor or the anti-PD-(L)1(IgG):TGFβR fusion protein specifically binds to human PD-1.

In one embodiment, the polypeptide comprising a PD-1 inhibitor and a TGFβR, or the anti-PD-(L)1(IgG):TGFβR fusion protein, is to be administered at a dose of about 1200 mg and is to be administered concurrently or sequentially with an ICOS binding protein, wherein the PD-1 inhibitor or the anti-PD-(L)1(IgG):TGFβR fusion protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20 wherein said PD-1 inhibitor or the anti-PD-(L)1(IgG):TGFβR fusion protein specifically binds to human PD-L1. In one embodiment, the polypeptide comprising a PD-1 inhibitor and a TGFβR, or the anti-PD-(L)1(IgG):TGFβR fusion protein, is to be administered at a dose of about 2400 mg and is to be administered concurrently or sequentially with an ICOS binding protein, wherein the PD-1 inhibitor or the anti-PD-(L)1(IgG):TGFβR fusion protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20 wherein said PD-1 inhibitor or the anti-PD-(L)1(IgG):TGFβR fusion protein specifically binds to human PD-L1. In one embodiment, the polypeptide comprising a PD-1 inhibitor and a TGFβR, or the anti-PD-(L)1(IgG):TGFβR fusion protein, is administered at a dose of 1200 mg. In one embodiment, the polypeptide comprising a PD-1 inhibitor and a TGFβR, or the anti-PD-(L)1(IgG):TGFβR fusion protein, is administered at a dose of 2400 mg. In another embodiment, the ICOS binding protein is administered at a dose of 24 mg, 48 mg, 80 mg or 160 mg; and the polypeptide comprising a PD-1 inhibitor and a TGFβR, or the anti-PD-(L)1(IgG):TGFβR fusion protein, is administered at a dose of 1200 mg. In another embodiment, the ICOS binding protein is administered at a dose of 24 mg, 48 mg, 80 mg or 160 mg; and the polypeptide comprising a PD-1 inhibitor and a TGFβR, or the anti-PD-(L)1(IgG):TGFβR fusion protein, is administered at a dose of 2400 mg. In one embodiment, the PD-1 inhibitor or the anti-PD-(L)1(IgG):TGFβR fusion protein comprises one or more of: CDRH1 as set forth in SEQ ID NO:13; CDRH2 as set forth in SEQ ID NO:14; CDRH3 as set forth in SEQ ID NO:15; CDRL1 as set forth in SEQ ID NO:16; CDRL2 as set forth in SEQ ID NO:17 and/or CDRL3 as set forth in SEQ ID NO:18 or a direct equivalent of each CDR wherein a direct equivalent has no more than two amino acid substitutions in said CDR. In one embodiment, the PD-1 inhibitor or the anti-PD-(L)1(IgG):TGFβR fusion protein comprises a heavy chain variable region comprising one or more of SEQ ID NO:13; SEQ ID NO:14; and SEQ ID NO:15 and wherein said PD-1 inhibitor or the anti-PD-(L)1(IgG):TGFβR fusion protein comprises a light chain variable region comprising one or more of SEQ ID NO:16; SEQ ID NO:17, and SEQ ID NO:18. In one embodiment, the PD-1 inhibitor or the anti-PD-(L)1(IgG):TGFβR fusion protein comprises a heavy chain variable region comprising SEQ ID NO:13; SEQ ID NO:14; and SEQ ID NO:15 and wherein said PD-1 inhibitor or the anti-PD-(L)1(IgG):TGFβR fusion protein comprises a light chain variable region comprising SEQ ID NO:16; SEQ ID NO:17, and SEQ ID NO:18. In one embodiment, the PD-1 inhibitor or the anti-PD-(L)1(IgG):TGFβR fusion protein comprises a V_{H} domain comprising the amino acid sequence set forth in SEQ ID NO:19 and a V_{L} domain comprising the amino acid sequence as set forth in SEQ ID NO:20. In one embodiment, the PD-1 inhibitor or the anti-PD-(L)1(IgG):TGFβR protein comprises a heavy chain comprising the amino acid sequence set forth in SEQ ID NO:21 and a light chain comprising the amino acid sequence as set forth in SEQ ID NO:22. In one embodiment, the polypeptide comprising a PD-1 inhibitor and a TGFβR, or the anti-PD-(L)1(IgG):TGFβR fusion protein, comprises human TGFβRII, or a fragment thereof capable of binding to TGFβ. In one embodiment, the polypeptide comprising a PD-1 inhibitor and a TGFβR, or the anti-PD-(L)1(IgG):TGFβR fusion protein, is bintrafusp alfa.

In another aspect, there is provided use of an ICOS binding protein in the manufacture of a medicament for treating cancer, wherein the ICOS binding protein is to be administered at a dose of about 0.08 mg to about 240 mg and is to be administered concurrently or sequentially with a polypeptide comprising a PD-1 inhibitor and a TGFβR, or an anti-PD-(L)1(IgG):TGFβR fusion protein, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the ICOS binding protein is to be administered at a dose of about 24 mg to about 160 mg and is to be administered concurrently or sequentially with a polypeptide comprising a PD-1 inhibitor and a TGFβR, or an anti-PD-(L)1(IgG):TGFβR fusion protein, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the ICOS binding protein is administered at a dose of 24 mg, 48 mg, 80 mg or 160 mg. In one embodiment, the ICOS binding protein is administered at a dose of 24 mg, 48 mg, 80 mg or 160 mg; and the polypeptide comprising a PD-1 inhibitor and a TGFβR, or the anti-PD-(L)1(IgG):TGFβR fusion protein, is administered at a dose of 1200 mg. In one embodiment, the ICOS binding protein is administered at a dose of 24 mg, 48 mg, 80 mg or 160 mg, and the polypeptide comprising a PD-1 inhibitor and a TGFβR, or the anti-PD-(L)1(IgG):TGFβR fusion protein, is administered at a dose of 2400 mg. In one embodiment, the polypeptide comprising a PD-1 inhibitor and a TGFβR, or the anti-PD-(L)1(IgG):TGFβR fusion protein, is bintrafusp alfa. In one embodiment, the ICOS binding protein comprises one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:4; CDRL2 as set forth in SEQ ID NO:5 and/or CDRL3 as set forth in SEQ ID NO:6 or a direct equivalent of each CDR wherein a direct equivalent has no more than two amino acid substitutions in said CDR. In one embodiment, the ICOS binding protein comprises a heavy chain variable region comprising one or more of SEQ ID NO:1; SEQ ID NO:2; and SEQ ID NO:3 and wherein said ICOS binding protein comprises a light chain variable region comprising one or more of SEQ ID NO:4; SEQ ID NO:5, and SEQ ID NO:6. In one embodiment, the ICOS binding protein comprises a heavy chain variable region comprising SEQ ID NO:1; SEQ ID NO:2; and SEQ ID NO:3 and wherein said ICOS binding protein comprises a light chain variable region comprising SEQ ID NO:4; SEQ ID NO:5, and SEQ ID NO:6. In one embodiment, the ICOS binding protein comprises a V_{H} domain comprising the amino acid sequence set forth in SEQ ID NO:7 and a V_{L} domain comprising the amino acid sequence as set forth in SEQ ID NO:8. In one embodiment, the ICOS binding protein comprises a heavy chain comprising the amino acid sequence set forth in SEQ ID NO:9 and a light chain comprising the amino acid sequence as set forth in SEQ ID NO:10.

In another aspect, there is provided use of a polypeptide comprising a PD-1 inhibitor and a TGFβR, or an anti-PD-(L)1(IgG):TGFβR fusion protein in the manufacture of a medicament for treating cancer, wherein the polypeptide comprising a PD-1 inhibitor and a TGFβR, or the anti-PD-(L)1(IgG):TGFβR fusion protein, is to be administered at a dose of about 500 mg to about 3000 mg and is to be administered concurrently or sequentially with an ICOS binding protein, wherein the PD-1 inhibitor or the anti-PD-(L)1(IgG):TGFβR fusion protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20 wherein said PD-1 inhibitor or anti-PD-(L)1(IgG):TGFβR fusion protein specifically binds to human PD-L1. In one embodiment, the polypeptide comprising a PD-1 inhibitor and a TGFβR, or the anti-PD-(L)1(IgG):TGFβR fusion protein, is to be administered at a dose of about 1200 mg and is to be administered concurrently or sequentially with an ICOS binding protein, wherein the PD-1 inhibitor or the anti-PD-(L)1(IgG):TGFβR fusion protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20, wherein said PD-1 inhibitor or anti-PD-(L)1(IgG):TGFβR fusion protein specifically binds to human PD-L1. In one embodiment, the polypeptide comprising a PD-1 inhibitor and a TGFβR, or the anti-PD-(L)1(IgG):TGFβR fusion protein, is to be administered at a dose of about 2400 mg and is to be administered concurrently or sequentially with an ICOS binding protein, wherein the PD-1 inhibitor or the anti-PD-(L)1(IgG):TGFβR fusion protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20 wherein said PD-1 inhibitor or anti-PD-(L)1(IgG):TGFβR fusion protein specifically binds to human PD-L1. In one embodiment, the ICOS binding protein is administered at a dose of 24 mg, 48 mg, 80 mg or 160 mg; and the polypeptide comprising a PD-1 inhibitor and a TGFβR, or the anti-PD-(L)1(IgG):TGFβR fusion protein, is administered at a dose of 1200 mg. In one embodiment, the ICOS binding protein is administered at a dose of 24 mg, 48 mg, 80 mg or 160 mg; and the polypeptide comprising a PD-1 inhibitor and a TGFβR, or the anti-PD-(L)1(IgG):TGFβR fusion protein, is administered at a dose of 2400 mg. In one embodiment, the PD-1 inhibitor or the anti-PD-(L)1(IgG):TGFβR fusion protein comprises one or more of: CDRH1 as set forth in SEQ ID NO:13; CDRH2 as set forth in SEQ ID NO:14; CDRH3 as set forth in SEQ ID NO:15; CDRL1 as set forth in SEQ ID NO:16; CDRL2 as set forth in SEQ ID NO:17 and/or CDRL3 as set forth in SEQ ID NO:18 or a direct equivalent of each CDR wherein a direct equivalent has no more than two amino acid substitutions in said CDR. In one embodiment, the PD-1 inhibitor or the anti-PD-(L)1(IgG):TGFβR fusion protein comprises a heavy chain variable region comprising one or more of SEQ ID NO:13; SEQ ID NO:14; and SEQ ID NO:15 and wherein said PD-1 inhibitor or anti-PD-(L)1(IgG):TGFβR fusion protein comprises a light chain variable region comprising one or more of SEQ ID NO:16; SEQ ID NO:17, and SEQ ID NO:18. In one embodiment, the PD-1 inhibitor or the anti-PD-(L)1(IgG):TGFβR fusion protein comprises a heavy chain variable region comprising SEQ ID NO:13; SEQ ID NO:14; and SEQ ID NO:15 and wherein said PD-1 inhibitor or anti-PD-(L)1(IgG):TGFβR fusion protein comprises a light chain variable region comprising SEQ ID NO:16; SEQ ID NO:17, and SEQ ID NO:18. In one embodiment, the PD-1 inhibitor or the anti-PD-(L)1(IgG):TGFβR fusion protein comprises a V_{H} domain comprising the amino acid sequence set forth in SEQ ID NO:19 and a V_{L} domain comprising the amino acid sequence as set forth in SEQ ID NO:20. In one embodiment, the PD-1 inhibitor, or the anti-PD-(L)1(IgG):TGFβR fusion protein, comprises a heavy chain comprising the amino acid sequence set forth in SEQ ID NO:21 and a light chain comprising the amino acid sequence as set forth in SEQ ID NO:22. In one embodiment, the polypeptide comprising a PD-1 inhibitor and a TGFβR, or the anti-PD-(L)1(IgG):TGFβR fusion protein, comprises human TGFβRII, or a fragment thereof capable of binding to TGFβ. In one embodiment, the polypeptide comprising a PD-1 inhibitor and a TGFβR, or the anti-PD-(L)1(IgG):TGFβR fusion protein, is bintrafusp alfa.

In one aspect, there is provided a pharmaceutical kit comprising about 0.08 mg to about 240 mg of an ICOS binding protein, and a polypeptide comprising a PD-1 inhibitor and a TGFβR, or an anti-PD-(L)1(IgG):TGFβR fusion protein, wherein the ICOS binding protein comprises a V_{H} domain comprising an amino acid sequence at least 90% identical to the amino acid sequence set forth in SEQ ID NO:7 and/or a V_{L} domain comprising an amino acid sequence at least 90% identical to the amino acid sequence as set forth in SEQ ID NO:8 wherein said ICOS binding protein specifically binds to human ICOS. In one embodiment, the kit comprises 24 mg, 48 mg, 80 mg or 160 mg of an ICOS binding protein. In one embodiment, the kit comprises about 500 mg to about 3000 mg of a polypeptide comprising a PD-1 inhibitor and a TGFβR, or an anti-PD-(L)1(IgG):TGFβR fusion protein. In one embodiment, the kit comprises 1200 mg of a polypeptide comprising a PD-1 inhibitor and a TGFβR, or an anti-PD-(L)1(IgG):TGFβR fusion protein. In one embodiment, the kit comprises 2400 mg of a polypeptide comprising a PD-1 inhibitor and a TGFβR, or an anti-PD-(L)1(IgG):TGFβR fusion protein. In another embodiment, the kit comprises 24 mg, 48 mg, 80 mg or 160 mg of an ICOS binding protein; and 1200 mg of a polypeptide comprising a PD-1 inhibitor and a TGFβR, or an anti-PD-(L)1(IgG):TGFβR fusion protein. In another embodiment, the kit comprises 24 mg, 48 mg, 80 mg or 160 mg of an ICOS binding protein, and 2400 mg of a polypeptide comprising a PD-1 inhibitor and a TGFβR, or an anti-PD-(L)1(IgG):TGFβR fusion protein. In one embodiment, the polypeptide comprising a PD-1 inhibitor and a TGFβR, or the anti-PD-(L)1(IgG):TGFβR fusion protein is bintrafusp alfa. In one embodiment, the ICOS binding protein comprises one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:4; CDRL2 as set forth in SEQ ID NO:5 and/or CDRL3 as set forth in SEQ ID NO:6 or a direct equivalent of each CDR wherein a direct equivalent has no more than two amino acid substitutions in said CDR. In one embodiment, the ICOS binding protein comprises a heavy chain variable region comprising one or more of SEQ ID NO:1; SEQ ID NO:2; and SEQ ID NO:3 and wherein said ICOS binding protein comprises a light chain variable region comprising one or more of SEQ ID NO:4; SEQ ID NO:5, and SEQ ID NO:6. In one embodiment, the ICOS binding protein comprises a heavy chain variable region comprising SEQ ID NO:1; SEQ ID NO:2; and SEQ ID NO:3 and wherein said ICOS binding protein comprises a light chain variable region comprising SEQ ID NO:4; SEQ ID NO:5, and SEQ ID NO:6. In one embodiment, the ICOS binding protein comprises a V_{H} domain comprising the amino acid sequence set forth in SEQ ID NO:7 and a V_{L} domain comprising the amino acid sequence as set forth in SEQ ID NO:8. In one embodiment, the ICOS binding protein comprises a heavy chain comprising the amino acid sequence set forth in SEQ ID NO:9 and a light chain comprising the amino acid sequence as set forth in SEQ ID NO:10.

In one aspect, there is provided a pharmaceutical kit comprising about 500 mg to about 3000 mg of a polypeptide comprising a PD-1 inhibitor and a TGFβR, or an anti-PD-(L)1(IgG):TGFβR fusion protein, and an ICOS binding protein, wherein the PD-1 inhibitor or the anti-PD-(L)1(IgG):TGFβR fusion protein comprises a V_{H} domain comprising an amino acid sequence at least 90% identical to the amino acid sequence set forth in SEQ ID NO:19 and/or a V_{L} domain comprising an amino acid sequence at least 90% identical to the amino acid sequence as set forth in SEQ ID NO:20 and wherein said PD-1 inhibitor or anti-PD-(L)1(IgG):TGFβR fusion protein specifically binds to human PD-L1. In one embodiment, the kit comprises about 0.08 mg to about 240 mg of an ICOS binding protein. In one embodiment, the kit comprises 24 mg, 48 mg, 80 mg or 160 mg of an ICOS binding protein. In one embodiment, the kit comprises 1200 mg of a polypeptide comprising a PD-1 inhibitor and a TGFβR, or an anti-PD-(L)1(IgG):TGFβR fusion protein. In one embodiment, the kit comprises 2400 mg of a polypeptide comprising a PD-1 inhibitor and a TGFβR, or an anti-PD-(L)1(IgG):TGFβR fusion protein. In another embodiment, the kit comprises 24 mg, 48 mg, 80 mg or 160 mg of an ICOS binding protein; and 1200 mg of a polypeptide comprising a PD-1 inhibitor and a TGFβR, or an anti-PD-(L)1(IgG):TGFβR fusion protein. In another embodiment, the kit comprises 24 mg, 48 mg, 80 mg or 160 mg of an ICOS binding protein; and 2400 mg of a polypeptide comprising a PD-1 inhibitor and a TGFβR, or an anti-PD-(L)1(IgG):TGFβR fusion protein. In one embodiment, the PD-1 inhibitor or the anti-PD-(L)1(IgG):TGFβR fusion protein comprises one or more of: CDRH1 as set forth in SEQ ID NO:13; CDRH2 as set forth in SEQ ID NO:14; CDRH3 as set forth in SEQ ID NO:15; CDRL1 as set forth in SEQ ID NO:16; CDRL2 as set forth in SEQ ID NO:17 and/or CDRL3 as set forth in SEQ ID NO:18 or a direct equivalent of each CDR wherein a direct equivalent has no more than two amino acid substitutions in said CDR. In one embodiment, the PD-1 inhibitor or the anti-PD-(L)1(IgG):TGFβR fusion protein comprises a heavy chain variable region comprising one or more of SEQ ID NO:13; SEQ ID NO:14; and SEQ ID NO:15 and wherein said PD-1 inhibitor or the anti-PD-(L)1(IgG):TGFβR fusion protein comprises a light chain variable region comprising one or more of SEQ ID NO:16; SEQ ID NO:17, and SEQ ID NO:18. In one embodiment, the PD-1 inhibitor or the anti-PD-(L)1(IgG):TGFβR fusion protein comprises a heavy chain variable region comprising SEQ ID NO:13; SEQ ID NO:14; and SEQ ID NO:15 and wherein said PD-1 inhibitor or the anti-PD-(L)1(IgG):TGFβR fusion protein comprises a light chain variable region comprising SEQ ID NO:16; SEQ ID NO: 17, and SEQ ID NO: 18. In one embodiment, the PD-1 inhibitor or the anti-PD-(L)1(IgG):TGFβR fusion protein comprises a V_{H} domain comprising the amino acid sequence set forth in SEQ ID NO:19 and a V_{L} domain comprising the amino acid sequence as set forth in SEQ ID NO:20. In one embodiment, the PD-1 inhibitor or the anti-PD-(L)1(IgG):TGFβR fusion protein comprises a heavy chain comprising the amino acid sequence set forth in SEQ ID NO:21 and a light chain comprising the amino acid sequence as set forth in SEQ ID NO:22. In one embodiment, the polypeptide comprising a PD-1 inhibitor and a TGFβR or the anti-PD-(L)1(IgG):TGFβR fusion protein comprises human TGFβRII, or a fragment thereof capable of binding to TGFβ. In one embodiment, the polypeptide comprising a PD-1 inhibitor and a TGFβR or the anti-PD-(L)1(IgG):TGFβR fusion protein is bintrafusp alfa.

In one aspect, there is provided a method of treating cancer, the method comprising administering to a subject (e.g. a human) an ICOS binding protein at a dose wherein the median plasma concentration of the ICOS binding protein is between 100 μg/ml and 0.1 μg/ml for at least 7 days after the first dose.

In one aspect, there is provided an ICOS binding protein for use in the treatment of cancer, wherein ICOS binding protein is administered at a dose wherein the median plasma concentration of the ICOS binding protein is between 100 μg/ml and 0.1 μg/ml for at least 7 days after the first dose.

In another aspect, there is provided use of an ICOS binding protein in the manufacture of a medicament for treating cancer, wherein the ICOS binding protein is administered at a dose wherein the median plasma concentration of the ICOS binding protein is between 100 μg/ml and 0.1 μg/ml for at least 7 days after the first dose.

In one embodiment, the ICOS binding protein is administered at a dose wherein the median plasma concentration of the ICOS binding protein is between 100 μg/ml, 10 μg/ml, 1 μg/ml or 0.1 μg/ml and 10 μg/ml, 1 μg/ml or 0.1 μg/ml for at least 1, 2.5, 4.5, 7, 14 or 21 days after the first dose.

In one embodiment, the ICOS binding protein is administered at a dose wherein the median plasma concentration of the ICOS binding protein is between 100 μg/ml, 90 μg/ml, 80 μg/ml, 70 μg/ml, 60 μg/ml, 50 μg/ml, 40 μg/ml, 30 μg/ml, 20 μg/ml, 10 μg/ml, 9 μg/ml, 8 μg/ml, 7 μg/ml, 6 μg/ml, 5 μg/ml, 4 μg/ml, 3 μg/ml, 2 μg/ml, 1 μg/ml, 0.9 μg/ml, 0.8 μg/ml, 0.7 μg/ml, 0.6 μg/ml, 0.5 μg/ml, 0.4 μg/ml, 0.3 μg/ml or 0.2 μg/ml and 90 μg/ml, 80 μg/ml, 70 μg/ml, 60 μg/ml, 50 μg/ml, 40 μg/ml, 30 μg/ml, 20 μg/ml, 10 μg/ml, 9 μg/ml, 8 μg/ml, 7 μg/ml, 6 μg/ml, 5 μg/ml, 4 μg/ml, 3 μg/ml, 2 μg/ml, 1 μg/ml, 0.9 μg/ml, 0.8 μg/ml, 0.7 μg/ml, 0.6 μg/ml, 0.5 μg/ml, 0.4 μg/ml, 0.3 μg/ml, 0.2 μg/ml or 0.1 μg/ml, for at least 1, 2, 2.5, 3, 4, 4.5, 5, 6, 7, 8, 9, 10, 11, 12, 13, 14, 15, 16, 17, 18, 19, 20 or 21 days after the first dose.

In one embodiment, the human is administered an ICOS binding protein at a dose wherein the median plasma concentration of the ICOS binding protein is between 10 μg/ml and 1 μg/ml at 21 days after the first dose. In one embodiment, the human is administered an ICOS binding protein at a dose wherein the median plasma concentration of the ICOS binding protein is between 10 μg/ml and 0.1 μg/ml at 21 days after the first dose.

In one embodiment, the human is administered an ICOS binding protein at a dose wherein the median plasma concentration of the ICOS binding protein is between 100 μg/ml and 1 μg/ml at 21 days after the first dose. In one embodiment, the human is administered an ICOS binding protein at a dose wherein the median plasma concentration of the ICOS binding protein is between 100 μg/ml and 10 μg/ml at 21 days after the first dose.

In one aspect, there is provided a method of treating cancer, the method comprising administering to a subject (e.g. a human) an ICOS binding protein at a dose wherein ICOS receptor saturation or occupancy in the subject is at or above around 50% for at least 7 days after the first dose.

In one aspect, there is provided an ICOS binding protein for use in the treatment of cancer, wherein the ICOS binding protein is administered to a subject (e.g. a human) at a dose wherein ICOS receptor saturation or occupancy in the subject is at or above around 50% for at least 7 days after the first dose.

In another aspect, there is provided use of an ICOS binding protein in the manufacture of a medicament for treating cancer, wherein the ICOS binding protein is administered to a human at a dose wherein ICOS receptor saturation or occupancy in the human is at or above around 50% for at least 7 days after first dose.

In one embodiment, the human is administered an ICOS binding protein at a dose wherein ICOS receptor saturation or occupancy in the human is at or above around 50%, 55%, 60%, 65%, 70%, 75%, 80%, 85%, 90% or 95% for at least 1, 2, 3, 4, 5, 6, 7, 8, 9, 10, 11, 12, 13, 14, 15, 16, 17, 18, 19, 20 or 21 days after first dose.

In one aspect, there is provided a method of treating cancer, the method comprising administering to a subject (e.g. a human) an ICOS binding protein at a dose wherein peripheral CD4+ or CD8+ T cell receptor occupancy is at or above 50% for at least 7 days after the first dose.

In one aspect, there is provided an ICOS binding protein for use in the treatment of cancer, wherein the ICOS binding protein is administered to a human at a dose wherein peripheral CD4+ or CD8+ T cell receptor occupancy is at or above 50% for at least 7 days after the first dose.

In another aspect, there is provided use of an ICOS binding protein in the manufacture of a medicament for treating cancer, wherein the ICOS binding protein is administered to a human at a dose wherein peripheral CD4⁺ or CD8+ T cell receptor occupancy is at or above 50% for at least 7 days after the first dose.

Peak CD4⁺ Receptor Occupancy (RO) corresponds to the ICOS binding protein maximum plasma concentration. Peak CD8⁺ Receptor Occupancy (RO) corresponds to the ICOS binding protein maximum plasma concentration.

In one embodiment, the ICOS binding protein is administered at a dose wherein peripheral CD4⁺ or CD8⁺ T cell receptor occupancy is at or above around 50%, 55%, 60%, 65%, 70%, 75%, 80%, 85%, 90% or 95% for at least 1, 2, 3, 4, 5, 6, 7, 8, 9, 10, 11, 12, 13, 14, 15, 16, 17, 18, 19, 20 or 21 days after the first dose.

In one embodiment, the ICOS binding protein is administered at a dose wherein peripheral CD4⁺ or CD8⁺ T cell receptor occupancy is at or above around 60%, for at least 21 days after the first dose. In one embodiment, the ICOS binding protein is administered at a dose wherein peripheral CD4⁺ or CD8⁺ T cell receptor occupancy is at or above around 70%, for at least 21 days after the first dose.

In one embodiment, the ICOS binding protein is administered at a dose wherein peripheral CD4⁺ or CD8⁺ T cell receptor occupancy is at or above around 80%, for at least 21 days after the first dose.

In one embodiment, the ICOS binding protein is administered at a dose wherein peripheral CD4⁺ or CD8⁺ T cell receptor occupancy is at or above around 90%, for at least 21 days after the first dose.

In one aspect, there is provided a pharmaceutical composition comprising an ICOS binding protein, wherein said composition provides an Area Under the Curve (AUC) value of 37 mg/mL x day to 255 mg/mL x day of the ICOS binding protein after a single dose. In one embodiment, said composition further provides a polypeptide comprising a PD-1 inhibitor and a TGFβR, or an anti-PD-(L)1(IgG):TGFβR fusion protein. In one embodiment, said composition provides an AUC value of 62 mg/mL x day to 220 mg/mL x day of the ICOS binding protein after a single dose.

In one embodiment, diterpenoids, such as paclitaxel, nab-paclitaxel or docetaxel; vinca alkaloids, such as vinblastine, vincristine, or vinorelbine; platinum coordination complexes, such as cisplatin or carboplatin; nitrogen mustards such as cyclophosphamide, melphalan, or chlorambucil; alkyl sulfonates such as busulfan; nitrosoureas such as carmustine; triazenes such as dacarbazine; actinomycins such as dactinomycin; anthrocyclins such as daunorubicin or doxorubicin; bleomycins; epipodophyllotoxins such as etoposide or teniposide; antimetabolite anti-neoplastic agents such as fluorouracil, pemetrexed, methotrexate, cytarabine, mecaptopurine, thioguanine, or gemcitabine; methotrexate; camptothecins such as irinotecan or topotecan; rituximab; ofatumumab; trastuzumab; cetuximab; bexarotene; sorafenib; erbB inhibitors such as lapatinib, erlotinib or gefitinib; pertuzumab; ipilimumab; tremelimumab; nivolumab; pembrolizumab; FOLFOX; capecitabine; FOLFIRI; bevacizumab; atezolizumab; selicrelumab; obinotuzumab or any combinations thereof is/are further administered concurrently or sequentially with the ICOS binding protein and/or the polypeptide comprising a PD-1 inhibitor and a TGFβR, or the anti-PD-(L)1(IgG):TGFβR fusion protein.

In one embodiment, chemotherapy is further administered concurrently or sequentially with the ICOS binding protein and/or the polypeptide comprising a PD-1 inhibitor and a TGFβR, or the anti-PD-(L)1(IgG):TGFβR fusion protein. In one embodiment, chemotherapy is further administered concurrently or sequentially with ICOS binding protein and the polypeptide comprising a PD-1 inhibitor and a TGFβR, or the anti-PD-(L)1(IgG):TGFβR fusion protein. In one embodiment, the chemotherapy is platinum-based chemotherapy. In one embodiment, the chemotherapy is platinum-based chemotherapy and fluorouracil. In one embodiment, the platinum-based chemotherapy is paclitaxel, nab-paclitaxel, docetaxel, cisplatin, carboplatin or any combination thereof. In one embodiment, the platinum-based chemotherapy is fluorouracil, cisplatin, carboplatin or any combination thereof. In one embodiment, chemotherapy is a platinum doublet of cisplatin or carboplatin with any one of pemetrexed, paclitaxel, gemcitabine, or fluorouracil. In one embodiment chemotherapy is further administered concurrently or sequentially with ICOS binding protein and the polypeptide comprising a PD-1 inhibitor and a TGFβR, or the anti-PD-(L)1(IgG):TGFβR fusion protein to PD-1 inhibitor/PD-1 binding protein/PD-L1 binding protein naïve patients.

In one embodiment, the ICOS binding protein; the polypeptide comprising a PD-1 inhibitor and a TGFβR, or the anti-PD-(L)1(IgG):TGFβR fusion protein; and chemotherapy are administered every 3 weeks for 6 cycles and then the ICOS binding protein and polypeptide comprising a PD-1 inhibitor and a TGFβR, or anti-PD-(L)1(IgG):TGFβR fusion protein, is administered every 3 weeks for 35 cycles.

In one embodiment, the ICOS binding protein and the polypeptide comprising a PD-1 inhibitor and a TGFβR, or the anti-PD-(L)1(IgG):TGFβR fusion protein, is administered concurrently or sequentially to PD-L1 positive patients.

In one embodiment, radiotherapy is further administered concurrently or sequentially with the ICOS binding protein and/or the PD-1 inhibitor and/or the TGF-β Inhibitor (e.g. TGFβR). In one embodiment, radiotherapy is further administered concurrently or sequentially with the ICOS binding protein and/or the anti-PD-(L)1(IgG):TGFβR. In some embodiments, the radiotherapy is selected from the group consisting of systemic radiation therapy, external beam radiation therapy, image-guided radiation therapy, tomotherapy, stereotactic radio surgery, stereotactic body radiation therapy, and proton therapy. In some embodiments, the radiotherapy comprises external-beam radiation therapy, internal radiation therapy (brachytherapy), or systemic radiation therapy. See, e.g., Amini et al., Radiat Oncol. “Stereotactic body radiation therapy (SBRT) for lung cancer patients previously treated with conventional radiotherapy: a review” 9:210 (2014); Baker et al., Radiat Oncol. “A critical review of recent developments in radiotherapy for non-small cell lung cancer” 11(1):115 (2016); Ko et al., Clin Cancer Res “The Integration of Radiotherapy with Immunotherapy for the Treatment of Non-Small Cell Lung Cancer” (24) (23) 5792-5806; and, Yamoah et al., Int J Radiat Oncol Biol Phys “Radiotherapy Intensification for Solid Tumors: A Systematic Review of Randomized Trials” 93(4): 737-745 (2015).

In some embodiments, the radiotherapy comprises external-beam radiation therapy, and the external bean radiation therapy comprises intensity-modulated radiation therapy (IMRT), image-guided radiation therapy (IGRT), tomotherapy, stereotactic radiosurgery, stereotactic body radiation therapy, proton therapy, or other charged particle beams.

In some embodiments, the radiotherapy comprises stereotactic body radiation therapy.

### Cancer

Combinations and methods of the invention may be used in the treatment of cancer.

By the term “treating” and grammatical variations thereof as used herein, is meant therapeutic therapy. In reference to a particular condition, treating means: (1) to ameliorate, or lessen the severity of, the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms or signs, effects or side effects associated with the condition or treatment thereof, (4) to slow the progression of the condition, that is to say prolong survival, or one or more of the biological manifestations of the condition and/or (5) to cure said condition or one or more of the biological manifestations of the condition by eliminating or reducing to undetectable levels one or more of the biological manifestations of the condition for a period of time considered to be a state of remission for that manifestation without additional treatment over the period of remission. One skilled in the art will understand the duration of time considered to be remission for a particular disease or condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As used herein, the terms “cancer”, “neoplasm”, “malignancy”, and “tumor” are used interchangeably and, in either the singular or plural form, refer to cells that have undergone a malignant transformation that makes them pathological to the host organism. Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g. by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient.

In one aspect, the invention relates to a method for treating or lessening the severity of a cancer. In one embodiment, the cancer is selected from: brain cancer, glioblastomas, glioma (such as diffuse intrinsic pontine glioma), Bannayan-Zonana syndrome, Cowden disease, Lhermitte-Duclos disease, breast cancer (e.g. inflammatory breast cancer), Wilm's tumor, ependymoma, medulloblastoma, cardiac tumors, colon cancer, colorectal cancer, head and neck cancer (e.g. squamous cell carcinoma of the head and neck, cancer of the mouth (i.e. oral cancer), salivary gland cancer, buccal cancer, pharyngeal cancer, oropharyngeal cancer, nasopharangeal cancer, hypopharyngeal cancer, laryngeal cancer), eye cancer (e.g. retinoblastoma), lung cancer (e.g. non-small cell lung cancer, small cell cancer), liver cancer (i.e. hepatocellular cancer), skin cancer (e.g. basal cell carcinoma, merkel cell carcinoma, squamous cell carcinoma), melanoma, ovarian cancer, pancreatic cancer, bile duct cancer, gallbladder cancer, prostate cancer, sarcoma (e.g. soft tissue sarcoma, Ewing's sarcoma, Kaposi sarcoma, rhabdomyosarcoma), bone cancer, osteosarcoma, giant cell tumor of bone, thyroid cancer, parathyroid cancer, thymoma, blood cancer (which may be broadly categorised as leukemias, lymphomas or myelomas, and include examples such as lymphoblastic T-cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T-cell leukemia, plasmacytoma, immunoblastic large cell leukemia, mantle cell leukemia, multiple myeloma megakaryoblastic leukemia, multiple myeloma, acute megakaryocytic leukemia, promyelocytic leukemia, erythroleukemia, malignant lymphoma, Hodgkin's lymphoma, non-Hodgkin's lymphoma, lymphoblastic T cell lymphoma, Burkitt's lymphoma, and follicular lymphoma), neuroblastoma, pituitary tumor, adrenocortical cancer, anal cancer (i.e. rectal cancer), bladder cancer, urothelial cancer, urethral cancer, vaginal cancer, vulvar cancer, cervical cancer, endometrial cancer, uterine cancer, fallopian tube cancer, renal cancer (i.e. kidney cancer, e.g. renal cell carcinoma), mesothelioma (e.g. malignant pleural mesothelioma), esophageal cancer (e.g. esophageal squamous cell carcinoma), gastric cancer (i.e. stomach cancer), gastroinstestinal carcinoid tumor, GIST (gastrointestinal stromal tumor), appendicial cancer, penile cancer, testicular cancer, germ cell tumors.

In one embodiment, the cancer exhibits microsatellite instability (MSI). Microsatellite instability (“MSI”) is or comprises a change that in the DNA of certain cells (such as tumor cells) in which the number of repeats of microsatellites (short, repeated sequences of DNA) is different than the number of repeats that was contained in the DNA from which it was inherited. Microsatellite instability arises from a failure to repair replication-associated errors due to a defective DNA mismatch repair (MMR) system. This failure allows persistence of mismatch mutations all over the genome, but especially in regions of repetitive DNA known as microsatellites, leading to increased mutational load. It has been demonstrated that at least some tumors characterized by MSI-H have improved responses to certain anti-PD-1 agents (Le et al. (2015) N. Engl. 1. Med. 372(26):2509-2520; Westdorp et al. (2016) Cancer Immunol. Immunother. 65(10): 1249-1259).

In some embodiments, a cancer has a microsatellite instability status of high microsatellite instability (e.g. MSI-H status). In some embodiments, a cancer has a microsatellite instability status of low microsatellite instability (e.g. MSI-L status). In some embodiments, a cancer has a microsatellite instability status of microsatellite stable (e.g. MSS status). In some embodiments microsatellite instability status is assessed by a next generation sequencing (NGS)-based assay, an immunohistochemistry (IHC)-based assay, and/or a PCR-based assay. In some embodiments, microsatellite instability is detected by NGS. In some embodiments, microsatellite instability is detected by IHC. In some embodiments, microsatellite instability is detected by PCR.

In some embodiments, the cancer is associated with a high tumor mutation burden (TMB). In some embodiments, the cancer is associated with high TMB and MSI-H. In some embodiments, the cancer is associated with high TMB and MSI-L or MSS. In some embodiments, the cancer is endometrial cancer associated with high TMB. In some related embodiments, the endometrial cancer is associated with high TMB and MSI-H. In some related embodiments, the endometrial cancer is associated with high TMB and MSI-L or MSS.

In some embodiments, a cancer is a mismatch repair deficient (dMMR) cancer. Microsatellite instability may arise from a failure to repair replication-associated errors due to a defective DNA mismatch repair (MMR) system. This failure allows persistence of mismatch mutations all over the genome, but especially in regions of repetitive DNA known as microsatellites, leading to increased mutational load that may improve responses to certain therapeutic agents.

In some embodiments, a cancer is a hypermutated cancer. In some embodiments, a cancer harbors a mutation in polymerase epsilon (POLE). In some embodiments, a cancer harbors a mutation in polymerase delta (POLD).

In some embodiments, a cancer is endometrial cancer (e.g. MSI-H or MSS/MSI-L endometrial cancer). In some embodiments, a cancer is a MSI-H cancer comprising a mutation in POLE or POLD (e.g. a MSI-H non-endometrial cancer comprising a mutation in POLE or POLD).

In some embodiments, the cancer is an advanced cancer. In some embodiments, the cancer is a metastatic cancer. In some embodiments, the cancer is a recurrent cancer (e.g. a recurrent gynecological cancer such as recurrent epithelial ovarian cancer, recurrent fallopian tube cancer, recurrent primary peritoneal cancer, or recurrent endometrial cancer). In one embodiment, the cancer is recurrent or advanced.

In one embodiment, the cancer is selected from: appendiceal cancer, bladder cancer, breast cancer, cervical cancer, colorectal cancer, endometrial cancer, esophageal cancer (in particular esophageal squamous cell carcinoma), fallopian tube cancer, gastric cancer, glioma (such as diffuse intrinsic pontine glioma), head and neck cancer (in particular head and neck squamous cell carcinoma and oropharyngeal cancer), leukemia (in particular acute lymphoblastic leukemia, acute myeloid leukemia) lung cancer (in particular non small cell lung cancer), lymphoma (in particular Hodgkin's lymphoma, non-Hodgkin's lymphoma), melanoma, mesothelioma (in particular malignant pleural mesothelioma), Merkel cell carcinoma, neuroblastoma, oral cancer, osteosarcoma, ovarian cancer, prostate cancer, renal cancer, salivary gland tumor, sarcoma (in particular Ewing's sarcoma or rhabdomyosarcoma) squamous cell carcinoma, soft tissue sarcoma, thymoma, thyroid cancer, urothelial cancer, uterine cancer, vaginal cancer, vulvar cancer or Wilms tumor. In a further embodiment, the cancer is selected from: appendiceal cancer, bladder cancer, cervical cancer, colorectal cancer, esophageal cancer, head and neck cancer, melanoma, mesothelioma, non-small-cell lung cancer, prostate cancer and urothelial cancer. In a further embodiment, the cancer is selected from cervical cancer, endometrial cancer, head and neck cancer (in particular head and neck squamous cell carcinoma and oropharyngeal cancer), lung cancer (in particular non small cell lung cancer), lymphoma (in particular non-Hodgkin's lymphoma), melanoma, oral cancer, thyroid cancer, urothelial cancer or uterine cancer. In another embodiment, the cancer is selected from head and neck cancer (in particular head and neck squamous cell carcinoma and oropharyngeal cancer), lung cancer (in particular non small cell lung cancer), urothelial cancer, melanoma or cervical cancer.

In one embodiment, the human has a solid tumor. In one embodiment, the solid tumor is advanced solid tumor. In one embodiment, the cancer is selected from head and neck cancer, squamous cell carcinoma of the head and neck (SCCHN or HNSCC), gastric cancer, melanoma, renal cell carcinoma (RCC), esophageal cancer, non-small cell lung carcinoma, prostate cancer, colorectal cancer, ovarian cancer and pancreatic cancer. In one embodiment, the cancer is selected from the group consisting of: colorectal cancer, cervical cancer, bladder cancer, urothelial cancer, head and neck cancer, melanoma, mesothelioma, non-small cell lung carcinoma, prostate cancer, esophageal cancer, and esophageal squamous cell carcinoma. In one aspect the human has one or more of the following: SCCHN, colorectal cancer, esophageal cancer, cervical cancer, bladder cancer, breast cancer, head and neck cancer, ovarian cancer, melanoma, renal cell carcinoma (RCC), esophageal squamous cell carcinoma, non-small cell lung carcinoma, mesothelioma (e.g. pleural malignant mesothelioma), and prostate cancer.

In another aspect the human has a liquid tumor such as diffuse large B cell lymphoma (DLBCL), multiple myeloma, chronic lymphoblastic leukemia, follicular lymphoma, acute myeloid leukemia and chronic myelogenous leukemia.

In one embodiment, the cancer is head and neck cancer. In one embodiment, the cancer is HNSCC. Squamous cell carcinoma is a cancer that arises from particular cells called squamous cells. Squamous cells are found in the outer layer of skin and in the mucous membranes, which are the moist tissues that line body cavities such as the airways and intestines. Head and neck squamous cell carcinoma (HNSCC) develops in the mucous membranes of the mouth, nose, and throat. HNSCC is also known as SCCHN and squamous cell carcinoma of the head and neck.

HNSCC can occur in the mouth (oral cavity), the middle part of the throat near the mouth (oropharynx), the space behind the nose (nasal cavity and paranasal sinuses), the upper part of the throat near the nasal cavity (nasopharynx), the voicebox (larynx), or the lower part of the throat near the larynx (hypopharynx). Depending on the location, the cancer can cause abnormal patches or open sores (ulcers) in the mouth and throat, unusual bleeding or pain in the mouth, sinus congestion that does not clear, sore throat, earache, pain when swallowing or difficulty swallowing, a hoarse voice, difficulty breathing, or enlarged lymph nodes.

HNSCC can metastasize to other parts of the body, such as the lymph nodes, lungs or liver.

Tobacco use and alcohol consumption are the two most important risk factors for the development of HNSCC, and their contributions to risk are synergistic. In addition, the human papillomavirus (HPV), especially HPV-16, is now a well-established independent risk factor. Patients with HNSCC have a relatively poor prognosis. Recurrent/metastatic (R/M) HNSCC is especially challenging, regardless of human papillomavirus (HPV) status, and currently, few effective treatment options are available in the art. HPV-negative HNSCC is associated with a locoregional relapse rate of 19-35% and a distant metastatic rate of 14-22% following standard of care, compared with rates of 9-18% and 5-12%, respectively, for HPV-positive HNSCC. The median overall survival for patients with R/M disease is 10-13 months in the setting of first-line chemotherapy and 6 months in the second-line setting. The current standard of care is platinum-based doublet chemotherapy with or without cetuximab. Second-line standard of care options include cetuximab, methotrexate, and taxanes. All of these chemotherapeutic agents are associated with significant side effects, and only 10-13% of patients respond to treatment. HNSCC regressions from existing systemic therapies are transient and do not add significantly increased longevity, and virtually all patients succumb to their malignancy.

In one embodiment, the cancer is head and neck cancer. In one embodiment the cancer is head and neck squamous cell carcinoma (HNSCC). In one embodiment, the cancer is recurrent/metastatic (R/M) HNSCC. In one embodiment, the cancer is recurring/refractory (R/R) HNSCC. In one embodiment, the cancer is HPV-negative or HPV-positive HNSCC. In one embodiment, the cancer is a locally advanced HNSCC. In one embodiment, the cancer is (R/M) HNSCC in PD-L1 CPS (Combined Positive Score) positive (CPS ≥1) patients. The combined positive score is as determined by an FDA-approved test. PD-L1 CPS is the number of PD-L1 staining cells (tumor cells, lymphocytes, macrophages) divided by the total number of viable tumor cells, multiplied by 100. In one embodiment, PD-L1 CPS is determined using PharmDx 22C3. In one embodiment, the cancer is HNSCC in PD-1 binding protein/PD-L1 binding protein experienced or PD-1 binding protein/PD-L1 binding protein naïve patients. In one embodiment, the cancer is HNSCC in PD-1 binding protein/PD-L1 binding protein experienced or PD-1 binding protein/PD-L1 binding protein naïve patients.

In one embodiment, the head and neck cancer is oropharyngeal cancer. In one embodiment, the head and neck cancer is an oral cancer (i.e. a mouth cancer).

In one embodiment, the cancer is lung cancer. In some embodiments, the lung cancer is a squamous cell carcinoma of the lung. In some embodiments, the lung cancer is small cell lung cancer (SCLC). In some embodiments, the lung cancer is non-small cell lung cancer (NSCLC), such as squamous NSCLC. In some embodiments, the lung cancer is an ALK-translocated lung cancer (e.g. ALK-translocated NSCLC). In some embodiments, the cancer is NSCLC with an identified ALK translocation. In some embodiments, the lung cancer is an EGFR-mutant lung cancer (e.g. EGFR-mutant NSCLC). In some embodiments, the cancer is NSCLC with an identified EGFR mutation.

In one embodiment, the cancer is melanoma. In some embodiments, the melanoma is an advanced melanoma. In some embodiments, the melanoma is a metastatic melanoma. In some embodiments, the melanoma is a MSI-H melanoma. In some embodiments, the melanoma is a MSS melanoma. In some embodiments, the melanoma is a POLE-mutant melanoma. In some embodiments, the melanoma is a POLD-mutant melanoma. In some embodiments, the melanoma is a high TMB melanoma.

In one embodiment, the cancer is colorectal cancer. In some embodiments, the colorectal cancer is an advanced colorectal cancer. In some embodiments, the colorectal cancer is a metastatic colorectal cancer. In some embodiments, the colorectal cancer is a MSI-H colorectal cancer. In some embodiments, the colorectal cancer is a MSS colorectal cancer. In some embodiments, the colorectal cancer is a POLE-mutant colorectal cancer. In some embodiments, the colorectal cancer is a POLD-mutant colorectal cancer. In some embodiments, the colorectal cancer is a high TMB colorectal cancer.

In some embodiments, the cancer is a gynecologic cancer (i.e. a cancer of the female reproductive system such as ovarian cancer, fallopian tube cancer, cervical cancer, vaginal cancer, vulvar cancer, uterine cancer, or primary peritoneal cancer, or breast cancer). In some embodiments, cancers of the female reproductive system include, but are not limited to, ovarian cancer, cancer of the fallopian tube(s), peritoneal cancer, and breast cancer.

In some embodiments, the cancer is ovarian cancer (e.g. serous or clear cell ovarian cancer). In some embodiments, the cancer is fallopian tube cancer (e.g. serous or clear cell fallopian tube cancer). In some embodiments, the cancer is primary peritoneal cancer (e.g. serous or clear cell primary peritoneal cancer).

In some embodiments, the ovarian cancer is an epithelial carcinoma. Epithelial carcinomas make up 85% to 90% of ovarian cancers. While historically considered to start on the surface of the ovary, new evidence suggests at least some ovarian cancer begins in special cells in a part of the fallopian tube. The fallopian tubes are small ducts that link a woman's ovaries to her uterus that are a part of a woman's reproductive system. In a normal female reproductive system, there are two fallopian tubes, one located on each side of the uterus. Cancer cells that begin in the fallopian tube may go to the surface of the ovary early on. The term “ovarian cancer” is often used to describe epithelial cancers that begin in the ovary, in the fallopian tube, and from the lining of the abdominal cavity, call the peritoneum. In some embodiments, the cancer is or comprises a germ cell tumor. Germ cell tumors are a type of ovarian cancer develops in the egg-producing cells of the ovaries. In some embodiments, a cancer is or comprises a stromal tumor. Stromal tumors develop in the connective tissue cells that hold the ovaries together, which sometimes is the tissue that makes female hormones called estrogen. In some embodiments, the cancer is or comprises a granulosa cell tumor. Granulosa cell tumors may secrete estrogen resulting in unusual vaginal bleeding at the time of diagnosis. In some embodiments, a gynecologic cancer is associated with homologous recombination repair deficiency/homologous repair deficiency (HRD) and/or BRCA1/2 mutation(s). In some embodiments, a gynecologic cancer is platinum-sensitive. In some embodiments, a gynecologic cancer has responded to a platinum-based therapy. In some embodiments, a gynecologic cancer has developed resistance to a platinum-based therapy. In some embodiments, a gynecologic cancer has at one time shown a partial or complete response to platinum-based therapy (e.g. a partial or complete response to the last platinum-based therapy or to the penultimate platinum-based therapy). In some embodiments, a gynecologic cancer is now resistant to platinum-based therapy.

In some embodiments, the cancer is breast cancer. Usually breast cancer either begins in the cells of the milk producing glands, known as the lobules, or in the ducts. Less commonly breast cancer can begin in the stromal tissues. These include the fatty and fibrous connective tissues of the breast. Over time the breast cancer cells can invade nearby tissues such the underarm lymph nodes or the lungs in a process known as metastasis. The stage of a breast cancer, the size of the tumor and its rate of growth are all factors which determine the type of treatment that is offered. Treatment options include surgery to remove the tumor, drug treatment which includes chemotherapy and hormonal therapy, radiation therapy and immunotherapy. The prognosis and survival rate varies widely; the five year relative survival rates vary from 98% to 23% depending on the type of breast cancer that occurs. Breast cancer is the second most common cancer in the world with approximately 1.7 million new cases in 2012 and the fifth most common cause of death from cancer, with approximately 521,000 deaths. Of these cases, approximately 15% are triple-negative, which do not express the estrogen receptor, progesterone receptor (PR) or HER2. In some embodiments, triple negative breast cancer (TNBC) is characterized as breast cancer cells that are estrogen receptor expression negative (<1% of cells), progesterone receptor expression negative (<1% of cells), and HER2-negative.

In some embodiments, the cancer is estrogen receptor(ER)-positive breast cancer, ER-negative breast cancer, PR-positive breast cancer, PR-negative breast cancer, HER2-positive breast cancer, HER2-negative breast cancer, BRCA1/2-positive breast cancer, BRCA1/2-negative cancer, or TNBC. In some embodiments, the breast cancer is a metastatic breast cancer. In some embodiments, the breast cancer is an advanced breast cancer. In some embodiments, the cancer is a stage II, stage III or stage IV breast cancer. In some embodiments, the cancer is a stage IV breast cancer. In some embodiments, the breast cancer is a triple negative breast cancer.

In one embodiment, the cancer is endometrial cancer. Endometrial carcinoma is the most common cancer of the female genital, tract accounting for 10-20 per 100,000 person-years. The annual number of new cases of endometrial cancer (EC) is estimated at about 325 thousand worldwide. Further, EC is the most commonly occurring cancer in post-menopausal women. About 53% of endometrial cancer cases occur in developed countries. In 2015, approximately 55,000 cases of EC were diagnosed in the U.S. and no targeted therapies are currently approved for use in EC. There is a need for agents and regimens that improve survival for advanced and recurrent EC in 1L and 2L settings. Approximately 10,170 people are predicted to die from EC in the U.S. in 2016. The most common histologic form is endometrioid adenocarcinoma, representing about 75-80% of diagnosed cases. Other histologic forms include uterine papillary serous (less than 10%), clear cell 4%, mucinous 1%, squamous less than 1% and mixed about 10%.

From the pathogenetic point of view, EC falls into two different types, so-called types I and II. Type I tumors are low-grade and estrogen-related endometrioid carcinomas (EEC) while type II are non-endometrioid (NEEC) (mainly serous and clear cell) carcinomas. The World Health Organization has updated the pathologic classification of EC, recognizing nine different subtypes of EC, but EEC and serous carcinoma (SC) account for the vast majority of cases. EECs are estrogen-related carcinomas, which occur in perimenopausal patients, and are preceded by precursor lesions (endometrial hyperplasia/endometrioid intraepithelial neoplasia). Microscopically, lowgrade EEC (EEC 1-2) contains tubular glands, somewhat resembling the proliferative endometrium, with architectural complexity with fusion of the glands and cribriform pattern. High-grade EEC shows solid pattern of growth. In contrast, SC occurs in postmenopausal patients in absence of hyperestrogenism. At the microscope, SC shows thick, fibrotic or edematous papillae with prominent stratification of tumor cells, cellular budding, and anaplastic cells with large, eosinophilic cytoplasms. The vast majority of EEC are low grade tumors (grades 1 and 2), and are associated with good prognosis when they are restricted to the uterus. Grade 3 EEC (EEC3) is an aggressive tumor, with increased frequency of lymph node metastasis. SCs are very aggressive, unrelated to estrogen stimulation, mainly occurring in older women. EEC 3 and SC are considered high-grade tumors. SC and EEC3 have been compared using the surveillance, epidemiology and End Results (SEER) program data from 1988 to 2001. They represented 10% and 15% of EC respectively, but accounted for 39% and 27% of cancer death respectively. Endometrial cancers can also be classified into four molecular subgroups: (1) ultramutated/POLE-mutant; (2) hypermutated MSI+ (e.g., MSI-H or MSI-L); (3) copy number low/micro satellite stable (MSS); and (4) copy number high/serous-like. Approximately 28% of cases are MSI-high. (Murali, Lancet Oncol. (2014). In some embodiments, the patient has a mismatch repair deficient subset of 2L endometrial cancer. In some embodiments, the endometrial cancer is metastatic endometrial cancer. In some embodiments, the patient has a MSS endometrial cancer. In some embodiments, the patient has a MSI-H endometrial cancer.

In one embodiment, the cancer is cervical cancer. In some embodiments, the cervical cancer is an advanced cervical cancer. In some embodiments, the cervical cancer is a metastatic cervical cancer. In some embodiments, the cervical cancer is a MSI-H cervical cancer. In some embodiments, the cervical cancer is a MSS cervical cancer. In some embodiments, the cervical cancer is a POLE-mutant cervical cancer. In some embodiments, the cervical cancer is a POLD-mutant cervical cancer. In some embodiments, the cervical cancer is a high TMB cervical cancer.

In one embodiment, the cancer is uterine cancer. In some embodiments, the uterine cancer is an advanced uterine cancer. In some embodiments, the uterine cancer is a metastatic uterine cancer. In some embodiments, the uterine cancer is a MSI-H uterine cancer. In some embodiments, the uterine cancer is a MSS uterine cancer. In some embodiments, the uterine cancer is a POLE-mutant uterine cancer. In some embodiments, the uterine cancer is a POLD-mutant uterine cancer.

In some embodiments, the uterine cancer is a high TMB uterine cancer.

In one embodiment, the cancer is urothelial cancer. In some embodiments, the urothelial cancer is an advanced urothelial cancer. In some embodiments, the urothelial cancer is a metastatic urothelial cancer. In some embodiments, the urothelial cancer is a MSI-H urothelial cancer. In some embodiments, the urothelial cancer is a MSS urothelial cancer. In some embodiments, the urothelial cancer is a POLE-mutant urothelial cancer. In some embodiments, the urothelial cancer is a POLD-mutant urothelial cancer. In some embodiments, the urothelial cancer is a high TMB urothelial cancer.

In one embodiment, the cancer is thyroid cancer. In some embodiments, the thyroid cancer is an advanced thyroid cancer. In some embodiments, the thyroid cancer is a metastatic thyroid cancer. In some embodiments, the thyroid cancer is a MSI-H thyroid cancer. In some embodiments, the thyroid cancer is a MSS thyroid cancer. In some embodiments, the thyroid cancer is a POLE-mutant thyroid cancer. In some embodiments, the thyroid cancer is a POLD-mutant thyroid cancer. In some embodiments, the thyroid cancer is a high TMB thyroid cancer.

Tumors may be a hematopoietic (or hematologic or hematological or blood-related) cancer, for example, cancers derived from blood cells or immune cells, which may be referred to as “liquid tumors”. Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, monoclonal gammopathy of undetermined (or unknown or unclear) significance (MGUS) and Waldenstrom's macroglobulinemia; lymphomas such as non-Hodgkin's lymphoma, Hodgkin's lymphoma, and the like.

The cancer may be any cancer in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia. Myeloid malignancies also include myeloproliferative disorders (MPD) which include, but are not limited to, chronic myelogenous (or myeloid or myelocytic) leukemia (CML), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia.

In one embodiment, the cancer is non-Hodgkin's lymphoma. Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate-grade (or aggressive) or high-grade (very aggressive). Indolent B cell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large B cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphomas (T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenström's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

In one embodiment, the treatment is first-line or second line treatment of HNSCC. In one embodiment, the treatment is first-line or second line treatment of recurrent/metastatic HNSCC. In one embodiment the treatment is first line treatment of recurrent/metastatic (1L R/M) HNSCC. In one embodiment, the treatment is first line treatment of 1L R/M HNSCC in a PD-L1 CPS (combined positive score) positive (CPS ≥1) patients. In one embodiment the treatment is second line treatment of recurrent/metastatic (2L R/M) HNSCC.

In one embodiment, the treatment is first-line, second-line, third-line, fourth-line or fifth-line treatment of PD-1/PD-L1-naïve HNSCC. In one embodiment, the treatment first-line, second-line, third-line, fourth-line or fifth-line treatment of PD-1/PD-L1 experienced HNSCC.

In some embodiments, the treatment of cancer is first-line treatment of cancer. In one embodiment, the treatment of cancer is second-line treatment of cancer. In some embodiments, the treatment is third-line treatment of cancer. In some embodiments, the treatment is fourth-line treatment of cancer. In some embodiments, the treatment is fifth-line treatment of cancer. In some embodiments, prior treatment to said second-line, third-line, fourth-line or fifth-line treatment of cancer comprises one or more of radiotherapy, chemotherapy, surgery or radiochemotherapy.

In one embodiment, the prior treatment comprises treatment with diterpenoids, such as paclitaxel, nab-paclitaxel or docetaxel; vinca alkaloids, such as vinblastine, vincristine, or vinorelbine; platinum coordination complexes, such as cisplatin or carboplatin; nitrogen mustards such as cyclophosphamide, melphalan, or chlorambucil; alkyl sulfonates such as busulfan; nitrosoureas such as carmustine; triazenes such as dacarbazine; actinomycins such as dactinomycin; anthrocyclins such as daunorubicin or doxorubicin; bleomycins; epipodophyllotoxins such as etoposide or teniposide; antimetabolite anti-neoplastic agents such as fluorouracil, methotrexate, cytarabine, mecaptopurine, thioguanine, or gemcitabine; methotrexate; camptothecins such as irinotecan or topotecan; rituximab; ofatumumab; trastuzumab; cetuximab; bexarotene; sorafenib; erbB inhibitors such as lapatinib, erlotinib or gefitinib; pertuzumab; ipilimumab; nivolumab; FOLFOX; capecitabine; FOLFIRI; bevacizumab; atezolizumab; selicrelumab; obinotuzumab or any combinations thereof. In one embodiment, prior treatment to said second line treatment, third-line, fourth-line or fifth-line treatment of cancer comprises ipilimumab and nivolumab. In one embodiment, prior treatment to said second line treatment, third-line, fourth-line or fifth-line treatment of cancer comprises FOLFOX, capecitabine, FOLFIRI/bevacizumab and atezolizumab/selicrelumab. In one embodiment, prior treatment to said second line treatment, third-line, fourth-line or fifth-line treatment of cancer comprises carboplatin/Nab-paclitaxel. In one embodiment, prior treatment to said second line treatment, third-line, fourth-line or fifth-line treatment of cancer comprises nivolumab and electrochemotherapy. In one embodiment, prior treatment to said second line treatment, third-line, fourth-line or fifth-line treatment of cancer comprises radiotherapy, cisplatin and carboplatin/paclitaxel.

In one embodiment, the treatment is first-line or second line treatment of head and neck cancer (in particular head and neck squamous cell carcinoma and oropharyngeal cancer). In one embodiment, the treatment is first-line or second line treatment of recurrent/metastatic HNSCC. In one embodiment the treatment is first line treatment of recurrent/metastatic (1L R/M) HNSCC. In one embodiment, the treatment is first line treatment of 1L R/M HNSCC in a PD-L1 CPS (combined positive score) positive (CPS ≥1) patients. In one embodiment the treatment is second line treatment of recurrent/metastatic (2L R/M) HNSCC.

In one embodiment, the treatment is first-line, second-line, third-line, fourth-line or fifth-line treatment of PD-1/PD-L1-naïve HNSCC. In one embodiment, the treatment first-line, second-line, third-line, fourth-line or fifth-line treatment of PD-1/PD-L1 experienced HNSCC.

In some embodiments, the treatment results in one or more of increased tumor infiltrating lymphocytes including cytotoxic T cells, helper T cell and NK cells, increased T cells, increased granzyme B+ cells, reduced proliferating tumor cells and increased activated T cells as compared to levels prior to treatment (e.g. baseline level). Activated T cells may be observed by greater OX40 and human leukocyte antigen DR expression. In some embodiments, treatment results in upregulation of PD-1 and/or PD-L1 as compared to levels prior to treatment (e.g. baseline level).

In one embodiment, the methods of the present invention further comprise administering at least one neo-plastic agent or cancer adjuvant to said human. The methods of the present invention may also be employed with other therapeutic methods of cancer treatment.

Typically, any anti-neoplastic agent or cancer adjuvant that has activity versus a tumor, such as a susceptible tumor being treated may be co-administered in the treatment of cancer in the present invention. Examples of such agents can be found in Cancer Principles and Practice of Oncology by V. T. Devita, T. S. Lawrence, and S. A. Rosenberg (editors), 10th edition (Dec. 5, 2014), Lippincott Williams & Wilkins Publishers.

In one embodiment, the human has previously been treated with one or more different cancer treatment modalities. In some embodiments, at least some of the patients in the cancer patient population have previously been treated with one or more therapies, such as surgery, radiotherapy, chemotherapy or immunotherapy. In some embodiments, at least some of the patients in the cancer patient population have previously been treated with chemotherapy (e.g. platinum-based chemotherapy). For example, a patient who has received two lines of cancer treatment can be identified as a 2L cancer patient (e.g. a 2L NSCLC patient). In some embodiments, a patient has received two lines or more lines of cancer treatment (e.g. a 2L+ cancer patient such as a 2L+ endometrial cancer patient). In some embodiments, a patient has not been previously treated with an antibody therapy, such as an anti-PD-1 therapy. In some embodiments, a patient previously received at least one line of cancer treatment (e.g. a patient previously received at least one line or at least two lines of cancer treatment). In some embodiments, a patient previously received at least one line of treatment for metastatic cancer (e.g. a patient previously received one or two lines of treatment for metastatic cancer). In some embodiments, a subject is resistant to treatment with an agent that inhibits PD-1. In some embodiments, a subject is refractory to treatment with an agent that inhibits PD-1. In some embodiments, a method described herein sensitizes the subject to treatment with an agent that inhibits PD-1.

It will be noted that embodiments of the method of treatment of cancer are also taken as embodiments of the ICOS binding protein and/or the polypeptide comprising a PD-1 inhibitor and a TGFβR or the anti-PD-(L)1(IgG):TGFβR fusion protein for use in the treatment of cancer or use of an ICOS binding protein and/or the polypeptide comprising a PD-1 inhibitor and a TGFβR or the anti-PD-(L)1(IgG):TGFβR fusion protein in the manufacture of a medicament for treating cancer and reciprocals thereof, in so far as it relates to dosages, treatment regimens and effects of said dosages and treatment regimens. It will also be noted that embodiments of the method of treatment of cancer, the ICOS binding protein and/or the polypeptide comprising a PD-1 inhibitor and a TGFβR or the anti-PD-(L)1(IgG):TGFβR fusion protein for use in the treatment of cancer or use of an ICOS binding protein and/or the polypeptide comprising a PD-1 inhibitor and a TGFβR or the anti-PD-(L)1(IgG):TGFβR fusion protein in the manufacture of a medicament for treating cancer are also taken as embodiments of the pharmaceutical composition, pharmaceutical formulation or pharmaceutical kit in so far as it relates to dosages, treatment regimens and effects of said dosages and treatment regimens.

### Pharmaceutical Compositions/Routes of Administration/Dosages

Antigen binding proteins as described herein may be incorporated into pharmaceutical compositions for use in the treatment of the human diseases described herein. In one embodiment, the pharmaceutical composition comprises an antigen binding protein in combination with one or more pharmaceutically acceptable carriers and/or excipients.

Such compositions comprise a pharmaceutically acceptable carrier as known and called for by acceptable pharmaceutical practice.

Pharmaceutical compositions may be administered by injection or continuous infusion (examples include, but are not limited to, intravenous, intraperitoneal, intradermal, subcutaneous, intramuscular, intraocular, and intraportal). In one embodiment, the composition is suitable for intravenous administration. Pharmaceutical compositions may be suitable for topical administration (which includes, but is not limited to, epicutaneous, inhaled, intranasal or ocular administration) or enteral administration (which includes, but is not limited to, oral, vaginal, or rectal administration).

The pharmaceutical composition may be included in a kit containing the antigen binding proteins together with other medicaments, and/or with instructions for use. For convenience, the kit may comprise the reagents in predetermined amounts with instructions for use. The kit may also include devices used for administration of the pharmaceutical composition.

The terms “individual”, “subject” and “patient” are used herein interchangeably. In one embodiment, the subject is an animal. In another embodiment, the subject is a mammal, such as a primate, for example a marmoset or monkey. In another embodiment, the subject is a human (i.e. a human patient). “Subject” is defined broadly to include any patient in need of treatment, for example, a patient in need of cancer treatment. The subject in need of cancer treatment may include patients from a variety of stages including newly diagnosed, relapsed, refractory, progressive disease, remission, and others. The subject in need of cancer treatment may also include patients who have undergone stem cell transplant or who are considered transplant ineligible.

Subjects may be pre-screened in order to be selected for treatment with the combinations described herein. In one embodiment, a sample from the subject is tested for expression of PD-L1 prior to treatment with the combinations described herein.

### Kits

In one aspect, the invention provides a kit comprising:

(i) an ICOS binding protein;

(ii) a PD-1 inhibitor;

(iii) a TGF-β inhibitor; and alternatively comprising,

(iv) instructions for using (i), (ii) and (iii) in combination in the treatment of a cancer in a human.

In another aspect, the invention provides a kit comprising:

(i) an ICOS binding protein;

(ii) a polypeptide comprising a PD-1 inhibitor and a TGFβR; and alternatively comprising,

(iii) instructions for using (i) and (ii) in combination in the treatment of a cancer in a human.

In a further aspect, the invention provides a kit comprising:

(i) an ICOS binding protein;

(ii) an anti-PD-(L)1(IgG):TGFβR fusion protein; and alternatively comprising,

(iii) instructions for using (i) and (ii) in combination in the treatment of a cancer in a human.

In some aspects, the invention provides a kit comprising:

(i) an ICOS binding protein comprising a heavy chain amino acid sequence comprising a CDRH1 of SEQ ID NO:1, a CDRH2 of SEQ ID NO:2, and a CDRH3 of SEQ ID NO:3, and a light chain amino acid sequence comprising a CDRL1 of SEQ ID NO:4, a CDRL2 of SEQ ID NO:5, and a CDRL3 of SEQ ID NO:6;

(ii) an anti-PD-(L)1(IgG):TGFβR fusion protein comprising: (a) a PD-L1 binding protein comprising a heavy chain amino acid sequence comprising a CDRH1 of SEQ ID NO:13, a CDRH2 of SEQ ID NO:14, and a CDRH3 of SEQ ID NO:15, and a light chain amino acid sequence comprising a CDRL1 of SEQ ID NO:16, a CDRL2 of SEQ ID NO:17, and a CDRL3 of SEQ ID NO:18; and (b) human TGFβRII, or a fragment thereof capable of binding to TGF-β; and

(iii) instructions for using (i) and (ii) in combination in the treatment of a cancer in a human.

In some aspects, the kit is for use in the treatment of cancer.

In some embodiments, the ICOS binding protein and the polypeptide comprising a PD-1 inhibitor and a TGFβR or the anti-PD-(L)1(IgG):TGFβR fusion protein are each individually formulated in their own pharmaceutical compositions with one or more pharmaceutically acceptable carriers.

In some aspects, the invention provides a kit for use in the treatment of cancer comprising:

(i) an ICOS binding protein comprising a heavy chain amino acid sequence comprising a CDRH1 of SEQ ID NO:1, a CDRH2 of SEQ ID NO:2, and a CDRH3 of SEQ ID NO:3, and a light chain amino acid sequence comprising a CDRL1 of SEQ ID NO:4, a CDRL2 of SEQ ID NO:5, and a CDRL3 of SEQ ID NO:6;

(ii) instructions for use in the treatment of cancer when combined with a anti-PD-(L)1(IgG):TGFβR fusion protein.

In some aspects, the invention provides a kit for use in the treatment of cancer comprising:

(i) an anti-PD-(L)1(IgG):TGFβR fusion protein comprising: (a) a PD-L1 binding protein comprising a heavy chain amino acid sequence comprising a CDRH1 of SEQ ID NO:13, a CDRH2 of SEQ ID NO:14, and a CDRH3 of SEQ ID NO:15, and a light chain amino acid sequence comprising a CDRL1 of SEQ ID NO:16, a CDRL2 of SEQ ID NO:17, and a CDRL3 of SEQ ID NO:18; and (b) human TGFβRII, or a fragment thereof capable of binding to TGF-β;

(ii) instructions for use in the treatment of cancer when combined with an ICOS binding protein.

In one embodiment, the kit for use in the treatment of cancer comprises:

(i) an ICOS binding protein at a concentration of 10 mg/mL; and

(ii) a polypeptide comprising a PD-1 inhibitor and a TGFβR or an anti-PD-(L)1(IgG):TGFβR fusion protein at a concentration of about 20 mg/mL to about 125 mg/mL, such as about 20 mg/mL to about 50 mg/mL, in particular 10 mg/mL, 20 mg/mL, 30 mg/mL, 40 mg/mL or 50 mg/mL.

In some embodiments of all of the above kit aspects, the PD-1 inhibitor is a PD-1 binding protein or a PD-L1 binding protein. In further embodiments of the above kit aspects, the anti-PD-(L)1(IgG):TGFβR fusion protein comprises (a) human TGFβRII, or a fragment thereof capable of binding to TGF-β; and (b) an anti-PD-L1 antibody or an antigen-binding fragment thereof, or an anti-PD-1 antibody or an antigen-binding fragment thereof.

In particular embodiments of all of the above kit aspects, the ICOS binding protein is feladilimab. In particular embodiments of all of the above kit aspects, the polypeptide comprising a PD-1 inhibitor and a TGFβR, or the anti-PD-(L)1(IgG):TGFβR fusion protein, is bintrafusp alfa.

### EXAMPLES

### Example 1. Evaluation of Anti-ICOS Agonist Antibody in Combination with M7824 (PD-L1-TGFβRII Trap) in the EMT6 Murine Solid Tumor Model

### 1.1 Animals

Female BALB/c mice (BALB/cAnNHsd), 6 weeks old, were purchased from Envigo. All animals were maintained at a site which complies with the recommendations of the Guide for Care and Use of Laboratory Animals concerning restraint, husbandry, surgical procedures, feed and fluid regulation, and veterinary care.

### 1.2 Cell Line Culture

The EMT6 murine mammary carcinoma cell line was purchased from American Type Culture Collection (ATCC, CRL-2755) and cultured in CELLSTAR Tissue Culture Flasks (Greiner Bio-one, part #660175) at 37° C. and 5% C02 (HERAcell Vios 160i, ThermoScientific, S/N 41975756). Cells were expanded, aliquoted, and cryopreserved at vapor phase of LN2 for future use. Cell stocks were confirmed by Charles River Laboratory (PCR mouse pathogen panel) to be negative for mouse pathogens. One aliquot was thawed and cultured for an additional three passages before tumor inoculation.

### 1.3 Tumor Inoculation

Cells used for inoculation were harvested during log-phase (growth) and resuspended in cold 1×PBS. Each mouse was injected subcutaneously (S.C.) in the right flank with 1×10⁵ EMT6 cells (0.1 mL cell suspension).

### 1.4 Measurement

Mice were identified using microchip detection (S.C. injected, BMDS, Cat # IMI-500). Tumor volume was measured by digital caliper, and body weight was measured using a balance (Meterlo Toledo). Measurement data was collected using the Study Director Software Package (Studylog Systems version 4.2, South San Francisco, Calif., USA). Tumor volume was calculated using the formula:

Tumor Volume (mm³)=0.52×l×w² (where w=width and l=length, in mm, of the tumor).

### 1.5 Randomization

Before initiation of treatment, mice were randomized into individual groups (˜day seven post-tumor-inoculation, tumor size of 100-150 mm³) using a stratified sampling method in the StudyLog software. Statistical analysis was conducted (ANOVA) to ensure even tumor size distribution between groups (P value >0.99).

### 1.6 Test Agents and Treatment

Antibodies were diluted to the desired concentration in sterile 1×PBS (in 100 μL where possible). The antibodies used were: Mouse IgG1 Isotype Control (mIgG1, Bioxcell, Cat # BE0083); anti-ICOS mouse IgG1 (Absolute antibody, Cat #AB00814-1.1); human IgG1 Isotype Control (hIgG1, Merck KGaA, Lot # PPB-1336); Anti-PD-L1 (Merck KGaA, Lot # PPB-6677); TGFβRII-Trap control (hIgG1 Trap, Merck KGaA, Lot # PPB-1684) which was used for TGF-β-inhibition only; and M7824 (PD-L1-TGFβRII-Trap, Merck KGaA, Lot # PPB-5827).

The first dose was designated as study Day 0. Tumor-bearing mice were dosed per the treatment plan summarized in the study design tables.

### 1.7 Observations and Endpoints

Mice were checked for any effects of tumor growth and treatments on behavior such as mobility, food and water consumption, body weight gain/loss, or any other abnormalities. All observed clinical signs and mortality were recorded. Tumor size and body weight were measured 2-3 times per week, and the individual animal was euthanized when the tumor reached the pre-determined endpoint (tumor volume of 2500 mm³, ulceration, bodyweight loss>20%) or at the end of the study, whichever came first.

### 1.8 Statistical Analysis

Tumor growth trends: Trend model is a linear mixed model. Treatment time trends are modeled as natural splines with 2, 3, or 4 degrees-of-freedom. If there are days when fewer than half the treatments have volume data, then volumes recorded after the last such day are ignored for trend modelling. This approach focuses on the main period of volume measurements and helps avoid a few late measurements driving the trend analysis.

Adjusted AUC: an integrated (across time) measure of tumor burden, adjusted for the number of days on the study of tumor volume. Adjusted AUCs are analyzed by a nonparametric ANOVA (ANOVA on the ranks), followed by a false discovery rate (FDR) multiplicity adjustment. Significance is defined as FDR<=0.05.

Kaplan-Meier (KM) survival analysis: The method is carried out to estimate the survival probability of different treatment groups at a given time. The median time to endpoint and its corresponding 95% confidence interval is reported. Whether or not KM survival curves are statistically different between any two groups is then tested by log-rank test. p-values are adjusted for multiplicity using the FDR (false discovery rate) method. Significance is defined as FDR<=0.05. R analysis software is used.

### 1.9 Study Design 1—Results

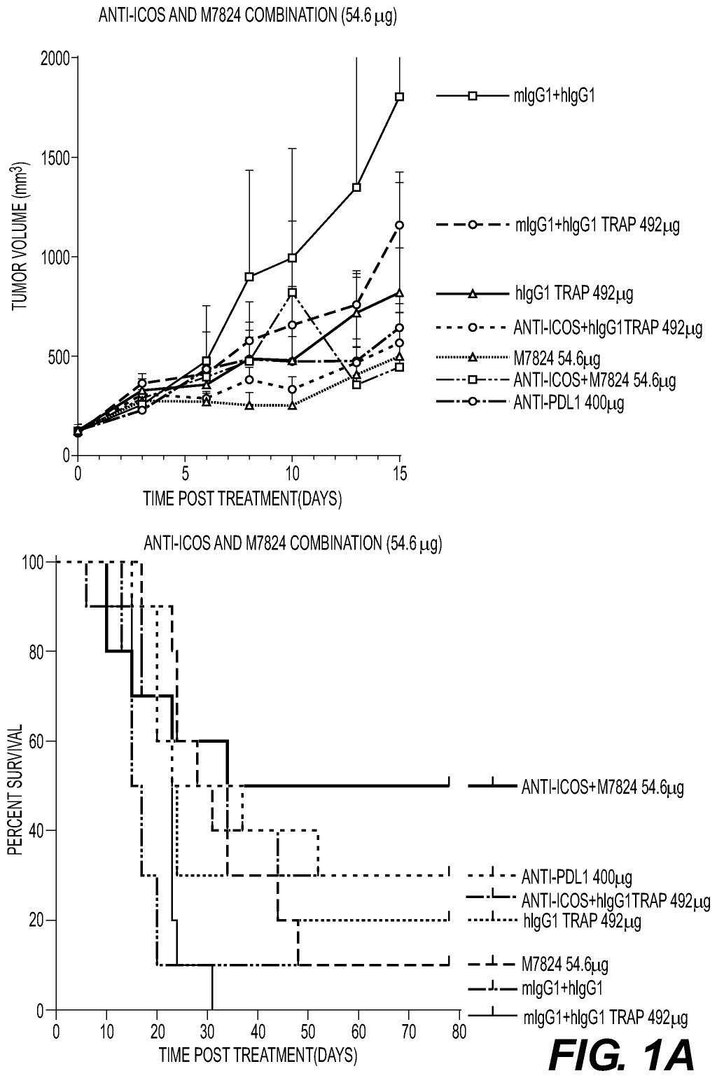

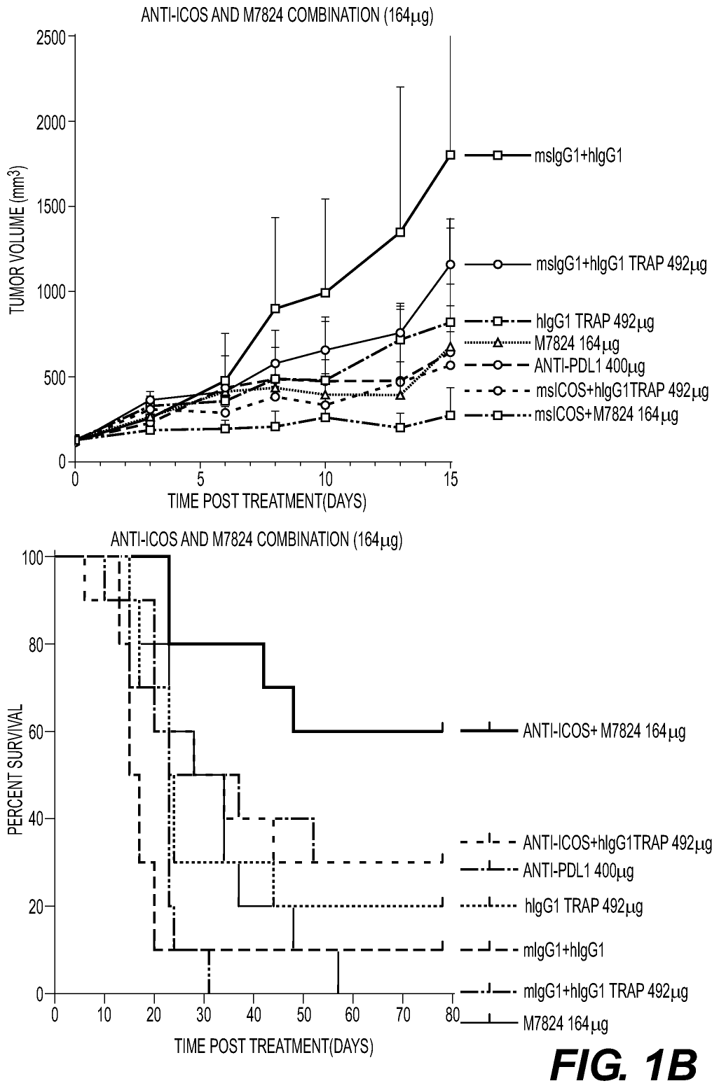

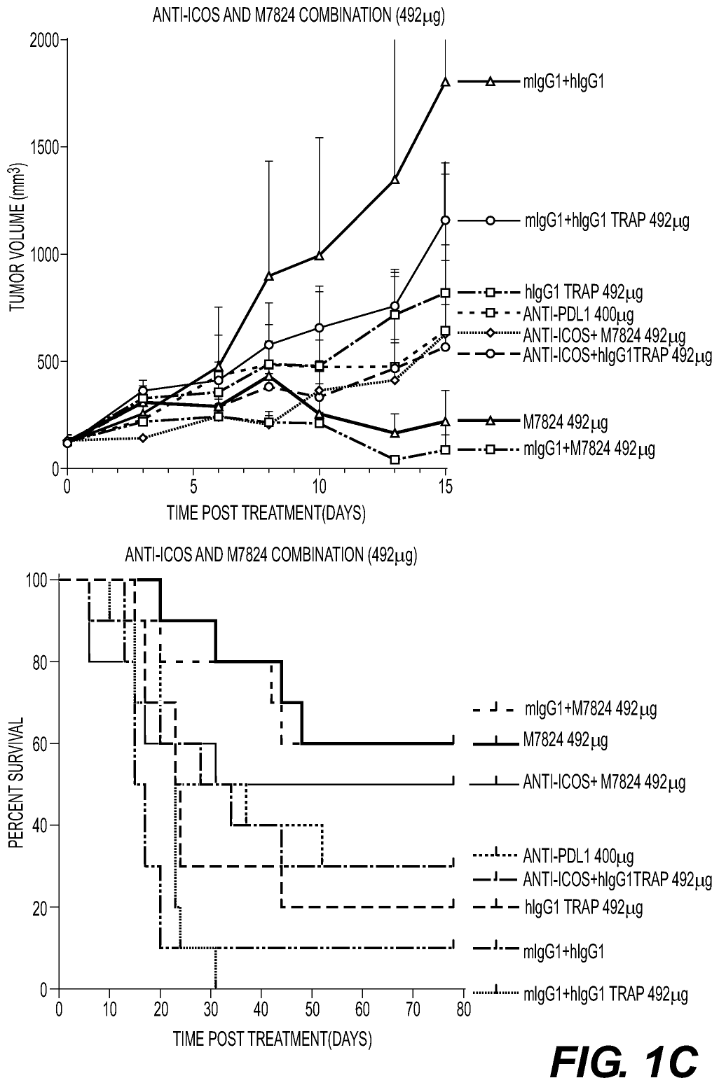

The experiment was run using study design 1, summarised in Table 2, with results shown in FIG. 1.

TABLE 2
Antibody study design 1

| | | | | Dosing |
|---|---|---|---|---|
| Group | Treatment | Tumor | # Mice | (study day) |
| 1 | 1X PBS (vehicle control) | EMT6 | 10 | Twice a week |
| | | | | for 3 weeks |
| 2 | mIgG1 (10 μg) + | EMT6 | 10 | Twice a week |
| | hIgG1 (400 μg) | | | for 3 weeks; |
| | | | | (0, 2, 5) |
| 3 | TGFβRII-Trap control | EMT6 | 10 | (0, 2, 5) |
| | (492 μg) | | | |
| 4 | PD-L1-TGFβRII-Trap | EMT6 | 10 | (0, 2, 5) |
| | (54.6 μg) | | | |
| 5 | PD-L1-TGFβRII-Trap | EMT6 | 10 | (0, 2, 5) |
| | (164 μg) | | | |
| 6 | PD-L1-TGFβRII-Trap | EMT6 | 10 | (0, 2, 5) |
| | (492 μg) | | | |
| 7 | Anti-PD-L1 (400 μg) | EMT6 | 10 | (0, 2, 5) |
| 8 | mIgG1 (10 μg) + | EMT6 | 10 | Twice a week |
| | TGFβRII-Trap control | | | for 3 weeks; |
| | (492 μg) | | | (0, 2, 5) |
| 9 | Anti-ICOS (10 μg) | EMT6 | 10 | Twice a week |
| | | | | for 3 weeks |
| 10 | Anti-ICOS (10 μg) + | EMT6 | 10 | Twice a week |
| | hIgG1 (400 μg) | | | for 3 weeks; |
| | | | | (0, 2, 5) |
| 11 | Anti-ICOS (10 μg) + | EMT6 | 10 | Twice a week |
| | TGFβRII-Trap control | | | for 3 weeks; |
| | (492 μg) | | | (0, 2, 5) |
| 12 | mIgG1 (10 μg) + | EMT6 | 10 | Twice a week |
| | PD-L1-TGFβRII-Trap | | | for 3 weeks; |
| | (492 μg) | | | (0, 2, 5) |
| 13 | Anti-ICOS (10 μg) + | EMT6 | 10 | Twice a week |
| | PD-L1-TGFβRII-Trap | | | for 3 weeks; |
| | (54.6 μg) | | | (0, 2, 5) |
| 14 | Anti-ICOS (10 μg) + | EMT6 | 10 | Twice a week |
| | PD-L1-TGFβRII-Trap | | | for 3 weeks; |
| | (164 μg) | | | (0, 2, 5) |
| 15 | Anti-ICOS (10 μg) + | EMT6 | 10 | Twice a week |
| | PD-L1-TGFβRII-Trap | | | for 3 weeks; |
| | (492 μg) | | | (0, 2, 5) |

Here, FDR p-values were used for statistical analysis. The results for tumor volume and tumor-free survival curves are presented in FIG. 1. ICOS antibody alone demonstrated moderate tumor growth inhibition (TGI) and prolonged tumor free-survival (group 9, 50%; group 10, 40%; group 11, 30%). Although not statistically significant, a trend of tumor growth delay and improvement of overall survival observed relative to isotype control. The anti-tumor efficacy of anti-PD-L1 was modest, with a 20% increase of tumor free-survival relative to isotype control (group 2).

Although M7824 treatment at all three doses tested (54.6 μg, 164 μg, and 492 μg) did not delay tumor growth and improve the survival time at statistical significance, a trend of TGI and increase of tumor free-survival was observed with high dose M7824 (492 μg, group 5 and group 12, 30%) therapy as relative to TGFβRII-Trap control (TGF-β-inhibition only).

Notably, anti-ICOS antibody, in combination with 164 μg M7824, improved anti-tumor efficacy and resulted in a 30% increase of tumor-free survival relative to ICOS or anti-PD-L1 monotherapy. However, statistical significance was not reached for any of the combination groups relative to anti-ICOS or anti-PD-L1 monotherapy, potentially due to the study size. Additional efficacy and pharmacodynamic (PD) studies are required to confirm these observations and determine the mechanistic underpinnings for the combination effect.

As M7824 is a human antibody, all doses of M7824 (3 doses) were administered within the first study week (on day 0, 2, 5) in an attempt to avoid anti-drug antibodies (ADA). No treatment-related deaths were observed during the study. With the outlined dosing strategy, M7824 and ICOS antibodies were well-tolerated, as shown in the consistent body weight increase.

### 1.10 Study Design 2—Results

A further experiment was run using the same methods discussed above using another study design (study design 2) which is summarised in Table 3.

TABLE 3
Antibody study design 2

| | | | | Dosing |
|---|---|---|---|---|
| Group | Treatment | Tumor | # Mice | (study day) |
| 1 | 1X PBS (vehicle control) | EMT6 | 10 | Twice a week |
| | | | | for 3 weeks |
| 2 | mIgG1 100 μg + | EMT6 | 10 | Twice a week |
| | hIgG1 133 μg | | | for 3 weeks; |
| | | | | (0, 2, 5) |
| 3 | mIgG1 100 μg + | EMT6 | 10 | Twice a week |
| | anti-PD-L1 133 μg | | | for 3 weeks; |
| | | | | (0, 2, 5) |
| 4 | mIgG1 100 μg + | EMT6 | 10 | Twice a week |
| | TGFβRII-Trap control | | | for 3 weeks; |
| | 164 μg | | | (0, 2, 5) |
| 5 | PD-L1-TGFβRII-Trap | EMT6 | 10 | (0, 2, 5) |
| | 164 μg | | | |
| 6 | mIgG1 100 μg + | EMT6 | 10 | Twice a week |
| | PD-L1-TGFβRII-Trap | | | for 3 weeks; |
| | 164 μg | | | (0, 2, 5) |
| 7 | Anti-ICOS 100 μg + | EMT6 | 10 | Twice a week |
| | TGFβRII-Trap control | | | for 3 weeks; |
| | 164 μg | | | (0, 2, 5) |
| 8 | Anti-ICOS 10 μg + | EMT6 | 10 | Twice a week |
| | TGFβRII-Trap control | | | for 3 weeks; |
| | 164 μg | | | (0, 2, 5) |
| 9 | Anti-ICOS 1 μg + | EMT6 | 10 | Twice a week |
| | TGFβRII-Trap control | | | for 3 weeks; |
| | 164 μg | | | (0, 2, 5) |
| 10 | Anti-ICOS 100 μg + | EMT6 | 10 | Twice a week |
| | PD-L1-TGFβRII-Trap | | | for 3 weeks; |
| | 164 μg | | | (0, 2, 5) |
| 11 | Anti-ICOS 10 μg + | EMT6 | 10 | Twice a week |
| | PD-L1-TGFβRII-Trap | | | for 3 weeks; |
| | 164 μg | | | (0, 2, 5) |
| 12 | Anti-ICOS 1 μg + | EMT6 | 10 | Twice a week |
| | PD-L1-TGFβRII-Trap | | | for 3 weeks; |
| | 164 μg | | | (0, 2, 5) |

ICOS antibody alone (1 μg and 10 μg; groups 9 and 8, respectively) demonstrated tumor growth inhibition (TGI) and prolonged tumor free-survival (1 μg, 50%; 10 μg, 20%). ICOS antibody alone (100 μg; group 7) did not show any anti-tumor efficacy. The lack of efficacy of 100 μg ICOS antibody is in line with our other in vivo studies (not shown) and reflects the agonist activity of the ICOS antibody. Agonists to a target usually demonstrates a bell-shaped curve response and this is reflected in this and our other in vivo studies, which show lower efficacy at lower or higher doses of ICOS antibody (0.1 μg, 0.01 μg, 100 μg, 200 μg).

A 20% increase in tumor free-survival of anti-PD-L1 (group 3) relative to isotype control (group 4) was observed.

There was anti-tumor efficacy of M7824 treatment at 164 μg (groups 5 and 6), with a 10% increase in tumor free-survival relative to isotype control (group 4) and 10% decline comparing to the anti-PDL1 alone (group 3).

A trend of TGI and increasing of tumor free-survival was observed with 100 μg ICOS antibody and 164 μg M7824 (group 10), and 10 μg ICOS antibody and 164 μg M7824 (group 11) at 50% and 40%, respectively, as relative to TGFβRII-Trap control (TGFβ-inhibition only, group 4) at 10%. However, ICOS antibody (1 μg) in combination with M7824 (group 12) did not show additional benefit relative to ICOS alone.

As M7824 is a human antibody, all doses of M7824 (3 doses) were administered within the first study week (on day 0, 2, 5) in an attempt to avoid anti-drug antibodies (ADA). No treatment-related deaths were observed during the study. With the outlined dosing strategy, M7824 and ICOS antibodies were well tolerated, as shown by the consistent body weight increase.

### Example 2. Combination Therapy Human Clinical Trial Protocol Development

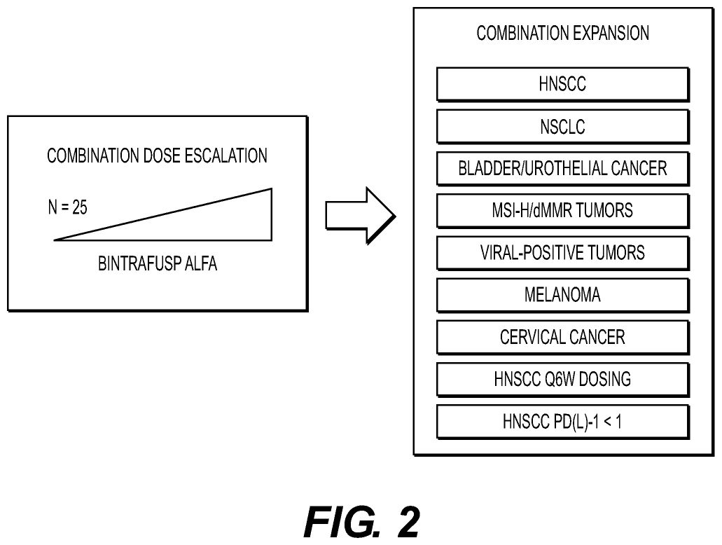

H2L5 hIgG4PE is an anti-Inducible T cell Co-Stimulator (ICOS) receptor agonist antibody intended for the treatment of cancers of different histology. H2L5 hIgG4PE comprises CDR sequences as set out in SEQ ID NOS: 1-6, variable heavy chain and variable light chain sequences as set out in SEQ ID NO:7 and SEQ ID NO: 8, respectively, and heavy chain and light chain sequences as set out in SEQ ID NO:9 and SEQ ID NO:10, respectively. It is expected to be active in combination with agents which prime or modulate tumor immunity. The study design as it relates to the bintrafusp alfa combination is summarised in FIG. 2.

### 2.1 Study Design

H2L5 hIgG4PE will be tested in combination with bintrafusp alfa. The study will investigate doses of 24 mg and 80 mg of H2L5 hIgG4PE Q3W and bintrafusp alfa dose of 2400 mg Q3W.

These combinations evaluated will be investigated in subjects with selected, relapsed and/or refractory solid tumors. Approximately 25 subjects will be enrolled in each cohort.

In dose expansion phases, a Bayesian adaptive design with independent tumor type modeling will be implemented.

2.1.1 H2L5 hIgG4PE Combination with Bintrafusp Alfa

The combination cohorts will each have a dose escalation phase testing two different doses of H2L5 hIgG4PE, 24 mg (Dose Level 1) or 80 mg (Dose Level 2) with the combination partner at a fixed dose regimen for each Dose Level within each cohort of 25 subjects. Bintrafusp alfa combination therapy will begin with a fixed dose schedule of 2400 mg Q3W administered intravenously.

The goal for each cohort will be to determine the recommended Phase 2 dose (RP2D) based on a combination of safety and pharmacodynamic data including tissue level analysis based on biopsy samples. Alternate schedules or dose levels may be explored if data emerge supporting their investigation even after a RP2D is defined.

For each cohort of 25 total subjects, 3 subjects will be enrolled at the first dose level. If no dose-limiting toxicity (DLT) is observed among the 3 subjects, then a Dose Escalation discussion with the study investigators will occur. If DLT is observed among the 3 subjects, the cohort will be expanded to 6 subjects. If no further DLT is observed among the six subjects, then a Dose Escalation discussion with the study investigators will occur. If a second DLT is observed, then the H2L5 hIgG4PE dose will be de-escalated to a lower dose to be determined in discussion between the study team and investigators with a likely target of 0.1 mg/kg. The Dose Escalation Plan is summarized in Table 4.

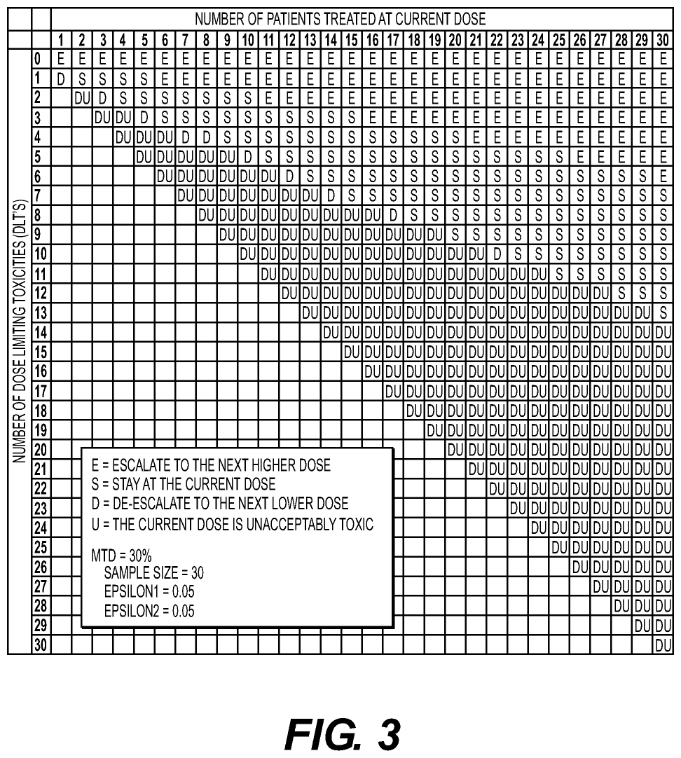

Dose decision rules will follow the modified Toxicity Probability Interval (mTPI) method with FIG. 3 depicting the dose-finding actions escalation decisions based on DLT observed within a cohort. Safety, tolerability, PK, pharmacodynamic measures, and anti-tumor activity will be considered in determining RP2D of H2L5 hIgG4PE in combination.

Because each cohort is limited to 25 subjects, the number enrolled in the PK/pharmacodynamic phase will be 25 minus the number of subjects enrolled in the dose escalation phase. For example, if a total of 3 subjects are enrolled at each of two dose levels, the total number of subjects in dose escalation is 6. Subtracting 6 from 25 will then allow up to 19 subjects to be enrolled in the PK/pharmacodynamic phase. Another scenario could be that the total number of subjects enrolled in dose escalation is 3 at one dose level and 6 at the second dose level, so the dose escalation total is 9, which would allow up to 16 subjects to be enrolled in the PK/pharmacodynamic phase.

TABLE 4
Dose Escalation Plan for Combination Therapies

| | | | | Additional |
|---|---|---|---|---|
| | | | | subjects for PK/ |
| | | | | pharmacodynamics |
| | | | | such that the |
| | H2L5 | | | total for all |
| Dose | hIgG4PE | Combination | N for safety | subjects in the |
| Level | dose | Partner | clearance | cohort is ≤25 |
| 1 | 24 mg | Fixed dose | 3-6 | 6-19 |
| | | regimen | | |
| 2 | 80 mg | Fixed dose | 3-6 | 6-19 |
| | | regimen | | |

If the combination doses in the starting dose cohort are not tolerable, lower doses of H2L5 hIgG4PE may be evaluated.

Additional subjects can be enrolled at one or both of the dose levels following safety clearance at that dose to generate PK/pharmacodynamic data to validate the dose at a tissue level. The PK/pharmacodynamic data will depend on obtaining evaluable tissue samples at baseline and on study at week 6. Based on prior experience, more subjects must be enrolled than samples required for analysis in order to account for non-evaluable or unobtainable tissue samples. All subjects in the PK/pharmacodynamic phase are also included in the anti-drug antibody (ADA) cohorts and assessed for anti-tumor activity based on imaging and immune-related Response Evaluation Criteria in Solid Tumors (irRECIST) criteria as anti-tumor activity is a pharmacodynamic outcome.

The study population in the dose escalation/safety run-in phases of the study are adults with advanced/recurrent solid tumors of the following type: bladder/urothelial cancer, cervical cancer, colorectal cancer (includes appendiceal carcinoma), esophageal cancer with squamous cell histology, head and neck cancer, melanoma, malignant pleural mesothelioma, non-small-cell lung cancer, and prostate cancer. Each cohort may enroll subjects with one specific tumor type selected from the aforementioned list at any time or enroll subjects based on additional features such as prior treatment history (i.e. anti-PD-1/L1 therapy), tumors exhibiting a specific molecular/genetic alteration (i.e. PD-L1 expression), or pathology (i.e. squamous).

2.1.2 Dose Limiting Toxicity

The severity of all toxicities will be graded using National Cancer Institute—Common Terminology Criteria for Adverse Events (NCI-CTCAE) (version 4.0) [NCI, 2010]. The DLT observation period is 28 days in length and begins on the day H2L5 hIgG4PE is first administrated to the subject.

A DLT is defined as an adverse event (AE) that meets at least one of the criteria listed in Table 5 and is considered by the investigator to be clinically relevant and attributed (probably, or possibly) to the study treatment during the 28-day DLT observation period. An AE considered related to the underlying disease under study it is not defined as a DLT.

TABLE 5
Dose-Limiting Toxicity Criteria

| Toxicity | DLT Definition |
|---|---|
| Hematologic | Febrile neutropenia as defined by CTCAE v4 |
| | Grade 4 neutropenia of >7 days in duration |
| | or requiring G-CSF |
| | Grade 4 anemia of any duration |
| | Grade 4 thrombocytopenia of any duration or |
| | Grade 3 thrombocytopenia with bleeding |
| Non-hematologic | Grade 4 toxicity |
| | Grade 3 pneumonitis of any duration |
| | Grade 3 toxicity that does not resolve |
| | to ≤Grade 1 or baseline within 3 days |
| | despite optimal supportive care |
| | Any Grade 2 ocular toxicity requiring systemic |
| | steroids, or any ≥Grade 3 ocular toxicity |
| | Following events are not considered DLTs |
| | Grade 3 and Grade 4 asymptomatic electrolyte |
| | abnormalities that are corrected within 24 |
| | hours without clinical sequelae |
| | Grade 3 nausea, vomiting, or fatigue that |
| | resolves to ≤Grade 1 within 7 days with |
| | optimal supportive care |
| | Grade 3 and Grade 4 infusion reactions in |
| | subjects not receiving prophylaxis for |
| | infusion related reactions (IRRs) (refer |
| | to Section Error! Reference source not |
| | found, for details on IRR management) |
| Other | Toxicity that results in permanent |
| | discontinuation of H2L5 hIgG4PE monotherapy |
| | or H2L5 hIgG4PE and agent in combination |
| | during the first four weeks of treatment |
| | Grade 3/Grade 4 toxicity that results in a |
| | subject not receiving the expected doses of |
| | a regimen in Cycle 1, defined by 21 days |
| | Any other toxicity considered to be dose-limiting |
| | that occurs beyond four weeks will be considered |
| | in the selection of the dose to recommend for |
| | expansion cohorts |
| | Any other event which in the judgment of the |
| | investigator and GSK Medical Monitor is |
| | considered to be a DLT |

a. Note:
Suggested toxicity management guidelines may include systemic corticosteroids for immune-related toxicities; if systemic corticosteroids use delays administration of the second dose of study treatment and the event does not otherwise meet the DLT criteria for non-hematologic toxicity, the dose delay will not be considered a DLT.

If a subject experiences a DLT during the DLT observation period, the subject may resume dosing at the same or lower dose provided the toxicity did not meet study treatment discontinuation criteria and following approval by the Sponsor.

2.1.3 Intra-Subject Dose Escalation

Intra-subject dose escalations may be considered on a case-by-case basis provided the subject has completed at least one treatment cycle without the occurrence of drug-related ≥Grade 2 AE or serious adverse events (SAEs) of any severity Grade in the first 28 days of treatment. For the expansion phases in which Week 6 on-treatment biopsy was mandatory, approval for intra-subject escalation also requires acquisition of this biopsy. Additionally, all subjects at the next higher dose level/levels must have completed the DLT observation period with maximum tolerated dose (MTD) not reached. Subjects may dose-escalate to the highest cleared dose. Individual subjects may dose-escalate multiple times provided that the above criteria are met at each intra-subject dose escalation step.

2.1.4 Dose Expansion Phase

Any dose level(s)/doses in the dose escalation phases may be selected for expansion in order to collect additional data on safety, PK, pharmacodynamic activity, and preliminary clinical activity.

Each expansion cohort will include subjects defined by a single tumor type as indicated in FIG. 2 or characterized by other features such as prior treatment with an immune checkpoint inhibitor, a molecular/genetic alteration (MSI-H/dMMR), or pathology. Subjects may be stratified by prior PD-1/L1 treatment history (i.e. naïve or experienced; best response).

The Steering Committee will review the totality of data available for the study to inform on the dose level indications for any of the expansion cohorts.

2.1.4.1. PK/Pharmacodynamic Dose Expansion Cohorts

Any dose level or levels may be expanded beyond the expected 3 subjects enrolled in dose escalation phase in order to collect additional data on safety, PK, pharmacodynamic activity, and preliminary efficacy. Subjects can only be enrolled at previously cleared dose levels. Subjects enrolled in PK/pharmacodynamic cohorts may have the dose escalated to a higher cleared dose level (i.e. not exceeding the MTD) once the necessary PK/pharmacodynamic procedures have been completed. Model-based designs may be employed for each PK/pharmacodynamic dose expansion cohort in order to sufficiently explore parameters critical (i.e. safety, tolerability, and efficacy) in establishing the biologically optimal doses of the agents in the combination.

2.1.5 Study Treatment and Duration

Each part and phase of the study includes a screening period, a treatment period, and a follow-up period. For subjects who meet all eligibility criteria and register into the study, the maximum duration of treatment with H2L5 hIgG4PE is expected to be two years, up to 35 cycles. The maximum follow-up period for safety assessments will be 90 days from the date of the last dose of study treatment. The expected maximum follow-up period for survival and subsequent anti-cancer therapy will be two years from the date of the last dose of study treatment. Subjects who discontinue study treatment due to achieving confirmed complete response (CR) (refer to Section 2.2.3 for additional requirements) will be followed for progression (refer to Section 2.2.3 for details on the frequency of these assessments).

Subjects participating in the bintrafusp alfa combination cohort will receive H2L5 hIgG4PE 24 or 80 mg dose (refer to Table 6 for fixed doses) in combination with bintrafusp alfa administered as an IV infusion at 2400 mg Q3W.

2.1.6 Dose Justification

2.1.6.1 H2L5 hIgG4PE Starting Dose in Bintrafusp alfa

The H2L5 hIgG4PE doses of 24 mg and 80 mg were selected based on the preliminary ICOS receptor occupancy pharmacodynamic analysis in the periphery which showed high receptor occupancy levels on CD4 and CD8 T cells over the 21-day dosing cycle starting at 0.3 m/kg (˜24 mg); close to total receptor saturation was observed at 1 mg/kg (˜80 mg) dose level. Based on prior clinical and non-clinical data, no overlapping toxicities are expected. Also, based on established pharmacology, no drug-drug interactions are expected.

2.1.6.2 H2L5 hIgG4PE Dosing Frequency

Since select partner agents may be dosed less frequently than every three weeks, alternative extended dosing schedules would provide additional convenience and flexibility to patients and clinicians beyond a Q3W option. Hence, a six-weekly (Q6W) dosing schedule for H2L5 hIgG4PE will be explored, specifically in randomized schedule optimization cohorts for subjects with PD-1/L1 Naive HNSCC. Two doses for initial Q6W schedule exploration, 48 and 160 mg, are selected to provide matching cumulative exposures corresponding to respective Q3W regimens in the Q3W HNSCC dose-randomized cohorts (0.3 and 1 mg/kg). Preliminary PK simulations suggest a doubling of dose and interval for H2L5 hIgG4PE (e.g. 0.3 mg/kg Q3W to 48 mg Q6W) is expected to provide similar cumulative AUC with an approximate doubling of end-of-infusion Cmax and marginally lower end-of-cycle trough concentrations (˜43% at steady-state). The typical Cmax for 160 mg Q6W will be maintained below thresholds established with the Q3W regimens.

2.1.6.3 H2L5 hIgG4PE Fixed Dose Rationale

Fixed doses may be tested in the dose escalation with bintrafusp alfa, assuming a typical median weight of 80 kg.

Preliminary population PK simulations indicate that using fixed dosing would result in a similar range of exposures as that of body weight-based dosing. Also, fixed dosing offers the advantage of reduced dosing errors, reduced drug wastage, shorten preparation time, and improve ease of administration. Thus, switching to a fixed dose based on a reference body weight of 80 kg is reasonable and appropriate.

The fixed dose equivalents of the weight-based H2L5 hIgG4PE dose levels using 80 kg weight are presented in Table 6.

TABLE 6
H2L5 hIgG4PE Fixed Dose Calculations

| | H2L5 | H2L5 |
|---|---|---|
| Dose | hIgG4PE | hIgG4PE |
| Level | (mg/kg) | (mg) |
| 1 | 0.001 | 0.08 |
| 2 | 0.003 | 0.24 |
| 3 | 0.01 | 0.8 |
| 4 | 0.03 | 2.4 |
| 5 | 0.1 | 8.0 |
| 6 | 0.3 | 24.0 |
| 7 | 0.6 | 48.0 |
| 8 | 1.0 | 80.0 |
| 9 | 2.0 | 160.0 |
| 10 | 3.0 | 240.0 |

2.1.6.4 Bintrafusp alfa (anti-PD-L1-TGFβ Trap) Dose Rationale

The dose for M7824 (bintrafusp alfa) in this study is 2400 mg administered as an intravenous infusion once every 3 weeks. Since H2L5 IgG4PE is administered every 3 weeks, the same dosing interval for M7824 is preferred for convenience and compliance.

2.2 Selection of Study Population and Withdrawal Criteria

2.2.1 Inclusion Criteria

For a subject to be eligible for inclusion in this study all the following criteria must be fulfilled:

1. Capable of giving signed, written informed consent

2. Male or female, age ≥18 years (at the time consent is obtained).

3. Histological or cytological documentation of an invasive malignancy that was diagnosed as locally advanced/metastatic or relapsed/refractory and is of one of the following tumor types:
Bladder/urothelial cancer of the upper and lower urinary tract
Cervical
Colorectal (includes appendix)
Esophagus, squamous cell
Head and Neck Carcinoma
Melanoma
MPM
NSCLC
Prostate
MSI-H/dMMR tumor
HPV-positive or EBV-positive tumor

4. Disease that has progressed after standard therapy for the specific tumor type, or for which standard therapy has proven to be ineffective, intolerable, or is considered inappropriate, or if no further standard therapy exists.
Subjects must not have received more than 5 prior lines of therapy for advanced disease including both standards of care and investigational therapies.
Subjects who received prior anti-PD-1/L1 therapy must fulfill the following requirements:
   Have achieved a complete response [CR], partial response [PR]) and stable disease [SD] and subsequently had disease progression while still on PD 1/L1 therapy;
   Have received at least 2 doses of an approved PD-1/L1 inhibitor (by any regulatory authority);
   Have demonstrated disease progression as defined by RECIST v1.1 within 18 weeks from the last dose of the PD-1/L1 inhibitor. The initial evidence of disease progression is to be confirmed by a second assessment no less than four weeks from the date of the first documented PD (the confirmatory scan could be the baseline eligibility scan for this study).

5. Archival tumor tissue obtained at any time from the initial diagnosis to study entry; a fresh tumor biopsy using a procedure that is safe for the subject on a lesion not previously irradiated unless lesion progressed will be required if archival tissue is unavailable.

6. Agree to undergo a pre-treatment and on-treatment biopsy and have disease amenable to biopsy required in PK/pharmacodynamic, dose randomized HNSCC, Melanoma dose expansion and Biomarker cohorts.

7. Measurable disease per RECIST version 1.1 (refer to Section 2.6). Palpable lesions that are not measurable by radiographic or photographic evaluations may not be utilized as the only measurable lesion. Any measurable lesion biopsied at Screening cannot be followed as a target/index lesion unless agreed upon by GSK.

8. Eastern Cooperative Oncology Group (ECOG) performance status (PS) 0-1 (refer to Section 2.7).

9. Life expectancy of at least 12 weeks.

10. Adequate organ function as defined in Table 7:

TABLE 7
Definitions for Adequate Organ Function

| | |
|---|---|
| System | Laboratory Values |

Hematologic^{b}

| | |
|---|---|
| Absolute neutrophil | ≥1.5 × 10⁹/L |
| count (ANC) | |
| Hemoglobin | ≥9 g/dL |
| Platelets | ≥100 × 10⁹/L |

Hepatic

| | |
|---|---|
| Total bilirubin | ≤1.5 × upper limit of normal (ULN) |
| For subjects with | ≤3.0 × ULN |
| Gilbert's Syndrome | |
| (only if direct | |
| bilirubin ≤35%) | |
| Alanine | ≤2.5 × ULN; |
| aminotransferase | or ≤5 × ULN for subjects with documented |
| (ALT) | liver metastases |

Renal

| | |
|---|---|
| Calculated creatinine | ≥30 mL/min |
| clearance^{c} | |

Cardiac

| | |
|---|---|
| Ejection fraction | ≥50% by echocardiogram^{d} |

a. Absolute Lymphocyte Count will be included in the baseline assessment, but no range limit requirement for the eligibility.
^{b}Estimated CrCl should be calculated using the Chronic Kidney Disease Epidemiology Collaboration (CKD-EPI) formula.
^{c}Multigated acquisition scan (MUGA) is acceptable if ECHO is not available (refer to Echocardiograms section, below)

11. QT duration corrected for heart rate by Fridericia's formula (QTcF)<450 milliseconds (msec) or QTcF <480 msec for subjects with bundle branch block. The QTcF is the QT interval corrected for heart rate according to Fridericia's formula, machine-read or manually over-read.

12. A female subject is eligible to participate if she is not pregnant (as confirmed by a negative serum beta-human chorionic gonadotrophin [β-hCG] test in females of reproductive potential) and not lactating, or at least one of the following conditions applies:

a) Non reproductive potential, defined as:
Pre-menopausal females with one of the following: Documented tubal ligation, Documented hysteroscopic tubal occlusion procedure with follow-up confirmation of bilateral tubal occlusion, Hysterectomy, Documented Bilateral Oophorectomy
Postmenopausal defined as 12 months of spontaneous amenorrhea. Females on hormone replacement therapy (HRT) and whose menopausal status is in doubt will be required to use one of the highly effective contraception methods if they wish to continue their HRT during the study. Otherwise, they must discontinue HRT to allow confirmation of post-menopausal status prior to study enrolment.

b) Reproductive potential and agrees to follow highly effective methods for avoiding pregnancy from 30 days prior to the first dose of study medication and until 120 days after the last dose of study treatment.

13. Male subjects with female partners of child bearing potential must agree to use a highly effective method of contraception from time of first dose of study treatment until 120 days after the last dose of study treatment.

14. Documented Human Papilloma Virus (HPV)/Epstein-Barr (EBV)-positive tumor as determined by a local laboratory for viral-positive expansion cohorts only

15. Documented MSI-H or dMMR-positive tumor as determined by local laboratory for combination MSI-H/dMMR expansion cohorts only.

16. PD-L1 CPS <1 using the FDA approved PD-L1 IHC 22C3 pharmDx assay by central laboratory testing for HNSCC PD-L1 CPS <1 Cohort. Documented test result from FDA approved PD-L1 IHC 22C3 pharmDx assay in local laboratory, if available, may be accepted in lieu of the central laboratory test result.

17. Defined PD-L1 expression using the Ventana PD-L1 (SP263) IHC assay by central testing for enrollment in the PK/PD cohort with combination studies.

2.2.2 Exclusion Criteria

A subject will not be eligible for inclusion in this study if any of the following criteria apply:

1. Prior treatment with the following therapies:
Anti-cancer therapy within 30 days or 5 half-lives of the drug, whichever is shorter. At least 14 days must have elapsed between the last dose of prior anti-cancer agent and the first dose of study drug is administered.
Prior radiation therapy: permissible if at least one non-irradiated measurable lesion is available for assessment according to RECIST version 1.1 or if a solitary measurable lesion was irradiated, objective progression is documented. A wash out of at least two weeks before start of study drug for radiation of any intended use to the extremities for bone metastases and 4 weeks for radiation to the chest, brain, or visceral organs is required.
Investigational therapy within 30 days or 5 half-lives of the investigational product (whichever is shorter). At least 14 days must have elapsed between the last dose of investigational agent and the first dose of study drug is administered.

2. Prior allogeneic or autologous bone marrow transplantation or other solid organ transplantation.

3. Toxicity from previous anti-cancer treatment that includes:
≥Grade 3 toxicity considered related to prior immunotherapy and that led to treatment discontinuation.
Toxicity related to prior treatment that has not resolved to ≤Grade 1 (except alopecia, endocrinopathy managed with replacement therapy, and peripheral neuropathy which must be ≤Grade 2).

4. Invasive malignancy or history of invasive malignancy other than disease under study within the last two years, except as noted below:
Any other invasive malignancy for which the subject was definitively treated, has been disease-free for ≤2 years and in the opinion of the principal investigator and GSK Medical Monitor will not affect the evaluation of the effects of the study treatment on the currently targeted malignancy, may be included in this clinical trial.
Curatively treated non-melanoma skin cancer.

5. Central nervous system (CNS) metastases, with the following exception:
Subjects who have previously-treated CNS metastases, are asymptomatic, and have no requirement for steroids at least 14 days prior to first dose of study drug. Note: Subjects with carcinomatous meningitis or leptomeningeal spread are excluded regardless of clinical stability.

6. Received transfusion of blood products (including platelets or red blood cells) or administration of colony stimulating factors (including granulocyte colony-stimulating factor [G-CSF], granulocyte-macrophage colony-stimulating factor, recombinant erythropoietin) within 14 days prior to the first dose of H2L5 hIgG4PE.

7. Major surgery 54 weeks before the first dose of study treatment. Subjects must have also fully recovered from any surgery (major or minor) and/or its complications before initiating study treatment.

8. Active autoimmune disease that has required systemic treatment within the last two years (i.e. with use of disease modifying agents, corticosteroids or immunosuppressive drugs). Note: Replacement therapy (e.g. thyroxine or physiologic corticosteroid replacement therapy for adrenal or pituitary insufficiency, etc.) is not considered a form of systemic treatment.

9. Concurrent medical condition requiring the use of systemic immunosuppressive medications within 7 days before the first dose of study treatment. Physiologic doses of corticosteroids for treatment of endocrinopathies or steroids with minimal systemic absorption, including topical, inhaled, or intranasal corticosteroids may be continued if the subject is on a stable dose.

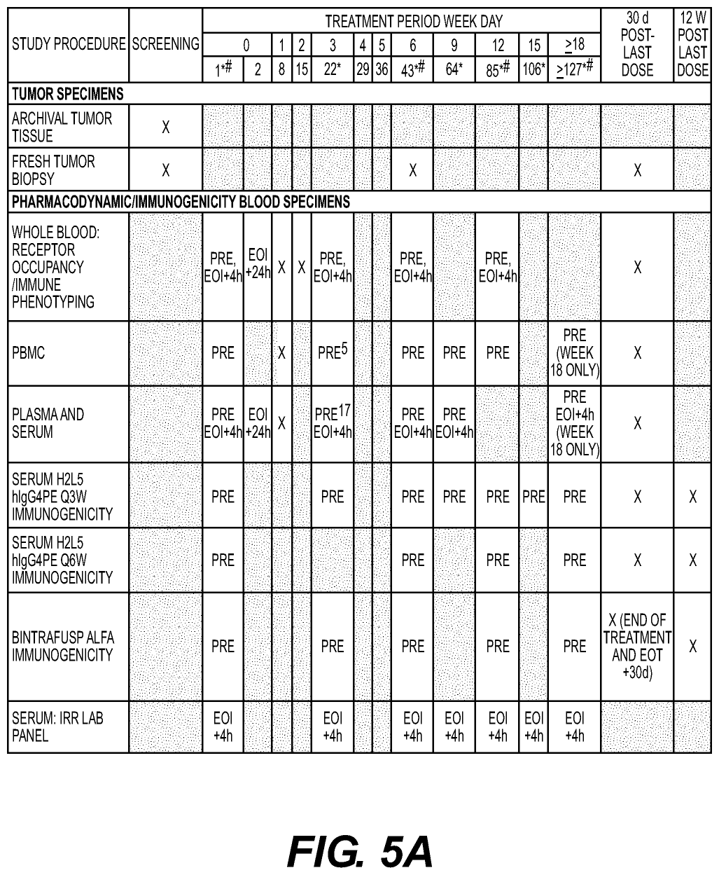

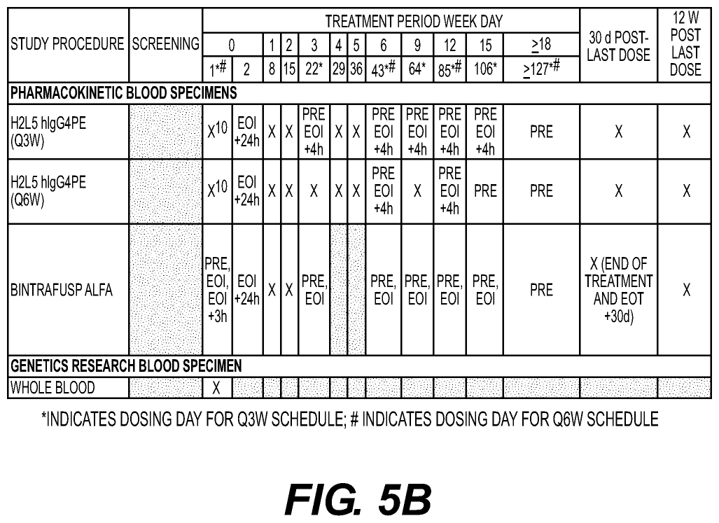

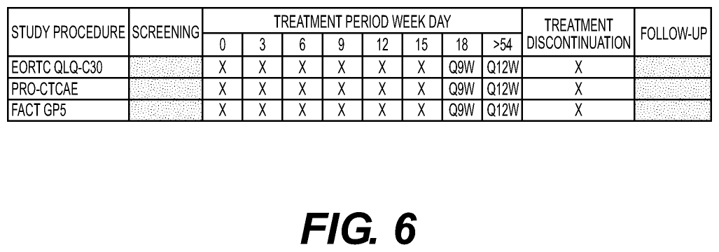

10. Active infection requiring systemic therapy, known human immunodeficiency virus infection, or positive test for hepatitis B active infection or hepatitis C active infection (refer to FIG. 5 for details).

11. Current active liver or biliary disease (with the exception of Gilbert's syndrome or asymptomatic gallstones, liver metastases, or otherwise stable chronic liver disease per investigator assessment). Note: Stable chronic liver disease should generally be defined by the absence of ascites, encephalopathy, coagulopathy, hypoalbuminemia, esophageal or gastric varices, persistent jaundice, or cirrhosis.

12. Recent history (within the past 6 months) of acute diverticulitis, inflammatory bowel disease, intra-abdominal abscess, or gastrointestinal obstruction that required surgery

13. Receipt of any live vaccine within 4 weeks prior to first dose of study treatment.

14. Recent history of allergen desensitization therapy within 4 weeks of starting study treatment.

15. History of severe hypersensitivity to monoclonal antibodies or to the chemotherapies under investigation including any ingredient used in the formulation.

16. History or evidence of cardiac abnormalities including any of the following:
Recent (within the past 6 months) history of serious uncontrolled cardiac arrhythmia or clinically significant electrocardiogram abnormalities including second degree (Type II) or third degree atrioventricular block.
Cardiomyopathy, myocardial infarction, acute coronary syndromes (including unstable angina pectoris), coronary angioplasty, stenting, or bypass grafting within the past 6 months before enrollment.
Congestive heart failure (Class II, III, or IV) as defined by the New York Heart Association functional classification system.
Recent (within the past 6 months) history of symptomatic pericarditis.

17. History (current and past) of idiopathic pulmonary fibrosis, pneumonitis (for past pneumonitis exclusion only if steroids were required for treatment), interstitial lung disease, or organizing pneumonia. Note: post-radiation changes in the lung related to prior radiotherapy and/or asymptomatic radiation-induced pneumonitis not requiring treatment may be permitted if agreed by the investigator and Medical Monitor.

18. Recent history (within 6 months) of uncontrolled symptomatic ascites or pleural effusions.

19. History of bleeding diathesis or recent major bleeding events (this exclusion criterion applies to subjects enrolled in the bintrafusp alfa combination cohort).

20. Any serious and/or unstable pre-existing medical, psychiatric disorder, or other condition that could interfere with the subject's safety, obtaining informed consent, or compliance to the study procedures.

21. Is or has an immediate family member (e.g. spouse, parent/legal guardian, sibling or child) who is an investigational site or sponsor staff directly involved with the trial, unless prospective IRB approval (by chair or designee) is given allowing exception to this criterion for a specific subject.

2.2.3 Withdrawal/Stopping Criteria

Subjects will receive study treatment for the scheduled time period, if applicable, unless one of the following events occurs earlier: disease progression (as determined by irRECIST), death, or unacceptable toxicity, including meeting stopping criteria for liver chemistry (refer to Section 2.2.3.1), or other criteria are met as defined in Section 2.2.3.2. Subjects with infusion delays >21 days due to toxicity should consider discontinuing study drug(s) unless the treating investigator and Sponsor/Medical Monitor agree there is strong evidence supporting continued treatment.

Subjects enrolled who require permanent discontinuation of one of the study agents in a given treatment combination due to toxicity must permanently discontinue both agents in that combination, unless continued treatment with the remaining agent is agreed upon by the treating investigator and Sponsor/Medical Monitor.

In addition, study treatment may be permanently discontinued for any of the following reasons:

a. Deviation(s) from the protocol

b. Request of the subject or proxy

c. Discretion of the investigator

d. Subject is lost to follow-up

e. Closure or termination of the study

The primary reason for discontinuation must be recorded in the subject's medical records and electronic case report form (eCRF).

If the subject voluntarily discontinues from treatment due to toxicity, ‘adverse event’ will be recorded as the primary reason for permanent discontinuation on the eCRF.

Once a subject has permanently discontinued from study treatment, the subject will not be allowed to be retreated.

The assessments required at the treatment discontinuation visit (TDV) must be completed within 30 days of the decision to permanently discontinue study drug(s) and prior to the start of subsequent anti-cancer therapy.

All subjects who discontinue from study treatment (early or permanent) for any reason will have safety assessments at the time of discontinuation and during post study treatment follow-up.

Subjects with a CR or PR require confirmation of response via imaging at least 4 weeks after the first imaging showed a CR or PR.

Early discontinuation of study treatment (early discontinuation of study treatment will not per se constitute permanent discontinuation) may be considered for subjects who have attained a confirmed complete response per RECIST 1.1 and who received study treatment for at least 24 weeks and had at least two treatments beyond the date when the initial CR was declared; these subjects will undergo disease assessments at a frequency of 12 weeks. These subjects may be permitted to resume study treatment upon disease progression; this retreatment is defined as a Second Course. In addition, subjects with RECISTv1.1 confirmed SD, PR, or CR who complete the 35 cycles of study treatment and study treatment is discontinued for this reason and not for other reasons such as disease progression or intolerability will undergo disease assessments at a frequency of 12 weeks: these subjects may be able to receive a second course of study treatment upon disease progression. For subjects to be eligible for a second course of study treatment, all following requirements must be met:
Experienced an investigator-determined radiographic disease progression by RECIST 1.1 after discontinuing the initial course of study treatment
No subsequent/new anti-cancer treatment was administered after the last dose of study treatment
Fulfilled all of the safety parameters listed in the inclusion criteria and none of the safety parameters listed in the exclusion criteria are met
The study is still ongoing

If study treatment is restarted, subjects will be required to resume assessments; in addition, limited PK and immunogenicity sampling is required.

All subjects who permanently discontinue study treatment for any reason will be followed for survival and new anti-cancer therapy (including radiotherapy) every 12 weeks until death, termination of the overall study or a cohort by the sponsor or until the subject has been followed for two years.

If subjects are unable or unwilling to attend clinic visits during follow-up, contact to assess survival may be made via another form of communication (e.g. telephone, email, etc.).

All subjects who permanently discontinue study treatment for reasons other than disease progression or consent withdrawal will be followed for progression or until the start of anti-cancer therapy whichever comes first. 2.2.3.1 Liver Chemistry Stopping Criteria

Liver chemistry stopping and increased monitoring criteria have been designed to assure subject safety and evaluate liver event etiology (in alignment with the Food and Drug Administration (FDA) premarketing clinical liver safety guidance).

If any of the criteria in Table 8 are met, all study drugs must be discontinued.

TABLE 8
Liver Chemistry Stopping Criteria
Liver Stopping Event for the subjects with ALT ≤2.5 ULN at the baseline value

| | |
|---|---|
| ALT-Increase | ALT ≥5 × ULN |
| ALT Increase | ALT ≥3 × ULN but <5 × ULN persists |
| | for ≥4 weeks |
| Bilirubin^{a,b} | ALT ≥3 × ULN and bilirubin ≥2 × ULN |
| | (>35% direct bilirubin) |
| International Normalized | ALT ≥3 × ULN and INR >1.5 |
| Ratio (INR)^{b} | |
| Cannot Monitor | ALT ≥3 × ULN but <5 × ULN and cannot |
| | be monitored weekly for ≥4 weeks |
| Symptomatic^{c} | ALT ≥3 × ULN associated with symptoms |
| | (new or worsening) believed to be related |
| | to liver injury or hypersensitivity |

Liver Stopping Event for Subjects with ALT >2.5 or ≤5 × ULN at Baseline Value

| | |
|---|---|
| ALT absolute | Both ALT ≥5 × ULN and ≥2 × baseline value |
| ALT Increase | Both ALT ≥3 × ULN and ≥1.5 × baseline |
| | value that persists for ≥4 weeks |
| Bilirubin^{a,b} | ALT ≥3 × ULN and bilirubin ≥2 × ULN |
| | (>35% direct bilirubin) |
| INR^{b} | ALT ≥3 × ULN and INR >1.5 |
| Cannot Monitor | Both ALT ≥3 × ULN and ≥1.5 × baseline |
| | value that cannot be monitored for 4 weeks |
| Symptomatic^{c} | Both ALT ≥3 × ULN and ≥1.5 × baseline |
| | value associated with symptoms (new or worsening) |
| | believed to be related to liver injury or |
| | hypersensitivity |

^{a}Serum bilirubin fractionation should be performed if testing is available. If serum bilirubin fractionation is not immediately available, discontinue study treatment if ALT ≥3 × ULN and bilirubin ≥2 × ULN. Additionally, if serum bilirubin fractionation testing is unavailable, record presence of detectable urinary bilirubin on dipstick, indicating direct bilirubin elevations and suggesting liver injury.
^{b}All events of ALT ≥3 × ULN and bilirubin ≥2 × ULN (>35% direct bilirubin) or ALT ≥3 × ULN and INR >1.5, which may indicate severe liver injury (possible ‘Hy's Law’), must be reported as an SAE (excluding studies of hepatic impairment or cirrhosis); INR measurement is not required and the threshold value stated will not apply to subjects receiving anticoagulants.
^{c}New or worsening symptoms believed to be related to liver injury (such as fatigue, nausea, vomiting, right upper quadrant pain or tenderness, or jaundice) or believed to be related to hypersensitivity (such as fever, rash or eosinophilia).

2.2.3.2 Stopping Rules for Clinical Deterioration

To adequately assess the anti-tumor effect of immunotherapeutic agents it is reasonable to allow subjects experiencing apparent progression as defined by RECIST 1.1 guidelines to continue to receive treatment until progression is confirmed at the next imaging assessment at least 4 weeks later as indicated by irRECIST guidelines. Nevertheless, these considerations should be balanced by clinical judgment as to whether the subject is clinically deteriorating and unlikely to receive any benefit from continued study treatment.

In cases where deterioration was assessed to have occurred after a clinical event that, in the investigator's opinion, is attributable to disease progression and is unlikely to reverse with continued study treatment or managed by supportive care (e.g. bisphosphonates and/or bone directed radiotherapy, thoracentesis, or paracentesis for accumulating effusions), study treatment should be discontinued. Examples of events that may, in the investigator's opinion, indicate a lack of clinical benefit include, but are not limited to, the following:
ECOG PS worsening of at least 2 points from baseline
Skeletal related events defined by the following: pathologic bone fracture in the region of cancer involvement; cancer related surgery to bone; and/or spinal cord or nerve root compression
Development of new CNS metastases
Any setting where the initiation of new antineoplastic therapy has been deemed beneficial to the subject even in the absence of any such documented clinical event.

2.2.4 Subject and Study Completion

For combinations with bintrafusp alfa and the dose escalation phases of the study, subjects will be considered as completing the study if they complete screening assessments, receive at least two doses of study treatment or receive one dose but experience a DLT, are observed during the 28 day DLT observation period, and complete the treatment discontinuation visit and the follow-up visit for safety or have died while receiving study treatment or during post-study treatment follow-up period for safety.

### 2.3 Study Treatment

2.3.1 Investigational Product and Other Study Treatment

Bintrafusp alfa (refer to Table 9) will be administered intravenously to subjects starting at least 30 minutes and no more than one hour following the end of the H2L5 hIgG4PE infusion under medical supervision of an investigator or designee.

All subjects are required to remain under observation at the study site for at least 1.5 hours post-infusion of the last study drug administered for the first two study treatment dosing visits. At subsequent study treatment dosing visits, for subjects who experience infusion-related reactions, the post-infusion observation time should remain as at least 1.5 hours; for subjects who do not experience infusion reactions, these subjects should remain under observation at the study site post-study treatment infusion for at least 30 minutes or as per the judgement of the investigator or as per institutional guidelines.

For drug administered by an investigator or designee, the dose of study treatment and study subject identification will be confirmed at the time of dosing by a member of the study site staff other than the person administering the study treatment. The specific time of study treatment administration (e.g. time of the week for first administration; time of the day for each administration) should take into consideration PK sampling time points, study visit procedures, and the post-infusion observation time interval. Infusions may be administered up to 72 hours before or after the planned date of treatment for administrative reasons only (e.g. scheduling an infusion around a holiday).

TABLE 9
Combination Study Products Description and Administration

| | Study Treatment |
|---|---|
| Product Name: | Bintrafusp alfa |
| Product | Humanized anti-PD-L1-TGFβ- trap |
| Description | fusion protein |
| Dosage form/ | 10 mg/mL solution |
| strength | |
| Planned | 2400 mg |
| dosage level(s) | |
| Route of | IV infusion |
| Administration | |
| Dosing | Administer diluted product/once Q3W |
| instructions/ | (refer to SRM for infusion time) |
| Frequency | |
| Manufacturer | Merck KGaA |

2.3.2 Treatment Assignment

Subjects enrolled in the study will be assigned to a combination treatment in an open-label fashion and according to the combination treatment cohorts open for accrual. Other expansion cohorts may investigate more than one dose level of H2L5 hIgG4PE; if implemented, subjects in this cohort will be randomly assigned to the selected dose levels.

2.3.3 Blinding

This is an open-label study.

2.3.4 Concomitant Medications and Non-Drug Therapies

Subjects will be instructed to inform the investigator prior to starting any new medications from the time of first dose of study treatment until discontinuation of study treatment. Any permitted concomitant medication(s), including non-prescription medication(s) and herbal product(s), taken during the study will be recorded in the eCRF. The minimum requirement for reporting is drug name, dose, dates of administration, and the reason for medication.

2.3.4.1 Permitted Medications and Non-Drug Therapies

Elective palliative surgery or radiation may be permitted on a case-by-case basis in consultation with GSK Medical Monitor.

The following medications are permitted as indicated:

a. Bisphosphonates and receptor activator of nuclear factor-kappaB ligand (RANKL) inhibitors (e.g. denosumab): subjects are required to have been on a stable dose for at least 4 weeks prior to receiving first dose of H2L5 hIgG4PE. Prophylactic use in subjects without evidence or history of bone metastasis is not permitted, except for the treatment of osteoporosis.

b. Growth factors: initiation of growth factors is not permitted during the first 4 weeks of study treatment, unless clinically indicated for toxicity management and agreed upon by the investigator and the GSK Medical Monitor.

c. Steroids: Subjects with pre-existing conditions requiring steroids are permitted to continue taking up to a maximum of 10 mg of prednisone or equivalent provided the subject has been on a stable dose for at least 28 days before first dose of H2L5 hIgG4PE; refer to exclusion criterion 9 in Section 2.2.2 for further requirements. Steroids used for chemotherapy premedication are permitted.

2.3.4.2. Prohibited Medications and Non-Drug Therapies

The following medications are prohibited before the first dose of study treatment (refer to Section 2.2.2 for specific time requirements) and while on treatment in this study:

a. Anti-cancer therapies (other than those used in this study) that include but are not limited to chemotherapy, immunotherapy, biologic therapy, hormonal therapy (other than physiologic replacement), surgery, and radiation therapy (other than palliative intervention as described in Section 2.3.4.1);

b. Any investigational drug (s) other than those referred to in this study;

c. Live vaccines such as intra-nasal flu vaccine.

### 2.4 Study Assessment and Procedures

This section lists the procedures and parameters of each planned study assessment. The exact timing of each assessment is listed in the Time and Events Tables depicted in FIGS. 4 and 5.

The following points must be noted:
If assessments are scheduled for the same nominal time, then the assessments should occur in the following order:
   1. 12-lead ECG
   2. Vital signs
   3. Blood draws (e.g. PK blood draws). Note: The timing of the assessments should allow the blood draw to occur at the exact nominal time.
The timing and number of planned study assessments, including safety, pharmacokinetic, pharmacodynamic/biomarker or others assessments may be altered during the course of the study based on emerging data (e.g. to obtain data closer to the time of peak plasma concentrations) to ensure appropriate monitoring.
No more than 500 mL of blood will be collected over the first four doses of study treatment.

2.4.1 Screening and Critical Baseline Assessments

The following demographic parameters will be captured: year of birth, sex, race and ethnicity.

Medical history including cardiovascular medical history/risk factors will be assessed as related to the inclusion/exclusion criteria listed in Section 2.2.1 and Section 2.2.2.

Disease characteristics including medical, surgical, and treatment history including radiotherapy, date of initial diagnosis, stage at initial diagnosis, histology, tumor genetic/genomic features, tumor viral status and current sites of disease will be taken as part of the medical history and disease status; scans from imaging studies performed prior to screening scans required for baseline lesion assessments may be requested. Details concerning prior anti-cancer therapy (e.g. systemic and radiation therapy) including best response to prior systemic therapy will be recorded for at least two prior lines of therapy (if available).

For subjects with PD-1/L1 treatment naïve HNSCC screening for enrollment to the HNSCC PD-L1 CPS <1 cohort only: PD-L1 protein expression using the PD-L1 IHC 22C3 pharmDx assay by local laboratory testing; if not available, central laboratory testing. An evaluable CPS score is required for eligibility; refer to Section 2.2.1 for CPS eligibility requirements.

Baseline lesion assessments required within 30 days prior to the first dose of H2L5 hIgG4PE include:
Computed Tomography (CT) scan with contrast of the chest, abdomen, and pelvis;
For subjects with head and neck cancer, a CT/Magnetic Resonance Imaging (MRI) of the head and neck area is required;
Clinical disease assessment for palpable/visible lesions;
Other areas as indicated by the subject's underlying disease present prior to screening.

Note: Although CT scan is preferred, MRI may be used as an alternative method of baseline disease assessment, especially for those subjects where a CT scan is contraindicated due to allergy to contrast, provided that the method used to document baseline status is used consistently throughout study treatment to facilitate direct comparison. Refer to RECIST version 1.1 guidelines for use of fluorodeoxyglucose-positron emission tomography (FDG-PET)/CT (Eisenhauer et al. Eur J Cancer. 2009; 45:228-247).

Refer to Section 2.4.2 for baseline documentation of target and non-target lesions.

Safety and laboratory assessments required at baseline include:
Physical examination
Performance Status
Vital Signs
Concomitant medication
   Recorded starting from screening through post-study follow-up.
   At a minimum, the drug name, route of administration, dose and frequency of dosing, along with start and stop dates should be recorded.
Electrocardiogram
Echocardiogram or MUGA
Laboratory assessments

Refer to Time and Events Tables in FIGS. 4 and 5 for additional details on assessments required at screening and prior to start of study treatment.

2.4.2 Evaluation of Anti-Cancer Activity

RECIST version 1.1 guidelines will be used to determine the overall tumor burden at screening, select target and non-target lesions, and in the disease assessments through the duration of the study (Eisenhauer, 2009).

As indicated in RECIST version 1.1 guidelines:
Lymph nodes that have a short axis of <10 mm are considered non-pathological and must not be recorded or followed.
Pathological lymph nodes with <15 mm, but 10 mm short axis are considered non-measurable.
Pathological lymph nodes with 15 mm short axis are considered measurable and can be selected as target lesions; however, lymph nodes should not be selected as target lesions when other suitable target lesions are available.
Measurable lesions up to a maximum of two lesions per organ and 5 lesions in total, representative of all involved organs, should be identified as target lesions, and recorded and measured at baseline. These lesions should be selected based on their size (lesions with the longest diameter) and their suitability for accurate repeated measurements (either by imaging techniques or clinically).

Note: Cystic lesions thought to represent cystic metastases must not be selected as target lesions when other suitable target lesions are available.

Note: Measurable lesions that have been previously irradiated and have not been shown to be progressing following irradiation must not be considered as target lesions.
Lytic bone lesions or mixed lytic-blastic lesions, with identifiable soft tissue components, that can be evaluated by CT or MRI) can be considered measurable. Bone scans, FDG-PET scans or X-rays are not considered adequate imaging techniques to measure bone lesions.
All other lesions (or sites of disease) must be identified as non-target and must also be recorded at baseline. Non-target lesions will be grouped by organ. Measurements of these lesions are not required, but the presence or absence of each must be noted throughout follow-up.

Disease assessment modalities may include imaging (e.g. CT scan, MRI, bone scan) and physical examination (as indicated for palpable/superficial lesions).

As indicated in Section 2.4.1, baseline disease assessment must be completed within 30 days prior to the first dose of H2L5 hIgG4PE. On-treatment disease assessments occur every 9 weeks until Week 54. After Week 54, disease assessments will be performed every 12 weeks then at the time of discontinuation of study treatment. At each post-baseline assessment, evaluation of the sites of disease (all target and non-target lesions) identified by the baseline scans is required. CT scans with contrast of the chest, abdomen, and pelvis, or if contra-indicated, MRI, is required at each post-baseline assessment. To ensure comparability between the baseline and subsequent assessments, the same method of assessment and the same technique will be used when assessing response.

For post-baseline assessments, a window of +7 days is permitted to allow for flexible scheduling. If the last radiographic assessment was more than 9 weeks prior to the subject's discontinuation from study treatment, or >12 weeks if after Week 54, a disease assessment should be obtained.

Subjects with disease progression by RECIST version 1.1 guidelines are required to have a confirmatory disease assessment at least 4 weeks after the date disease progression was declared in order to confirm disease progression by irRECIST guidelines.

Subjects whose disease responds (either CR or PR) must have a confirmatory disease assessment performed at least 4 weeks after the date of assessment during which the response was demonstrated. More frequent disease assessments may be performed at the discretion of the investigator. In the subjects who attain a confirmed CR and fulfil the requirement for early discontinuation of study treatment (refer to Section 2.2.3), disease assessments at a frequency of will be performed every 12 weeks until progression. If study treatment is resumed upon disease progression and following consultation with the Investigator and GSK Medical Monitor, imaging scans which indicated progression will serve as the baseline scans.

The visit level responses and treatment-based decisions will incorporate irRECIST guidelines as described in Section 2.6.

2.4.3 Physical Examinations

A complete physical examination will include, at a minimum, assessment of the Cardiovascular, Respiratory, Gastrointestinal and Neurological systems. Height (at Screening only) and weight will also be measured and recorded.

A brief physical examination will include at a minimum, assessments of the skin, lungs, cardiovascular system, and abdomen (liver and spleen). In the bintrafusp alfa combination cohort, a full skin examination specifically evaluating all skin surfaces and mucous membranes (eyes, nares, oropharynx, genitals, and perianal area) is required.

Investigators should pay special attention to clinical signs related to previous serious illnesses.

2.4.4 Performance Status

Performance status will be assessed using the ECOG scale as described in Section 2.7.

2.4.5 Vital Signs Vital signs will be measured in semi-supine position after 5 minutes of rest and will include temperature, systolic and diastolic blood pressure and pulse rate. In the case of an abnormal first reading, three readings of blood pressure and/or pulse rate must be taken, whereby the first reading should be rejected and the second and third averaged to give the measurement to be recorded in the eCRF.

Vital signs will be measured more frequently if warranted by clinical condition of the subject.

On days where vital signs are measured multiple times, temperature does not need to be repeated unless clinically indicated.

If a subject develops fever, the subject will be managed using fever management guidelines.

2.4.6 Electrocardiogram

12-lead electrocardiograms will be obtained using an ECG machine that automatically calculates the heart rate and measures PR, QRS, QT, and QTcF intervals; manual calculation of QTcF is permitted.

2.4.7 Echocardiograms

Echocardiograms will be performed at baseline to assess cardiac ejection fraction and cardiac valve morphology for the purpose of study eligibility. Additional ECHO assessments may be performed if clinically warranted. The evaluation of the echocardiography must include an evaluation for left ventricular ejection fraction (LVEF) and both right and left-sided valvular lesions. MUGA can be used in lieu of ECHO (if not available) in the assessment of LVEF; the same modality should be used in any subsequent assessments.

2.4.8 Biomarkers/Pharmacodynamic Markers

2.4.8.1 Blood Biomarkers

Blood samples will be collected and analyzed by flow cytometry to evaluate the binding of H2L5 hIgG4PE to the ICOS receptor.

The numbers of T cells, B cell, natural killer (NK) cells as well as the subsets of T cells, activation and proliferation status of T cells will be simultaneously evaluated by flow cytometry in the same blood sample. Blood samples will be collected for isolation of PBMCs and plasma. Plasma and serum samples will be used for the analyses of circulating soluble factors in relation to T cell activation and may be utilized for analysis of soluble ICOS or soluble ICOS-drug complexes depending on the availability of the assays. Circulating factors to be analyzed may include but are not limited to the presences of IFNγ, TNFα, IL-2, IL-4, IL-6, IL-10, IL-8, IL-13, IL-12p70, IL-21, and chemokines as well as antibodies against the tumor, self or viral antigens. Plasma samples may also be analyzed for cell-free DNA (cfDNA) or exosomes (ribonucleic Acid [RNA]) for novel markers of immune activation or response to treatment with H2L5 hIgG4PE as a monotherapy or in combination.

PBMCs isolated from whole blood will be preserved and stored for flow cytometry of additional cells such as immune regulatory populations which may include but are not limited to myeloid derived suppressor cells, subsequent functional analyses, assessment of T cell repertoires, their relationship to clinical responses and changes in response to treatment with H2L5 hIgG4PE. The functional state of PBMCs may be analyzed for expression of cytokines which may include, but not limited to, IFNγ, IL-2, IL-10, TNFα, Granzyme B, PD-1, TIM3, and CD107a. PBMCs may also be evaluated for genomic (deoxyribonucleic acid [DNA]) and gene expression (RNA or protein) alterations to determine treatment-related changes in immune-related signatures.

2.4.8.2 Tumor Tissue

Archival tumor tissue, as well as, fresh pre- and on-treatment biopsies will be collected. The fresh biopsies samples are required in the pharmacodynamic/PK cohorts. Baseline tumor tissue at screening, either archival or fresh biopsy, and on-treatment fresh biopsies at Week 6 are required for the HNSCC PD1/L1 treatment naïve PD-L1 CPS <1 and the HNSCC Q6W expansion cohorts.

Screening (archival or fresh) and on-treatment week 6 biopsy samples are required; required in subjects enrolled in the PK/pharmacodynamic cohort for the combination studies with bintrafusp alfa.

Additionally, the following screening tests will be evaluated in cohorts specified below:
PD-L1 IHC 22C3 pharmDx assay for the enrolment into HNSCC PD-L1 CPS <1 cohort only.
Ventana PD-L1 (SP263) IHC assay for the enrolment into the expansion cohort with bintrafusp alfa combination studies.

Tumor tissues collected at screening and on-treatment will also be evaluated by IHC, multiplex immunofluorescence technology or potentially other methods for expression of phenotypic and functional immune cell markers on tumor infiltrating lymphocytes (TIL) and other immune cells as well as immune signaling markers on tumor cells to understand the anti-tumor responses (including but not limited to PDL-1, ICOS, TIM-3, NY-ESO, TGF-beta) In addition, when possible, similar analyses will be performed on tumor tissue obtained upon progression. Additionally, tumor tissue may be sequenced to assess T cell receptor diversity (TCR diversity) as well as evaluated for any DNA/RNA/protein changes correlating with response.

### 2.5 Statistical Considerations and Data Analyses

2.5.1 Dose Escalation

Safety and tolerability of H2L5 hIgG4PE administered in combination with bintrafusp alfa will be evaluated using an adaptive mTPI approach (shown in FIG. 3). The mTPI design is an extension of the toxicity probability interval method and employs a simple beta-binomial hierarchic model (Ji et al. Clin Trials. 2010; 7:653-663). Decision rules are based on calculating the unit probability mass (UPM) of three intervals corresponding to under dosing, proper dosing, and overdosing in terms of toxicity. Specifically, the under-dosing interval is defined as (0, pT−ε1), the overdosing interval as (pT+ε2, 1), and the proper dosing interval as (pT−ε1, pT+ε2), where ε1 and ε2 are small fractions, such as 0.05, to account for the uncertainty around the true target toxicity. A sensitivity analysis showed that the mTPI design is robust to the specification of E values (Ji, 2010). In addition, ε1 and ε2 could take different values to reflect physician preference and the nature of the disease. For advanced diseases with few treatment options, higher toxicity rates might be considered acceptable, implying a specification of ε2>ε1. For less-advanced diseases, the two E values could be identical or ε1>ε2. The three dosing intervals are associated with three different dose-escalation decisions. The under-dosing interval corresponds to a dose escalation (E), overdosing corresponds to a dose de-escalation (D), and proper dosing corresponds to staying at the current dose (S). Given an interval and a probability distribution, the UPM of that interval is defined as the probability of the interval divided by the length of the interval. The mTPI design calculates the UPMs for the three dosing intervals, and the one with the largest UPM implies the corresponding dose-finding decision. That decision provides the dose level to be used for future subjects. For example, if the under-dosing interval has the largest UPM, decision E, to escalate, will be executed, and the next cohort of subjects will be treated at the next higher dose level. Analyses showed that the decision based on the UPM is optimal in that it minimized a subsequent expected loss (Ji, 2010). Under the mTPI design, a trial is terminated when either the lowest dose is above the MTD or a pre-specified maximum sample size is reached.

2.5.2 Dose Expansion

In the expansion cohorts, after a minimum of 10 subjects have been enrolled in one dose/dose level in a cohort, the number of observed responses as well as other available date will be used for futility analysis.

If data permit, clinical activity of H2L5 hIgG4PE administered alone also may be evaluated using a Bayesian hierarchical modelling approach as an exploratory analysis. The design permits the trial to be frequently monitored for clinical activity with the constraint of both Type I and Type II error rates (Berry, 2013).

2.5.3 Sample Size Considerations

To complete dose escalation/safety run-ins for H2L5 hIgG4PE in combination with bintrafusp alfa (refer to FIG. 2), it is estimated that approximately 241 subjects will be enrolled. Doses of H2L5 hIgG4PE to be studied will be guided by the mTPI design.

Simulations were conducted to determine the average sample size and percentage of times each dose would be selected as MTD under four different scenarios, considering the dose escalation phase of H2L5 hIgG4PE in combination with bintrafusp alfa (guided by mTPI design). Cohort size of 3 subjects was used with a cap of 6 subjects at a dose level (the trial will stop recruitment if the next dose has already 6 subjects), the maximum sample size of 12 subjects for the dose escalation and 15 subjects at RP2D for further exploration. A safety rule for early termination was used where posterior probability exceeds target toxicity probability by 95%. 1000 simulated studies were used to derive the operating characteristics in FACTS version 6.1 software. The average sample sizes over the simulated clinical trials under four scenarios were 9.1, 9.3, 8.9 and 8.0 respectively, totaling approximately 25 subjects for each combination.

Details of the scenarios are provided in Table 10. The dose combinations in the table are the pre-selected dose combinations that are projected to be used in the trial.

TABLE 10
Simulation Results Under Various Scenarios

| | Scenario 1: | | Scenario 2: | | Scenario 3: | | Scenario 4: | |
|---|---|---|---|---|---|---|---|---|
| | Low Toxicity | | Low Toxicity | | Moderate Toxicity | | High Toxicity | |
| | | Percent of | | Percent of | | Percent of | | Percent of |
| Dose H2L5 | | Trials | | Trials | | Trials | | Trials |
| hIgG4PE (mg) | True | Selecting | True | Selecting | True | Selecting | True | Selecting |
| in | DLT | Dose as | DLT | Dose as | DLT | Dose as | DLT | Dose as |
| combination | Rate | MTD (%) | Rate | MTD (%) | Rate | MTD (%) | Rate | MTD (%) |
| 24 | 0.01 | <0.01% | 0.05 | 3% | 0.20 | 38% | 0.40 | 83% |
| 80 | 0.05 | 99.9% | 0.10 | 97% | 0.30 | 62% | 0.50 | 17% |

In the expansion phases, the sample size of a cohort or cohorts may target approximately 30 subjects per cohort. The condition by which the sample size will increase depends on the outcome from interim analysis of the null/alternative hypotheses that was determined for a tumor type.

For each tumor indication expansion cohort, an interim analysis will be conducted after efficacy data at any dose level are available on a minimum of subjects (refer to Section 2.5.5); a separate decision will be made for each disease cohort and dose. The trial may continue to enroll the maximum planned sample size to provide a better estimate on the distribution of the response rate in the different doses and target populations.

The trial is not designed to stop early for efficacy but is designed to assess futility if the predictive probability of success is 10% or less. The type I error rate, power, and predictive probability for assessing futility were determined from stating the minimum and maximum sample size, futility stopping rate, and the optimizing criterion as minimizing the sample size under null hypothesis. A very weak informative prior distribution with a mean response rate equal to the target response rate is assumed. Thus, the predictive probability for the response rate will be primarily driven by the data. The detailed decision criteria for all cohorts are documented in Section 2.5.5.

For any PD-1/L1 experienced combination therapy expansion cohorts starting with 10 subjects in each cohort and allowing for a maximum sample size of 30 for each cohort, this design will have an overall type I error rate (a) 5%. Under null hypotheses with 10% overall response rate (ORR), the expected sample size of the design is 15 subjects per cohort; and probability of early termination (PET) is 35% by 10 subjects evaluated and 80% by 20 subjects evaluated. Under the alternative hypothesis, if the true response rate is 30%, the probability of success is 83%; the expected sample size of the design is 28 subjects in total and PET is 3% by 10 subjects and 13% by 20 subjects.

For the PD-1/L1 naïve combination expansion cohorts including HNSCC, NSCLC with PD-L1<50%, bladder/urothelial cancer, cervical, and viral-positive cancers, starting with 10 subjects in each cohort and allowing for a maximum sample size of 30 for each cohort, this design will have an overall type I error rate (a) 9.8%. Under null hypotheses with 20% ORR, the expected sample size of the design is 16 subjects per cohort; and probability of early termination (PET) is 38% by 10 subjects evaluated and 72% by 20 subjects evaluated. Under the alternative hypothesis, if the true response rate is 40%, the probability of success is 83%; the expected sample size of the design is 28 subjects in total and PET is 5% by 10 subjects evaluated and 12% by 20 subjects evaluated.

For the biomarker positive cohort, starting with 12 subjects and allowing for a maximum sample size of 40, will have an overall type I error rate (a) of 4%. Under the null hypothesis of 10% ORR, the expected sample size of the design is 26 subjects; and the PET is 28% by 12 subjects evaluated and 55% by 30 subjects evaluated. Under the alternative hypothesis, if the true response rate is 25%, the power is 80%; the expected sample size of the design is 39 subjects in total and the PET is 3% by 12 subjects evaluated and 5% by 30 subjects evaluated. The biomarker negative group will similarly allow for a maximum sample size of 40, and will follow enrolment/futility according to the biomarker positive group.

For the PD-1/L1 naïve combination expansion cohorts including NSCLC with PD-L1>50% and MSI-H/dMMR cancers, starting with 10 subjects in each cohort and allowing for a maximum sample size of 30 for each cohort, this design will have an overall type I error rate (a) 7.9%. Under null hypotheses with 30% ORR, the expected sample size of the design is 19 subjects per cohort; and probability of early termination (PET) is 15% by 10 subjects evaluated and 55% by 20 subjects evaluated. Under the alternative hypothesis, if the true response rate is 50%, the probability of success is 80%; the expected sample size of the design is 29 subjects in total and PET is 1.0% by 10 subjects evaluated and 6.2% by 20 subjects evaluated.

2.5.4 Data Analyses—xAnalysis Populations

All Treated Population will be defined as all subjects who receive at least one dose of H2L5 hIgG4PE. Safety and anti-cancer activity will be evaluated based on this analysis population.

Pharmacokinetic Population will be defined as all subjects from the All Treated Population for whom a PK sample is obtained and analyzed.

Pharmacodynamic Population will be defined as subjects in the All Treated Subjects Population for whom pre- and on-treatment paired and evaluable tumor biopsies or pre- and on-treatment blood samples were obtained and analyzed for biomarkers.

2.5.5 Interim Analysis

No formal interim analyses will be performed using the data generated during the dose escalation phases of the study. Available safety and PK/pharmacodynamic data will be reviewed after completion of each dose level. This review will support the decision to escalate to the dose level using the rules as described in Section 2.5.1. For dose expansion cohorts, continuous assessment of efficacy and safety will be performed after first interim analysis based upon a minimum of 10 subjects in at least one of the expansion cohort with available unconfirmed overall response data.

2.5.6 Pharmacokinetic Analyses

Validated analytical methods will be used to measure concentrations of bintrafusp alfa (M7824). The following pharmacokinetic parameters will be determined using noncompartmental method, if data permit, and may include but not be limited to:
maximum observed plasma concentration (Cmax)
time to Cmax (tmax)
Cmin
area under the plasma concentration-time curve (AUC(0-t), AUC(0-∞)) and AUC(0-τ))
apparent terminal phase elimination rate constant (λz) (single dose)
apparent terminal phase half-life (t½)
systemic clearance of parent drug (CL)
   2.6 Guidelines for Assessment of Disease, Disease Progression and Response Criteria—Adapted from RECIST Version 1.1

2.6.1 Assessment Guidelines

The same diagnostic method, including use of contrast when applicable, must be used throughout the study to evaluate a lesion. Contrast agents must be used in accordance with the Image Acquisition Guidelines.

All measurements must be taken and recorded in millimeters (mm), using a ruler or calipers.

Ultrasound is not a suitable modality of disease assessment. If new lesions are identified by ultrasound, confirmation by CT or MRI is required.

Fluorodeoxyglucose (FDG)-PET is generally not suitable for ongoing assessments of disease.

However FDG-PET can be useful in confirming new sites of disease where a positive FDG-PET scans correlates with the new site of disease present on CT/MRI or when a baseline FDG-PET was previously negative for the site of the new lesion. FDG-PET may also be used in lieu of a standard bone scan providing coverage allows interrogation of all likely sites of bone disease and FDG-PET is performed at all assessments.

If PET/CT is performed then the CT component can only be used for standard response assessments if performed to diagnostic quality, which includes the required anatomical coverage and prescribed use of contrast. The method of assessment must be noted as CT on the eCRF.

Clinical Examination: Clinically detected lesions will only be considered measurable when they are superficial (e.g. skin nodules). In the case of skin lesions, documentation by color photography, including a ruler/calipers to measure the size of the lesion, is required.

CT and MRI: Contrast enhanced CT with 5 mm contiguous slices is recommended. Minimum size of a measurable baseline lesion must be twice the slice thickness, with a minimum lesion size of 10 mm when the slice thickness is 5 mm. MRI is acceptable, but when used, the technical specification of the scanning sequences must be optimized for the evaluation of the type and site of disease and lesions must be measured in the same anatomic plane by use of the same imaging examinations.

Whenever possible, the same scanner should be used.

X-ray: In general, X-ray should not be used for target lesion measurements owing to poor lesion definition. Lesions on chest X-ray may be considered measurable if they are clearly defined and surrounded by aerated lung; however, chest CT is preferred over chest X-ray.

Brain Scan: If brain scans are required, then contrast enhanced MRI is preferable to contrast enhanced CT. 2.6.2 Guidelines for Evaluation of Disease Measurable and Non-Measurable Definitions are as follows: Measurable lesion: A non-nodal lesion that can be accurately measured in at least one dimension (longest dimension) of:
>10 mm with MRI or CT when the scan slice thickness is no greater than 5 mm. If the slice thickness is greater than 5 mm, the minimum size of a measurable lesion must be at least double the slice thickness (e.g., if the slice thickness is 10 mm, a measurable lesion must be 20 mm).
10 mm caliper/ruler measurement by clinical exam or medical photography.
20 mm by chest X-ray.
Additionally, lymph nodes can be considered pathologically enlarged and measurable if 15 mm in the short axis when assessed by CT or MRI (slice thickness recommended to be no more than 5 mm). At baseline and follow-up, only the short axis will be measured.

Non-measurable lesion: All other lesions including lesions too small to be considered measurable (longest diameter <10 mm or pathological lymph nodes with 10 mm and <15 mm short axis) as well as truly non-measurable lesions, which include: leptomeningeal disease, ascites, pleural or pericardial effusions, inflammatory breast disease, lymphangitic involvement of the skin or lung, abdominal masses/abdominal organomegaly identified by physical exam that is not measurable by reproducible imaging techniques.

Measurable disease: The presence of at least one measurable lesion. Palpable lesions that are not measurable by radiologic or photographic evaluations may not be utilized as the only measurable lesion.

Non-Measurable only disease: The presence of only non-measurable lesions. Note: non-measurable only disease is not allowed per protocol.

2.6.3 Immune-Related RECIST Response Criteria

Evaluation of target lesions are summarised in Table 11.

TABLE 11

| New, measurable^{a} lesions | Incorporated into tumor burden |
|---|---|
| New, non-measurable | Do not define progression (but preclude |
| lesions | CR) |
| irCR | Disappearance of all lesions in two |
| | consecutive observations not less than |
| | 4 weeks apart. Any pathological lymph |
| | nodes (whether target or non-target) |
| | must have reduction in short axis |
| | to <10 mm. |
| irPR | ≥30% decrease in tumor burden compared |
| | with baseline in two observations at |
| | least 4 weeks apart |
| irSD | 30% decrease in tumor burden compared |
| | with baseline cannot be established |
| | nor 20% increase compared with nadir |
| irPD^{b} | At least 20% increase in tumor burden |
| | compared with nadir (at any single time |
| | point) in two consecutive observations |
| | at least 4 weeks apart. |
| | In addition to the relative increase of |
| | 20%, the sum must also demonstrate an |
| | absolute increase of at least 5 mm. |

^{a}Measureable per RECIST v1.1.
^{b}Treatment decisions will be based upon the immune-related RECIST guidelines.

2.6.3.1 Anti-Tumor Response Based on Total Measurable Tumor Burden

For Modified RECIST based on RECIST v1.1 and Immune-Related RECIST [Wolchok et al. Clin Cancer Res 2009; 15(23): 7412-20; Nishino et al. Clin Cancer Res. 2013; 19:3936-3943], the initial target (“index”) and measurable new lesions are taken into account. At the baseline tumor assessment, the sum of the diameters in the plane of measurement of all target lesions (maximum of five lesions in total and a maximum of two lesions per organ representative of all involved organs) is calculated.

Note: If pathological lymph nodes are included in the sum of diameters, the short axis of the lymph node(s) is added into the sum. The short axis is the longest perpendicular diameter to the longest diameter of a lymph node or nodal mass. At each subsequent tumor assessment, the sum of diameters of the baseline target lesions and of new, measurable nodal and non-nodal lesions (10 mm), up to 2 new lesions per organ are added together to provide the total tumor burden:

Tumor Burden=Sum of diameters_{target lesions}+sum of diameters_{new, measurable lesions}

2.6.3.2 Time-Point Response Assessment Using the Immune-Related RECIST Criteria

Percentage changes in tumor burden per assessment time point describe the size and growth kinetics of both conventional and new, measurable lesions as they appear. At each tumor assessment, the response in index and new, measurable lesions is defined based on the change in tumor burden (after ruling out irPD). Decreases in tumor burden must be assessed relative to baseline measurements (i.e. the sum of diameters of all target lesions at screening).

2.6.3.3 Evaluation of non-target lesions

Definitions for assessment of response for non-target lesions are as follows:
Complete Response (CR): The disappearance of all non-target lesions. All lymph nodes identified as a site of disease at baseline must be non-pathological (e.g. <10 mm short axis).
Non-CR/Non-PD: The persistence of 1 or more non-target lesion(s) or lymph nodes identified as a site of disease at baseline 10 mm short axis.
Progressive Disease (PD): Unequivocal progression of existing non-target lesions.
Not Applicable (NA): No non-target lesions at baseline.
Not Evaluable (NE): Cannot be classified by one of the four preceding definitions.

Note: In the presence of measurable disease, progression on the basis of solely non-target disease requires substantial worsening such that even in the presence of SD or PR in target disease, the overall tumor burden has increased sufficiently to merit discontinuation of therapy. Furthermore, sites of non-target lesions, which are not assessed at a time point based on the assessment schedule, should be excluded from the response determination (e.g. non-target response does not have to be “Not Evaluable”).

2.6.3.4 New Lesions

New malignancies denoting disease progression must be unequivocal. Lesions identified in follow-up in an anatomical location not scanned at baseline are considered new lesions.

Any equivocal new lesions must continue to be followed. Treatment can continue at the discretion of the investigator until the next scheduled assessment. If at the next assessment, the new lesion is considered to be unequivocal, progression would be declared.

2.6.3.5 Evaluation of Overall Response

Table 12 presents the overall response at an individual disease assessment time-point accounting for all possible combinations of responses in target and non-target lesions with or without the appearance of new lesions for subjects with measurable disease at baseline.

TABLE 12
Evaluation of Overall Response for Subjects
with Measurable Disease at Baseline

| Target | | New | Overall |
|---|---|---|---|
| Lesions | Non-Target Lesions | Lesions | Response |
| CR | CR or NA | No | CR |
| CR | Non-CR/Non-PD or NE | No | PR |
| PR | Non-PD or NA or NE | No | PR |
| SD | Non-PD or NA or NE | No | SD |
| NE | Non-PD or NA or NE | No | NE |
| PD | Any | Yes or No | PD |
| Any | PD | Yes or No | PD |
| Any | Any | Yes | PD |

Abbreviations:
CR = Complete response,
PR = Partial response,
SD = Stable disease,
PD = Progressive disease,
NA = Not applicable, and
NE = Not Evaluable

2.6.3.6 Evaluation of Best Overall Response

The best overall response is the best response recorded from the start of the treatment until disease progression/recurrence and will be determined programmatically by GSK based on the investigators assessment of response at each time point.

To be assigned a status of SD, follow-up disease assessment must have met the SD criteria at least once after the first dose at a minimum interval of days as defined in the RAP.

If the minimum time for SD is not met, best response will depend on the subsequent assessments. For example, if an assessment of PD follows the assessment of SD and SD does not meet the minimum time requirement the best response will be PD. Alternatively, subjects lost to follow-up after an SD assessment not meeting the minimum time criteria will be considered not evaluable.

2.6.3.7 Confirmation Criteria

To be assigned a status of PR or CR, a confirmatory disease assessment must be performed no less than 4 weeks (28 days) after the criteria for response are first met.

### 2.7 ECOG Performance Status

Summary presented in Table 13.

TABLE 13
ECOG Performance Status

| Grade | Descriptions |
|---|---|
| 0 | Normal activity. Fully active, able to carry on |
| | all pre-disease performance without restriction. |
| 1 | Symptoms, but ambulatory. Restricted in physically |
| | strenuous activity, but ambulatory and able to carry |
| | out work of a light or sedentary nature (e.g., light |
| | housework, office work). |
| 2 | In bed <50% of the time. Ambulatory and capable of |
| | all self-care, but unable to carry out any work |
| | activities. Up and about more than 50% of waking hours. |
| 3 | In bed >50% of the time. Capable of only limited |
| | self-care, confined to bed or chair more than 50% |
| | of waking hours. |
| 4 | 100% bedridden. Completely disabled. Cannot carry on |
| | any self-care. Totally confined to bed or chair. |
| 5 | Dead. |

Oken et al. Am J Clin Oncol. 1982; 5: 649-655.

### 2.8 Events of Clinical Interest

These are selected events considered of clinical interest; they may be non-serious AEs or SAEs. Events of Clinical Interest are different from Adverse Events of Special Interest (AESI) in that an AESI is defined as an adverse event of potential immunologic etiology. Such events recently reported after treatment with other immune modulatory therapy include colitis, uveitis, hepatitis, pneumonitis, diarrhea, endocrine disorders, and specific cutaneous toxicities, as well as other events that may be immune mediated.

For the time period beginning with the administration of the first dose of study treatment through 30 days following discontinuation of study treatment, any ECI, or follow up to an ECI, whether or not related to the study drug(s), must be reported to the Sponsor. ECI include:

1. Overdose of study drug(s) that is not associated with clinical symptoms or abnormal laboratory results must be reported within 5 days.

2. An elevated aspartate aminotransferase (AST) or alanine aminotransferase (ALT) lab value that is greater than or equal to 3× the upper limit of normal and an elevated total bilirubin lab value that is greater than or equal to 2× the upper limit of normal and, at the same time, an alkaline phosphatase lab value that is less than 2× the upper limit of normal, as determined by way of protocol-specified laboratory testing or unscheduled laboratory testing. This ECI must be reported within 24 hours. These criteria are based upon available regulatory guidance documents. The purpose of the criteria is to specify a threshold of abnormal hepatic tests that may require an additional evaluation for an underlying etiology.

3. Infection with COVID-19 coronavirus, whether suspected based on exposure history and clinical signs and symptoms, or confirmed by laboratory test in the context of exposure history and clinical signs and symptoms. Reporting will follow WHO and GSK guidelines.

### 2.9 Genetic Research

2.9.1 Genetic Research Objectives and Analyses

The objectives of the genetic research are to investigate the relationship between genetic variants and:
Response to medicine, including H2L5 hIgG4PE, other immune therapy under investigation in this study, or any concomitant medicines;
Cancer susceptibility, severity, and progression and related conditions.

Genetic data may be generated while the study is underway or following completion of the study. Genetic evaluations may include focused candidate gene approaches and/or examination of a large number of genetic variants throughout the genome (whole genome analyses). Genetic analyses will utilize data collected in the study and will be limited to understanding the objectives highlighted above. Analyses may be performed using data from multiple clinical studies to investigate these research objectives.

Appropriate descriptive and/or statistical analysis methods will be used. A detailed description of any planned analyses will be documented in a Reporting and Analysis Plan (RAP) prior to initiation of the analysis. Planned analyses and results of genetic investigations will be reported either as part of the clinical RAP and study report, or in a separate genetics RAP and report, as appropriate.

2.9.2 Study Population

Any subject who is enrolled in the study can participate in genetic research. Any subject who has received an allogeneic bone marrow transplant must be excluded from the genetic research.

2.9.3 Study Assessments and Procedures

A key component of successful genetic research is the collection of samples during clinical studies. Collection of samples, even when no a priori hypothesis has been identified, may enable future genetic analyses to be conducted to help understand variability in disease and medicine response.

A 6 ml blood sample will be taken for DNA extraction. A blood sample is collected at the baseline visit, after the subject has been randomized and provided informed consent for genetic research. Instructions for collection and shipping of the genetic sample are described in the laboratory manual. The DNA from the blood sample may undergo quality control analyses to confirm the integrity of the sample. If there are concerns regarding the quality of the sample, then the sample may be destroyed. The blood sample is taken on a single occasion unless a duplicate sample is required due to an inability to utilize the original sample.

The genetic sample is labelled (or “coded”) with the same study specific number used to label other samples and data in the study. This number can be traced or linked back to the subject by the investigator or site staff. Coded samples do not carry personal identifiers (such as name or social security number).

Samples will be stored securely and may be kept for up to 15 years after the last subject completes the study, or GSK may destroy the samples sooner. GSK or those working with GSK (for example, other researchers) will only use samples collected from the study for the purpose stated in this protocol and in the informed consent form.

### 2.10 Preliminary Results

4 melanoma patients were dosed with 24 mg H2L5 hIgG4PE Q3W and 2400 mg bintrafusp alpha Q3W. After 3 months, 2 had Progressive Disease and 2 had Partial Response. All four cleared the dose limiting toxicity period of 28 days.

SEQUENCE LISTINGS

| SEQ ID | | |
|---|---|---|
| NO. | Sequence | Description |
| 1 | DYAMH | ICOS binding protein CDRH1 |
| 2 | LISIYSDHTNYNQKFQG | ICOS binding protein CDRH2 |
| 3 | NNYGNYGWYFDV | ICOS binding protein CDRH3 |
| 4 | SASSSVSYMH | ICOS binding protein CDRL1 |
| 5 | DTSKLAS | ICOS binding protein CDRL2 |
| 6 | FQGSGYPYT | ICOS binding protein CDRL3 |
| 7 | QVQLVQSGAEVKKPGSSVKVSCKASGYTFTDYAMHWVRQAP | ICOS humanized heavy chain |
| | GQGLEWMGLISIYSDHTNYNQKFQGRVTITADKSTSTAYMEL | variable region (H2) |
| | SSLRSEDTAVYYCGRNNYGNYGWYFDVWGQGTTVTVSS | |
| 8 | EIVLTQSPATLSLSPGERATLSCSASSSVSYMHWYQQKPGQAP | ICOS humanized light chain |
| | RLLIYDTSKLASGIPARFSGSGSGTDYTLTISSLEPEDFAVYYCF | variable region (L5) |
| | QGSGYPYTFGQGTKLEIK | |
| 9 | QVQLVQSGAEVKKPGSSVKVSCKASGYTFTDYAMHWVRQAP | ICOS humanized monoclonal |
| | GQGLEWMGLISIYSDHTNYNQKFQGRVTITADKSTSTAYMEL | antibody heavy chain |
| | SSLRSEDTAVYYCGRNNYGNYGWYFDVWGQGTTVTVSSAST | |
| | KGPSVFPLAPCSRSTSESTAALGCLVKDYFPEPVTVSWNSGALT | |
| | SGVHTFPAVLQSSGLYSLSSVVTVPSSSLGTKTYTCNVDHKPSN | |
| | TKVDKRVESKYGPPCPPCPAPEFEGGPSVFLFPPKPKDTLMISR | |
| | TPEVTCVVVDVSQEDPEVQFNWYVDGVEVHNAKTKPREEQFN | |
| | STYRVVSVLTVLHQDWLNGKEYKCKVSNKGLPSSIEKTISKAK | |
| | GQPREPQVYTLPPSQEEMTKNQVSLTCLVKGFYPSDIAVEWES | |
| | NGQPENNYKTTPPVLDSDGSFFLYSRLTVDKSRWQEGNVFSC | |
| | SVMHEALHNHYTQKSLSLSLGK | |
| 10 | EIVLTQSPATLSLSPGERATLSCSASSSVSYMHWYQQKPGQAP | ICOS humanized monoclonal |
| | RLLIYDTSKLASGIPARFSGSGSGTDYTLTISSLEPEDFAVYYCF | antibody light chain |
| | QGSGYPYTFGQGTKLEIKRTVAAPSVFIFPPSDEQLKSGTASVV | |
| | CLLNNFYPREAKVQWKVDNALQSGNSQESVTEQDSKDSTYSL | |
| | SSTLTLSKADYEKHKVYACEVTHQGLSSPVTKSFNRGEC | |
| 11 | MKSGLWYFFLFCLRIKVLTGEINGSANYEMFIFHNGGVQILCKY | Human ICOS (isoform 2) |
| | PDIVQQFKMQLLKGGQILCDLTKTKGSGNTVSIKSLKFCHSQLS | |
| | NNSVSFFLYNLDHSHANYYFCNLSIFDPPPFKVTLTGGYLHIYE | |
| | SQLCCQLKFWLPIGCAAFVVVCILGCILICWLTKKM | |
| 12 | MKSGLWYFFLFCLRIKVLTGEINGSANYEMFIFHNGGVQILCKY | Human ICOS (isoform 1) |
| | PDIVQQFKMQLLKGGQILCDLTKTKGSGNTVSIKSLKFCHSQLS | |
| | NNSVSFFLYNLDHSHANYYFCNLSIFDPPPFKVTLTGGYLHIYE | |
| | SQLCCQLKFWLPIGCAAFVVVCILGCILICWLTKKKYSSSVHDP | |
| | NGEYMFMRAVNTAKKSRLTDVTL | |
| 13 | SYIMM | Bintrafusp alfa CDRH1 |
| 14 | SIYPSGGITFYADTVKG | Bintrafusp alfa CDRH2 |
| 15 | IKLGTVTTVDY | Bintrafusp alfa CDRH3 |
| 16 | TGTSSDVGGYNVS | Bintrafusp alfa CDRL1 |
| 17 | DVSNRPS | Bintrafusp alfa CDRL2 |
| 18 | SSYTSSSTRV | Bintrafusp alfa CDRL3 |
| 19 | EVQLLESGGGLVQPGGSLRLSCAASGFTFSSYIMMWVRQAPG | Bintrafusp alfa heavy chain |
| | KGLEWVSSIYPSGGITFYADTVKGRFTISRDNSKNTLYLQMNS | variable region |
| | LRAEDTAVYYCARIKLGTVTTVDYWGQGTLVTVSS | |
| 20 | QSALTQPASVSGSPGQSITISCTGTSSDVGGYNYVSWYQQHP | Bintrafusp alfa light chain |
| | GKAPKLMIYDVSNRPSGVSNRFSGSKSGNTASLTISGLQAEDE | variable region |
| | ADYYCSSYTSSSTRVFGTGTKVTVL | |
| 21 | EVQLLESGGGLVQPGGSLRLSCAASGFTFSSYIMMWVRQAPG | Anti-PD-L1 monoclonal |
| | KGLEWVSSIYPSGGITFYADTVKGRFTISRDNSKNTLYLQMNS | antibody heavy chain |
| | LRAEDTAVYYCARIKLGTVTTVDYWGQGTLVTVSSASTKGPSV | |
| | FPLAPSSKSTSGGTAALGCLVKDYFPEPVTVSWNSGALTSGVH | |
| | TFPAVLQSSGLYSLSSVVTVPSSSLGTQTYICNVNHKPSNTKVD | |
| | KRVEPKSCDKTHTCPPCPAPELLGGPSVFLFPPKPKDTLMISRT | |
| | PEVTCVVVDVSHEDPEVKFNWYVDGVEVHNAKTKPREEQYNS | |
| | TYRVVSVLTVLHQDWLNGKEYKCKVSNKALPAPIEKTISKAKG | |
| | QPREPQVYTLPPSREEMTKNQVSLTCLVKGFYPSDIAVEWESN | |
| | GQPENNYKTTPPVLDSDGSFFLYSKLTVDKSRWQQGNVFSCS | |
| | VMHEALHNHYTQKSLSLSPG | |
| 22 | QSALTQPASVSGSPGQSITISCTGTSSDVGGYNYVSWYQQHP | Bintrafusp alfa monoclonal |
| | GKAPKLMIYDVSNRPSGVSNRFSGSKSGNTASLTISGLQAEDE | antibody light chain |
| | ADYYCSSYTSSSTRVFGTGTKVTVLGQPKANPTVTLFPPSSEEL | |
| | QANKATLVCLISDFYPGAVTVAWKADGSPVKAGVETTKPSKQS | |
| | NNKYAASSYLSLTPEQWKSHRSYSCQVTHEGSTVEKTVAPTEC | |
| | S | |
| 23 | EVQLLESGGGLVQPGGSLRLSCAASGFTFSSYIMMWVRQAPG | Bintrafusp alfa heavy chain, |
| | KGLEWVSSIYPSGGITFYADTVKGRFTISRDNSKNTLYLQMNS | including TGFβRII sequence |
| | LRAEDTAVYYCARIKLGTVTTVDYWGQGTLVTVSSASTKGPSV | |
| | FPLAPSSKSTSGGTAALGCLVKDYFPEPVTVSWNSGALTSGVH | |
| | TFPAVLQSSGLYSLSSVVTVPSSSLGTQTYICNVNHKPSNTKVD | |
| | KRVEPKSCDKTHTCPPCPAPELLGGPSVFLFPPKPKDTLMISRT | |
| | PEVTCVVVDVSHEDPEVKFNWYVDGVEVHNAKTKPREEQYNS | |
| | TYRVVSVLTVLHQDWLNGKEYKCKVSNKALPAPIEKTISKAKG | |
| | QPREPQVYTLPPSREEMTKNQVSLTCLVKGFYPSDIAVEWESN | |
| | GQPENNYKTTPPVLDSDGSFFLYSKLTVDKSRWQQGNVFSCS | |
| | VMHEALHNHYTQKSLSLSPGAGGGGSGGGGSGGGGSGGGGS | |
| | GIPPHVQKSVNNDMIVTDNNGAVKFPQLCKFCDVRFSTCDNQ | |
| | KSCMSNCSITSICEKPQEVCVAVWRKNDENITLETVCHDPKLP | |
| | YHDFILEDAASPKCIMKEKKKPGETFFMCSCSSDECNDNIIFSE | |
| | EYNTSNPD | |
| 24 | MGRGLLRGLWPLHIVLWTRIASTIPPHVQKSDVEMEAQKDEII | TGFβRII isoform A |
| | CPSCNRTAHPLRHINNDMIVTDNNGAVKFPQLCKFCDVRFSTC | |
| | DNQKSCMSNCSITSICEKPQEVCVAVWRKNDENITLETVCHDP | |
| | KLPYHDFILEDAASPKCIMKEKKKPGETFFMCSCSSDECNDNII | |
| | FSEEYNTSNPDLLLVIFQVTGISLLPPLGVAISVIIIFYCYRVNRQ | |
| | QKLSSTWETGKTRKLMEFSEHCAIILEDDRSDISSTCANNINH | |
| | NTELLPIELDTLVGKGRFAEVYKAKLKQNTSEQFETVAVKIFPYE | |
| | EYASWKTEKDIFSDINLKHENILQFLTAEERKTELGKQYWLITA | |
| | FHAKGNLQEYLTRHVISWEDLRKLGSSLARGIAHLHSDHTPCG | |
| | RPKMPIVHRDLKSSNILVKNDLTCCLCDFGLSLRLDPTLSVDDL | |
| | ANSGQVGTARYMAPEVLESRMNLENVESFKQTDVYSMALVLW | |
| | EMTSRCNAVGEVKDYEPPFGSKVREHPCVESMKDNVLRDRGR | |
| | PEIPSFWLNHQGIQMVCETLTECWDHDPEARLTAQCVAERFS | |
| | ELEHLDRLSGRSCSEEKIPEDGSLNTTK | |
| 25 | MGRGLLRGLWPLHIVLWTRIASTIPPHVQKSVNNDMIVTDNN | TGFβRII isoform B |
| | GAVKFPQLCKFCDVRFSTCDNQKSCMSNCSITSICEKPQEVCV | |
| | AVWRKNDENITLETVCHDPKLPYHDFILEDAASPKCIMKEKKK | |
| | PGETFFMCSCSSDECNDNIIFSEEYNTSNPDLLLVIFQVTGISLL | |
| | PPLGVAISVIIIFYCYRVNRQQKLSSTWETGKTRKLMEFSEHCA | |
| | IILEDDRSDISSTCANNINHNTELLPIELDTLVGKGRFAEVYKAK | |
| | LKQNTSEQFETVAVKIFPYEEYASWKTEKDIFSDINLKHENILQ | |
| | FLTAEERKTELGKQYWLITAFHAKGNLQEYLTRHVISWEDLRK | |
| | LGSSLARGIAHLHSDHTPCGRPKMPIVHRDLKSSNILVKNDLTC | |
| | CLCDFGLSLRLDPTLSVDDLANSGQVGTARYMAPEVLESRMNL | |
| | ENVESFKQTDVYSMALVLWEMTSRCNAVGEVKDYEPPFGSKV | |
| | REHPCVESMKDNVLRDRGRPEIPSFWLNHQGIQMVCETLTEC | |
| | WDHDPEARLTAQCVAERFSELEHLDRLSGRSCSEEKIPEDGSL | |
| | NTTK | |
| 26 | IPPHVQKSVNNDMIVTDNNGAVKFPQLCKFCDVRFSTCDNQK | TGFβRII extracellular domain |
| | SCMSNCSITSICEKPQEVCVAVWRKNDENITLETVCHDPKLPY | |
| | HDFILEDAASPKCIMKEKKKPGETFFMCSCSSDECNDNIIFSEE | |
| | YNTSNPD | |
| 27 | EVQLVESGGGLVQPGGSLRLSCAASGFTFSDSWIHWVRQAPG | MPDL3289A PDL1 mab VH |
| | KGLEWVAWISPYGGSTYYADSVKGRFTISADTSKNTAYLQMN | |
| | SLRAEDTAVYYCARRHWPGGFDYWGQGTLVTVSS | |
| 28 | DIQMTQSPSSLSASVGDRVTITCRASQDVSTAVAWYQQKPGK | MPDL3289A PDL1 mab VL |
| | APKLLIYSASFLYSGVPSRFSGSGSGTDFTLTISSLQPEDFATYY | |
| | CQQYLYHPATFGQGTKVEIKR | |
| 29 | EVQLVESGGGLVQPGGSLRLSCAASGFTFSDSWIHWVRQAPG | YW243.55S70 PDL1 mAb VH |
| | KGLEWVAWISPYGGSTYYADS | |
| | VKGRFTISADTSKNTAYLQMNSLRAEDTAVYYCARRHWPGGF | |
| | DYWGQGTLVTVSA | |
| 30 | GGGGSGGGGSGGGGSGGGGSG | (Gly4Ser)4Gly Linker |
| 31 | GFTFSDYWMD | 37A10S713 VH CDR1 |
| 32 | NIDEDGSITEYSPFVKG | 37A10S713 VH CDR2 |
| 33 | WGRFGFDS | 37A10S713 VH CDR3 |
| 34 | KSSQSLLSGSFNYLT | 37A10S713 VL CDR1 |
| 35 | YASTRHT | 37A10S713 VL CDR2 |
| 36 | HHHYNAPPT | 37A10S713 VL CDR3 |
| 37 | EVQLVESGGLVQPGGSLRLSCAASGFTFSDYWMDWVRQAPG | 37A10S713 heavy chain |
| | KGLVWVSNIDEDGSITEYSPFVKGRFTISRDNAKNTLYLQMNS | variable region |
| | LRAEDTAVYYCTRWGRFGFDSWGQGTLVTVSS | |
| 38 | DIVMTQSPDSLAVSLGERATINCKSSQSLLSGSFNYLTWYQQK | 37A10S713 light chain variable |
| | PGQPPKLLIFYASTRHTGVPDRFSGSGSGTDFTLTISSLQAEDV | region |
| | AVYYCHHHYNAPPTFGPGTKVDIK | |
| 39 | EVQLVESGGGLVKPGGSLRLSCAASGFTFSDYFMHWVRQAPG | ICOS.33 IgG1f S267E heavy |
| | KGLEWVGVIDTKSFNYATYYSDLVKGRFTISRDDSKNTLYLQM | chain variable region |
| | NSLKTEDTAVYYCTATIAVPYYFDYWGQGTLVTVSS | |
| 40 | DIQMTQSPSSLSASVGDRVTITCQASQDISNYLSWYQQKPGK | ICOS.33 IgG1f S267E light |
| | APKLLIYYTNLLAEGVPSRFSGSGSGTDFTFTISSLQPEDIATYY | chain variable region |
| | CQQYYNYRTFGPGTKVDIK | |
| 41 | EVQLVESGGGVVRPGGSLRLSCVASGVTFDDYGMSWVRQAPG | STIM003 heavy chain variable |
| | KGLEWVSGINWNGGDTDYSDSVKGRFTISRDNAKNSLYLQM | region |
| | NSLRAEDTALYYCARDFYGSGSYYHVPFDYWGQGILVTVSS | |
| 42 | EIVLTQSPGTLSLSPGERATLSCRASQSVSRSYLAWYQQKRGQ | STIM003 light chain variable |
| | APRLLIYGASSRATGIPDRFSGDGSGTDFTLSISRLEPEDFAVYY | region |
| | CHQYDMSPFTFGPGTKVDIK | |
| 43 | QVQLVQSGAEVKKPGASVKVSCKASGYTFTGYYMHWVRQAP | XENP23104 [ICOS]_H0.66_L0 |
| | GQGLEWMGWINPHSGETIYAQKFQGRVTMTRDTSISTAYMEL | heavy chain variable region |
| | SSLRSEDTAVYYCARTYYYDTSGYYHDAFDVWGQGTMVTVSS | |
| 44 | GYYMH | XENP23104 [ICOS]_H0.66_L0 |
| | | CDR.H1 |
| 45 | WINPHSGETIYAQKFQG | XENP23104 [ICOS]_H0.66_L0 |
| | | CDR.H2 |
| 46 | TYYYDTSGYYHDAFDV | XENP23104 [ICOS]_H0.66_L0 |
| | | CDR.H3 |
| 47 | DIQMTQSPSSVSASVGDRVTITCRASQGISRLLAWYQQKPGKA | XENP23104 [ICOS]_H0.66_L0 |
| | PKLLIYVASSLQSGVPSRFSGSGSGTDFTLTISSLQPEDFATYYC | light chain variable region |
| | QQANSFPWTFGQGTKVEIK | |
| 48 | RASQGISRLLA | XENP23104 [ICOS]_H0.66_L0 |
| | | CDRL1 |
| 49 | VASSLQS | XENP23104 [ICOS]_H0.66_L0 |
| | | CDRL2 |
| 50 | QQANSFPWT | XENP23104 [ICOS]_H0.66_L0 |
| | | CDRL3 |

1.-36. (canceled)

37. A method for the treatment of cancer in a subject in need thereof, comprising administering to the subject a therapeutically effective amount of a combination comprising:
(i) an ICOS binding protein; and,
(ii) an anti-PD-(L)1(IgG):TGFβR fusion protein.

38. The method as claimed in claim 37, wherein the anti-PD-(L)1(IgG):TGFβR fusion protein comprises: (a) human TGFβRII, or a fragment thereof capable of binding to TGF-β; and (b) an anti-PD-L1 antibody or an antigen-binding fragment thereof, or an anti-PD-1 antibody or an antigen-binding fragment thereof.

39. (canceled)

40. The method as claimed in claim 38, wherein the ICOS binding protein comprises a V_{H} comprising an amino acid sequence of SEQ ID NO:7 and a V_{L} comprising an amino acid sequence of SEQ ID NO:8.

41.-42. (canceled)

43. The method as claimed in claim 37, wherein the anti PD-1 inhibitor, the anti-PD-(L)1(IgG):TGFβR fusion protein, or the anti-PD-L1 antibody or an antigen-binding fragment thereof, comprises a V_{H} comprising an amino acid sequence of SEQ ID NO:19 and a V_{L} comprising an amino acid sequence of SEQ ID NO:20.

44. (canceled)

45. The method as claimed in claim 37, wherein the PD-1 inhibitor, the anti-PD-(L)1(IgG):TGFβR fusion protein, or the anti-PD-L1 antibody, comprises a heavy chain amino acid sequence at least about 90% identical to the amino acid sequence of SEQ ID NO:21 and/or a light chain amino acid sequence at least about 90% identical to the amino acid sequence of SEQ ID NO:22.

46. The method as claimed in claim 45, wherein the PD-1 inhibitor, the anti-PD-(L)1(IgG):TGFβR fusion protein, or the anti-PD-L1 antibody, comprises a heavy chain amino acid sequence of SEQ ID NO:21 and a light chain amino acid sequence of SEQ ID NO:22.

47. The method as claimed in claim 45, wherein the human TGFβRII comprises a sequence at least about 90% identical to the amino acid sequence of SEQ ID NO:26.

48.-49. (canceled)

50. A method for the treatment of cancer in a human subject in need thereof, comprising administering to the subject a therapeutically effective amount of a combination comprising: an ICOS binding protein comprising a heavy chain amino acid sequence comprising a CDRH1 of SEQ ID NO:1, a CDRH2 of SEQ ID NO:2, and a CDRH3 of SEQ ID NO:3; and a light chain amino acid sequence comprising a CDRL1 of SEQ ID NO:4, a CDRL2 of SEQ ID NO:5, and a CDRL3 of SEQ ID NO:6; and an anti-PD-(L)1(IgG):TGFβR fusion protein comprising:
(i) an anti-PD-L1 antibody or an antigen-binding fragment thereof, comprising a heavy chain amino acid sequence comprising a CDRH1 of SEQ ID NO:13, a CDRH2 of SEQ ID NO:14, and a CDRH3 of SEQ ID NO:15; and a light chain amino acid sequence comprising a CDRL1 of SEQ ID NO:16, a CDRL2 of SEQ ID NO:17, and a CDRL3 of SEQ ID NO:18; and
(ii) human TGFβRII, or a fragment thereof capable of binding to TGF-β.

51. The method as claimed in claim 50, wherein the ICOS binding protein is a monoclonal antibody or an antigen binding fragment thereof.

52. (canceled)

53. The method as claimed in claim 50, wherein the anti-PD-(L)1(IgG):TGFβR fusion protein comprises an anti-PD-L1 antibody that is an IgG1 monoclonal antibody.

54. (canceled)

55. The method as claimed in claim 50, wherein the cancer is selected from: appendiceal cancer, bladder cancer, breast cancer, cervical cancer, colorectal cancer, endometrial cancer, esophageal cancer, fallopian tube cancer, gastric cancer, glioma (such as diffuse intrinsic pontine glioma), head and neck cancer (in particular head and neck squamous cell carcinoma and oropharyngeal cancer), leukemia (in particular acute lymphoblastic leukemia, acute myeloid leukemia) lung cancer (in particular non small cell lung cancer (NSCLC)), lymphoma (in particular Hodgkin's lymphoma, non-Hodgkin's lymphoma), mesothelioma (in particular malignant pleural mesothelioma), melanoma, Merkel cell carcinoma, neuroblastoma, oral cancer, osteosarcoma, ovarian cancer, prostate cancer, renal cancer, salivary gland tumor, sarcoma (in particular Ewing's sarcoma or rhabdomyosarcoma) squamous cell carcinoma, soft tissue sarcoma, thymoma, thyroid cancer, urothelial cancer, uterine cancer, vaginal cancer, vulvar cancer and Wilms tumor.

56. The method as claimed in claim 55, wherein the cancer is selected from: cervical cancer, colorectal cancer, endometrial cancer, head and neck cancer (in particular head and neck squamous cell carcinoma and oropharyngeal cancer), lung cancer (in particular non small cell lung cancer), lymphoma (in particular non-Hodgkin's lymphoma), mesothelioma, melanoma, oral cancer, thyroid cancer, urothelial cancer and uterine cancer.

57. The method as claimed in claim 56, wherein the cancer is selected from: head and neck cancer (in particular head and neck squamous cell carcinoma and oropharyngeal cancer), lung cancer (in particular non small cell lung cancer), urothelial cancer, melanoma and cervical cancer.

58. The method as claimed in claim 50, wherein the ICOS binding protein and anti-PD-(L)1(IgG):TGFβR fusion protein are administered simultaneously.

59. The method as claimed in claim 50, wherein the ICOS binding protein and anti-PD-(L)1(IgG):TGFβR fusion protein are administered sequentially.

60. (canceled)

61. The method as claimed in claim 50, wherein the ICOS binding protein is administered at a dose of about 0.08 mg to about 240 mg.

62.-63. (canceled)

64. The method as claimed in claim 50, wherein the anti-PD-(L)1(IgG):TGFβR fusion protein is administered at a dose of about 500 mg to about 3000 mg.

65. The method as claimed in claim 50, wherein anti-PD-(L)1(IgG):TGFβR fusion protein is administered at a dose of 1200 mg or 2400 mg.

66. The method as claimed in claim 50, wherein the anti-PD-(L)1(IgG):TGFβR fusion protein is administered at a dose of about 2400 mg every three weeks.

67. The method as claimed in claim 50, wherein the anti-PD-(L)1(IgG):TGFβR fusion protein is administered at a dose of about 1200 mg every other week.

68.-76. (canceled)
